(19) United States
(12) Patent Application Publication
(10) Pub. No.: US 2021/0024499 A1
(43) Pub. Date: Jan. 28, 2021

(54) OXIDIZED BIS(3-INDOLYL)METHANES AND USES THEREOF

(71) Applicants: Sanford Burnam Prebys Medical Discovery Institute, La Jolla, CA (US); Xiamen University, Xiamen (CN)

(72) Inventors: Xiaokun Zhang, Xiamen (CN); Ying Su, Xiamen (CN); Xihua Cao, La Jolla, CA (US); Xuhuang Tu, La Jolla, CA (US); Xiaohui Chen, La Jolla, CA (US); Zhiping Zeng, Xiamen (CN)

(21) Appl. No.: 17/040,102

(22) PCT Filed: Mar. 22, 2019
(86) PCT No.: PCT/US2019/023663

§ 371 Date: Sep. 22, 2020

Related U.S. Application Data
(60) Provisional application No. 62/646,841, filed on Mar. 22, 2018.

(51) Int. Cl. C07D 403/06; A61P 35/00

(52) U.S. Cl. CPC C07D 403/06; A61P 35/00

(57) ABSTRACT

Disclosed herein are compounds capable of inducing apoptosis through promoting Nur77 interaction with Bcl-2 and through modulating mitochondrial activities. Also disclosed herein are compositions and methods using these compounds and compositions. Nur77 is a key regulator of Bcl-2 activities and is capable of converting Bcl-2 from an anti-apoptotic protein to a pro-apoptotic protein. Small molecules that directly modulate the Nur77/Bc1-2 apoptotic pathways are promising for treating diseases with abnormal levels of Bcl-2, Nur77, or combinations thereof, including cancers.

### RELATED APPLICATIONS

This application claims the benefit of U.S. Provisional Patent Application No. 62/646,841 entitled “OXIDIZED BIS(3-INDOLYL)METHANES AND USES THEREOF” filed on Mar. 22, 2018, which is incorporated herein by reference in its entirety.

### STATEMENT AS TO FEDERALLY SPONSORED RESEARCH

This invention was made with the support of the United States government under Contract numbers R01CA198982 and R01CA179379 by National Institutes of Health, and CBCRP20IB-0138 by California Breast Cancer Research Program. The government has certain rights in the invention.

### FIELD OF THE INVENTION

Described herein are modulators of the Nur77/Bcl-2 apoptotic pathway, methods of making such compounds, pharmaceutical compositions and medicaments comprising such compounds, and methods of using such compounds in the treatment of conditions, diseases, or disorders associated with the Nur77/Bcl-2 pathway, including cancers associated with abnormal levels of Bcl-2 and Nur77.

### BACKGROUND OF THE INVENTION

Bcl-2 is often abnormally expressed in tumor cells and plays a critical role in the development of diseases and cancers and the resistance of cancer cells to chemotherapeutic drugs and γ-irradiation. Nur77 (also called TR3 or NGFI-B), an orphan member of the nuclear receptor superfamily, can induce apoptosis by targeting mitochondria and through interaction with Bcl-2. The interaction between Nur77 and Bcl-2 serves to modulate important biological pathways including apoptotic pathways wherein the interaction converts Bcl-2 from an anti-apoptotic to a pro-apoptotic molecule by inducing Bcl-2 conformational change. The Nur77/Bcl-2 interaction-mediated biological activities likely play a critical role in determining the fate of cells. Thus, identification of agents targeting the Nur77/Bcl-2 pathway is therapeutically significant.

### SUMMARY OF THE INVENTION

Described herein are compounds capable of modulating the level Nur77/Bcl-2 interaction and compositions, and methods of using these compounds and compositions.

In one aspect, described herein are compounds that have the structure of Formula (I):

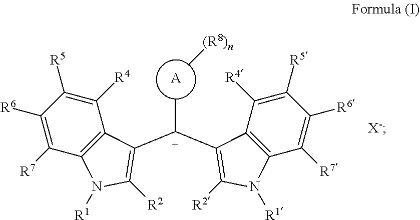

wherein:
Ring A is aryl or heteroaryl;
each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)H, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹⁰);
or two R⁸ on adjacent atoms are taken together with the atoms to which they are attached to form a cycloalkyl or heterocycloalkyl which is unsubstituted or substituted with one, two, or three halogen, C₁-C₆ alkyl, or C₁-C₆ haloalkyl;
n is 0-5;
R¹ and R^{1′} are each independently hydrogen, —S(═O)₂R^{a}, —S(═O)₂N(R^{b})₂, —C(═O)R^{b}, —C(═O)OR^{b}, —C(═O)N(R^{b})₂, C₁-C₈ alkyl, C₂-C₈ alkenyl, C₂-C₈ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl are independently unsubstituted or substituted with one, two, or three R¹¹;
R² and R^{2′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹²;
R⁴, R^{4′}, R⁵, R^{5′}, R⁶, R^{6′}, R⁷, and R^{7′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹³;
or R⁴ and R⁵, R⁵ and R⁶, R⁶ and R⁷, R^{4′} and R^{5′}, R^{5′} and R⁶′, or R^{6′} and R^{7′} are taken together with the atoms to which they are attached to form a cycloalkyl, heterocycloalkyl, aryl, or heteroaryl; wherein the cycloalkyl, heterocycloalkyl, aryl, or heteroaryl is unsubstituted or substituted with one, two, or three R¹³;
X⁻ is a suitable anion;
each R¹⁰, R¹¹, R¹², and R¹³ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, cycloalkyl, heterocycloalkyl, phenyl, benzyl, or monocyclic 5- or 6-membered heteroaryl; wherein cycloalkyl, heterocycle, phenyl, benzyl, and heteroaryl is unsubstituted or substituted with one, two, or three halogen, C₁-C₆ alkyl, or C₁-C₆ haloalkyl;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl; and
wherein when Ring A is phenyl and n is 0, at least one of R¹, R^{1′}, R², R², R⁴, R^{4′}, R⁵, R^{5′}, R⁶, R^{6′}, R⁷, and R^{7′} is not hydrogen.

Any combination of the groups described above or below for the various variables is contemplated herein. Throughout the specification, groups and substituents thereof are chosen by one skilled in the field to provide stable moieties and compounds.

In another aspect, described herein are methods of treating a disease in a mammal, wherein the disease comprises abnormal levels of Bcl-2, Nur77, or combinations thereof, the methods comprising administering the compounds described herein.

In another aspect, described herein are methods of inducing apoptosis in a cell, the methods comprising of contacting the cell with the compounds described herein.

In another aspect, described herein are methods for modulating Nur77 activity in a cell, the methods comprising of contacting the cell with the compounds described herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A shows PARP cleavage in HCT116 cells treated with 0.5 μM Compound 1 or Compound 1a for 6 hours as determined by Western blotting. FIG. 1B shows PARP cleavage in HCT116 cells treated with varying concentrations of Compound 1 for 6 hours as determined by Western blotting. FIG. 1C shows PARP cleavage in HeLa cells treated with varying concentrations of Compound 1 for 6 hours as determined by Western blotting. FIG. 1D shows PARP cleavage in SW480 colon cancer cells treated with the indicated concentration of Compound 1 for 6 hours as determined by Western blotting. FIG. 1E shows cell viability of MDA-MB-231, HS578T, BT549, HCC1937, MCF-7, T47D, and ZR-75-1 cells treated with various concentrations of Compound 1 for 24 hours, as assessed by colorimetric MTT assay.

FIG. 2 shows the level of cleaved caspase 3 in MDA-MB-231 cells treated with the indicated concentration of Compound 1 for 6 hours as determined by Western blotting.

FIG. 3A shows PARP cleavage in MDA-MB-231 cells treated with 0.75 μM Compound 1 for 6 hours as determined by Western blotting. It also shows reduced expression levels of mTOR marker p-4EBP1. FIG. 3B shows PARP cleavage in MDA-MB-231 cells treated with 0.5 pM Compounds 1, 1a, 28a, 28, 47a, 47, 48a, 48, 73a, 73, 39a, and 39 for 6 hours as determined by Western blotting. FIG. 3C shows PARP cleavage in MDA-MB-231 cells treated with 0.5 μM Compounds 70a, 70, 103a, 103, 89a, 89, 88a, and 88 for 6 hours as determined by Western blotting.

FIG. 4A shows mitochondrial dysfunction of MDA-MB-231 cells treated with the indicated concentration of Compound 1 for 6 hours as analyzed by flow cytometry. Cells were stained with JC-1. Aggregated JC-1, red fluorescence (PE), and monomeric JC-1, green fluorescence (FITC) were measured by flow cytometry. Statistical data were mean±SEM of 5 independent images. *P<0.1, ***P<0.001 (Student's t-test). FIG. 4B shows flow cytometry results of mitochondrial ROS production in MDA-MB-231 treated with the indicated concentration of Compound 1 for 6 hours.

FIG. 5 shows Western blotting of WCL and HM fractions prepared from HeLa cells treated with Compound 1 (0.5 μM) and/or BI2030 (1 μM) for 2 hours.

FIG. 6A shows tumor volume in nude mice injected with SW620 (2×10⁶ cells) administrated with the indicated dose of Compound 1 once a day. Tumors were measured every three days as shown (n=6). FIG. 6B shows tumor weight of nude mice bearing SW620 tumor 12 days after administration of Compound 1. ***P<0.001 (Student's t-test).

FIG. 7A shows inhibition of PyMT tumor growth by Compound 1. Female wild-type MMTV-PyMT mice of 12 weeks old were randomly divided into two groups (n=7), treated with daily oral doses of Compound 1 (5 mg/kg) for 18 days, and tumors were weighted (n=7). FIG. 7B shows inhibition of PyMT tumor growth by Compound 1 or Compound 28. Female wild-type MMTV-PyMT mice were treated with daily oral doses of Compound 1 or Compound 28 (3 mg/kg) for 18 days, and tumors were weighted (n=7).

FIG. 8 shows cell viability of MEFs and Nur77^{-/-}MEFs treated with the indicated concentration of Compound 1 for 6 hours, as assessed by colorimetric MTT assay. **P<0.01, ***P<0.001 (Student's t-test).

FIG. 9 shows CD spectra for the binding of Compound 1 to purified Nur77-LBD (10 mM).

FIG. 10 shows reporter transcriptional activity in the Dual-Luciferase Reporter Assay System of HCT116 cells transfected with Gal-4 reporter plasmid and Gal-4-RXR-LBD or Gal-4-RXR-LBD-E453,6A together with Myc-Nur77 treated with the indicated concentration of Compound 1 or 9-cis-RA.

FIG. 11 shows Western blotting results of GST-pull down assays wherein purified Nur77-LBD incubated with or without 1 μM Compound 1 was pulled down by GST or GST-Bcl-2 protein.

### DETAILED DESCRIPTION OF THE INVENTION

Nur77 (NR4A1) (also known as NGFI-B and TR3), an orphan member of the nuclear receptor superfamily, plays vital roles in cell proliferation, differentiation, apoptosis, development, metabolism and immunity. Profiling human tumor specimens revealed a critical role of Nur77 expression in the growth and metastasis of several primary inflammatory diseases and solid tumors. The death effect of Nur77 was initially recognized during studying the apoptosis of immature thymocytes, T-cell hybridomas. Later it was found that Nur77 mediates the death effect of the retinoid-related molecule AHPN (also called CD437) in cancer cells, and discovered a nongenomic action of Nur77 for apoptosis induction, in which Nur77 migrates from the nucleus to the cytoplasm, where it targets mitochondria to trigger cytochrome c release and apoptosis in cancer cells. Such an Nur77 mitochondrial apoptotic pathway is characterized by its interaction with Bcl-2, the anti-apoptotic Bcl-2 family member, and has since been demonstrated in various cancer types by a variety of death stimuli. The interaction between Nur77 and Bcl-2 serves to mediate Nur77 mitochondrial targeting. Moreover, the interaction converts Bcl-2 from an anti-apoptotic to a pro-apoptotic molecule by inducing Bcl-2 conformational change. Because Bcl-2 is often overexpressed in tumor cells and plays a critical role in tumorigenesis and the resistance of cancer cells to chemotherapeutic drugs and y-irradiation, this Nur77/Bcl-2 interaction-mediated apoptotic pathway likely plays a critical role in determining the destiny of cancer cells. As new biological functions of Bcl-2 including its role in autophagy and mTOR activation are emerging, the interaction is an important determinant of the development and progression of many human diseases such as those associated with abnormal inflammatory responses and cell growth. Thus, identification of agents targeting the Nur77/Bcl-2 pathway is therapeutically significant.

A Nur77-derived peptide with 9 amino acids (NuBCP-9), and its enantiomer, are Bcl-2-converting peptides which induce apoptosis of cancer cells in a Bcl-2 dependent manner in vitro and in animals (Kolluri SK, et al. Cancer Cell. 2008; 14:285-98). Strikingly, in vivo administration of NuBCP-9-based nanoparticles triggered complete regressions in the Ehrlich syngeneic mouse model of solid tumor (Kapoor S, et al. International Journal of Pharmaceutics. 2016; 511:876-89.24; Kumar M, et al. Cancer Res. 2014;74:3271-81.), demonstrating the efficacy and selectivity of targeting the Nur77-Bcl-2 apoptotic pathway. However, discovery of small molecules that directly bind Nur77 to activate the pathway has been a challenging. As an orphan nuclear receptor, Nur77 lacks a canonical ligand-binding pocket (LBP), which excludes small molecules from binding to Nur77 to regulate Nur77 functions via the canonical LBP binding mechanism.

Additionally, mTOR (mammalian target of rapamycin) is a major target for therapeutic intervention to treat cancer. It is increasingly apparent that mTOR signaling impacts most major cellular functions. mTOR controls the transcription of many genes and positively regulates mitochondrial activity. Because mTOR commonly deregulated in cancer, mTOR inhibtors can be used to treat cancers.

Indole-3-carbinol (I3C) is a key bioactive ingredient found in cruciferous vegetables such as broccoli, kale, cauliflower etc., and exhibits multiple antitumorigenic properties. In stomach, I3C is rapidly converted to condensation products including 3,3′-diindolymethane (DIM). DIM demonstrates anti-proliferative and anti-cancer activities in various cancer cells including prostate, breast, colorectal and pancreatic cancers.

Provided herein are oxidized salt forms of C-substituted DIM derivatives. These derivatives can dramatically enhance its apoptotic effect in cancer cells. Compounds disclosed herein bind Nur77 at submicromolar concentration and induces Nur77 and Bcl-2 dependent apoptosis. They also effectively inhibit the growth of tumor cells in animals and promote Nur77 mitochondrial targeting and its interaction with Bcl-2. Compounds described herein also inhibit the mTOR pathway.

### Compounds

Described herein are compounds capable of modulating the level of Nur77/Bcl-2 interaction. In one aspect, described herein is a compound of Formula (I):

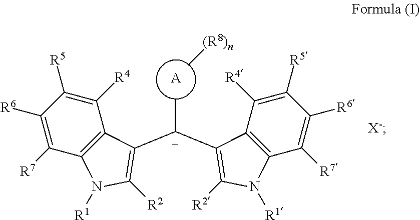

wherein:
Ring A is aryl or heteroaryl;
each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)H, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹⁰;
or two R⁸ on adjacent atoms are taken together with the atoms to which they are attached to form a cycloalkyl or heterocycloalkyl which is unsubstituted or substituted with one, two, or three halogen, C₁-C₆ alkyl, or C₁-C₆ haloalkyl;
n is 0-5;
R¹ and R^{1′} are each independently hydrogen, —S(═O)₂R^{a}, —S(═O)₂N(R^{b})₂, —C(═O)R^{b}, —C(═O)OR^{b}, —C(═O)N(R^{b})₂, C₁-C₈ alkyl, C₂-C₈ alkenyl, C₂-C₈ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl are independently unsubstituted or substituted with one, two, or three R¹¹;
R² and R^{2′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹²;
R⁴, R^{4′}, R⁵, R^{5′}, R⁶, R^{6′}, R⁷, and R^{7′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹³;
or R⁴ and R⁵, R⁵ and R⁶, R⁶ and R⁷, R^{4′} and R^{5′}, R^{5′} and R⁶′, or R^{6′} and R^{7′} are taken together with the atoms to which they are attached to form a cycloalkyl, heterocycloalkyl, aryl, or heteroaryl; wherein the cycloalkyl, heterocycloalkyl, aryl, or heteroaryl is unsubstituted or substituted with one, two, or three R¹³;
X⁻ is a suitable anion;
each R¹⁰, R¹¹, R¹², and R¹³ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, cycloalkyl, heterocycloalkyl, phenyl, benzyl, or monocyclic 5- or 6-membered heteroaryl; wherein cycloalkyl, heterocycle, phenyl, benzyl, and heteroaryl is unsubstituted or substituted with one, two, or three halogen, C₁-C₆ alkyl, or C₁-C₆ haloalkyl;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form a heterocycloalkyl which is unsubstituted or substituted with one, two, or three halogen, C₁-C₆ alkyl, or C₁-C₆ haloalkyl; and
wherein when Ring A is phenyl and n is 0, at least one of R¹, R^{1′}, R², R², R⁴, R^{4′}, R⁵, R^{5′}, R⁶, R^{6′}, R⁷, and R^{7′} is not hydrogen.

For any and all of the embodiments, substituents are selected from among a subset of the listed alternatives.

In some embodiments, X⁻ is a suitable monovalent anion. In some embodiments, X⁻ is a divalent anion. In some embodiments, X⁻ is a suitable polyvalent anion. In some embodiments, X⁻ is a suitable divalent or polyvalent anion and there is less than one equivalent of said anion. In some embodiments, X⁻ is a suitable anion selected from anions formed from inorganic acids and anions formed from organic acids. In some embodiments, there is less than one equivalent of X⁻. In some embodiments, there is more than one equivalent of X⁻. In some embodiments, X⁻ is monovalent and there are two equivalents of X⁻.

In some embodiments, X⁻ is a suitable anion selected from halides, chlorates, sulfates, nitrates, phosphates, carboxylates, sulfonates, and borates. In some embodiments, X⁻ is a suitable anion selected from acetate, benzoate, besylate, borate, bromide, camphorsulfonate, chloride, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, napsylate, nitrate, octadecanoate, oleate, oxalate, pamoate, phosphate, succinate, sulfate, sulfosalicylate, tartrate, tetrahydroxyborate, tetrafluoroborate, tosylate, or trifluoroacetate.

In some embodiments, X⁻ is a suitable anion selected from the group consisting of Cl⁻, Br⁻, I⁻, ClO₄⁻, HSO₄⁻, NO₃⁻, H₂PO₄⁻, HC(═O)O⁻, R¹⁴C(═O)O⁻, R¹⁵S(═O)₂O⁻, and BF₄⁻; and wherein R¹⁴ and R¹⁵ are independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, heteroalkyl, cycloalkyl, heterocycloalkyl, aryl, and heteroaryl is independently unsubstituted or substituted with 1-10 halogen, —OH, C₁-C₆ alkyl, or C₁-C₆ haloalkyl. In some embodiments, X⁻ is a suitable anion selected from the group consisting of Cl⁻, Br⁻, I⁻, ClO₄⁻, HSO₄⁻, NO₃⁻, H₂PO₄⁻, HC(═O)O⁻,R¹⁴C(═O)O⁻, R¹⁵S(═O)₂O⁻, and BF₄⁻; and wherein R¹⁴ and R¹⁵ are independently C₁-C₆ alkyl or aryl; wherein alkyl and aryl are independently unsubstituted or substituted with one, two, or three halogen, C₁-C₆ alkyl, or C₁-C₆ haloalkyl. In some embodiments, X⁻ is a suitable anion selected from the group consisting of Cl⁻, Br⁻, I⁻, ClO₄⁻, HSO₄⁻, NO₃⁻, H₂PO₄⁻, HC(═O)O⁻, CH₃C(═O)O⁻, CF₃C(═O)O⁻, C₆H₅C(═O)O⁻, CH₃S(═O)₂O⁻, CF₃S(═O)₂O⁻, C₆H₅S(═O)₂O⁻, p-CH₃—C₆H₄S(═O)₂O⁻, and BF₄⁻. In some embodiments, X⁻ is a suitable anion anion selected from the group consisting of Cl⁻, HSO₄⁻, CH₃S(═O)₂O⁻, and p-CH₃—C₆H₄S(═O)₂O⁻. In some embodiments, X⁻ is Cl⁻. In some embodiments, X⁻ is HSO₄⁻. In some embodiments, X⁻ is CH₃S(═O)₂O⁻. In some embodiments, p-CH₃—C₆H₄S(═O)₂O⁻.

In some embodiments, Ring A is aryl. In some embodiments, Ring A is phenyl.

In some embodiments, Ring A is polycyclic aryl. In some embodiments, Ring A is naphthyl or fluorenyl. In some embodiments, Ring A is naphthyl. In some embodiments, Ring A is fluorenyl.

In some embodiments, Ring A is heteroaryl.

In some embodiments, Ring A is monocyclic heteroaryl. In some embodiments, Ring A is pyrrolyl, furanyl, thiophenyl, pyrazolyl, imidazolyl, triazolyl, oxazolyl, isoxazolyl, thiazolyl, isothiazolyl, oxadiazolyl, or thiadiazolyl. In some embodiments, Ring A is furanyl or thiophenyl. In some embodiments, Ring A is pyridinyl, pyridazinyl, pyrimidinyl, pyrazinyl, or triazinyl. In some embodiments, Ring A is pyridinyl.

In some embodiments, Ring A is bicyclic heteroaryl. In some embodiments, Ring A is indolyle, isoindolyl, indolizinyl, indazolyl, benzimidazolyl, azaindolyl, azaindazolyl, purinyl, benzofuranyl, isobenzofuranyl, benzo[b]thiophenyl, benzo[c]thiophenyl, benzoxazolyl, benzisoxazolyl, benzthiazolyl, benzisothiazolyl, quinolinyl, isoquinolinyl, quinoxalinyl, phthalizine, quinazolinyl, cinnolinyl, naphthyridinyl, pyridopyrimidinyl, pyridopyrazinyl, or pteridinyl. In some embodiments, Ring A is indolyl, benzo[b]thiophenyl, or quinolinyl.

In some embodiments, each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)H, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹⁰. In some embodiments, each R⁸ is independently halogen, —CN, —OH, —OR^{a}, —NO₂, —N(R^{b})₂, —C(═O)H, —C(═O)R^{a}, —OC(═O)R^{a}, —C(═O)OR^{b}, —C(═O)N(R^{b})₂, —NR^{b}C(═O)R^{a}, C₁-C₆ alkyl, cycloalkyl, heterocycloalkyl, aryl, or heteroaryl; wherein each alkyl, cycloalkyl, heterocycloalkyl, aryl, and heteroaryl is unsubstituted or substituted with one, two, or three R¹⁰. In some embodiments, each R⁸ is independently halogen, —CN, —OH, —OR^{a}, —NO₂, —N(R^{b})₂, —C(═O)H, —C(═O)R^{a}, —C(═O)OR^{b}, —C(═O)N(R^{b})₂, C₁-C₆ alkyl, cycloalkyl, or aryl; wherein each alkyl, cycloalkyl, and aryl is unsubstituted or substituted with one, two, or three R¹⁰. In some embodiments, each R⁸ is independently halogen, —OH, —OR^{a}, —NO₂, —N(R^{b})₂, —C(═O)H, —C(═O)OR^{b}, C₁-C₆ alkyl, or aryl; wherein each alkyl or aryl is unsubstituted or substituted with one, two, or three R¹⁰. In some embodiments, each R⁸ is independently —F, —Cl, —Br, —OH, —OR^{a}, —NO₂, —N(R^{b})₂, —C(═O)H, —C(═O)OR^{b}, C₁-C₆ alkyl, or aryl; wherein each alkyl or aryl is unsubstituted or substituted with one, two, or three R¹⁰.

In some embodiments, each R¹⁰ is independently halogen, —CN, —OH, —OR^{a}, —NO₂, —N(R^{b})₂, —C(═O)R^{a}, —OC(═O)R^{a}, —C(═O)OR^{b}, —C(═O)N(R^{b})₂, —NR^{b}C(═O)R^{a}, C₁-C₆ alkyl, C₁-C₆ haloalkyl, cycloalkyl, heterocycloalkyl, phenyl, benzyl, or monocyclic 5- or 6-membered heteroaryl; wherein cycloalkyl, heterocycle, phenyl, benzyl, and heteroaryl is unsubstituted or substituted with one, two, or three halogen, C₁-C₆ alkyl, or C₁-C₆ haloalkyl. In some embodiments, each R¹⁰ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl. In some embodiments, each R¹⁰ is independently —F, —Cl, —Br, —CN, —OH, —OR^{a}, —N(R^{b})₂, —C(═O)OR^{b}, —C(═O)N(R^{b})₂, C₁-C₆ alkyl, or C₁-C₆ haloalkyl. In some embodiments, each R¹⁰ is independently —F, —Cl, C₁-C₆ alkyl, or C₁-C₆ haloalkyl. In some embodiments, each R¹⁰ is —F.

In some embodiments, each R⁸ is independently —F, —Cl, —OH, —OCH₃, —OCF₃, —NO₂, —N(Et)₂, —C(═O)H, —C(═O)OCH₃, methyl, ethyl, tert-butyl, —CF₃, or phenyl.

In some embodiments, n is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5. In some embodiments, n is 0, 1, 2, 3, 4, or 5. In some embodiments, n is 0, 1, 2, or 3. In some embodiments, n is 1. In some embodiments, n is 2. In some embodiments, n is 3.

In some embodiments, two R⁸ on adjacent atoms are taken together with the atoms to which they are attached to form a cycloalkyl or heterocycloalkyl. In some embodiments, two R⁸ on adjacent atoms are taken together with the atoms to which they are attached to form a heterocycloalkyl selected from 1,3-dioxolane or 1,4-dioxane.

In some embodiments,

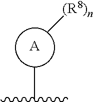

is

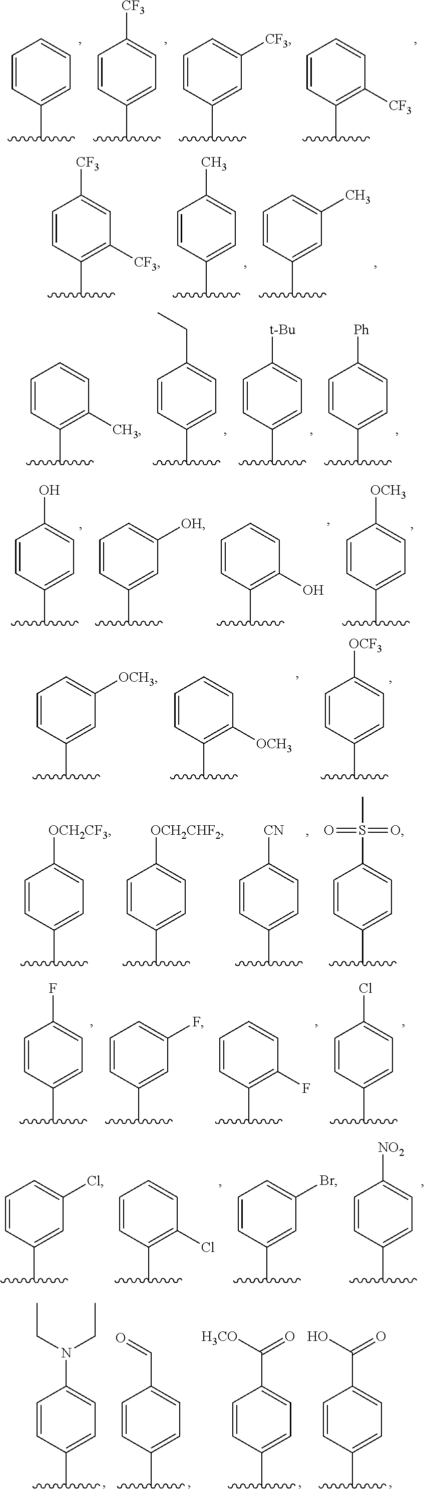

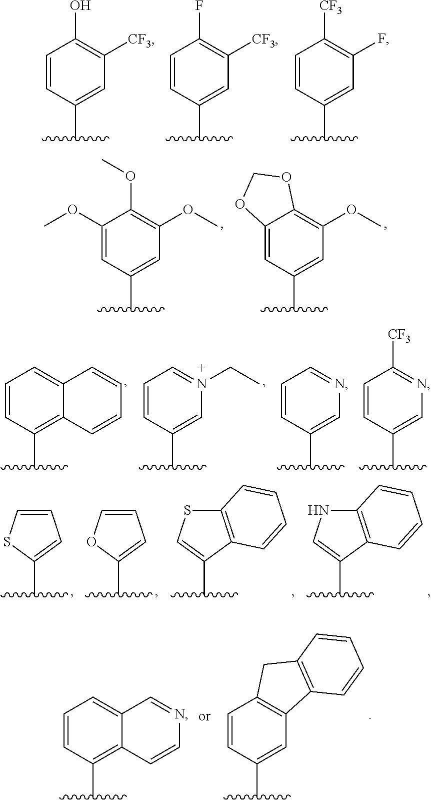

In some embodiments,

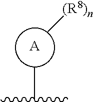

is

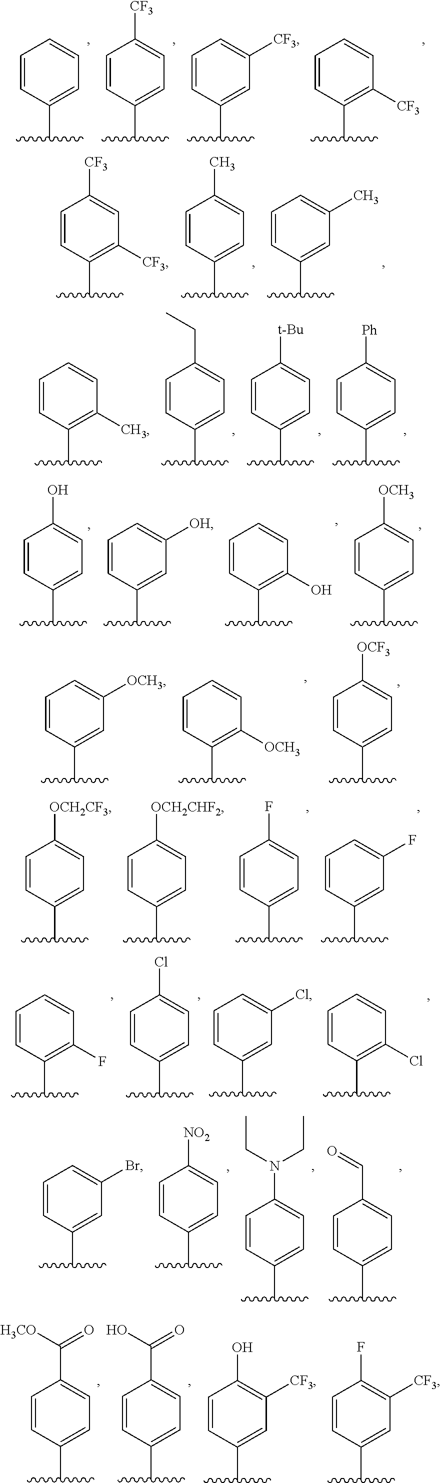

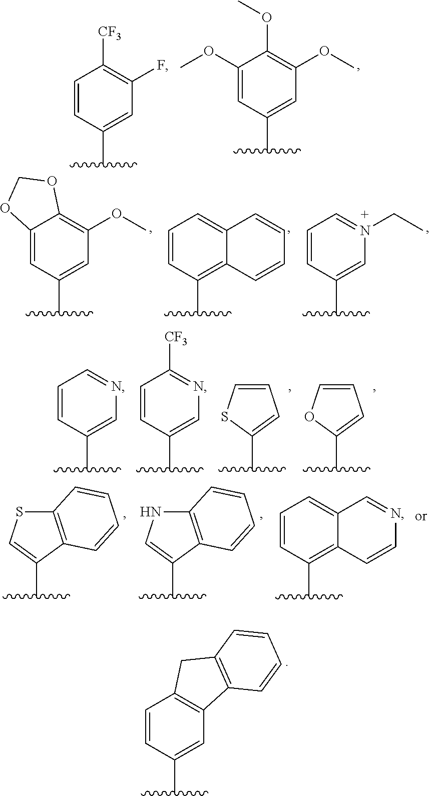

In some embodiments,

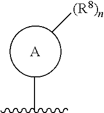

is

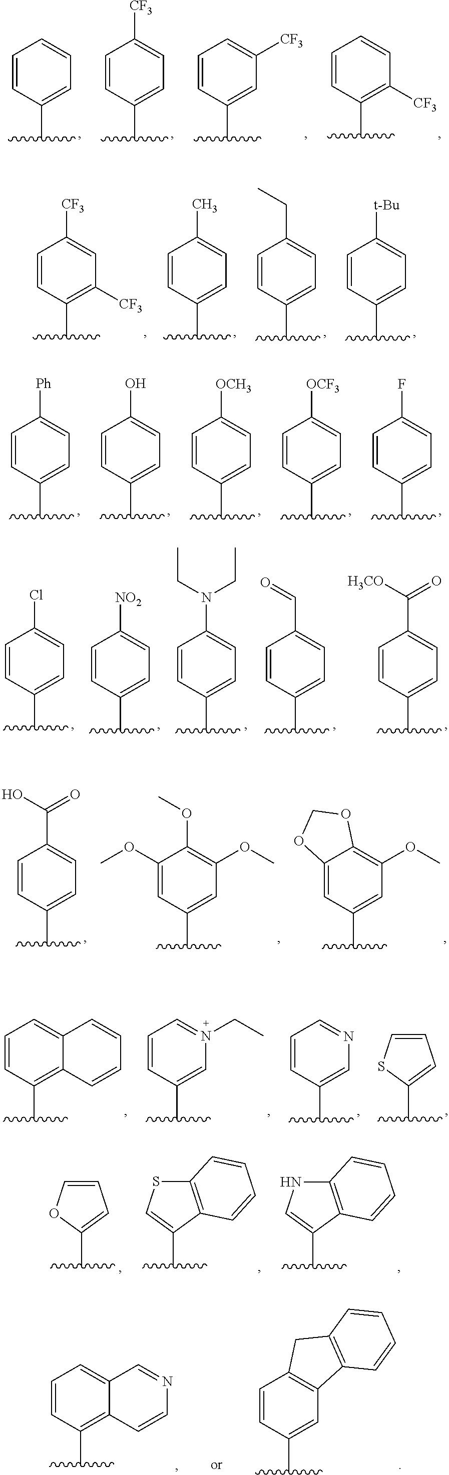

In some embodiments,

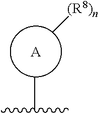

is

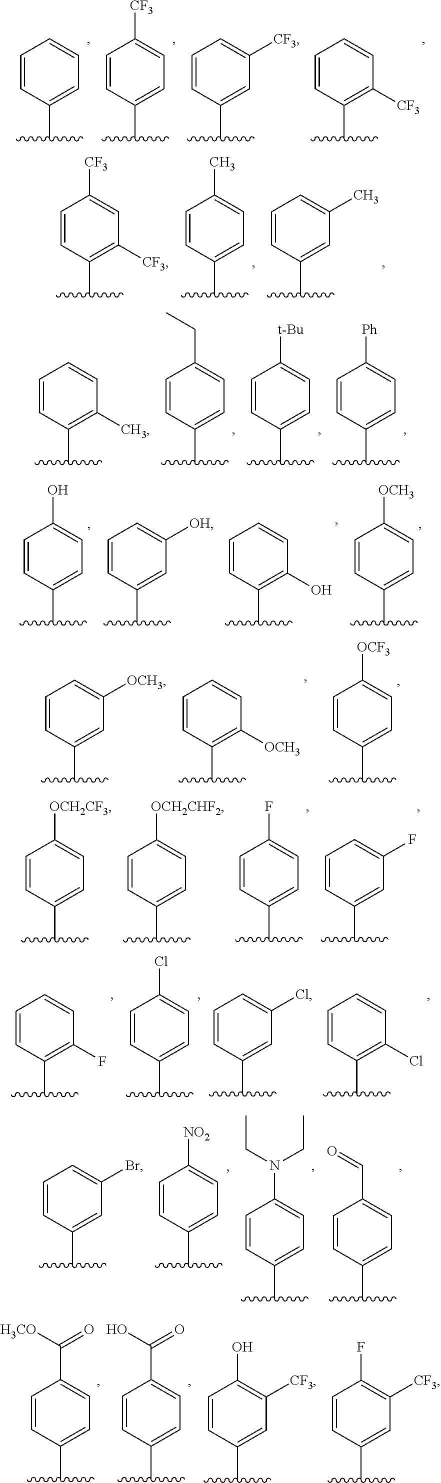

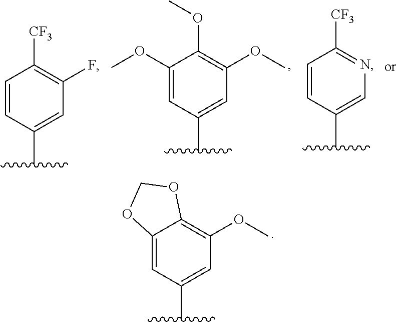

In some embodiments,

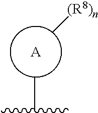

is

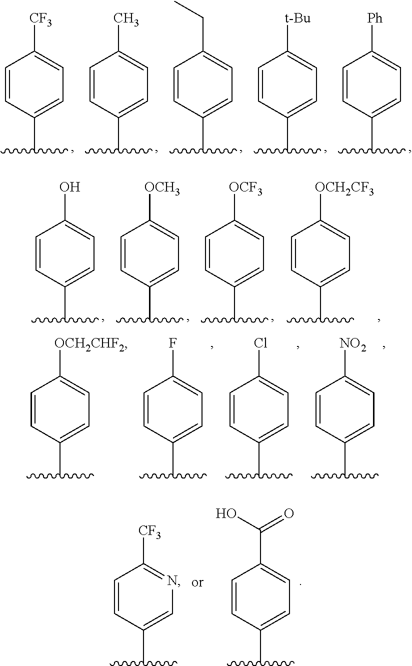

In some embodiments,

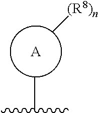

is

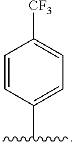 [SMILES: *c1ccc(C(F)(F)F)cc1]

In some embodiments, R¹and R^{1′} are each independently hydrogen, —S(═O)₂R^{a}, —S(═O)₂N(R^{b})₂, —C(═O)R^{b}, —C(═O)OR^{b}, —C(═O)N(R^{b})₂, C₁-C₈ alkyl, cycloalkyl, heterocycloalkyl, aryl, heteroaryl, —C₁-C₂ alkylene(aryl), —C₁-C₂ alkylene(heteroaryl), —C₁-C₂ alkylene(cycloalkyl), or —C₁-C₂ alkylene(heterocycloalkyl); wherein alkyl, alkylene, cycloalkyl, heterocycloalkyl, aryl, and heteroaryl are independently unsubstituted or substituted with one, two, or three R¹¹. In some embodiments, R¹ and R^{1′} are each independently hydrogen, —S(═O)₂R^{a}, —S(═O)₂N(R^{b})₂, —C(═O)R^{b}, —C(═O)OR^{b}, —C(═O)N(R^{b})₂. In some embodiments, R¹ and R^{1′} are each independently hydrogen, —S(═O)₂R^{a} or —C(═O)R^{b}. In some embodiments, R¹ and R^{1′} are each independently hydrogen, C₁-C₈ alkyl, cycloalkyl, heterocycloalkyl, aryl, heteroaryl, —C₁-C₂ alkylene(aryl), —C₁-C₂ alkylene(heteroaryl), —C₁-C₂ alkyl ene(cycloalkyl), or —C₁-C₂ alkylene(heterocycloalkyl); wherein alkyl, alkylene, cycloalkyl, heterocycloalkyl, aryl, and heteroaryl are independently unsubstituted or substituted with one, two, or three R¹¹. In some embodiments, R¹ and R^{1′} are each independently hydrogen, C₁-C₈ alkyl, cycloalkyl, heterocycloalkyl, aryl, heteroaryl, —CH₂-(aryl), —CH₂-(heteroaryl), —CH₂-(cycloalkyl), or —CH₂-(heterocycloalkyl); wherein alkyl, cycloalkyl, heterocycloalkyl, aryl, and heteroaryl are independently unsubstituted or substituted with one, two, or three R¹¹. In some embodiments, R¹ and R^{1′} are each independently hydrogen, C₁-C₈ alkyl, cycloalkyl, heterocycloalkyl, aryl, heteroaryl, —CH₂-(aryl), —CH₂-(heteroaryl), —CH₂-(cycloalkyl), or —CH₂-(heterocycloalkyl). In some embodiments, R¹ and R^{1′} are each independently hydrogen, C₁-C₈ alkyl, cycloalkyl, heterocycloalkyl, aryl, heteroaryl, —CH₂-(aryl), —CH₂-(heteroaryl), —CH₂-(cycloalkyl), or —CH₂-(heterocycloalkyl). In some embodiments, R¹ and R^{1′} are each independently hydrogen, C₁-C₈ alkyl, cycloalkyl, heterocycloalkyl, aryl, heteroaryl, —CH₂-(aryl), —CH₂-(heteroaryl), —CH₂-(cycloalkyl), or —CH₂-(heterocycloalkyl). In some embodiments, R¹ and R^{1′} are each independently hydrogen, C₁-C₈ alkyl, cycloalkyl, aryl, —CH₂-(aryl), or —CH₂-(heterocycloalkyl). In some embodiments, R¹ and R^{1′} are each independently hydrogen, C₁-C₈ alkyl, or aryl. In some embodiments, R¹ and R^{1 ′} are each independently hydrogen, methyl, ethyl, n-propyl, iso-propyl, n-butyl, iso-butyl, sec-butyl, tert-butyl, n-pentyl, iso-pentyl, n-hexyl, iso-hexyl, n-heptyl, n-octyl, or phenyl. In some embodiments, R¹ and R^{1′} are each independently hydrogen, methyl, ethyl, n-propyl, n-butyl, n-pentyl, or phenyl.

In some embodiments, R¹ and R^{1′} are the same. In some embodiments, R¹ and R^{1′} are different. In some embodiments, R¹ and R^{1′} are each hydrogen. In some embodiments, R¹ and R^{1′} are each methyl. In some embodiments, R¹ and R^{1′} are each n-pentyl. In some embodiments, R¹ and R^{1′} are each phenyl. In some embodiments, one of R¹ and R^{1′} is hydrogen. In some embodiments, one of R¹ and R^{1′} is hydrogen; and the other one of R¹ and R^{1′} is C₁-C₈ alkyl or aryl. In some embodiments, one of R¹ and R^{1′} is hydrogen; and the other one of R¹ and R^{1′} is methyl, ethyl, n-propyl, iso-propyl, n-butyl, iso-butyl, sec-butyl, tert-butyl, n-pentyl, iso-pentyl, n-hexyl, iso-hexyl, n-heptyl, n-octyl, or phenyl. In some embodiments, one of R¹ and R^{1′} is hydrogen; and the other one of R¹ and R^{1′} is methyl. In some embodiments, one of R¹ and R^{1′} is hydrogen; and the other one of R¹ and R^{1′} is ethyl. In some embodiments, one of R¹ and R^{1′} is hydrogen; and the other one of R¹ and R^{1′} is n-propyl. In some embodiments, one of R¹ and R^{1′} is hydrogen; and the other one of R¹ and R^{1′} is n-butyl. In some embodiments, one of R¹ and R^{1′} is hydrogen; and the other one of R¹ and R^{1′} is n-pentyl. In some embodiments, one of R¹ and R^{1′} is hydrogen; and the other one of R¹ and R^{1′} is phenyl.

In some embodiments, each R″ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl. In some embodiments, each R¹¹ is independently —F, —Cl, —Br, —CN, —OH, —OR^{a}, —N(R^{b})₂, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl.

In some embodiments, R² and R² are each independently hydrogen, halogen, —CN, —OH, —OR^{a}, —NO₂, —N(R^{b})₂, —C(═O)R^{a}, —OC(═O)R^{a}, —C(═O)OR^{b}, —OC(═O)OR^{b}, —C(═O)N(R^{b})₂, —NR^{b}C(═O)R^{a}, C₁-C₆ alkyl, cycloalkyl, heterocycloalkyl, aryl, or heteroaryl; wherein each alkyl, cycloalkyl, heterocycloalkyl, aryl, and heteroaryl is unsubstituted or substituted with one, two, or three R¹². In some embodiments, R² and R^{2′} are each independently hydrogen, halogen, —CN, —OR^{a}, —N(R^{b})₂, C₁-C₆ alkyl, cycloalkyl, or aryl; wherein each alkyl, cycloalkyl, and aryl is unsubstituted or substituted with one, two, or three R¹². In some embodiments, R² and R^{2′} are each independently hydrogen, halogen, —CN, —OR^{a}, —N(R^{b})₂, C₁-C₆ alkyl, cycloalkyl, or aryl. In some embodiments, R² and R^{2′} are each independently hydrogen, halogen, —CN, —OR^{a}, C₁-C₆ alkyl, cycloalkyl, or aryl. In some embodiments, R² and R^{2′} are each independently hydrogen, —F, —Cl, —Br, —CN, —OR^{a}, C₁-C₆ alkyl, cycloalkyl, or aryl. In some embodiments, R² and R^{2′} are each independently hydrogen or C₁-C₆ alkyl. In some embodiments, R² and R^{2′} are each independently hydrogen, methyl, ethyl, n-propyl, iso-propyl, n-butyl, iso-butyl, sec-butyl, tert-butyl, n-pentyl, iso-pentyl, n-hexyl. In some embodiments, R² and R^{2′} are each independently hydrogen or methyl.

In some embodiments, R² and R^{2′} are the same. In some embodiments, R² and R^{2′} are different. In some embodiments, R² and R^{2′} are each hydrogen. In some embodiments, R² and R^{2′} are each methyl. In some embodiments, one of R² and R^{2′} is hydrogen.

In some embodiments, each R¹² is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl. C₁-C₆ alkyl, or C₁-C₆ haloalkyl. In some embodiments, each R¹⁻² is independently —F, —Cl, —Br, —CN, —OH, —OR^{a}, —N(R^{b})₂, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl.

In some embodiments, R⁴, R^{4′}, R⁵, R^{5′}, R⁶, R^{6′}, R⁷, and R^{7′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹³. In some embodiments, R⁴, R^{4′}, R⁵, R^{5′}, R⁶, R^{6′}, R⁷, and R^{7′} are each independently hydrogen, halogen, —CN, —OH, —OR^{a}, —NO₂, —N(R^{b})₂, —C(═O)R^{a}, —OC(═O)R^{a}, —C(═O)OR^{b}, —C(═O)N(R^{b})₂, —NR^{b}C(═O)R^{a}, C₁-C₆ alkyl, cycloalkyl, heterocycloalkyl, aryl, or heteroaryl; wherein each alkyl, alkenyl, alkynyl, cycloalkyl, heterocycloalkyl, aryl, and heteroaryl is unsubstituted or substituted with one, two, or three R¹³. In some embodiments, R⁴, R^{4′}, R⁵, R^{5′}, R⁶, R^{6′}, R⁷, and R^{7′} are each independently hydrogen, halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, cycloalkyl, heterocycloalkyl, aryl, or heteroaryl; wherein each alkyl, cycloalkyl, heterocycloalkyl, aryl, and heteroaryl is unsubstituted or substituted with one, two, or three R¹³. In some embodiments, R⁴, R^{4′}, R⁵, R^{5′}, R⁶, R^{6′}, R⁷, and R^{7′} are each independently hydrogen, halogen, —CN, —OH, —OR^{a}, —N(R^{b})₂, —C(═O)OR^{b}, —C(═O)N(R^{b})₂, C₁-C₆ alkyl, cycloalkyl, or aryl; wherein each alkyl, cycloalkyl, and aryl is unsubstituted or substituted with one, two, or three R¹³ In some embodiments, R⁴, R^{4′}, R⁵, R^{5′}, R⁶, R^{6′}, R⁷, and R^{7′} are each independently hydrogen, halogen, —OH, —OR^{a}, —C(═O)OR^{b}, or C₁-C₆ alkyl which is unsubstituted or substituted with one, two, or three R¹³.

In some embodiments, R⁴, R^{4′}, R⁵, R^{5′}, R⁶, R^{6′}, R⁷, and R^{7′} are each independently hydrogen, halogen, —OH, —OR^{a}, —C(═O)OR^{b}, or C₁-C₆ alkyl. In some embodiments, R⁴, R^{4′}, R⁵, R^{5′}, R⁶, R^{6′}, R⁷, and R^{7′} are each independently hydrogen, halogen, —OH, —OR^{a}, —C(═O)OR^{b}, or C₁-C₄ alkyl. In some embodiments, R⁴, R^{4′}, R⁵, R^{5′}, R⁶, R^{6′}, R⁷, and R^{7′} are each independently hydrogen, —F, —Cl, —Br, —OH, —OR^{a}, —C(═O)OR^{b}, methyl, ethyl, n-propyl, iso-propyl, n-butyl, iso-butyl, sec-butyl, or tert-butyl. In some embodiments, R⁴, R^{4′}, R⁵, R^{5′}, R⁶, R^{6′}, R⁷, and R^{7′} are each independently hydrogen, —F, —Cl, —Br, —OH, —OCH₃, —OBn, —C(═O)OH, or methyl.

In some embodiments, R⁴ and R^{4′} are the same. In some embodiments, R⁴ and R^{4′} are different. In some embodiments, R⁴ and R^{4′} are each hydrogen. In some embodiments, one of R⁴ and R^{4′} is hydrogen.

In some embodiments, R⁵ and R^{5′} are the same. In some embodiments, R⁵ and R^{5′} are different. In some embodiments, R⁵ and R^{5′} are each hydrogen. In some embodiments, one of R⁵ and R^{5′} is hydrogen.

In some embodiments, R⁶ and R^{6′} are the same. In some embodiments, R⁶ and R^{6′} are different. In some embodiments, R⁶ and R^{6′} are each hydrogen. In some embodiments, one of R⁶ and R^{6′} is hydrogen.

In some embodiments, R⁷ and R^{7′} are the same. In some embodiments, R⁷ and R^{7′} are different. In some embodiments, R⁷ and R^{7′} are each hydrogen. In some embodiments, one of R⁷ and R^{7′} is hydrogen.

In some embodiments, R⁴ and R^{4′} are the same, R⁵ and R^{5′} are the same, R⁶ and R^{6′} are the same, and R⁷ and R^{7′} are the same.

In some embodiments, R⁴ and R⁵, R⁵ and R⁶, R⁶ and R⁷, R^{4′} and R^{5′}, R^{5′} and R⁶′, or R^{6′} and R^{7′} are taken together with the atoms to which they are attached to form a cycloalkyl, heterocycloalkyl, aryl, or heteroaryl; wherein the cycloalkyl, heterocycloalkyl, aryl, or heteroaryl is unsubstituted or substituted with one, two, or three R¹³. In some embodiments, R⁴ and R⁵, and R^{4′} and R^{5′} are taken together with the atoms to which they are attached to form a cycloalkyl, heterocycloalkyl, aryl, or heteroaryl; wherein the cycloalkyl, heterocycloalkyl, aryl, or heteroaryl is unsubstituted or substituted with one, two, or three R¹³. In some embodiments, R⁵ and R⁶, and R^{5′} and R^{6′} are taken together with the atoms to which they are attached to form a cycloalkyl, heterocycloalkyl, aryl, or heteroaryl; wherein the cycloalkyl, heterocycloalkyl, aryl, or heteroaryl is unsubstituted or substituted with one, two, or three R¹³. In some embodiments, R⁶ and R⁷, and R^{6′} and R^{7′} are taken together with the atoms to which they are attached to form a cycloalkyl, heterocycloalkyl, aryl, or heteroaryl; wherein the cycloalkyl, heterocycloalkyl, aryl, or heteroaryl is unsubstituted or substituted with one, two, or three R¹³. In some embodiments, R⁴ and R⁵, R⁵ and R⁶, R⁶ and R⁷, R^{4′} and R^{5′}, R^{5′} and R^{6′}, or R^{6′} and R^{7′} are taken together with the atoms to which they are attached to form a cycloalkyl or heterocycloalkyl; wherein the cycloalkyl or heterocycloalkyl is unsubstituted or substituted with one, two, or three R¹³. In some embodiments, R⁴ and R⁵, R⁵ and R⁶, R⁶ and R⁷, R^{4′} and R^{5′}, R^{5′} and R^{6′}, or R^{6′} and R^{7′} are taken together with the atoms to which they are attached to form an aryl or heteroaryl; wherein the aryl or heteroaryl is unsubstituted or substituted with one, two, or three R¹³.

In some embodiments, each R¹³ is independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl. In some embodiments, each R¹³ is independently —F, —Cl, —Br, —CN, —OH, —OR^{a}, —N(R^{b})₂, —C(═O)OR^{b}, —C(═O)N(R^{b})₂, C₁-C₆ alkyl, C₁-C₆ haloalkyl, or cycloalkyl which is unsubstituted or substituted with one, two, or three halogen, C₁-C₆ alkyl, or C₁-C₆ haloalkyl.

In some embodiments, the compound of Formula (I) is represented by Formula (IIa):

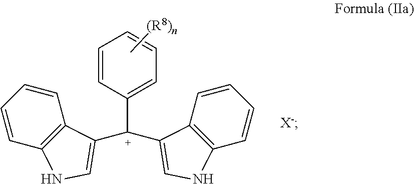

wherein:
n is 1-5.

In some embodiments, n is 1 to 2, 1 to 3, 1 to 4, 1 to 5, 2 to 3, 2 to 4, 2 to 5, 3 to 4, 3 to 5, or 4 to 5. In some embodiments, n is 1, 2, 3, 4, or 5. In some embodiments, n is 1, 2, or 3. In some embodiments, n is 1. In some embodiments, n is 2. In some embodiments, n is 3.

In some embodiments, the compound of Formula (I) is represented by Formula (IIb):

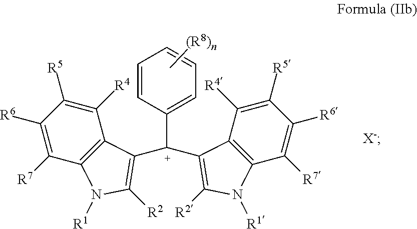

wherein:
at least one of R¹, R^{1′}, R², , R^{2′}, R⁴, R^{4′}, R⁵, R^{5′}, R⁶, R^{6′}, R⁷, and R^{7′} is not hydrogen.

In some embodiments, the compound of Formula (I) is represented by Formula (IIc), Formula (IId), Formula (IIe), or Formula (IIf):

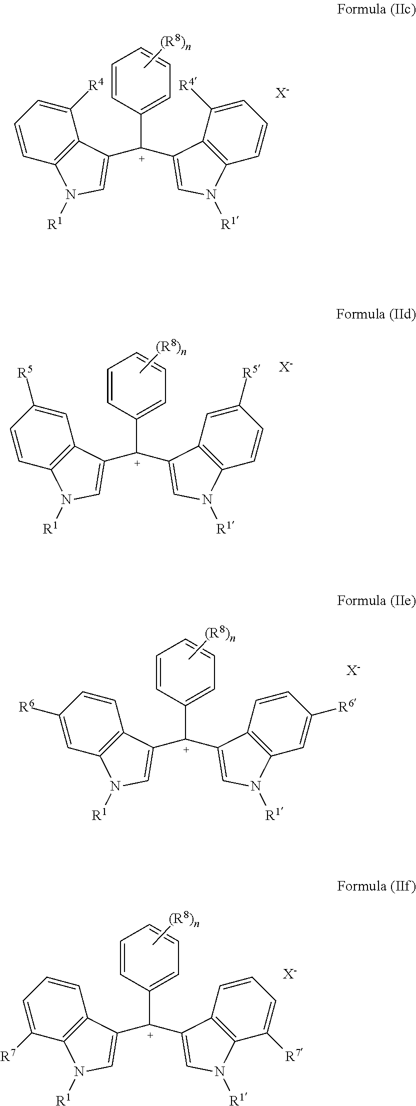

wherein:
R⁴, R^{4′}, R⁵, R^{5′}, R⁶, R^{6′}, R⁷, and R^{7′} are each independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is unsubstituted or substituted with one, two, or three R¹³.

In some embodiments, R⁴, R^{4′}, R⁵, R^{5′}, R⁶, R^{6′}, R⁷, and R^{7′} are each independently halogen, —CN, —OH, —OR^{a}, —NO₂, —N(R^{b})₂, —C(═O)R^{a}, —OC(═O)R^{a}, —C(═O)OR^{b}, —C(═O)N(R^{b})₂, —NR^{b}C(═O)R^{a}, C₁-C₆ alkyl, cycloalkyl, heterocycloalkyl, aryl, or heteroaryl; wherein each alkyl, alkenyl, alkynyl, cycloalkyl, heterocycloalkyl, aryl, and heteroaryl is unsubstituted or substituted with one, two, or three R¹³. In some embodiments, R⁴, R^{4′}, R⁵, R^{5′}, R⁶, R^{6′}, R⁷, and R^{7′} are each independently halogen, —CN, —OH, —OR^{a}, —NO₂, —N(R^{b})₂, —C(═O)R^{a}, —C(═O)OR^{b}, —C(═O)N(R^{b})₂, C₁-C₆ alkyl, cycloalkyl, heterocycloalkyl, aryl, or heteroaryl; wherein each alkyl, cycloalkyl, heterocycloalkyl, aryl, and heteroaryl is unsubstituted or substituted with one, two, or three R¹³. In some embodiments, R⁴, R^{4′}, R⁵, R^{5′}, R⁶, R^{6′}, R⁷, and R^{7′} are each independently halogen, —CN, —OH, —OR^{a}, —N(R^{b})₂, —C(═O)OR^{b}, —C(═O)N(R^{b})₂, C₁-C₆ alkyl, cycloalkyl, or aryl; wherein each alkyl, cycloalkyl, and aryl is unsubstituted or substituted with one, two, or three R¹³. In some embodiments, R⁴, R^{4′}, R⁵, R^{5′}, R⁶, R^{6′}, R⁷, and R^{7′} are each independently halogen, —OH, —OR^{a}, —C(═O)OR^{b}, or C₁-C₆ alkyl which is unsubstituted or substituted with one, two, or three R¹³.

In some embodiments, R⁴, R^{4′}, R⁵, R^{5′}, R⁶, R^{6′}, R⁷, and R^{7′} are each independently halogen, —OH, —OR^{a}, —C(═O)OR^{b}, or C₁-C₆ alkyl. In some embodiments, R⁴, R^{4′}, R⁵, R^{5′}, R⁶, R^{6′}, R⁷, and R^{7′} are each independently halogen, —OH, —OR^{a}, —C(═O)OR^{b}, or C₁-C₄ alkyl. In some embodiments, R⁴, R^{4′}, R⁵, R^{5′}, R⁶, R^{6′}, R⁷, and R^{7′} are each independently —F, —Cl, —Br, —OH, —OR^{a}, —C(═O)OR^{b}, methyl, ethyl, n-propyl, iso-propyl, n-butyl, iso-butyl, sec-butyl, or tert-butyl. In some embodiments, R⁴, R^{4′}, R⁵, R^{5′}, R⁶, R^{6′}, R⁷, and R^{7′} are each independently —F, —Cl, —Br, —OH, —OCH₃, —C(═O)OH, or methyl.

In some embodiments, the compound of Formula (I) is represented by Formula (IIIa), Formula (IIIc), Formula (IIId), Formula (IIIe), or Formula (IIIf):

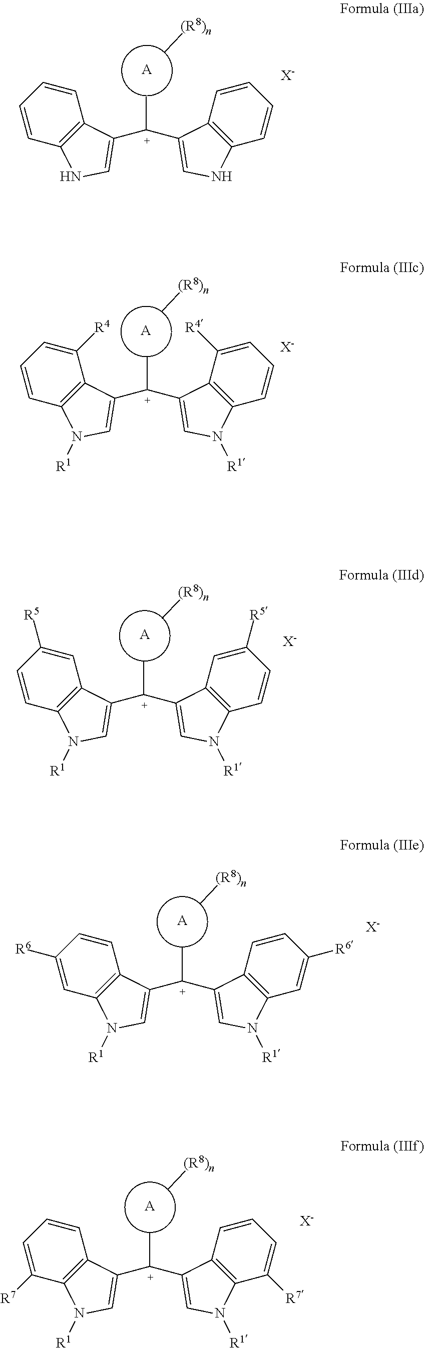

wherein:
Ring A is heteroaryl.

Any combination of the groups described above for the various variables is contemplated herein. Throughout the specification, groups and substituents thereof are chosen by one skilled in the field to provide stable moieties and compounds.

In some embodiments, compounds of Formula (I) include, but are not limited to, those in Table 1.

TABLE 1

| Cmpd | Structure | X⁻ | Name |
|---|---|---|---|
| 1 | 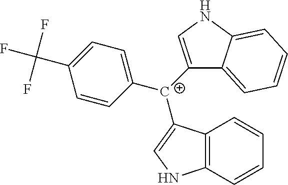 [SMILES: FC(F)(F)c1ccc([C+](c2c[nH]c3ccccc23)c2c[nH]c3ccccc23)cc1] | MeSO₃⁻ | bis(1H-indol-3-yl)(4- (trifluoromethyl)phenyl)methylium methanesulfonate |
| 2 | 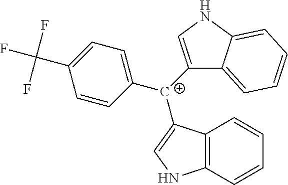 [SMILES: FC(F)(F)c1ccc([C+](c2c[nH]c3ccccc23)c2c[nH]c3ccccc23)cc1] | Cl⁻ | bis(1H-indol-3-yl)(4- (trifluoromethyl)phenyl)methylium chloride |
| 3 | 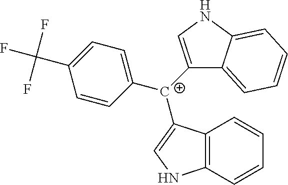 [SMILES: FC(F)(F)c1ccc([C+](c2c[nH]c3ccccc23)c2c[nH]c3ccccc23)cc1] | p-Me—PhSO₃⁻ | bis(1H-indol-3-yl)(4- (trifluoromethyl)phenyl)methylium p-toluenesulfonate |
| 4 | 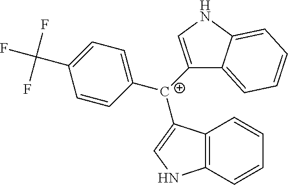 [SMILES: FC(F)(F)c1ccc([C+](c2c[nH]c3ccccc23)c2c[nH]c3ccccc23)cc1] | HSO₄⁻ | bis(1H-indol-3-yl)(4- (trifluoromethyl)phenyl)methylium hydrogensulfate |
| 5 | 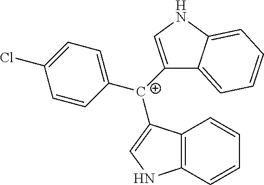 [SMILES: Clc1ccc([C+](c2c[nH]c3ccccc23)c2c[nH]c3ccccc23)cc1] | MeSO₃⁻ | bis(1H-indol-3-yl)(4- chlorophenyl)methylium methanesulfonate |
| 6 | 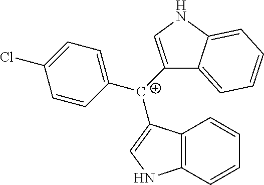 [SMILES: Clc1ccc([C+](c2c[nH]c3ccccc23)c2c[nH]c3ccccc23)cc1] | Cl⁻ | bis(1H-indol-3-yl)(4- chlorophenyl)methylium chloride |
| 7 | 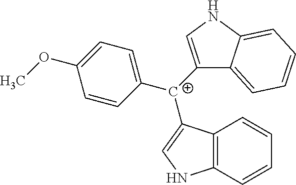 [SMILES: COc1ccc([C+](c2c[nH]c3ccccc23)c2c[nH]c3ccccc23)cc1] | MeSO₃⁻ | bis(1H-indol-3-yl)(4- methoxyphenyl)methylium methanesulfonate |
| 8 | 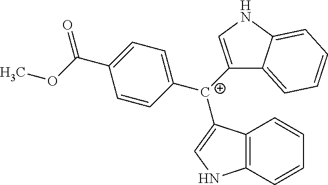 [SMILES: COC(=O)c1ccc([C+](c2c[nH]c3ccccc23)c2c[nH]c3ccccc23)cc1] | MeSO₃⁻ | bis(1H-indol-3-yl)(4- (methoxycarbonyl)phenyl)methylium methanesulfonate |
| 9 | 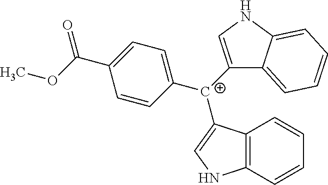 [SMILES: COC(=O)c1ccc([C+](c2c[nH]c3ccccc23)c2c[nH]c3ccccc23)cc1] | Cl⁻ | bis(1H-indol-3-yl)(4- (methoxycarbonyl)phenyl)methylium chloride |
| 10 | 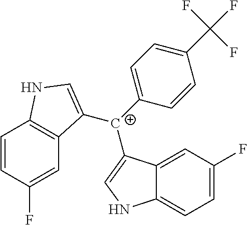 [SMILES: Fc1ccc2[nH]cc([C+](c3ccc(C(F)(F)F)cc3)c3c[nH]c4ccc(F)cc34)c2c1] | MeSO₃⁻ | bis(5-fluoro-1H-indol-3-yl)(4- (trifluoromethyl)phenyl)methylium methanesulfonate |
| 11 | 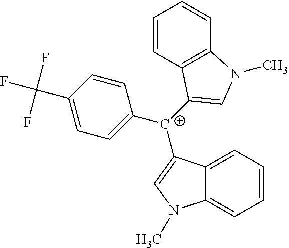 [SMILES: Cn1cc([C+](c2ccc(C(F)(F)F)cc2)c2cn(C)c3ccccc23)c2ccccc21] | MeSO₃⁻ | bis(1-methyl-1H-indol-3-yl)(4- (trifluoromethyl)phenyl)methylium methanesulfonate |
| 12 | 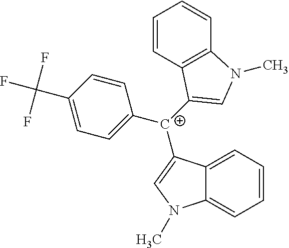 [SMILES: Cn1cc([C+](c2ccc(C(F)(F)F)cc2)c2cn(C)c3ccccc23)c2ccccc21] | Cl⁻ | bis(1-methyl-1H-indol-3-yl)(4- (trifluoromethyl)phenyl)methylium chloride |
| 13 | 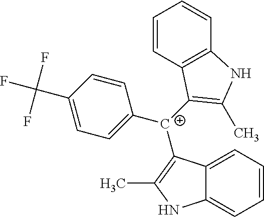 [SMILES: Cc1[nH]c2ccccc2c1[C+](c1ccc(C(F)(F)F)cc1)c1c(C)[nH]c2ccccc12] | MeSO₃⁻ | bis(2-methyl-1H-indol-3-yl)(4- (trifluoromethyl)phenyl)methylium methanesulfonate |
| 14 | 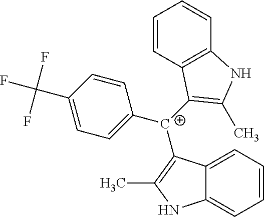 [SMILES: Cc1[nH]c2ccccc2c1[C+](c1ccc(C(F)(F)F)cc1)c1c(C)[nH]c2ccccc12] | Cl⁻ | bis(2-methyl-1H-indol-3-yl)(4- (trifluoromethyl)phenyl)methylium chloride |
| 15 | 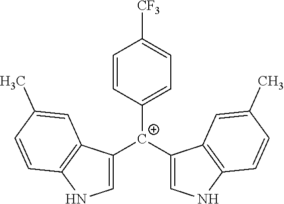 [SMILES: Cc1ccc2[nH]cc([C+](c3ccc(C(F)(F)F)cc3)c3c[nH]c4ccc(C)cc34)c2c1] | MeSO₃⁻ | bis(5-methyl-1H-indol-3-yl)(4- (trifluoromethyl)phenyl)methylium methanesulfonate |
| 16 | 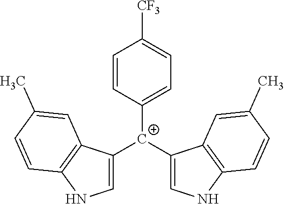 [SMILES: Cc1ccc2[nH]cc([C+](c3ccc(C(F)(F)F)cc3)c3c[nH]c4ccc(C)cc34)c2c1] | Cl⁻ | bis(5-methyl-1H-indol-3-yl)(4- (trifluoromethyl)phenyl)methylium chloride |
| 17 | 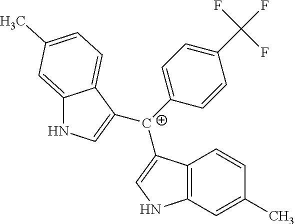 | MeSO₃⁻ | bis(6-methyl-1H-indol-3-yl)(4- (trifluoromethyl)phenyl)methylium methanesulfonate |
| 18 | 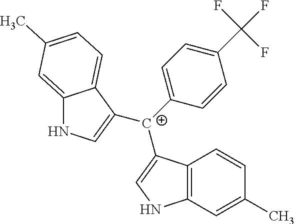 | Cl⁻ | bis(6-methyl-1H-indol-3-yl)(4- (trifluoromethyl)phenyl)methylium chloride |
| 19 | 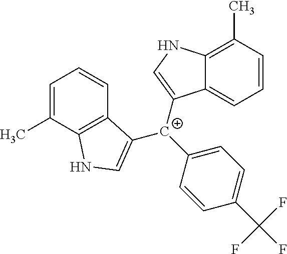 [SMILES: Cc1cccc2c([C+](c3ccc(C(F)(F)F)cc3)c3c[nH]c4c(C)cccc34)c[nH]c12] | MeSO₃⁻ | bis(7-methyl-1H-indol-3-yl)(4- (trifluoromethyl)phenyl)methylium methanesulfonate |
| 20 | 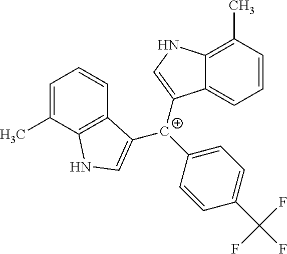 [SMILES: Cc1cccc2c([C+](c3ccc(C(F)(F)F)cc3)c3c[nH]c4c(C)cccc34)c[nH]c12] | Cl⁻ | bis(7-methyl-1H-indol-3-yl)(4- (trifluoromethyl)phenyl)methylium chloride |
| 21 | 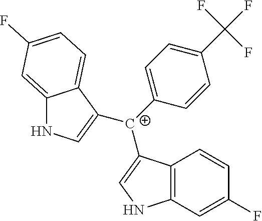 [SMILES: Fc1ccc2c([C+](c3ccc(C(F)(F)F)cc3)c3c[nH]c4cc(F)ccc34)c[nH]c2c1] | MeSO₃⁻ | bis(6-fluoro-1H-indol-3-yl)(4- (trifluoromethyl)phenyl)methylium methanesulfonate |
| 22 | 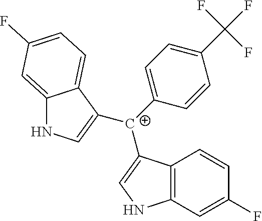 [SMILES: Fc1ccc2c([C+](c3ccc(C(F)(F)F)cc3)c3c[nH]c4cc(F)ccc34)c[nH]c2c1] | Cl⁻ | bis(6-fluoro-1H-indol-3-yl)(4- (trifluoromethyl)phenyl)methylium chloride |
| 23 | 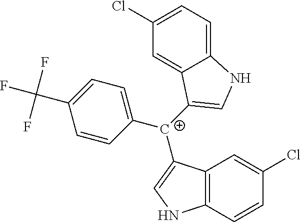 [SMILES: FC(F)(F)c1ccc([C+](c2c[nH]c3ccc(Cl)cc23)c2c[nH]c3ccc(Cl)cc23)cc1] | MeSO₃⁻ | bis(5-chloro-1H-indol-3-yl)(4- (trifluoromethyl)phenyl)methylium methanesulfonate |
| 24 | 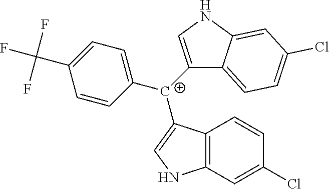 [SMILES: FC(F)(F)c1ccc([C+](c2c[nH]c3cc(Cl)ccc23)c2c[nH]c3cc(Cl)ccc23)cc1] | MeSO₃⁻ | bis(6-chloro-1H-indol-3-yl)(4- (trifluoromethyl)phenyl)methylium methanesulfonate |
| 25 | 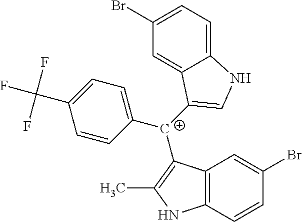 [SMILES: Cc1[nH]c2ccc(Br)cc2c1[C+](c1ccc(C(F)(F)F)cc1)c1c[nH]c2ccc(Br)cc12] | MeSO₃⁻ | bis(5-bromo-1H-indol-3-yl)(4- (trifluoromethyl)phenyl)methylium methanesulfonate |
| 26 | 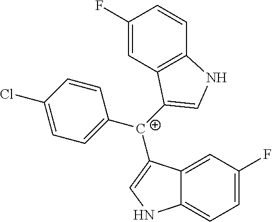 [SMILES: Fc1ccc2[nH]cc([C+](c3ccc(Cl)cc3)c3c[nH]c4ccc(F)cc34)c2c1] | MeSO₃⁻ | bis(5-fluoro-1H-indol-3-yl)(4- chlorophenyl)methylium methanesulfonate |
| 27 | 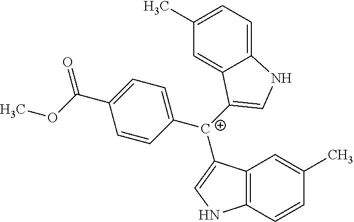 | MeSO₃⁻ | bis(5-methyl-1H-indol-3-yl)(4- (methoxycarbonyl)phenyl)methylium methanesulfonate |
| 28 | 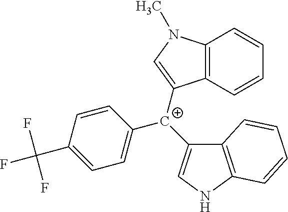 [SMILES: Cn1cc([C+](c2ccc(C(F)(F)F)cc2)c2c[nH]c3ccccc23)c2ccccc21] | MeSO₃⁻ | (1-methyl-1H-indol-3-yl)(1H-indol-3- yl)(4- (trifluoromethyl)phenyl)methylium methanesulfonate |
| 29 | 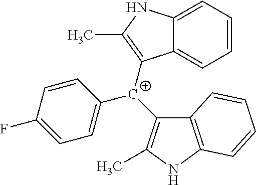 [SMILES: Cc1[nH]c2ccccc2c1[C+](c1ccc(F)cc1)c1c(C)[nH]c2ccccc12] | Cl⁻ | bis(2-methyl-1H-indol-3-yl)(4- fluorophenyl)methylium chloride |
| 30 | 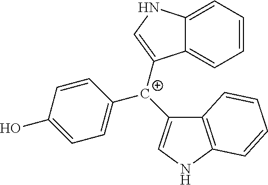 [SMILES: Oc1ccc([C+](c2c[nH]c3ccccc23)c2c[nH]c3ccccc23)cc1] | Cl⁻ | bis(1H-indol-3-yl)(4- hydroxyphenyl)methylium chloride |
| 31 | 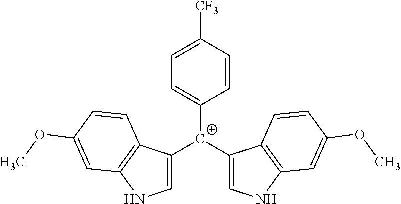 | Cl⁻ | bis(6-methoxy-1H-indol-3-yl)(4- (trifluoromethyl)phenyl)methylium chloride |
| 32 | 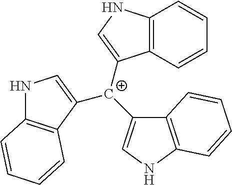 [SMILES: c1ccc2c([C+](c3c[nH]c4ccccc34)c3c[nH]c4ccccc34)c[nH]c2c1] | Cl⁻ | tris(1H-indol-3-yl)methylium chloride |
| 33 | 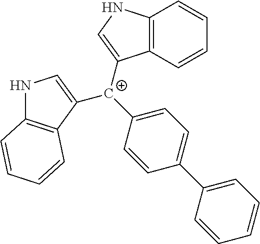 [SMILES: c1ccc(-c2ccc([C+](c3c[nH]c4ccccc34)c3c[nH]c4ccccc34)cc2)cc1] | MeSO₃⁻ | bis(1H-indol-3-yl)((1,1′-biphenyl)-4- yl)methylium methanesulfonate |
| 34 | 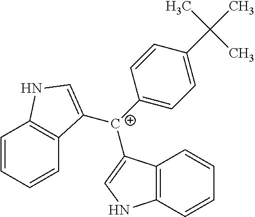 [SMILES: CC(C)(C)c1ccc([C+](c2c[nH]c3ccccc23)c2c[nH]c3ccccc23)cc1] | MeSO₃⁻ | bis(1H-indol-3-yl)(4-tert- butylphenyl)methylium methanesulfonate |
| 35 | 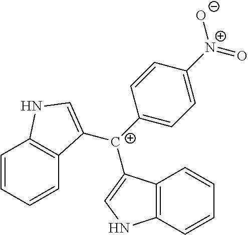 [SMILES: O=[N+]([O-])c1ccc([C+](c2c[nH]c3ccccc23)c2c[nH]c3ccccc23)cc1] | MeSO₃⁻ | bis(1H-indol-3-yl)(4- nitrophenyl)methylium methanesulfonate |
| 36 | 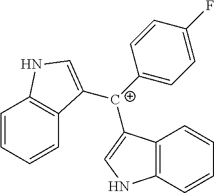 [SMILES: Fc1ccc([C+](c2c[nH]c3ccccc23)c2c[nH]c3ccccc23)cc1] | MeSO₃⁻ | bis(1H-indol-3-yl)(4- fluorophenyl)methylium methanesulfonate |
| 37 | 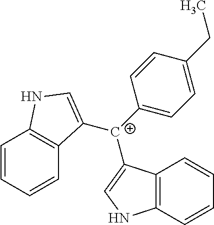 [SMILES: CCc1ccc([C+](c2c[nH]c3ccccc23)c2c[nH]c3ccccc23)cc1] | MeSO₃⁻ | bis(1H-indol-3-yl)(4- ethylphenyl)methylium methanesulfonate |
| 38 | 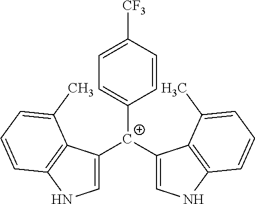 [SMILES: Cc1cccc2[nH]cc([C+](c3ccc(C(F)(F)F)cc3)c3c[nH]c4cccc(C)c34)c12] | Cl⁻ | bis(4-methyl-1H-indol-3-yl)(4- (trifluoromethyl)phenyl)methylium chloride |
| 39 | 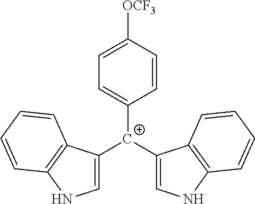 [SMILES: FC(F)(F)Oc1ccc([C+](c2c[nH]c3ccccc23)c2c[nH]c3ccccc23)cc1] | Cl⁻ | bis(1H-indol-3-yl)(4- (trifluoromethoxy)phenyl)methylium chloride |
| 40 | 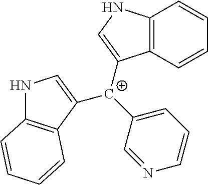 [SMILES: c1cncc([C+](c2c[nH]c3ccccc23)c2c[nH]c3ccccc23)c1] | Cl⁻ | bis(1H-indol-3-yl)(pyridin-3- yl)methylium chloride |
| 41 | 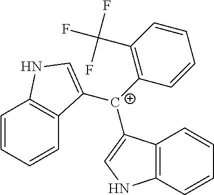 [SMILES: FC(F)(F)c1ccccc1[C+](c1c[nH]c2ccccc12)c1c[nH]c2ccccc12] | Cl⁻ | bis(1H-indol-3-yl)(2- (trifluoromethyl)phenyl)methylium chloride |
| 42 | 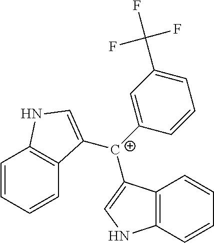 [SMILES: FC(F)(F)c1cccc([C+](c2c[nH]c3ccccc23)c2c[nH]c3ccccc23)c1] | Cl⁻ | bis(1H-indol-3-yl)(3- (trifluoromethyl)phenyl)methylium chloride |
| 43 | 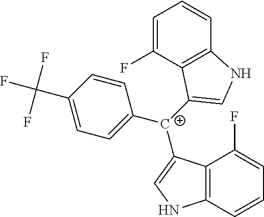 [SMILES: Fc1cccc2[nH]cc([C+](c3ccc(C(F)(F)F)cc3)c3c[nH]c4cccc(F)c34)c12] | Cl⁻ | bis(4-fluoro-1H-indol-3-yl)(4- (trifluoromethyl)phenyl)methylium chloride |
| 44 | 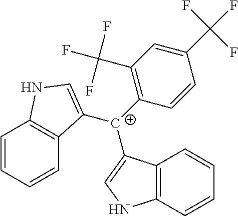 [SMILES: FC(F)(F)c1ccc([C+](c2c[nH]c3ccccc23)c2c[nH]c3ccccc23)c(C(F)(F)F)c1] | Cl⁻ | bis(1H-indol-3-yl)(2,4- bis(trifluoromethyl)phenyl)methylium chloride |
| 45 | 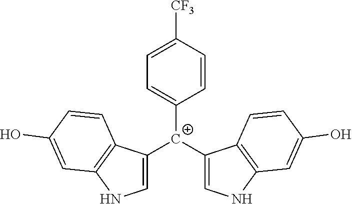 [SMILES: Oc1ccc2c([C+](c3ccc(C(F)(F)F)cc3)c3c[nH]c4cc(O)ccc34)c[nH]c2c1] | Cl⁻ | bis(6-hydroxy-1H-indol-3-yl)(4- (trifluoromethyl)phenyl)methylium chloride |
| 46 | 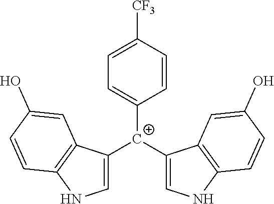 [SMILES: Oc1ccc2[nH]cc([C+](c3ccc(C(F)(F)F)cc3)c3c[nH]c4ccc(O)cc34)c2c1] | Cl⁻ | bis(5-hydroxy-1H-indol-3-yl)(4- (trifluoromethyl)phenyl)methylium chloride |
| 47 | 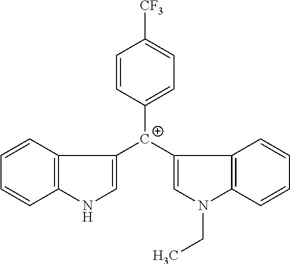 [SMILES: CCn1cc([C+](c2ccc(C(F)(F)F)cc2)c2c[nH]c3ccccc23)c2ccccc21] | Cl⁻ | (1-ethyl-1H-indol-3-yl)(1H-indol-3- yl)(4- (trifluoromethyl)phenyl)methylium chloride |
| 48 | 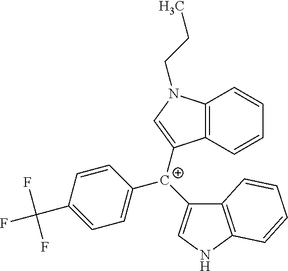 [SMILES: CCCn1cc([C+](c2ccc(C(F)(F)F)cc2)c2c[nH]c3ccccc23)c2ccccc21] | Cl⁻ | (1-propyl-1H-indol-3-yl)(1H-indol-3- yl)(4- (trifluoromethyl)phenyl)methylium chloride |
| 49 | 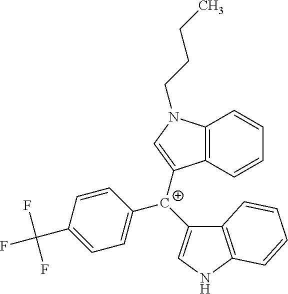 [SMILES: CCCCn1cc([C+](c2ccc(C(F)(F)F)cc2)c2c[nH]c3ccccc23)c2ccccc21] | Cl⁻ | (1-butyl-1H-indol-3-yl)(1H-indol-3- yl)(4- (trifluoromethyl)phenyl)methylium chloride |
| 50 | 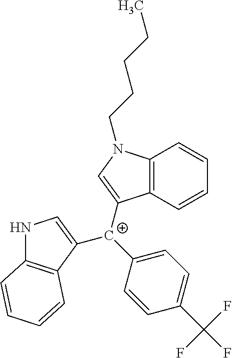 [SMILES: CCCCCn1cc([C+](c2ccc(C(F)(F)F)cc2)c2c[nH]c3ccccc23)c2ccccc21] | Cl⁻ | (1-pentyl-1H-indol-3-yl)(1H-indol-3- yl)(4- (trifluoromethyl)phenyl)methylium chloride |
| 51 | 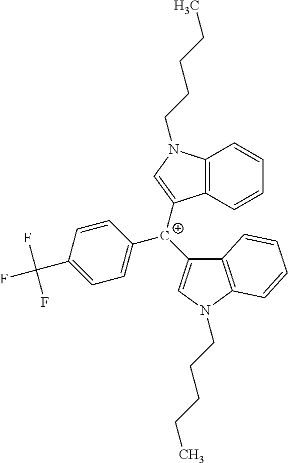 [SMILES: CCCCCn1cc([C+](c2ccc(C(F)(F)F)cc2)c2cn(CCCCC)c3ccccc23)c2ccccc21] | Cl⁻ | bis(1-pentyl-1H-indol-3-yl)(4- (trifluoromethyl)phenyl)methylium chloride |
| 52 | 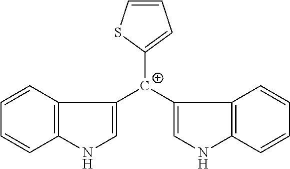 [SMILES: c1csc([C+](c2c[nH]c3ccccc23)c2c[nH]c3ccccc23)c1] | Cl⁻ | bis(1H-indol-3-yl)(thiophen-2- yl)methylium chloride |
| 53 | 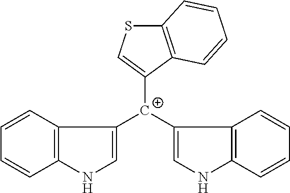 [SMILES: c1ccc2c([C+](c3c[nH]c4ccccc34)c3csc4ccccc34)c[nH]c2c1] | Cl⁻ | bis(1H-indol-3-yl)(1-benzothiophen- 3-yl)methylium chloride |
| 54 | 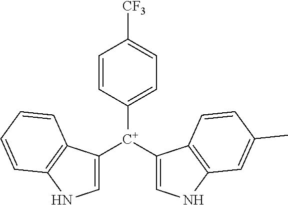 [SMILES: Cc1ccc2c([C+](c3ccc(C(F)(F)F)cc3)c3c[nH]c4ccccc34)c[nH]c2c1] | Cl⁻ | (6-methyl-1H-indol-3-yl)(1H-indol-3- yl)(4- (trifluoromethyl)phenyl)methylium chloride |
| 55 | 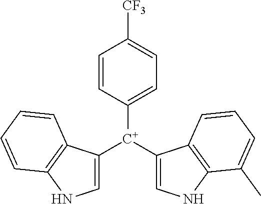 [SMILES: Cc1cccc2c([C+](c3ccc(C(F)(F)F)cc3)c3c[nH]c4ccccc34)c[nH]c12] | Cl⁻ | (7-methyl-1H-indol-3-yl)(1H-indol-3- yl)(4- (trifluoromethyl)phenyl)methylium chloride |
| 56 | 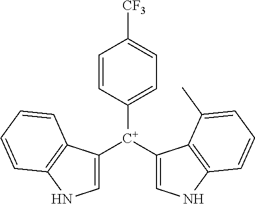 [SMILES: Cc1cccc2[nH]cc([C+](c3ccc(C(F)(F)F)cc3)c3c[nH]c4ccccc34)c12] | Cl⁻ | (4-methyl-1H-indol-3-yl)(1H-indol-3- yl)(4- (trifluoromethyl)phenyl)methylium chloride |
| 57 | 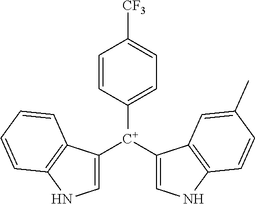 [SMILES: Cc1ccc2[nH]cc([C+](c3ccc(C(F)(F)F)cc3)c3c[nH]c4ccccc34)c2c1] | Cl⁻ | (5-methyl-1H-indol-3-yl)(1H-indol-3- yl)(4- (trifluoromethyl)phenyl)methylium chloride |
| 58 | 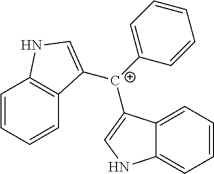 [SMILES: c1ccc([C+](c2c[nH]c3ccccc23)c2c[nH]c3ccccc23)cc1] | MeSO₃⁻ | bis(1H-indol-3-yl)(phenyl)methylium methanesulfonate |
| 59 | 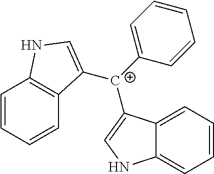 [SMILES: c1ccc([C+](c2c[nH]c3ccccc23)c2c[nH]c3ccccc23)cc1] | Cl⁻ | bis(1H-indol-3-yl)(phenyl)methylium chloride |
| 60 | 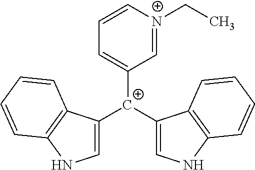 [SMILES: CC[n+]1cccc([C+](c2c[nH]c3ccccc23)c2c[nH]c3ccccc23)c1] | 2 Cl⁻ | 3-(bis(1H-indol-3-yl)metheyliumyl)- 1-ethylpyridin-1-ium dichloride |
| 61 | 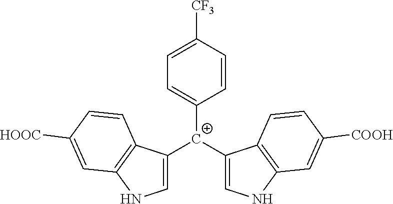 [SMILES: O=C(O)c1ccc2c([C+](c3ccc(C(F)(F)F)cc3)c3c[nH]c4cc(C(=O)O)ccc34)c[nH]c2c1] | Cl⁻ | bis(6-carboxy-1H-indol-3-yl)(4- (trifluoromethyl)phenyl)methylium chloride |
| 62 | 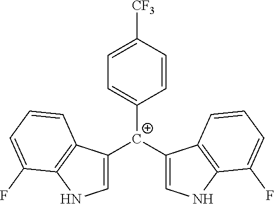 [SMILES: Fc1cccc2c([C+](c3ccc(C(F)(F)F)cc3)c3c[nH]c4c(F)cccc34)c[nH]c12] | Cl⁻ | bis(7-fluoro-1H-indol-3-yl)(4- (trifluoromethyl)phenyl)methylium chloride |
| 63 | 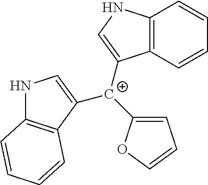 [SMILES: c1coc([C+](c2c[nH]c3ccccc23)c2c[nH]c3ccccc23)c1] | Cl⁻ | bis(1H-indol-3-yl)(furan-2- yl)methylium chloride |
| 64 | 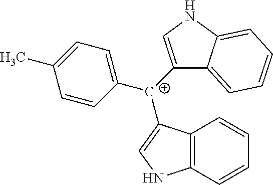 [SMILES: Cc1ccc([C+](c2c[nH]c3ccccc23)c2c[nH]c3ccccc23)cc1] | MeSO₃⁻ | bis(1H-indol-3-yl)(4- methylphenyl)methylium methanesulfonate |
| 65 | 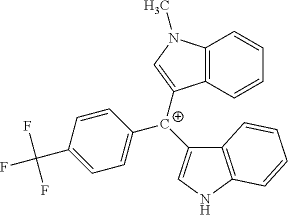 [SMILES: Cn1cc([C+](c2ccc(C(F)(F)F)cc2)c2c[nH]c3ccccc23)c2ccccc21] | Cl⁻ | (1-methyl-1H-indol-3-yl)(1H-indol-3- yl)(4- (trifluoromethyl)phenyl)methylium chloride |
| 66 | 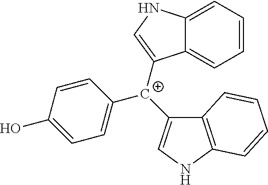 [SMILES: Oc1ccc([C+](c2c[nH]c3ccccc23)c2c[nH]c3ccccc23)cc1] | MeSO₃⁻ | bis(1H-indol-3-yl)(4- hydroxyphenyl)methylium methanesulfonate |
| 67 | 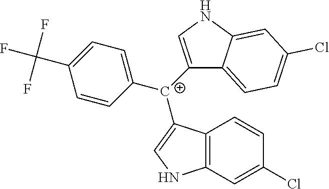 [SMILES: FC(F)(F)c1ccc([C+](c2c[nH]c3cc(Cl)ccc23)c2c[nH]c3cc(Cl)ccc23)cc1] | Cl⁻ | bis(6-chloro-1H-indol-3-yl)(4- (trifluoromethyl)phenyl)methylium chloride |
| 68 | 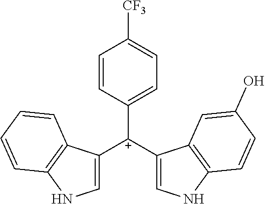 [SMILES: Oc1ccc2[nH]cc([C+](c3ccc(C(F)(F)F)cc3)c3c[nH]c4ccccc34)c2c1] | Cl⁻ | (5-hydroxy-1H-indol-3-yl)(1H-indol- 3-yl)(4- (trifluoromethyl)phenyl)methylium chloride |
| 69 | 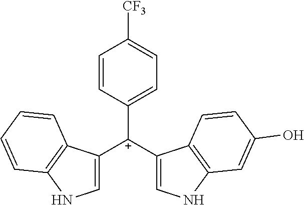 [SMILES: Oc1ccc2c([C+](c3ccc(C(F)(F)F)cc3)c3c[nH]c4ccccc34)c[nH]c2c1] | Cl⁻ | (6-hydroxy-1H-indol-3-yl)(1H-indol- 3-yl)(4- (trifluoromethyl)phenyl)methylium chloride |
| 70 | 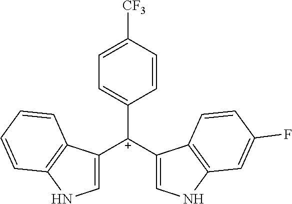 [SMILES: Fc1ccc2c([C+](c3ccc(C(F)(F)F)cc3)c3c[nH]c4ccccc34)c[nH]c2c1] | Cl⁻ | (6-fluoro-1H-indol-3-yl)(1H-indol-3- yl)(4- (trifluoromethyl)phenyl)methylium chloride |
| 71 | 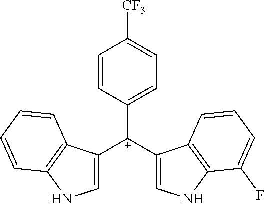 [SMILES: Fc1cccc2c([C+](c3ccc(C(F)(F)F)cc3)c3c[nH]c4ccccc34)c[nH]c12] | Cl⁻ | (7-fluoro-1H-indol-3-yl)(1H-indol-3- yl)(4- (trifluoromethyl)phenyl)methylium chloride |
| 72 | 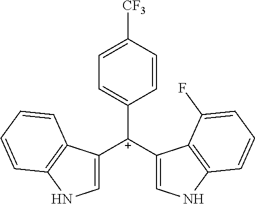 [SMILES: Fc1cccc2[nH]cc([C+](c3ccc(C(F)(F)F)cc3)c3c[nH]c4ccccc34)c12] | Cl⁻ | (4-fluoro-1H-indol-3-yl)(1H-indol-3- yl)(4- (trifluoromethyl)phenyl)methylium chloride |
| 73 | 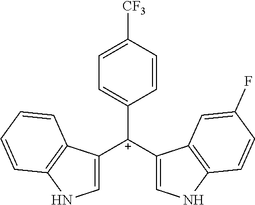 [SMILES: Fc1ccc2[nH]cc([C+](c3ccc(C(F)(F)F)cc3)c3c[nH]c4ccccc34)c2c1] | Cl⁻ | (5-fluoro-1H-indol-3-yl)(1H-indol-3- yl)(4- (trifluoromethyl)phenyl)methylium chloride |
| 74 | 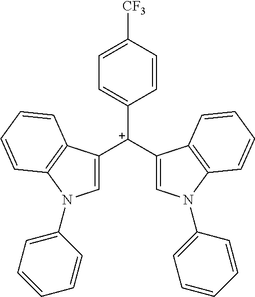 [SMILES: FC(F)(F)c1ccc([C+](c2cn(-c3ccccc3)c3ccccc23)c2cn(-c3ccccc3)c3ccccc23)cc1] | Cl⁻ | bis(1-phenyl-1H-indol-3-yl)(4- (trifluoromethyl)phenyl)methylium chloride |
| 75 | 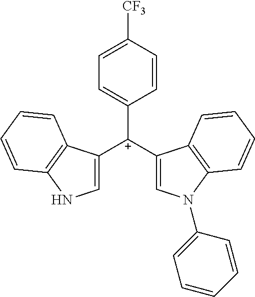 [SMILES: FC(F)(F)c1ccc([C+](c2c[nH]c3ccccc23)c2cn(-c3ccccc3)c3ccccc23)cc1] | Cl⁻ | (1H-indol-3-yl)(1-phenyl-1H-indol-3- yl)(4- (trifluoromethyl)phenyl)methylium chloride |
| 76 | 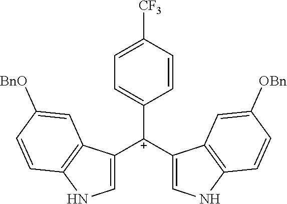 [SMILES: FC(F)(F)c1ccc([C+](c2c[nH]c3ccc(OCc4ccccc4)cc23)c2c[nH]c3ccc(OCc4ccccc4)cc23)cc1] | Cl⁻ | bis(5-(benzyloxy)-1H-indol-3-yl)(4- (trifluoromethyl)phenyl)methylium chloride |
| 77 | 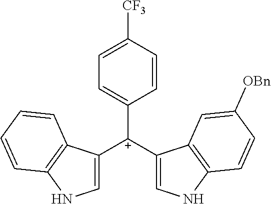 [SMILES: FC(F)(F)c1ccc([C+](c2c[nH]c3ccccc23)c2c[nH]c3ccc(OCc4ccccc4)cc23)cc1] | Cl⁻ | (5-(benzyloxy)-1H-indol-3-yl)(1H- indol-3-yl)(4- (trifluoromethyl)phenyl)methylium chloride |
| 78 | 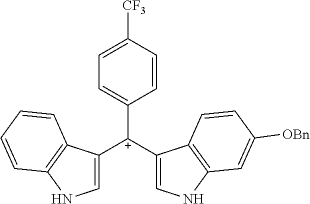 [SMILES: FC(F)(F)c1ccc([C+](c2c[nH]c3ccccc23)c2c[nH]c3cc(OCc4ccccc4)ccc23)cc1] | Cl⁻ | (6-(benzyloxy)-1H-indol-3-yl)(1H- indol-3-yl)(4- (trifluoromethyl)phenyl)methylium chloride |
| 79 | 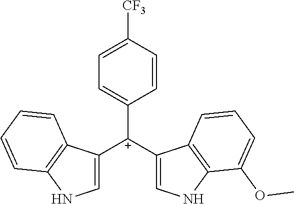 [SMILES: COc1cccc2c([C+](c3ccc(C(F)(F)F)cc3)c3c[nH]c4ccccc34)c[nH]c12] | Cl⁻ | (1H-indol-3-yl)(7-methoxy-1H-indol- 3-yl)(4- (trifluoromethyl)phenyl)methylium chloride |
| 80 | 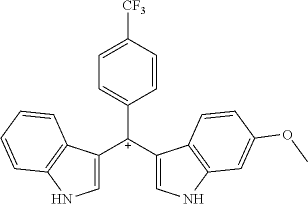 [SMILES: COc1ccc2c([C+](c3ccc(C(F)(F)F)cc3)c3c[nH]c4ccccc34)c[nH]c2c1] | Cl⁻ | (1H-indol-3-yl)(6-methoxy-1H-indol- 3-yl)(4- (trifluoromethyl)phenyl)methylium chloride |
| 81 | 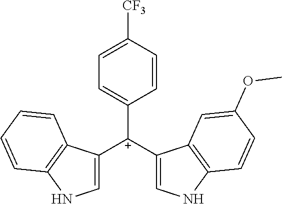 [SMILES: COc1ccc2[nH]cc([C+](c3ccc(C(F)(F)F)cc3)c3c[nH]c4ccccc34)c2c1] | Cl⁻ | (1H-indol-3-yl)(5-methoxy-1H-indol- 3-yl)(4- (trifluoromethyl)phenyl)methylium chloride |
| 82 | 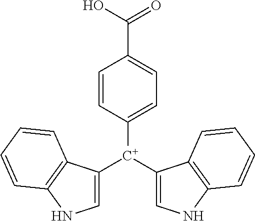 [SMILES: O=C(O)c1ccc([C+](c2c[nH]c3ccccc23)c2c[nH]c3ccccc23)cc1] | MeSO₃⁻ | bis(1H-indol-3-yl)(4- carboxyphenyl)methylium methanesulfonate |
| 83 | 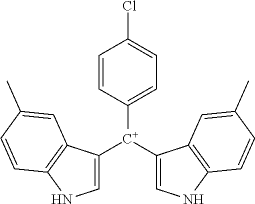 [SMILES: Cc1ccc2[nH]cc([C+](c3ccc(Cl)cc3)c3c[nH]c4ccc(C)cc34)c2c1] | MeSO₃⁻ | bis(5-methyl-1H-indol-3-yl)(4- chlorophenyl)methylium methanesulfonate |
| 84 | 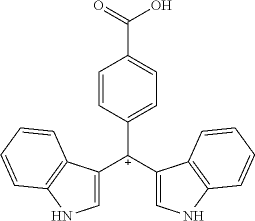 [SMILES: O=C(O)c1ccc([C+](c2c[nH]c3ccccc23)c2c[nH]c3ccccc23)cc1] | Cl⁻ | Bis(1H-indol-3-yl)(4- carboxyphenyl)methylium chloride |
| 85 | 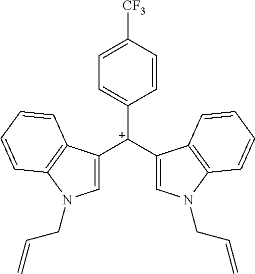 [SMILES: C=CCn1cc([C+](c2ccc(C(F)(F)F)cc2)c2cn(CC=C)c3ccccc23)c2ccccc21] | Cl⁻ | bis(1-allyl-1H-indol-3-yl)(4- (trifluoromethyl)phenyl)methylium chloride |
| 86 | 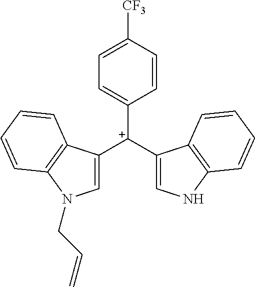 [SMILES: C=CCn1cc([C+](c2ccc(C(F)(F)F)cc2)c2c[nH]c3ccccc23)c2ccccc21] | Cl⁻ | (1-allyl-1H-indol-3-yl)(1H-indol-3- yl)(4- (trifluoromethyl)phenyl)methylium chloride |
| 87 | 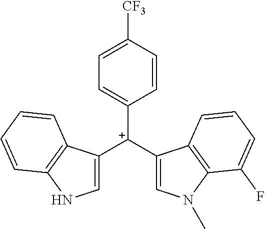 [SMILES: Cn1cc([C+](c2ccc(C(F)(F)F)cc2)c2c[nH]c3ccccc23)c2cccc(F)c21] | Cl⁻ | (7-fluoro-1-methyl-1H-indol-3- yl)(1H-indol-3-yl)(4- (trifluoromethyl)phenyl)-methylium chloride |
| 88 | 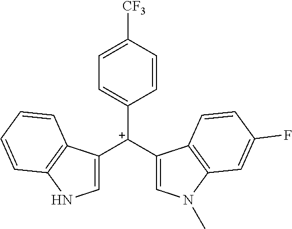 [SMILES: Cn1cc([C+](c2ccc(C(F)(F)F)cc2)c2c[nH]c3ccccc23)c2ccc(F)cc21] | Cl⁻ | (6-fluoro-1-methyl-1H-indol-3- yl)(1H-indol-3-yl)(4- (trifluoromethyl)phenyl)- methylium chloride |
| 89 | 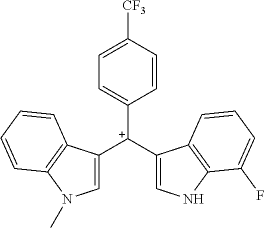 [SMILES: Cn1cc([C+](c2ccc(C(F)(F)F)cc2)c2c[nH]c3c(F)cccc23)c2ccccc21] | Cl⁻ | (7-fluoro-1H-indol-3-yl)(1-methyl- 1H-indol-3-yl)(4- (trifluoromethyl)phenyl)- methylium chloride |
| 90 | 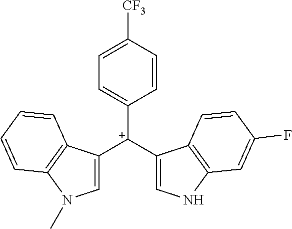 [SMILES: Cn1cc([C+](c2ccc(C(F)(F)F)cc2)c2c[nH]c3cc(F)ccc23)c2ccccc21] | Cl⁻ | (6-fluoro-1H-indol-3-yl)(1-methyl- 1H-indol-3-yl)(4- (trifluoromethyl)phenyl)- methylium chloride |
| 91 | 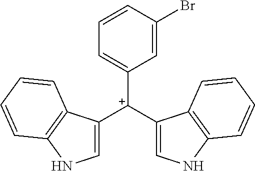 [SMILES: Brc1cccc([C+](c2c[nH]c3ccccc23)c2c[nH]c3ccccc23)c1] | Cl⁻ | (3-bromophenyl)di(1H-indol-3- yl)methylium chloride |
| 92 | 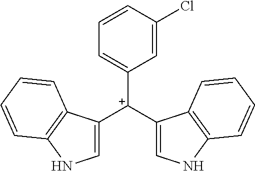 [SMILES: Clc1cccc([C+](c2c[nH]c3ccccc23)c2c[nH]c3ccccc23)c1] | Cl⁻ | (3-chlorophenyl)di(1H-indol-3- yl)methylium chloride |
| 93 | 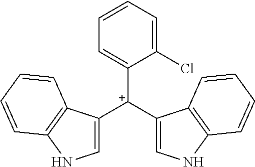 [SMILES: Clc1ccccc1[C+](c1c[nH]c2ccccc12)c1c[nH]c2ccccc12] | Cl⁻ | (2-chlorophenyl)di(1H-indol-3- yl)methylium chloride |
| 94 | 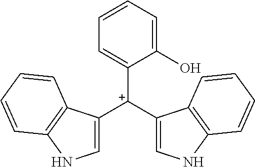 [SMILES: Oc1ccccc1[C+](c1c[nH]c2ccccc12)c1c[nH]c2ccccc12] | Cl⁻ | (2-hydroxyphenyl)di(1H-indol-3- yl)methylium chloride |
| 95 | 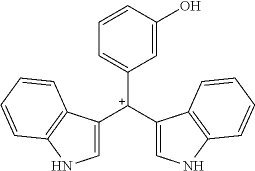 [SMILES: Oc1cccc([C+](c2c[nH]c3ccccc23)c2c[nH]c3ccccc23)c1] | Cl⁻ | (3-hydroxyphenyl)di(1H-indol-3- yl)methylium chloride |
| 96 | 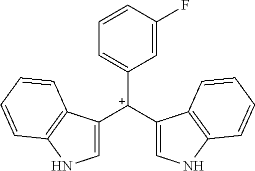 [SMILES: Fc1cccc([C+](c2c[nH]c3ccccc23)c2c[nH]c3ccccc23)c1] | Cl⁻ | (3-fluorophenyl)di(1H-indol-3- yl)methylium chloride |
| 97 | 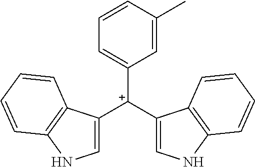 [SMILES: Cc1cccc([C+](c2c[nH]c3ccccc23)c2c[nH]c3ccccc23)c1] | Cl⁻ | di(1H-indol-3-yl)(m-tolyl)methylium chloride |
| 98 | 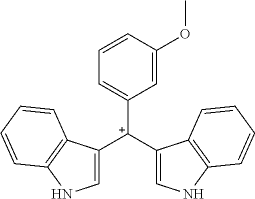 [SMILES: COc1cccc([C+](c2c[nH]c3ccccc23)c2c[nH]c3ccccc23)c1] | Cl⁻ | di(1H-indol-3-yl)(3- methoxyphenyl)methylium chloride |
| 99 | 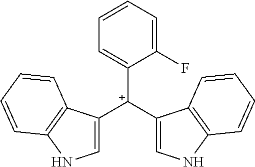 [SMILES: Fc1ccccc1[C+](c1c[nH]c2ccccc12)c1c[nH]c2ccccc12] | Cl⁻ | (2-fluorophenyl)di(1H-indol-3- yl)methylium chloride |
| 100 | 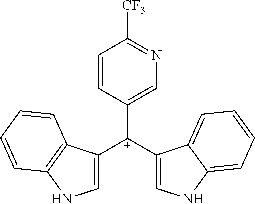 [SMILES: FC(F)(F)c1ccc([C+](c2c[nH]c3ccccc23)c2c[nH]c3ccccc23)cn1] | Cl⁻ | Di(1H-indol-3-yl)(6- (trifluoromethyl)pyridin-3- yl)methylium chloride |
| 101 | 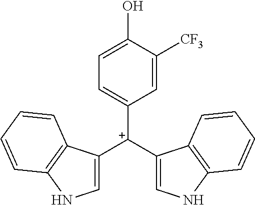 [SMILES: Oc1ccc([C+](c2c[nH]c3ccccc23)c2c[nH]c3ccccc23)cc1C(F)(F)F] | Cl⁻ | (4-hydroxy-3- (trifluoromethyl)phenyl)di(1H-indol- 3-yl)methylium chloride |
| 102 | 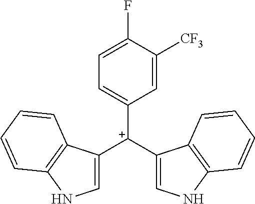 [SMILES: Fc1ccc([C+](c2c[nH]c3ccccc23)c2c[nH]c3ccccc23)cc1C(F)(F)F] | Cl⁻ | (4-fluoro-3- (trifluoromethyl)phenyl)di(1H-indol- 3-yl)methylium chloride |
| 103 | 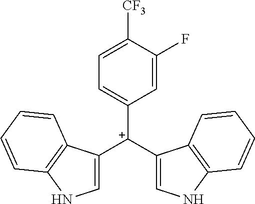 [SMILES: Fc1cc([C+](c2c[nH]c3ccccc23)c2c[nH]c3ccccc23)ccc1C(F)(F)F] | Cl⁻ | (3-fluoro-4- (trifluoromethyl)phenyl)di(1H-indol- 3-yl)methylium chloride |
| 104 | 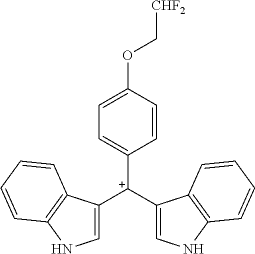 [SMILES: FC(F)COc1ccc([C+](c2c[nH]c3ccccc23)c2c[nH]c3ccccc23)cc1] | Cl⁻ | (4-(2,2-difluoroethoxy)phenyl)di(1H- indol-3-yl)methylium chloride |
| 105 | 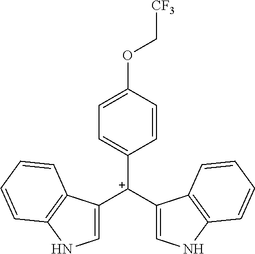 [SMILES: FC(F)(F)COc1ccc([C+](c2c[nH]c3ccccc23)c2c[nH]c3ccccc23)cc1] | Cl⁻ | di(1H-indol-3-yl)(4-(2,2,2- trifluoroethoxy)phenyl)methylium chloride |
| 106 | 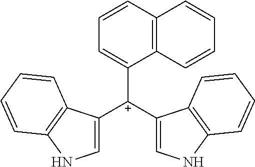 [SMILES: c1ccc2c([C+](c3c[nH]c4ccccc34)c3c[nH]c4ccccc34)cccc2c1] | Cl⁻ | Di(1H-indol-3-yl)(naphthalen-1- yl)methylium chloride |
| 107 | 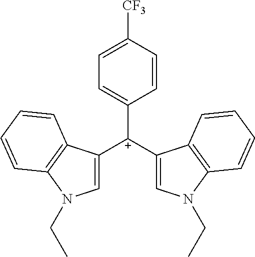 [SMILES: CCn1cc([C+](c2ccc(C(F)(F)F)cc2)c2cn(CC)c3ccccc23)c2ccccc21] | Cl⁻ | Bis(1-ethyl-1H-indol-3-yl)(4- (trifluoromethyl)phenyl)methylium chloride |
| 108 | 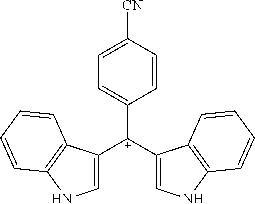 [SMILES: N#Cc1ccc([C+](c2c[nH]c3ccccc23)c2c[nH]c3ccccc23)cc1] | Cl⁻ | (4-Cyanophenyl)di(1H-indol-3- yl)methylium chloride |
| 109 | 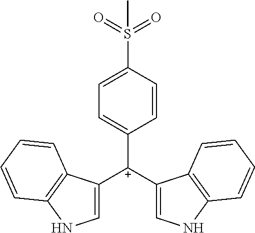 [SMILES: CS(=O)(=O)c1ccc([C+](c2c[nH]c3ccccc23)c2c[nH]c3ccccc23)cc1] | Cl⁻ | Di(1H-indol-3-yl)(4- (methylsulfonyl)phenyl)methylium chloride |

In some embodiments, compounds of Formula (I) include, but are not limited to, compounds comprising cations of a structure in Table 2 and a suitable anion. In some embodiments, the suitable anion is a suitable monovalent anion. In some embodiments, the suitable anion is a divalent anion. In some embodiments, the suitable anion is a polyvalent anion. In some embodiments, the suitable anion is a divalent or polyvalent anion and there is less than one equivalent of said anion. In some embodiments, the suitable anion is selected from anions formed from inorganic acids and anions formed from organic acids. In some embodiments, the suitable anion is selected from halides, chlorates, sulfates, nitrates, phosphates, carboxylates, sulfonates, and borates. In some embodiments, the suitable anion is selected from acetate, benzoate, besylate, borate, bromide, camphorsulfonate, chloride, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, napsylate, nitrate, octadecanoate, oleate, oxalate, pamoate, phosphate, succinate, sulfate, sulfosalicylate, tartrate, tetrahydroxyborate, tetrafluoroborate, tosylate, or trifluoroacetate.

TABLE 2

| Structure | Name |
|---|---|
| 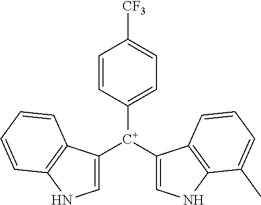 [SMILES: Cc1cccc2c([C+](c3ccc(C(F)(F)F)cc3)c3c[nH]c4ccccc34)c[nH]c12] | (7-methyl-1H-indol-3-yl)(1H-indol-3-yl)(4- (trifluoromethyl)phenyl)methylium |
| 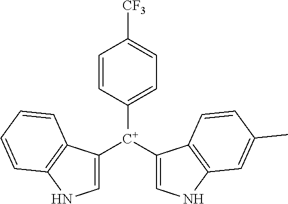 [SMILES: Cc1ccc2c([C+](c3ccc(C(F)(F)F)cc3)c3c[nH]c4ccccc34)c[nH]c2c1] | (6-methyl-1H-indol-3-yl)(1H-indol-3-yl)(4- (trifluoromethyl)phenyl)methylium |
| 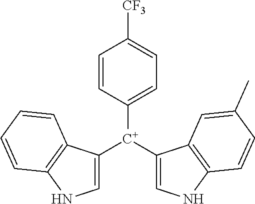 [SMILES: Cc1ccc2[nH]cc([C+](c3ccc(C(F)(F)F)cc3)c3c[nH]c4ccccc34)c2c1] | (5-methyl-1H-indol-3-yl)(1H-indol-3-yl)(4- (trifluoromethyl)phenyl)methylium |
| 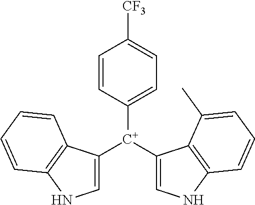 [SMILES: Cc1cccc2[nH]cc([C+](c3ccc(C(F)(F)F)cc3)c3c[nH]c4ccccc34)c12] | (4-methyl-1H-indol-3-yl)(1H-indol-3-yl)(4- (trifluoromethyl)phenyl)methylium |
| 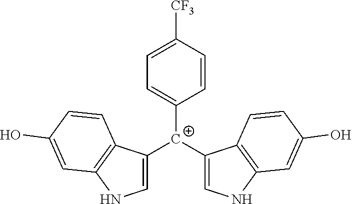 [SMILES: Oc1ccc2c([C+](c3ccc(C(F)(F)F)cc3)c3c[nH]c4cc(O)ccc34)c[nH]c2c1] | bis(6-hydroxy-1H-indol-3-yl)(4- (trifluoromethyl)phenyl)methylium |
| 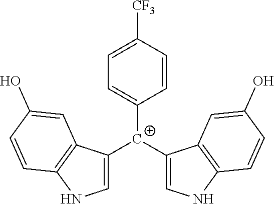 [SMILES: Oc1ccc2[nH]cc([C+](c3ccc(C(F)(F)F)cc3)c3c[nH]c4ccc(O)cc34)c2c1] | bis(5-hydroxy-1H-indol-3-yl)(4- (trifluoromethyl)phenyl)methylium |
| 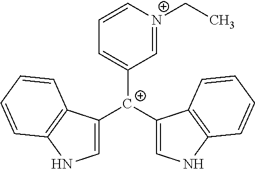 [SMILES: CC[n+]1cccc([C+](c2c[nH]c3ccccc23)c2c[nH]c3ccccc23)c1] | 3-(bis(1H-indol-3-yl)methyliumyl)-1-ethylpyridin-1- ium |
| 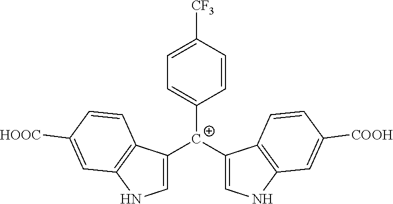 [SMILES: O=C(O)c1ccc2c([C+](c3ccc(C(F)(F)F)cc3)c3c[nH]c4cc(C(=O)O)ccc34)c[nH]c2c1] | bis(6-carboxy-1H-indol-3-yl)(4- (trifluoromethyl)phenyl)methylium |
| 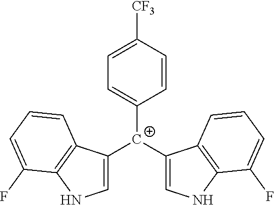 [SMILES: Fc1cccc2c([C+](c3ccc(C(F)(F)F)cc3)c3c[nH]c4c(F)cccc34)c[nH]c12] | bis(7-fluoro-1H-indol-3-yl)(4- (trifluoromethyl)phenyl)methylium |
| 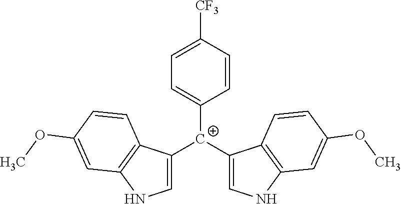 | bis(6-methoxy-1H-indol-3-yl)(4- (trifluoromethyl)phenyl)methylium |
| 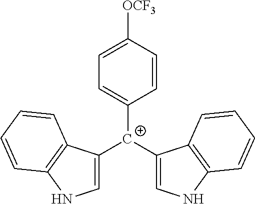 [SMILES: FC(F)(F)Oc1ccc([C+](c2c[nH]c3ccccc23)c2c[nH]c3ccccc23)cc1] | bis(1H-indol-3-yl)(4- (trifluoromethoxy)phenyl)methylium |
| 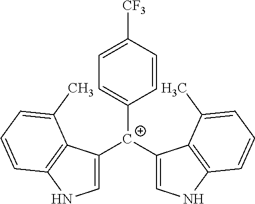 [SMILES: Cc1cccc2[nH]cc([C+](c3ccc(C(F)(F)F)cc3)c3c[nH]c4cccc(C)c34)c12] | bis(4-methyl-1H-indol-3-yl)(4- (trifluoromethyl)phenyl)methylium |
| 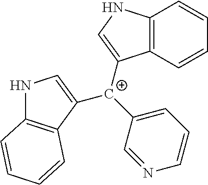 [SMILES: c1cncc([C+](c2c[nH]c3ccccc23)c2c[nH]c3ccccc23)c1] | bis(1H-indol-3-yl)(pyridin-3-yl)methylium |
| 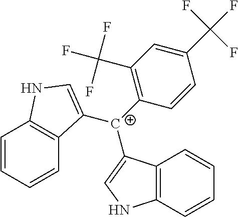 [SMILES: FC(F)(F)c1ccc([C+](c2c[nH]c3ccccc23)c2c[nH]c3ccccc23)c(C(F)(F)F)c1] | bis(1H-indol-3-yl)(2,4- bis(trifluoromethyl)phenyl)methylium |
| 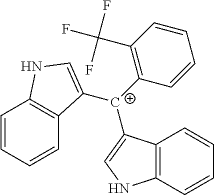 [SMILES: FC(F)(F)c1ccccc1[C+](c1c[nH]c2ccccc12)c1c[nH]c2ccccc12] | bis(1H-indol-3-yl)(2- (trifluoromethyl)phenyl)methylium |
| 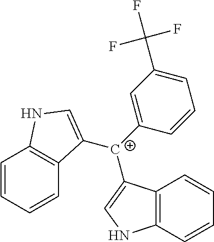 [SMILES: FC(F)(F)c1cccc([C+](c2c[nH]c3ccccc23)c2c[nH]c3ccccc23)c1] | bis(1H-indol-3-yl)(3- (trifluoromethyl)phenyl)methylium |
| 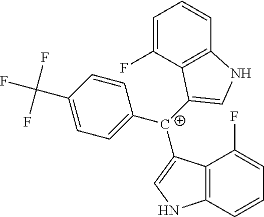 [SMILES: Fc1cccc2[nH]cc([C+](c3ccc(C(F)(F)F)cc3)c3c[nH]c4cccc(F)c34)c12] | bis(4-fluoro-1H-indol-3-yl)(4- (trifluoromethyl)phenyl)methylium |
| 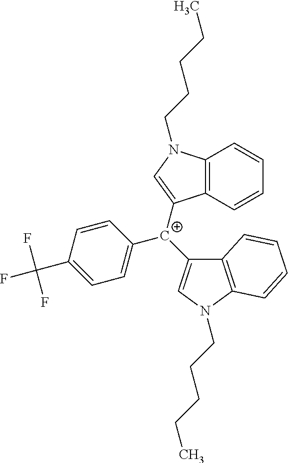 [SMILES: CCCCCn1cc([C+](c2ccc(C(F)(F)F)cc2)c2cn(CCCCC)c3ccccc23)c2ccccc21] | bis(1-pentyl-1H-indol-3-yl)(4- (trifluoromethyl)phenyl)methylium |
| 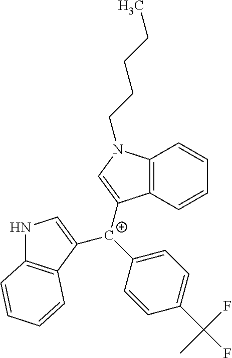 [SMILES: CCCCCn1cc([C+](c2ccc(C(C)(F)F)cc2)c2c[nH]c3ccccc23)c2ccccc21] | (1-pentyl-1H-indol-3-yl)(1H-indol-3-yl)(4- (trifluoromethyl)phenyl)methylium |
| 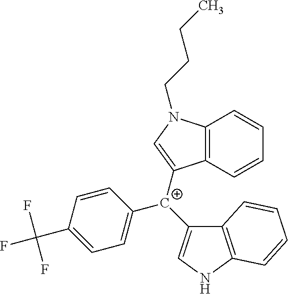 [SMILES: CCCCn1cc([C+](c2ccc(C(F)(F)F)cc2)c2c[nH]c3ccccc23)c2ccccc21] | (1-butyl-1H-indol-3-yl)(1H-indol-3-yl)(4- (trifluoromethyl)phenyl)methylium |
| 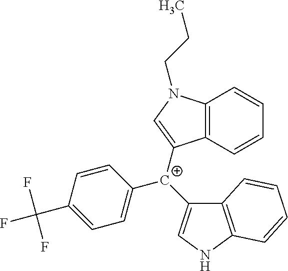 [SMILES: CCCn1cc([C+](c2ccc(C(F)(F)F)cc2)c2c[nH]c3ccccc23)c2ccccc21] | (1-propyl-1H-indol-3-yl)(1H-indol-3-yl)(4- (trifluoromethyl)phenyl)methylium |
| 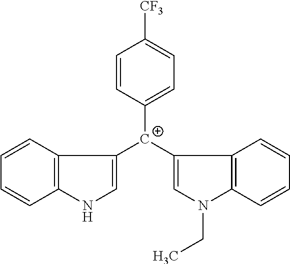 [SMILES: CCn1cc([C+](c2ccc(C(F)(F)F)cc2)c2c[nH]c3ccccc23)c2ccccc21] | (1-ethyl-1H-indol-3-yl)(1 H-indol-3-yl)(4- (trifluoromethyl)phenyl)methylium |
| 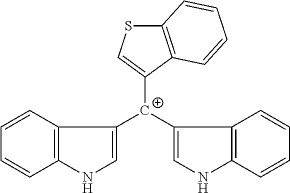 [SMILES: c1ccc2c([C+](c3c[nH]c4ccccc34)c3csc4ccccc34)c[nH]c2c1] | bis(1H-indol-3-yl)(1-benzothiophen-3-yl)methylium |
| 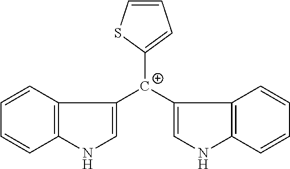 [SMILES: c1csc([C+](c2c[nH]c3ccccc23)c2c[nH]c3ccccc23)c1] | bis(1H-indol-3-yl)(thiophen-2-yl)methylium |
| 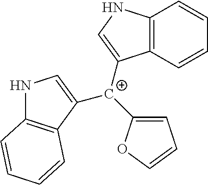 [SMILES: c1coc([C+](c2c[nH]c3ccccc23)c2c[nH]c3ccccc23)c1] | bis(1H-indol-3-yl)(furan-2-yl)methylium |
| 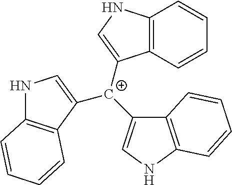 [SMILES: c1ccc2c([C+](c3c[nH]c4ccccc34)c3c[nH]c4ccccc34)c[nH]c2c1] | tris(1H-indol-3-yl)methylium |
| 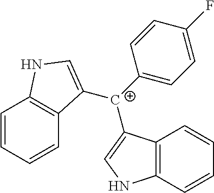 [SMILES: Fc1ccc([C+](c2c[nH]c3ccccc23)c2c[nH]c3ccccc23)cc1] | bis(1H-indol-3-yl)(4-fluorophenyl)methylium |
| 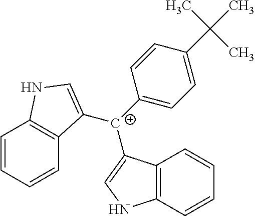 [SMILES: CC(C)(C)c1ccc([C+](c2c[nH]c3ccccc23)c2c[nH]c3ccccc23)cc1] | bis(1H-indol-3-yl)(4-tert-butylphenyl)methylium |
| 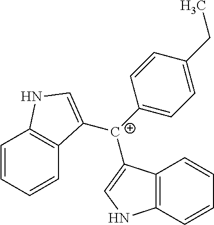 [SMILES: CCc1ccc([C+](c2c[nH]c3ccccc23)c2c[nH]c3ccccc23)cc1] | bis(1H-indol-3-yl)(4-ethylphenyl)methylium |
| 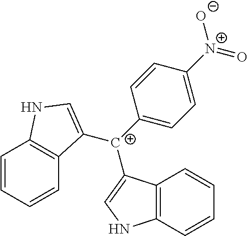 [SMILES: O=[N+]([O-])c1ccc([C+](c2c[nH]c3ccccc23)c2c[nH]c3ccccc23)cc1] | bis(1H-indol-3-yl)(4-nitrophenyl)methylium |
| 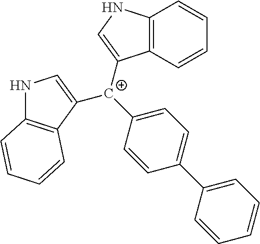 [SMILES: c1ccc(-c2ccc([C+](c3c[nH]c4ccccc34)c3c[nH]c4ccccc34)cc2)cc1] | bis(1H-indol-3-yl)((1,1′-biphenyl)-4-yl)methylium |
| 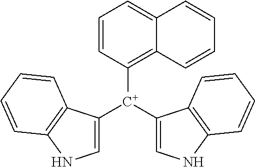 [SMILES: c1ccc2c([C+](c3c[nH]c4ccccc34)c3c[nH]c4ccccc34)cccc2c1] | bis(1H-indol-3-yl)(naphthalen-1-yl)methylium |
| 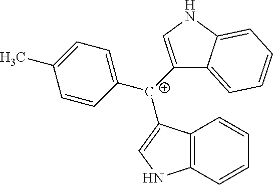 [SMILES: Cc1ccc([C+](c2c[nH]c3ccccc23)c2c[nH]c3ccccc23)cc1] | bis(1H-indol-3-yl)(4-methylphenyl)methylium |
| 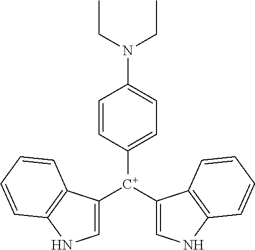 [SMILES: CCN(CC)c1ccc([C+](c2c[nH]c3ccccc23)c2c[nH]c3ccccc23)cc1] | bis(1H-indol-3-yl)(4-(diethylamino)phenyl)methylium |
| 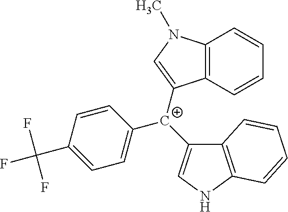 [SMILES: Cn1cc([C+](c2ccc(C(F)(F)F)cc2)c2c[nH]c3ccccc23)c2ccccc21] | (1-methyl-1H-indol-3-yl)(1H-indol-3-yl)(4- (trifluoromethyl)phenyl)methylium |
| 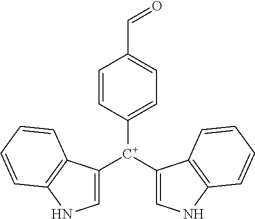 [SMILES: O=Cc1ccc([C+](c2c[nH]c3ccccc23)c2c[nH]c3ccccc23)cc1] | bis(1H-indol-3-yl)(4-formylphenyl)methylium |
| 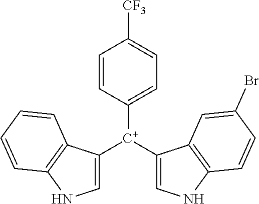 [SMILES: FC(F)(F)c1ccc([C+](c2c[nH]c3ccccc23)c2c[nH]c3ccc(Br)cc23)cc1] | (5-bromo-1H-indol-3-yl)(1H-indol-3-yl)(4- (trifluoromethyl)phenyl)methylium |
| 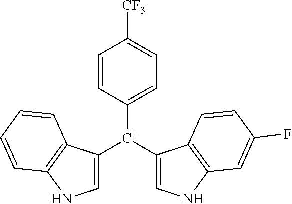 [SMILES: Fc1ccc2c([C+](c3ccc(C(F)(F)F)cc3)c3c[nH]c4ccccc34)c[nH]c2c1] | (6-fluoro-1H-indol-3-yl)(1H-indol-3-yl)(4- (trifluoromethyl)phenyl)methylium |
| 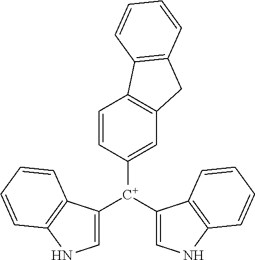 [SMILES: c1ccc2c(c1)Cc1cc([C+](c3c[nH]c4ccccc34)c3c[nH]c4ccccc34)ccc1-2] | bis(1H-indol-3-yl)(9H-fluoren-2-yl)methylium |
| 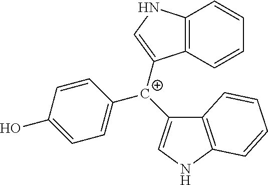 [SMILES: Oc1ccc([C+](c2c[nH]c3ccccc23)c2c[nH]c3ccccc23)cc1] | bis(1H-indol-3-yl)(4-hydroxyphenyl)methylium |
| 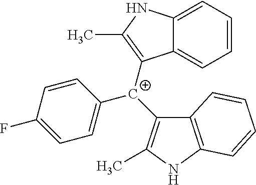 [SMILES: Cc1[nH]c2ccccc2c1[C+](c1ccc(F)cc1)c1c(C)[nH]c2ccccc12] | bis(2-methyl-1H-indol-3-yl)(4-fluorophenyl)methylium |
| 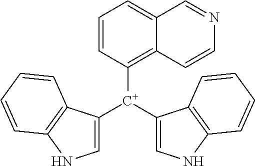 [SMILES: c1cc([C+](c2c[nH]c3ccccc23)c2c[nH]c3ccccc23)c2ccncc2c1] | bis(1H-indol-3-yl)(isoquinolin-5-yl)methylium |
| 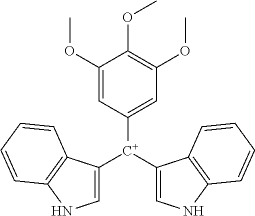 [SMILES: COc1cc([C+](c2c[nH]c3ccccc23)c2c[nH]c3ccccc23)cc(OC)c1OC] | bis(1H-indol-3-yl)(3,4,5-trimethoxyphenyl)methylium |
| 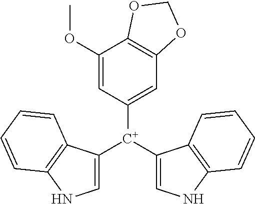 [SMILES: COc1cc([C+](c2c[nH]c3ccccc23)c2c[nH]c3ccccc23)cc2c1OCO2] | bis(1H-indol-3-yl)(7-methoxybenzo[d][1,3]dioxol-5- yl)methylium |
| 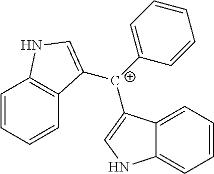 [SMILES: c1ccc([C+](c2c[nH]c3ccccc23)c2c[nH]c3ccccc23)cc1] | bis(1H-indol-3-yl)(phenyl)methylium |
| 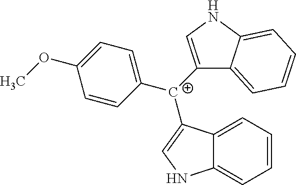 [SMILES: COc1ccc([C+](c2c[nH]c3ccccc23)c2c[nH]c3ccccc23)cc1] | bis(1H-indol-3-yl)(4-methoxyphenyl)methylium |
| 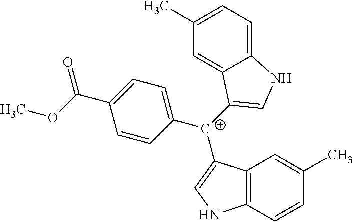 | bis(5-methyl-1H-indol-3-yl)(4- (methoxycarbonyl)phenyl)methylium |
| 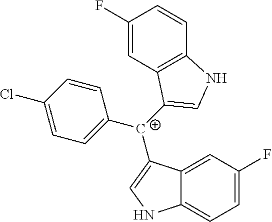 [SMILES: Fc1ccc2[nH]cc([C+](c3ccc(Cl)cc3)c3c[nH]c4ccc(F)cc34)c2c1] | bis(5-fluoro-1H-indol-3-yl)(4-chlorophenyl)methylium |
| 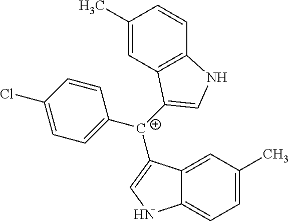 [SMILES: Cc1ccc2[nH]cc([C+](c3ccc(Cl)cc3)c3c[nH]c4ccc(C)cc34)c2c1] | bis(5-methyl-1H-indol-3-yl)(4-chlorophenyl)methylium |
| 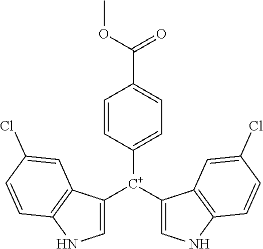 [SMILES: COC(=O)c1ccc([C+](c2c[nH]c3ccc(Cl)cc23)c2c[nH]c3ccc(Cl)cc23)cc1] | bis(5-chloro-1H-indol-3-yl)(4- (methoxycarbonyl)phenyl)methylium |
| 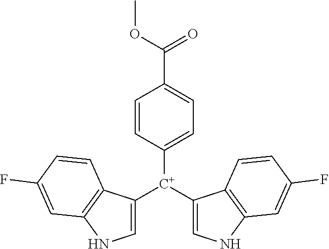 [SMILES: COC(=O)c1ccc([C+](c2c[nH]c3cc(F)ccc23)c2c[nH]c3cc(F)ccc23)cc1] | bis(6-fluoro-1H-indol-3-yl)(4- (methoxycarbonyl)phenyl)methylium |
| 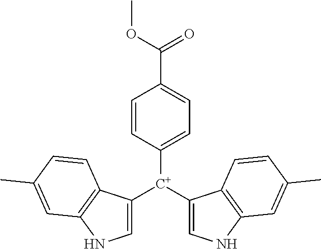 | bis(6-methyl-1H-indol-3-yl)(4- (methoxycarbonyl)phenyl)methylium |
| 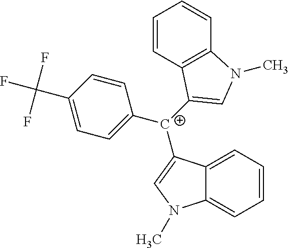 [SMILES: Cn1cc([C+](c2ccc(C(F)(F)F)cc2)c2cn(C)c3ccccc23)c2ccccc21] | bis(1-methyl-1H-indol-3-yl)(4- (trifluoromethyl)phenyl)methylium |
| 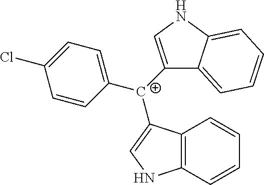 [SMILES: Clc1ccc([C+](c2c[nH]c3ccccc23)c2c[nH]c3ccccc23)cc1] | bis(1H-indol-3-yl)(4-chlorophenyl)methylium |
| 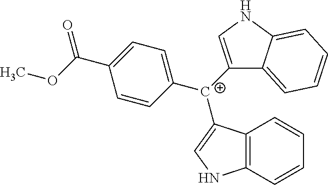 [SMILES: COC(=O)c1ccc([C+](c2c[nH]c3ccccc23)c2c[nH]c3ccccc23)cc1] | bis(1H-indol-3-yl)(4- (methoxycarbonyl)phenyl)methylium |
| 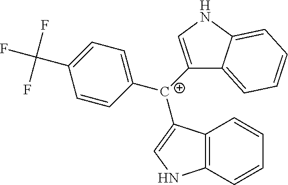 [SMILES: FC(F)(F)c1ccc([C+](c2c[nH]c3ccccc23)c2c[nH]c3ccccc23)cc1] | bis(1H-indol-3-yl)(4- (trifluoromethyl)phenyl)methylium |
| 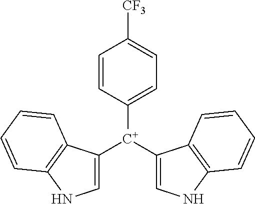 [SMILES: FC(F)(F)c1ccc([C+](c2c[nH]c3ccccc23)c2c[nH]c3ccccc23)cc1] | bis(1H-indol-3-yl)(4- (trifluoromethyl)phenyl)methylium |
| 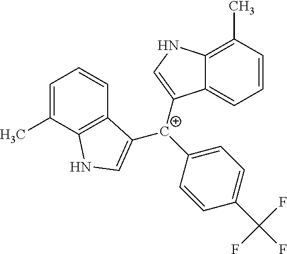 [SMILES: Cc1cccc2c([C+](c3ccc(C(F)(F)F)cc3)c3c[nH]c4c(C)cccc34)c[nH]c12] | bis(7-methyl-1H-indol-3-yl)(4- (trifluoromethyl)phenyl)methylium |
| 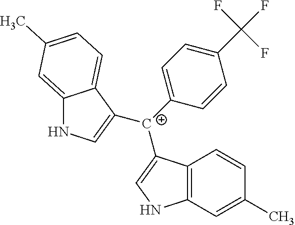 | bis(6-methyl-1H-indol-3-yl)(4- (trifluoromethyl)phenyl)methylium |
| 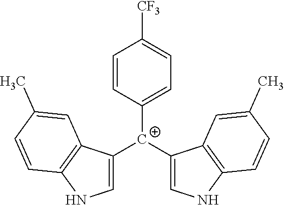 [SMILES: Cc1ccc2[nH]cc([C+](c3ccc(C(F)(F)F)cc3)c3c[nH]c4ccc(C)cc34)c2c1] | bis(5-methyl-1H-indol-3-yl)(4- (trifluoromethyl)phenyl)methylium |
| 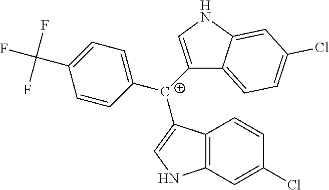 [SMILES: FC(F)(F)c1ccc([C+](c2c[nH]c3cc(Cl)ccc23)c2c[nH]c3cc(Cl)ccc23)cc1] | bis(6-chloro-1H-indol-3-yl)(4- (trifluoromethyl)phenyl)methylium |
| 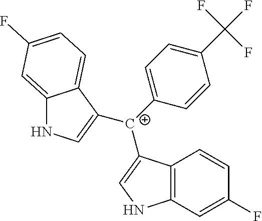 [SMILES: Fc1ccc2c([C+](c3ccc(C(F)(F)F)cc3)c3c[nH]c4cc(F)ccc34)c[nH]c2c1] | bis(6-fluoro-1H-indol-3-yl)(4- (trifluoromethyl)phenyl)methylium |
| 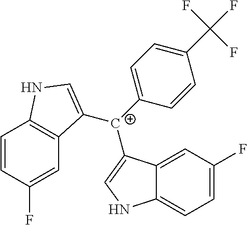 [SMILES: Fc1ccc2[nH]cc([C+](c3ccc(C(F)(F)F)cc3)c3c[nH]c4ccc(F)cc34)c2c1] | bis(5-fluoro-1H-indol-3-yl)(4- (trifluoromethyl)phenyl)methylium |
| 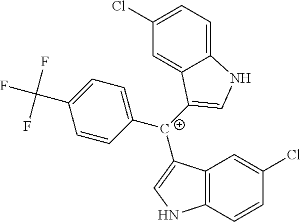 [SMILES: FC(F)(F)c1ccc([C+](c2c[nH]c3ccc(Cl)cc23)c2c[nH]c3ccc(Cl)cc23)cc1] | bis(5-chloro-1H-indol-3-yl)(4- (trifluoromethyl)phenyl)methylium |
| 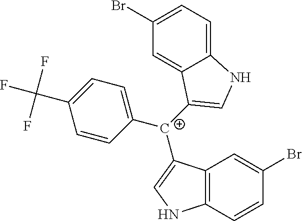 [SMILES: FC(F)(F)c1ccc([C+](c2c[nH]c3ccc(Br)cc23)c2c[nH]c3ccc(Br)cc23)cc1] | bis(5-bromo-1H-indol-3-yl)(4- (trifluoromethyl)phenyl)methylium |
| 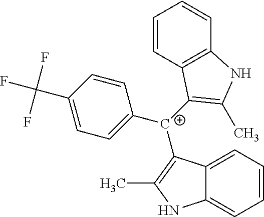 [SMILES: Cc1[nH]c2ccccc2c1[C+](c1ccc(C(F)(F)F)cc1)c1c(C)[nH]c2ccccc12] | bis(2-methyl-1H-indol-3-yl)(4- (trifluoromethyl)phenyl)methylium |
| 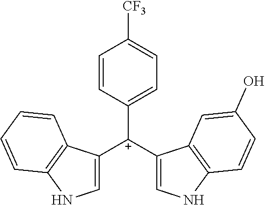 [SMILES: Oc1ccc2[nH]cc([C+](c3ccc(C(F)(F)F)cc3)c3c[nH]c4ccccc34)c2c1] | (5-hydroxy-1H-indol-3-yl)(1H-indol-3-yl)(4- (trifluoromethyl)phenyl)methylium |
| 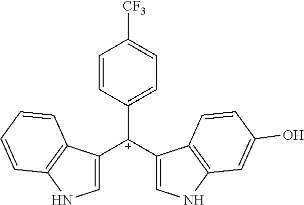 [SMILES: Oc1ccc2c([C+](c3ccc(C(F)(F)F)cc3)c3c[nH]c4ccccc34)c[nH]c2c1] | (6-hydroxy-1H-indol-3-yl)(1H-indol-3-yl)(4- (trifluoromethyl)phenyl)methylium |
| 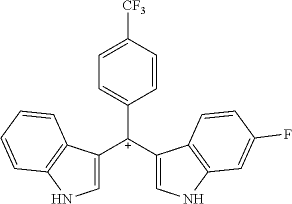 [SMILES: Fc1ccc2c([C+](c3ccc(C(F)(F)F)cc3)c3c[nH]c4ccccc34)c[nH]c2c1] | (6-fluoro-1H-indol-3-yl)(1H-indol-3-yl)(4- (trifluoromethyl)phenyl)methylium |
| 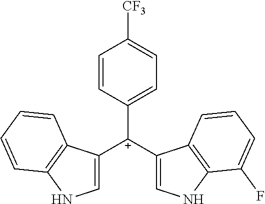 [SMILES: Fc1cccc2c([C+](c3ccc(C(F)(F)F)cc3)c3c[nH]c4ccccc34)c[nH]c12] | (7-fluoro-1H-indol-3-yl)(1H-indol-3-yl)(4- (trifluoromethyl)phenyl)methylium |
| 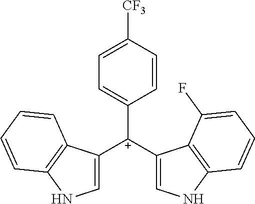 [SMILES: Fc1cccc2[nH]cc([C+](c3ccc(C(F)(F)F)cc3)c3c[nH]c4ccccc34)c12] | (4-fluoro-1H-indol-3-yl)(1H-indol-3-yl)(4- (trifluoromethyl)phenyl)methylium |
| 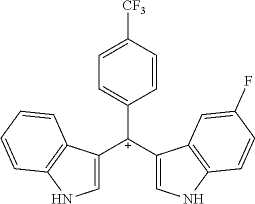 [SMILES: Fc1ccc2[nH]cc([C+](c3ccc(C(F)(F)F)cc3)c3c[nH]c4ccccc34)c2c1] | (5-fluoro-1H-indol-3-yl)(1H-indol-3-yl)(4- (trifluoromethyl)phenyl)methylium |
| 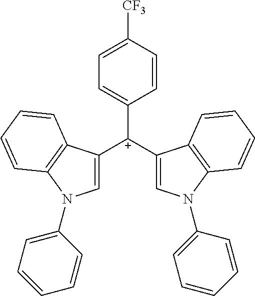 [SMILES: FC(F)(F)c1ccc([C+](c2cn(-c3ccccc3)c3ccccc23)c2cn(-c3ccccc3)c3ccccc23)cc1] | bis(1-phenyl-1H-indol-3-yl)(4- (trifluoromethyl)phenyl)methylium |
| 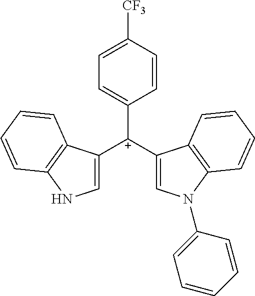 [SMILES: FC(F)(F)c1ccc([C+](c2c[nH]c3ccccc23)c2cn(-c3ccccc3)c3ccccc23)cc1] | (1H-indol-3-yl)(1-phenyl-1H-indol-3-yl)(4- (trifluoromethyl)phenyl)methylium |
| 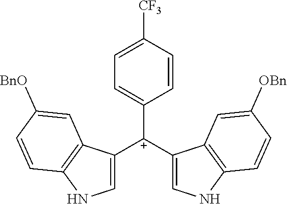 [SMILES: FC(F)(F)c1ccc([C+](c2c[nH]c3ccc(OCc4ccccc4)cc23)c2c[nH]c3ccc(OCc4ccccc4)cc23)cc1] | bis(5-(benzyloxy)-1H-indol-3-yl)(4- (trifluoromethyl)phenyl)methylium |
| 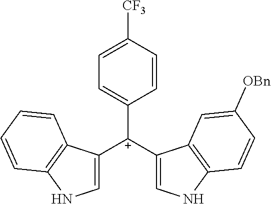 [SMILES: FC(F)(F)c1ccc([C+](c2c[nH]c3ccccc23)c2c[nH]c3ccc(OCc4ccccc4)cc23)cc1] | (5-(benzyloxy)-1H-indol-3-yl)(1H-indol-3-yl)(4- (trifluoromethyl)phenyl)methylium |
| 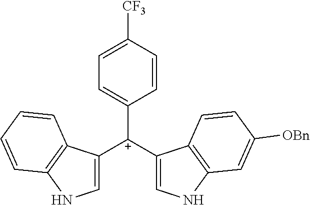 [SMILES: FC(F)(F)c1ccc([C+](c2c[nH]c3ccccc23)c2c[nH]c3cc(OCc4ccccc4)ccc23)cc1] | (6-(benzyloxy)-1H-indol-3-yl)(1H-indol-3-yl)(4- (trifluoromethyl)phenyl)methylium |
| 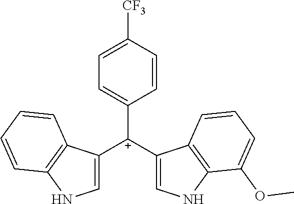 [SMILES: COc1cccc2c([C+](c3ccc(C(F)(F)F)cc3)c3c[nH]c4ccccc34)c[nH]c12] | (1H-indol-3-yl)(7-methoxy-1H-indol-3-yl)(4- (trifluoromethyl)phenyl)methylium |
| 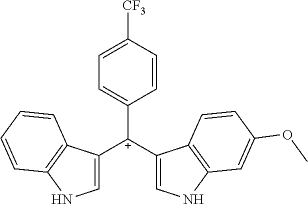 [SMILES: COc1ccc2c([C+](c3ccc(C(F)(F)F)cc3)c3c[nH]c4ccccc34)c[nH]c2c1] | (1H-indol-3-yl)(6-methoxy-1H-indol-3-yl)(4- (trifluoromethyl)phenyl)methylium |
| 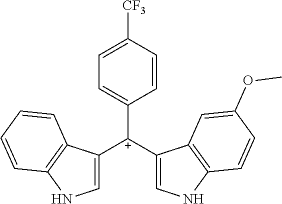 [SMILES: COc1ccc2[nH]cc([C+](c3ccc(C(F)(F)F)cc3)c3c[nH]c4ccccc34)c2c1] | (1H-indol-3-yl)(5-methoxy-1H-indol-3-yl)(4- (trifluoromethyl)phenyl)methylium |
| 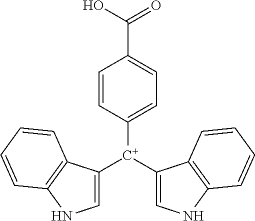 [SMILES: O=C(O)c1ccc([C+](c2c[nH]c3ccccc23)c2c[nH]c3ccccc23)cc1] | bis(1H-indol-3-yl)(4-carboxyphenyl)methylium |
| 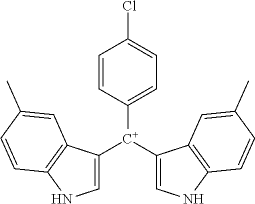 [SMILES: Cc1ccc2[nH]cc([C+](c3ccc(Cl)cc3)c3c[nH]c4ccc(C)cc34)c2c1] | bis(5-methyl-1H-indol-3-yl)(4-chlorophenyl)methylium |
| 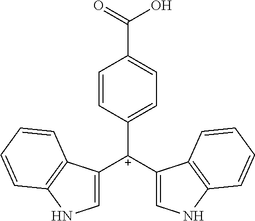 [SMILES: O=C(O)c1ccc([C+](c2c[nH]c3ccccc23)c2c[nH]c3ccccc23)cc1] | Bis(1H-indol-3-yl)(4-carboxyphenyl)methylium |
| 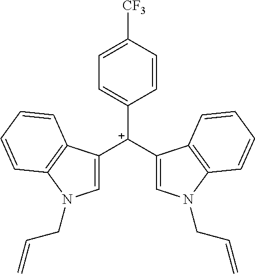 [SMILES: C=CCn1cc([C+](c2ccc(C(F)(F)F)cc2)c2cn(CC=C)c3ccccc23)c2ccccc21] | bis(1-allyl-1H-indol-3-yl)(4- (trifluoromethyl)phenyl)methylium |
| 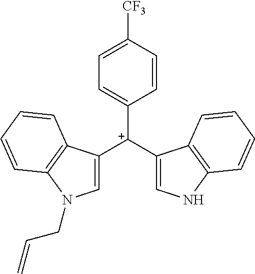 [SMILES: C=CCn1cc([C+](c2ccc(C(F)(F)F)cc2)c2c[nH]c3ccccc23)c2ccccc21] | (1-allyl-1H-indol-3-yl)(1H-indol-3-yl)(4- (trifluoromethyl)phenyl)methylium |
| 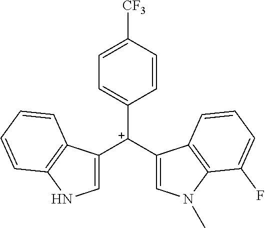 [SMILES: Cn1cc([C+](c2ccc(C(F)(F)F)cc2)c2c[nH]c3ccccc23)c2cccc(F)c21] | (7-fluoro-1-methyl-1H-indol-3-yl)(1H-indol-3-yl)(4- (trifluoromethyl)phenyl)-methylium |
| 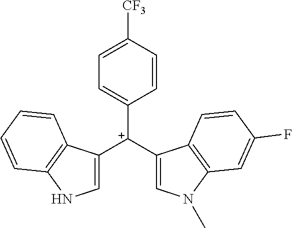 [SMILES: Cn1cc([C+](c2ccc(C(F)(F)F)cc2)c2c[nH]c3ccccc23)c2ccc(F)cc21] | (6-fluoro-1-methyl-1H-indol-3-yl)(1H-indol-3-yl)(4- (trifluoromethyl)phenyl)-methylium |
| 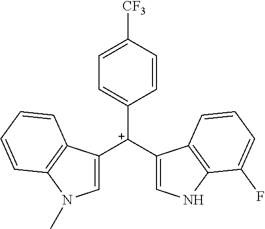 [SMILES: Cn1cc([C+](c2ccc(C(F)(F)F)cc2)c2c[nH]c3c(F)cccc23)c2ccccc21] | (7-fluoro-1H-indol-3-yl)(1-methyl-1H-indol-3-yl)(4- (trifluoromethyl)phenyl)-methylium |
| 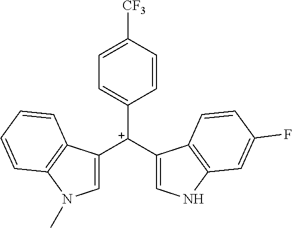 [SMILES: Cn1cc([C+](c2ccc(C(F)(F)F)cc2)c2c[nH]c3cc(F)ccc23)c2ccccc21] | (6-fluoro-1H-indol-3-yl)(1-methyl-1H-indol-3-yl)(4- (trifluoromethyl)phenyl)-methylium |
| 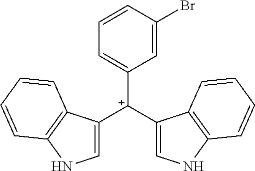 [SMILES: Brc1cccc([C+](c2c[nH]c3ccccc23)c2c[nH]c3ccccc23)c1] | (3-bromophenyl)di(1H-indol-3-yl)methylium |
| 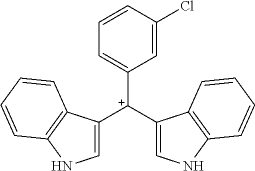 [SMILES: Clc1cccc([C+](c2c[nH]c3ccccc23)c2c[nH]c3ccccc23)c1] | (3-chlorophenyl)di(1H-indol-3-yl)methylium |
| 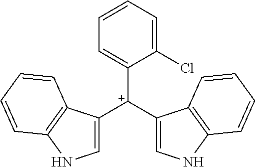 [SMILES: Clc1ccccc1[C+](c1c[nH]c2ccccc12)c1c[nH]c2ccccc12] | (2-chlorophenyl)di(1H-indol-3-yl)methylium |
| 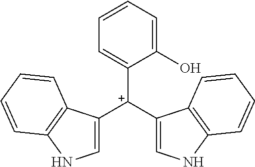 [SMILES: Oc1ccccc1[C+](c1c[nH]c2ccccc12)c1c[nH]c2ccccc12] | (2-hydroxyphenyl)di(1H-indol-3-yl)methylium |
| 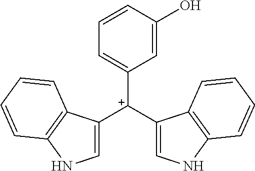 [SMILES: Oc1cccc([C+](c2c[nH]c3ccccc23)c2c[nH]c3ccccc23)c1] | (3-hydroxyphenyl)di(1H-indol-3-yl)methylium |
| 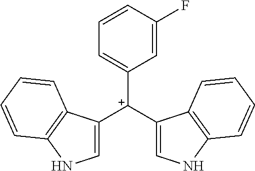 [SMILES: Fc1cccc([C+](c2c[nH]c3ccccc23)c2c[nH]c3ccccc23)c1] | (3-fluorophenyl)di(1H-indol-3-yl)methylium |
| 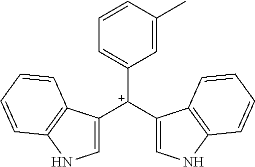 [SMILES: Cc1cccc([C+](c2c[nH]c3ccccc23)c2c[nH]c3ccccc23)c1] | di(1H-indol-3-yl)(m-tolyl)methylium |
| 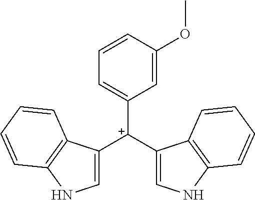 [SMILES: COc1cccc([C+](c2c[nH]c3ccccc23)c2c[nH]c3ccccc23)c1] | di(1H-indol-3-yl)(3-methoxyphenyl)methylium |
| 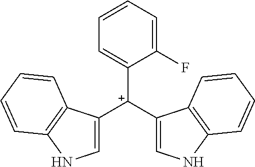 [SMILES: Fc1ccccc1[C+](c1c[nH]c2ccccc12)c1c[nH]c2ccccc12] | (2-fluorophenyl)di(1H-indol-3-yl)methylium |
| 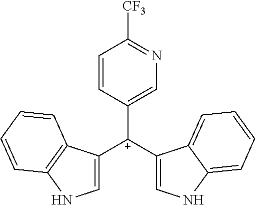 [SMILES: FC(F)(F)c1ccc([C+](c2c[nH]c3ccccc23)c2c[nH]c3ccccc23)cn1] | Di(1H-indol-3-yl)(6-(trifluoromethyl)pyridin-3- yl)methylium |
| 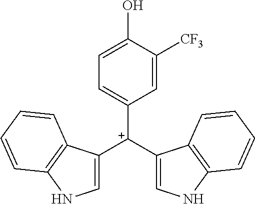 [SMILES: Oc1ccc([C+](c2c[nH]c3ccccc23)c2c[nH]c3ccccc23)cc1C(F)(F)F] | (4-hydroxy-3-(trifluoromethyl)phenyl)di(1H-indol-3- yl)methylium |
| 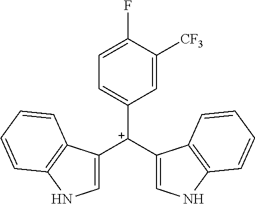 [SMILES: Fc1ccc([C+](c2c[nH]c3ccccc23)c2c[nH]c3ccccc23)cc1C(F)(F)F] | (4-fluoro-3-(trifluoromethyl)phenyl)di(1H-indol-3- yl)methylium |
| 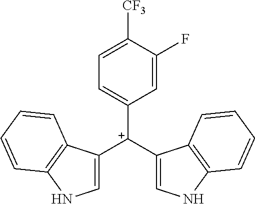 [SMILES: Fc1cc([C+](c2c[nH]c3ccccc23)c2c[nH]c3ccccc23)ccc1C(F)(F)F] | (3-fluoro-4-(trifluoromethyl)phenyl)di(1H-indol-3- yl)methylium |
| 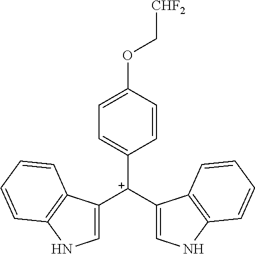 [SMILES: FC(F)COc1ccc([C+](c2c[nH]c3ccccc23)c2c[nH]c3ccccc23)cc1] | (4-(2,2-difluoroethoxy)phenyl)di(1H-indol-3- yl)methylium |
| 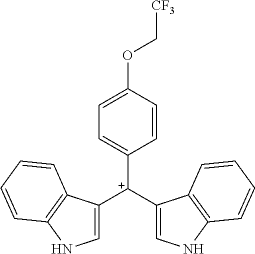 [SMILES: FC(F)(F)COc1ccc([C+](c2c[nH]c3ccccc23)c2c[nH]c3ccccc23)cc1] | di(1H-indol-3-yl)(4-(2,2,2- trifluoroethoxy)phenyl)methylium |
| 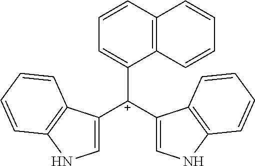 [SMILES: c1ccc2c([C+](c3c[nH]c4ccccc34)c3c[nH]c4ccccc34)cccc2c1] | Di(1H-indol-3-yl)(naphthalen-1-yl)methylium |
| 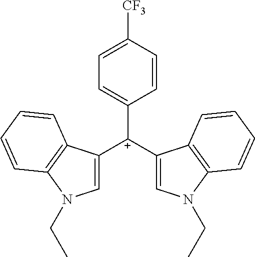 [SMILES: CCn1cc([C+](c2ccc(C(F)(F)F)cc2)c2cn(CC)c3ccccc23)c2ccccc21] | Bis(1-ethyl-1H-indol-3-yl)(4- (trifluoromethyl)phenyl)methylium |
| 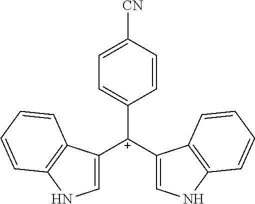 [SMILES: N#Cc1ccc([C+](c2c[nH]c3ccccc23)c2c[nH]c3ccccc23)cc1] | (4-Cyanophenyl)di(1H-indol-3-yl)methylium |
| 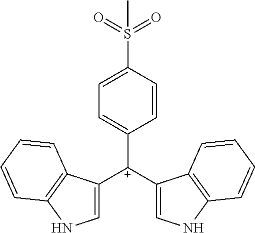 [SMILES: CS(=O)(=O)c1ccc([C+](c2c[nH]c3ccccc23)c2c[nH]c3ccccc23)cc1] | Di(1H-indol-3-yl)(4-(methylsulfonyl)phenyl)methylium |

In some embodiments, compounds of Formula (I) are synthesized from the corresponding unoxidized C-substituted DIM derivatives, as demonstrated in the examples and schemes disclosed herein. In some embodiments, the corresponding C-substituted DIM derivatives are treated with an acid. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is an inorganic acid such as hydrochloric acid, hydrobromic acid, or sulfuric acid. In some embodiments, the acid is an organic acid, such as methanesulfonic acid, trifluoromethanesulfonic acid, benzene sulfonic acid, p-toluenesulfonic acid, acetic acid, trifluoroacetic acid, or benzoic acid. In some embodiments, the acid is an organic acid such as methanesulfonic acid or p-toluenesulfonic acid. In some embodiments, the corresponding C-substituted DIM derivatives are treated with a metal or organic oxidant. In some embodiments, the oxidant is an organic oxidant such as Ph₃C⁺BF₄⁻ and the like. In other embodiments, the oxidant is a metal based oxidant such as pyridinium dichromate, pyridinium chlorochromate, K₂Cr₂O₇, FeCl₃, and the like.

C-substituted DIM derivatives that can be oxidized to yield compounds of Formula (I) include known C-substituted DIM derivatives such as those disclosed in U.S. Pat. Nos. 7,232,843, 7,709,520, 8,148,547, 8,389,563, and 8,580,843.

In some embodiments, C-substituted DIM derivatives that can be oxidized to yield compounds of Formula (I) include, but are not limited to, those in Table 3.

TABLE 3

| Structure | Name |
|---|---|
| 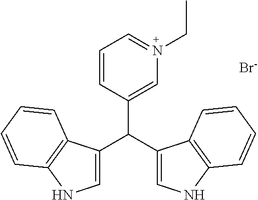 | 3-[bis(1H-indol-3-yl)methyl]-1- ethylpyridin-1-ium bromide |
| 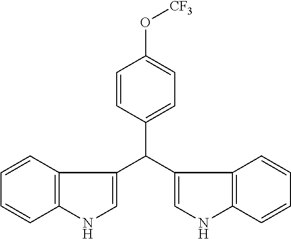 [SMILES: FC(F)(F)Oc1ccc(C(c2c[nH]c3ccccc23)c2c[nH]c3ccccc23)cc1] | 3-[(1H-indol-3-yl)[4- (trifluoromethoxy)phenyl]methyl]-1H- indole |
| 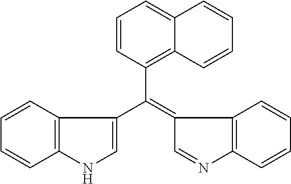 [SMILES: C1=Nc2ccccc2/C1=C(\c1cccc2ccccc12)c1c[nH]c2ccccc12] | (3Z)-3-[(1H-indol-3-yl)(naphalen-1- yl)methylidene]-3H-indole |
| 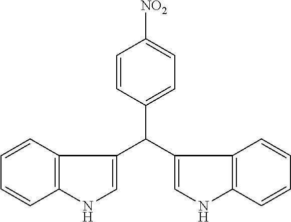 [SMILES: O=[N+]([O-])c1ccc(C(c2c[nH]c3ccccc23)c2c[nH]c3ccccc23)cc1] | 3-[(1H-indol-3-yl)(4- nitrophenyl)methyl]-1H-indole |
| 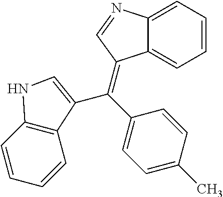 [SMILES: Cc1ccc(/C(=C2/C=Nc3ccccc32)c2c[nH]c3ccccc23)cc1] | (3Z)-3-[(1H-indol-3-yl)(4- methylphenyl)methylidene]-3H-indole |
| 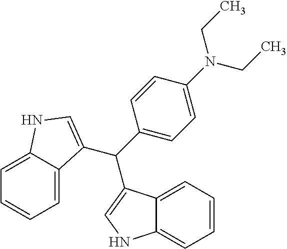 [SMILES: CCN(CC)c1ccc(C(c2c[nH]c3ccccc23)c2c[nH]c3ccccc23)cc1] | 4-[bis(1H-indol-3-yl)methyl]-N,N- diethylaniline |
| 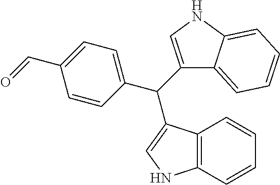 [SMILES: O=Cc1ccc(C(c2c[nH]c3ccccc23)c2c[nH]c3ccccc23)cc1] | 4-[bis(1H-indol-3- yl)methyl]benzaldehyde |
| 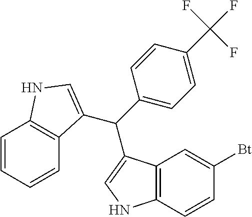 | 5-bromo-3-[(1H-indol-3-yl)[4- (trifluoromethyl)phenyl]methyl]-1H- indole |
| 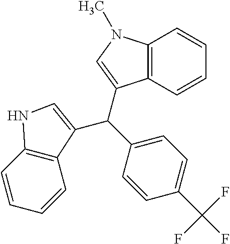 [SMILES: Cn1cc(C(c2ccc(C(F)(F)F)cc2)c2c[nH]c3ccccc23)c2ccccc21] | 3-[(1H-indol-3-yl)[4- (trifluoromethyl)phenyl]methyl]-1- methyl-1H-indole |
| 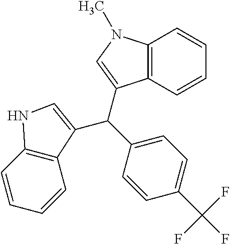 [SMILES: Cn1cc(C(c2ccc(C(F)(F)F)cc2)c2c[nH]c3ccccc23)c2ccccc21] | 3-[(1H-indole-3-yl[4- (trifluoromethyl)phenyl]methyl]-1- methyl-1H-indole |
| 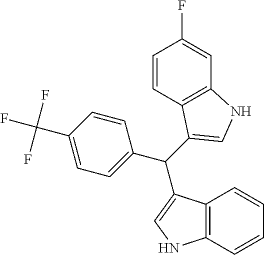 [SMILES: Fc1ccc2c(C(c3ccc(C(F)(F)F)cc3)c3c[nH]c4ccccc34)c[nH]c2c1] | 6-fluoro-3-[(1H-indol-3-yl)[4- (trifluromethyl)phenyl]-methyl]-1H- indole |
| 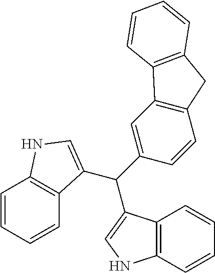 [SMILES: c1ccc2c(c1)Cc1ccc(C(c3c[nH]c4ccccc34)c3c[nH]c4ccccc34)cc1-2] | 3-[(9H-fluoren-3-yl)(1H-indol-3- yl)methyl]-1H-indole |
| 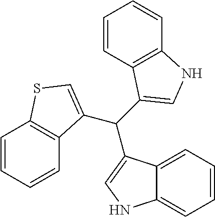 [SMILES: c1ccc2c(C(c3c[nH]c4ccccc34)c3csc4ccccc34)c[nH]c2c1] | 3-[(1-benzothiophen-3-yl)(1H-indol-3- yl)methyl]-1H-indole |
| 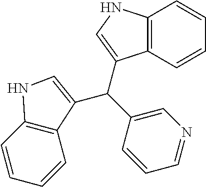 [SMILES: c1cncc(C(c2c[nH]c3ccccc23)c2c[nH]c3ccccc23)c1] | 3-[(1H-indol-3-yl)(pyridin-3- yl)methyl]-1H-indole |
| 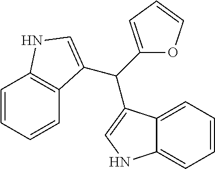 [SMILES: c1coc(C(c2c[nH]c3ccccc23)c2c[nH]c3ccccc23)c1] | 3-[(furan-2-yl)(1H-indol-3-yl)methyl]- 1H-indole |
| 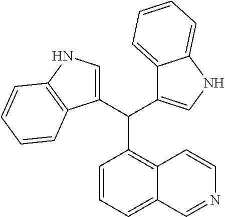 [SMILES: c1cc(C(c2c[nH]c3ccccc23)c2c[nH]c3ccccc23)c2ccncc2c1] | 5-[bis(1H-indol-3- yl)methyl]isoquinazoline |
| 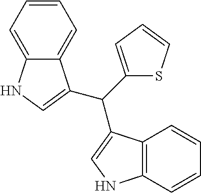 [SMILES: c1csc(C(c2c[nH]c3ccccc23)c2c[nH]c3ccccc23)c1] | 3-[(1H-indol-3-yl)(thiophen-2- yl)methyl]-1H-indole |
| 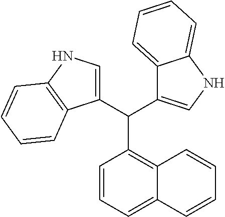 [SMILES: c1ccc2c(C(c3c[nH]c4ccccc34)c3c[nH]c4ccccc34)cccc2c1] | 3-[(1H-indol-3-yl)(naphthalen-1- yl)methyl]-1H-indole |
| 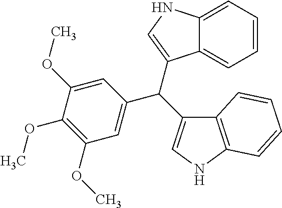 [SMILES: COc1cc(C(c2c[nH]c3ccccc23)c2c[nH]c3ccccc23)cc(OC)c1OC] | 3-[(1H-indol-3-yl)(3,4,5- trimethoxyphenyl)methyl]-1H-indole |
| 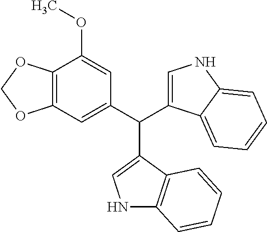 [SMILES: COc1cc(C(c2c[nH]c3ccccc23)c2c[nH]c3ccccc23)cc2c1OCO2] | 3-[(1H-indol-3-yl)(7-methoxy-2H-1,3- benzodioxol-5-yl)methyl]-1H-indole |
| 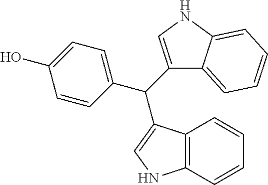 [SMILES: Oc1ccc(C(c2c[nH]c3ccccc23)c2c[nH]c3ccccc23)cc1] | 4-[bis(1H-indol-3-yl)methyl]phenol |
| 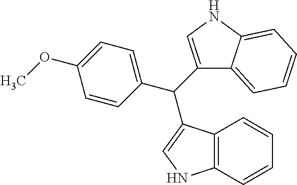 [SMILES: COc1ccc(C(c2c[nH]c3ccccc23)c2c[nH]c3ccccc23)cc1] | 3-[(1H-indol-3-yl)(4- methoxyphenyl)methyl]-1H-indole |
| 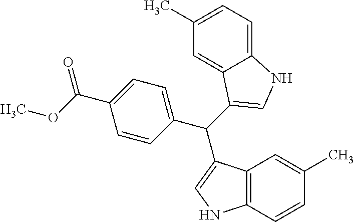 | methyl 4-[bis(5-methyl-1H-indol-3- yl)methyl]benzoate |
| 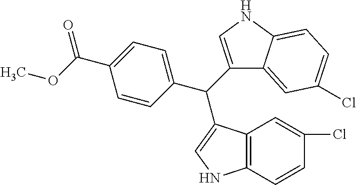 [SMILES: COC(=O)c1ccc(C(c2c[nH]c3ccc(Cl)cc23)c2c[nH]c3ccc(Cl)cc23)cc1] | methyl 4-[bis(5-chloro-1H-indol-3- yl)methyl]benzoate |
| 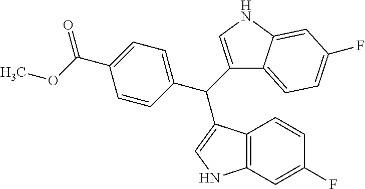 [SMILES: COC(=O)c1ccc(C(c2c[nH]c3cc(F)ccc23)c2c[nH]c3cc(F)ccc23)cc1] | methyl 4-[bis(6-fluoro-1H-indol-3- yl)methyl]benzoate |
| 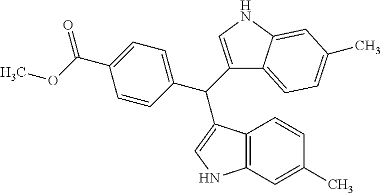 | methyl 4-[bis(6-methyl-1H-indol-3- yl)methyl]benzoate |
| 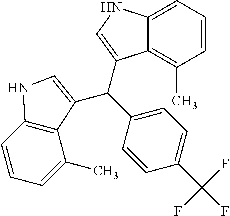 [SMILES: Cc1cccc2[nH]cc(C(c3ccc(C(F)(F)F)cc3)c3c[nH]c4cccc(C)c34)c12] | 4-methyl-3-[(4-methyl-1H-indol-3- yl)[4-(trifluoromethyl)phenyl]methyl]- 1H-indole |
| 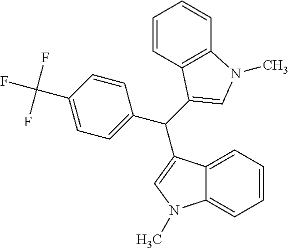 [SMILES: Cn1cc(C(c2ccc(C(F)(F)F)cc2)c2cn(C)c3ccccc23)c2ccccc21] | 1-methyl-3-[(1-methyl-1H-indol-3- yl)[4-(trifluoromethyl)phenyl]methyl]- 1H-indole |
| 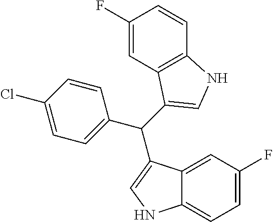 [SMILES: Fc1ccc2[nH]cc(C(c3ccc(Cl)cc3)c3c[nH]c4ccc(F)cc34)c2c1] | 3-[(4-chlorophenyl)(5-fluoro-1H-indol- 3-yl)methyl]-5-fluoro-1H-indole |
| 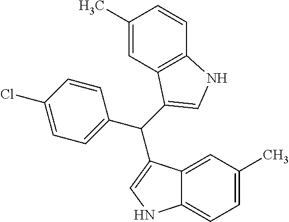 [SMILES: Cc1ccc2[nH]cc(C(c3ccc(Cl)cc3)c3c[nH]c4ccc(C)cc34)c2c1] | 3-[(4-chlorophenyl)(5-methyl-1H-indol- 3-yl)methyl]-5-methyl-1H-indole |
| 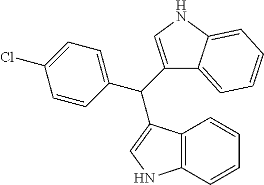 [SMILES: Clc1ccc(C(c2c[nH]c3ccccc23)c2c[nH]c3ccccc23)cc1] | 3-[(4-chlorophenyl)(1H-indol-3- yl)methyl]-1H-indole |
| 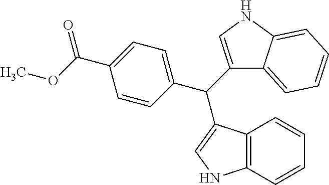 [SMILES: COC(=O)c1ccc(C(c2c[nH]c3ccccc23)c2c[nH]c3ccccc23)cc1] | methyl 4-[bis(1H-indol-3- yl)methyl]benzoate |
| 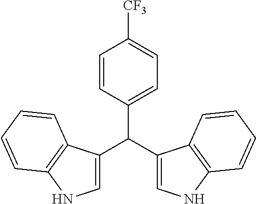 [SMILES: FC(F)(F)c1ccc(C(c2c[nH]c3ccccc23)c2c[nH]c3ccccc23)cc1] | 3,3′-((4- (trifluoromethyl)phenyl)methylene)bis(1H- indole) |
| 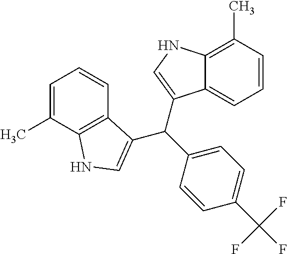 [SMILES: Cc1cccc2c(C(c3ccc(C(F)(F)F)cc3)c3c[nH]c4c(C)cccc34)c[nH]c12] | 7-methyl-3-[(7-methyl-1H-indol-3- yl)[4-(trifluoromethyl)phenyl]methyl]- 1H-indole |
| 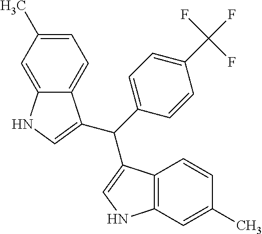 [SMILES: Cc1ccc2c(C(c3ccc(C(F)(F)F)cc3)c3c[nH]c4cc(C)ccc34)c[nH]c2c1] | 6-methyl-3-[(6-methyl-1H-indol-3- yl)[4-(trifluoromethyl)phenyl]methyl]- 1H-indole |
| 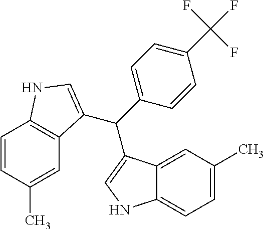 [SMILES: Cc1ccc2[nH]cc(C(c3ccc(C(F)(F)F)cc3)c3c[nH]c4ccc(C)cc34)c2c1] | 5-methyl-3-[(5-methyl-1H-indol-3- yl)[4-(trifluoromethyl)phenyl]methyl]- 1H-indole |
| 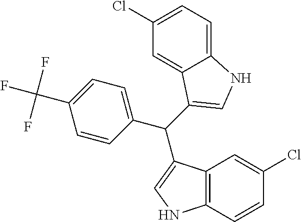 [SMILES: FC(F)(F)c1ccc(C(c2c[nH]c3ccc(Cl)cc23)c2c[nH]c3ccc(Cl)cc23)cc1] | 5-chloro-3-[(5-chloro-1H-indol-3-yl)[4- (trifluoromethyl)phenyl]-methyl]-1H- indole |
| 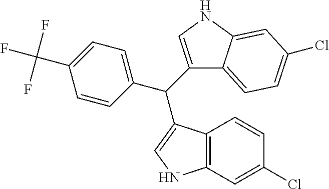 [SMILES: FC(F)(F)c1ccc(C(c2c[nH]c3cc(Cl)ccc23)c2c[nH]c3cc(Cl)ccc23)cc1] | 6-chloro-3-[(6-chloro-1H-indol-3-yl)[4- (trifluoromethyl)phenyl]methyl]-1H- indole |
| 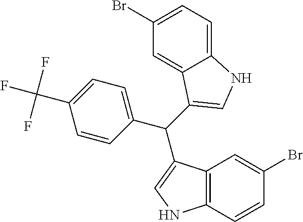 [SMILES: FC(F)(F)c1ccc(C(c2c[nH]c3ccc(Br)cc23)c2c[nH]c3ccc(Br)cc23)cc1] | 5-bromo-3-[(5-bromo-1H-indol-3-yl)[4- (trifluoromethyl)phenyl]methyl]-1H- indole |
| 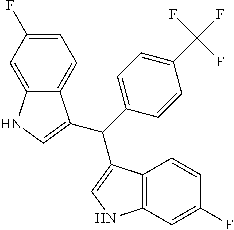 [SMILES: Fc1ccc2c(C(c3ccc(C(F)(F)F)cc3)c3c[nH]c4cc(F)ccc34)c[nH]c2c1] | 6-fluoro-3-[(6-fluoro-1H-indol-3-yl)[4- (trifluoromethyl)phenyl]methyl]-1H- indole |
| 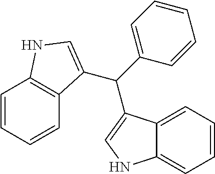 [SMILES: c1ccc(C(c2c[nH]c3ccccc23)c2c[nH]c3ccccc23)cc1] | 3-[(1H-indol-3-yl)(phenyl)methyl]-1H- indole |
| 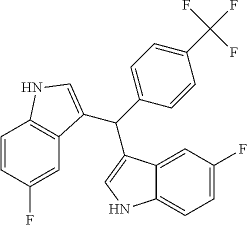 [SMILES: Fc1ccc2[nH]cc(C(c3ccc(C(F)(F)F)cc3)c3c[nH]c4ccc(F)cc34)c2c1] | 5-fluoro-3-[(5-fluoro-1H-indol-3-yl)[4- (trifluoromethyl)phenyl]methyl]-1H- indole |
| 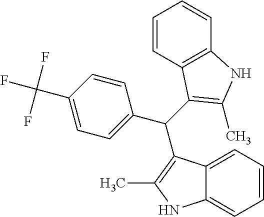 [SMILES: Cc1[nH]c2ccccc2c1C(c1ccc(C(F)(F)F)cc1)c1c(C)[nH]c2ccccc12] | 2-methyl-3-[(2-methyl-1H-indol-3- yl)[4-(trifluoromethyl)phenyl]methyl]- 1H-indole |

In some embodiments, the suitable anion is a suitable monovalent anion. In some embodiments, the suitable anion is selected from anions formed from inorganic acids and anions formed from organic acids. In some embodiments, the suitable anion is selected from halides, chlorates, sulfates, nitrates, phosphates, carboxylates, sulfonates, and borates.

### Further Forms of Compounds

In some aspects, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶Cl, and ¹²⁵I In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound disclosed herein with acids to facilitate ion exchange.

Compounds described herein may be formed as, and/or used as, pharmaceutically acceptable salts. The type of pharmaceutical acceptable salts, include, but are not limited to, acid addition salts, formed by reacting the unoxidized form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like. Further examples and experimental details for the formation of the oxidized DIM salts can be found in the methods of synthesis and examples below.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Methods of Synthesis

In some embodiments, the syntheses of compounds described herein are accomplished using means described in the chemical literature, using the methods described herein, or by a combination thereof In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

In one aspect, the compounds described herein can be synthesized as exemplified in Scheme 1.

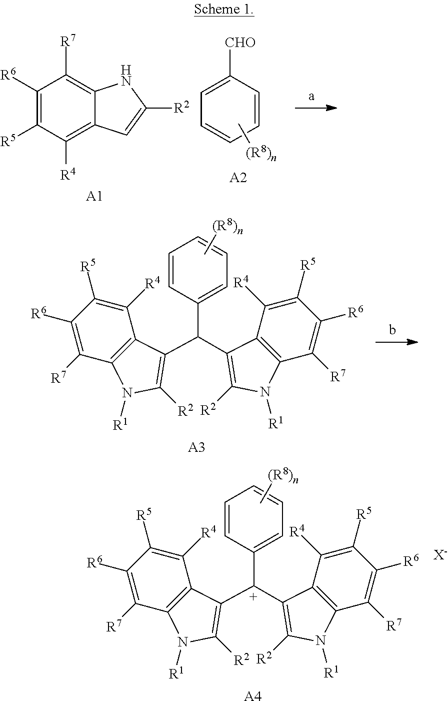

### Reagents and Conditions: (a) Lewis Acid; (b) Activated Carbon, H—X

Briefly, indole derivative A1 and benzaldehyde derivative A2 are treated with a lewis acid to yield intermediate A3. In some embodiments, the Lewis acid is a metal salt selected from aluminum salts, boron salts, cerium salts, iron salts, tin salts, titanium salts, and the like. In some embodiments, the Lewis acid is a cerium salt. In some embodiments, the Lewis acid is CeCl₃.7H₂O. In some embodiments, the Cerium salt is preactivated by treatment with NaL Intermediate A3 is then treated with an acid in the presence of activated carbon to yield oxidized DIM salt product A4. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is hydrochloric acid, hydrobromic acid, or sulfuric acid. In some embodiments, the acid is an organic acid, such as methanesulfonic acid, trifluoromethanesulfonic acid, benzene sulfonic acid, p-toluenesulfonic acid, acetic acid, trifluoroacetic acid, or benzoic acid. In some embodiments, the acid is methanesulfonic acid or p-toluenesulfonic acid.

In another aspect, the compounds described herein can be synthesized as exemplified in Scheme 2.

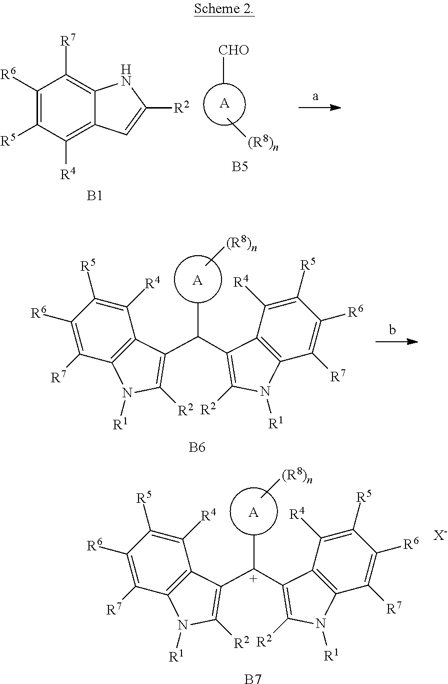

### Reagents and Conditions: (a) Acid; (b) Pyridinium Dichromate, H—X

Briefly, indole derivative Bl and aryl aldehyde derivative B5 are treated with an acid to yield intermediate B6. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is hydrochloric acid. Intermediate B6 is then treated with pyridinium dichromate in the presence of an acid to yield oxidized DIM salt product B4. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is an inorganic acid such as hydrochloric acid, hydrobromic acid, or sulfuric acid. In some embodiments, the acid is an organic acid, such as methanesulfonic acid, trifluoromethanesulfonic acid, benzene sulfonic acid, p-toluenesulfonic acid, acetic acid, trifluoroacetic acid, or benzoic acid. In some embodiments, the acid is an organic acid such as methanesulfonic acid or p-toluenesulfonic acid.

In another aspect, the compounds described herein can be synthesized as exemplified in Scheme 3.

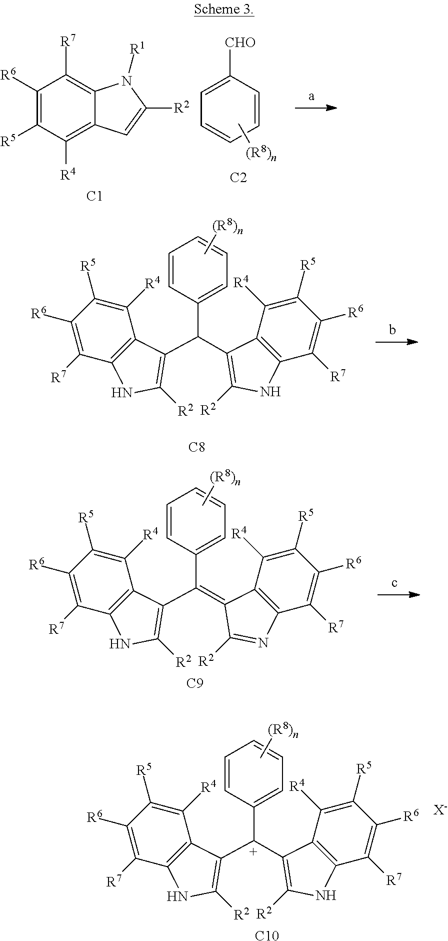

### Reagents and Conditions: (a) Acid; (b) DDQ; (c) H—X

Briefly, indole derivative C1, wherein R¹ is H, and benzaldehyde derivative C2 are treated with an acid to yield intermediate C8. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is hydrochloric acid. In some embodiments, intermediate C8 is treated with an acid to yield product C4 as in Scheme 1 above (step b). In other embodiments, intermediate C8 is treated with DDQ to yield intermediate C9. Intermediate C9 is then treated with an acid to yield oxidized DIM salt product C10. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is an inorganic acid such as hydrochloric acid, hydrobromic acid, or sulfuric acid. In some embodiments, the acid is an organic acid, such as methanesulfonic acid, trifluoromethanesulfonic acid, benzene sulfonic acid, p-toluenesulfonic acid, acetic acid, trifluoroacetic acid, or benzoic acid. In some embodiments, the acid is an organic acid such as methanesulfonic acid or p-toluenesulfonic acid.

In another aspect, in order to access asymmetrical oxidized DIM salt products, the compounds described herein can be synthesized as exemplified in Scheme 4.

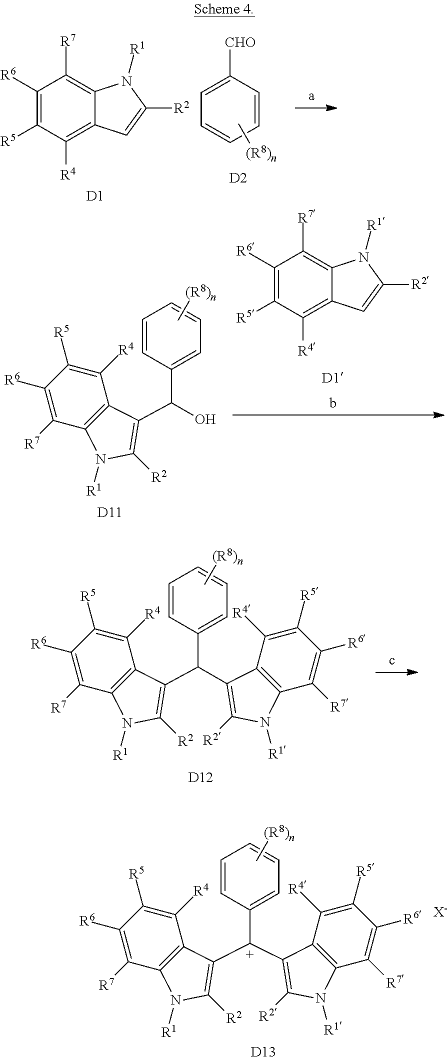

Reagents and Conditions: (a) Tetramethylguanidine; (b) 2,2,2-trifluoroethanol, Reflux; (c) Pyridinium Dichromate, H—X

Briefly, indole derivative D1 and aryl aldehyde derivative D5 are treated with tetramethylguanidine to yield intermediate D11. Intermediate D11 is then treated with a second indole derivative D1′ which yields intermediate D12. Intermediate D12 is then treated with pyridinium dichromate in the presence of an acid to yield oxidized DIM salt product D13. In some embodiments, the acid is an inorganic acid, such as hydrofluoric acid, hydrochloric acid, hydrobromic acid, hydroiodic acid, sulfuric acid, nitric acid, phosphoric acid, boric acid, or perchloric acid. In some embodiments, the acid is an inorganic acid such as hydrochloric acid, hydrobromic acid, or sulfuric acid. In some embodiments, the acid is an organic acid, such as methanesulfonic acid, trifluoromethanesulfonic acid, benzene sulfonic acid, p-toluenesulfonic acid, acetic acid, trifluoroacetic acid, or benzoic acid. In some embodiments, the acid is an organic acid such as methanesulfonic acid or p-toluenesulfonic acid.

In some other embodiments, bis-indoles are oxidized to the DIM salt product through treatment with a metal or organic oxidant. In some embodiments, the oxidant is an organic oxidant such as Ph₃C⁺ BF₄⁻ and the like. In other embodiments, the oxidant is a metal based oxidant such as pyridinium dichromate, pyridinium chlorochromate, K₂Cr₂O₇, FeCl₃, and the like.

In some embodiments, DIM salt products undergo ion exchange. In some embodiments, the ion exchange is performed with ion exchange column chromatography. In some embodiments, the ion is exchanged such that the final product is a chloride, bromide, or iodide salt. In some embodiments, the ion exchange column chromatography is performed with NaCl and the product is the chloride salt.

In some embodiments, DIM salt products include, but are not limited to, compounds comprising a suitable anion. In some embodiments, the suitable anion is a suitable monovalent anion. In some embodiments, the suitable anion is a divalent anion. In some embodiments, the suitable anion is a polyvalent anion. In some embodiments, the suitable anion is a divalent or polyvalent anion and there is less than one equivalent of said anion. In some embodiments, the suitable anion is selected from anions formed from inorganic acids and anions formed from organic acids. In some embodiments, the suitable anion is selected from halides, chlorates, sulfates, nitrates, phosphates, carboxylates, sulfonates, and borates. In some embodiments, the suitable anion is selected from acetate, benzoate, besylate, borate, bromide, camphorsulfonate, chloride, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, napsylate, nitrate, octadecanoate, oleate, oxalate, pamoate, phosphate, succinate, sulfate, sulfosalicylate, tartrate, tetrahydroxyborate, tetrafluoroborate, tosylate, or trifluoroacetate.

It is well understood by those in the art that other analogous procedures and reagents could be used, and that these Schemes are only meant as non-limiting examples.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. Whenever it appears herein, a numerical range such as “C₁-C₆ alkyl” or “C₁-₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkyl group may be optionally substituted as described below.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized 7c-electron system containing 4n+2π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

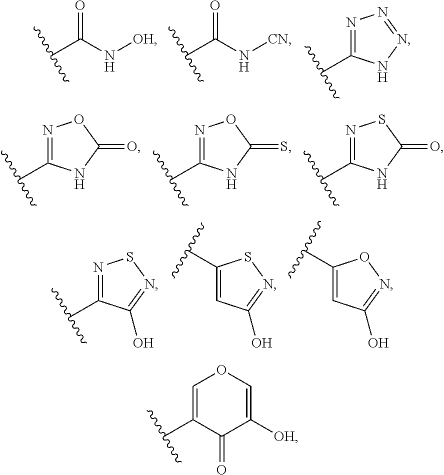

and the like.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cycicoalkyl is cyclopropyl, cyclobutyl, cyclopentyl or cyclohexyl. In some embodiments, the monocyclic cycicoalkyl is cyclopentyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆ alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

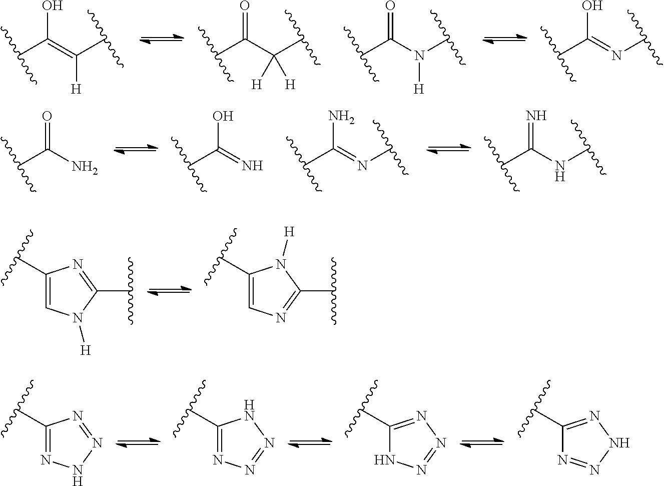

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (I) and a co-agent, are both administered to a patient simultaneously in the form of a single entity or dosage. The term “non-fixed combination” means that the active ingredients, e.g., a compound of Formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The term “subject” or “patient” encompasses mammals. Examples of mammals include, but are not limited to, humans. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds disclosed herein are administered topically to the skin.

In another aspect, the compounds disclosed herein are administered by inhalation.

In another aspect, the compounds disclosed herein are formulated for intranasal administration. Such formulations include nasal sprays, nasal mists, and the like.

In another aspect, the compounds disclosed herein are formulated as eye drops.

In any of the aforementioned aspects are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound disclosed herein, including further embodiments in which (i) the compound is administered once; (ii) the compound is administered to the mammal multiple times over the span of one day; (iii) the compound is administered continually; or (iv) the compound is administered continuously.

In any of the aforementioned aspects are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In one aspect, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Treating Disease

In another aspect, described herein is a method of treating a disease in a mammal, wherein the disease comprises abnormal levels of Bcl-2, Nur77, or combinations thereof, the method comprising administering a compound or composition described herein. In some embodiments, said abnormal levels comprise elvevated levels of Bcl-2, Nur77, or combinations thereof In some embodiments, said abnormal levels comprise descreased levels of Bcl-2, Nur77, or combinations thereof. In some embodiments, said abnormal levels refer to activity levels or expression levels of Bcl-2, Nur77, or combinations thereof. In some embodiments, said abnormal levels refer to the activity levels of the Bcl-2 dependent apoptotic pathway.

In some embodiments, the disease is cancer. In some embodiments, the cancer is selected from the group consisting of adrenal cortical cancer, anal cancer, bile duct cancer, bone cancer, bone metastasis, brain cancer, cervical cancer, non-Hodgkin's lymphoma, rectum cancer, esophageal cancer, eye cancer, gallbladder cancer, gastrointestinal carcinoid tumor, gestational trophoblastic disease, Hodgkin's disease, Kaposi's sarcoma, kidney cancer, laryngeal and hypopharyngeal cancer, leukemia, liver cancer, lung cancer, lung carcinoid tumor, malignant mesothelioma, metastatic cancer, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, prostate cancer, breast cancer, colorectal cancer, colon cancer, bladder cancer, penile cancer, pituitary cancer, retinoblastoma, salivary gland cancer, sarcoma, skin cancer, stomach cancer, testicular cancer, thymus cancer, thyroid cancer, uterine sarcoma, vaginal cancer, vulva cancer, and Wilm's tumor. In some embodiments, the cancer is resistant to chemotherapy, radiotherapy, or combinations thereof. In some embodiments, the cancer comprises elevated levels of Bcl-2, Nur77, or combinations thereof. In some embodiments, the cancer is resistant to chemotherapy, radiotherapy, or combinations thereof due to abnormal levels of Bcl-2, Nur77, or combinations thereof In some embodiments, the cancer is selected from the group consisting of prostate cancer, breast cancer, colorectal cancer, or pancreatic cancer.

In some embodiments, the compound binds to Nur77. In some embodiments, the compound is a modulator of Nur77. In some embodiments, the compound is an agonist of Nur77. In some embodiments, the compound promotes mitochondrial targeting of Nur77. In some embodiments, the compound modulates mitochondrial activities. In some embodiments, the compound induces interaction of Nur77 and Bcl-2.

In some embodiments, the mammal is human.

In some embodiments, the mammal is a chimp, a monkey, a dog, a pig, a rat, or a mouse. In some embodiments, the mammal is a transgenic mammal. In some embodiments, the mammal comprises a xenograft. In some embodiments, the xenograft comprises human cancer cells or a human tumor.

### Methods of Inducing Apoptosis

In another aspect, described herein is a method of inducing apoptosis in a cell, the method comprising contacting the cell a compound described herein.

In some embodiments, the compound binds to Nur77. In some embodiments, the compound is a modulator of Nur77. In some embodiments, the compound is an agonist of Nur77. In some embodiments, the compound promotes mitochondrial targeting of Nur77. In some embodiments, the compound modulates mitochondrial activities in the cell. In some embodiments, the compound induces interaction of Nur77 and Bcl-2.

In some embodiments, the cell is a cancer cell. In some embodiments, the cancer cell is selected from the group consisting of adrenal cortical cancer cell, anal cancer cell, bile duct cancer cell, bone cancer cell, bone metastasis cell, brain cancer cell, cervical cancer cell, non-Hodgkin's lymphoma cell, rectum cancer cell, esophageal cancer cell, eye cancer cell, gallbladder cancer cell, gastrointestinal carcinoid tumor cell, gestational trophoblastic disease cell, Hodgkin's disease cell, Kaposi's sarcoma cell, kidney cancer cell, laryngeal and hypopharyngeal cancer cell, leukemia cell, liver cancer cell, lung cancer cell, lung carcinoid tumor cell, malignant mesothelioma cell, metastatic cancer cell, multiple myeloma cell, myelodysplastic syndrome cell, nasal cavity and paranasal cancer cell, nasopharyngeal cancer cell, neuroblastoma cell, oral cavity and oropharyngeal cancer cell, osteosarcoma cell, ovarian cancer cell, pancreatic cancer cell, prostate cancer cell, breast cancer cell, colorectal cancer cell, colon cancer cell, bladder cancer cell, penile cancer cell, pituitary cancer cell, retinoblastoma cell, salivary gland cancer cell, sarcoma cell, skin cancer cell, stomach cancer cell, testicular cancer cell, thymus cancer cell, thyroid cancer cell, uterine sarcoma cell, vaginal cancer cell, vulva cancer cell, and Wilm's tumor cell. In some embodiments, the cancer cell is selected from the group consisting of prostate cancer cell, breast cancer cell, colorectal cancer cell, or pancreatic cancer cell.

In some embodiments, the cell is a human or non-human mammalian cell. In some embodiments, the cell is a human cell. In some embodiments, the cell is a chimp cell, a monkey cell, a dog cell, a pig cell, a rat cell, or a mouse cell.

In some embodiments, the cell is in vivo or in vitro. In some embodiments, the cell is in vivo. In some embodiments, the cell is in vitro.

In some embodiments, the cell comprises abnormal levels of Bcl-2, Nur77, or combinations thereof. In some embodiments, said abnormal levels comprise elvevated levels of Bcl-2, Nur77, or combinations thereof. In some embodiments, said abnormal levels comprise descreased levels of Bcl-2, Nur77, or combinations thereof. In some embodiments, said abnormal levels refer to activity levels or expression levels of Bcl-2, Nur77, or combinations thereof. In some embodiments, said abnormal levels refer to the activity levels of the Bcl-2 dependent apoptotic pathway.

### Methods for Modulating Nur77 Activity

In another aspect, described herein is a method for modulating Nur77 activity in a cell, the method comprising contacting the cell with a compound described herein. In some embodiments, the compound induces activation of Nur77. In some embodiments, the compound promotes mitochondrial targeting of Nur77. In some embodiments, the compound modulates mitochondrial activities in the cell. In some embodiments, wherein the compound induces interaction of Nur77 and Bcl-2.

The method of any one of claims 83-87, wherein the cell is a cancer cell.

In some embodiments, the cell is a cancer cell. In some embodiments, the cancer cell is selected from the group consisting of adrenal cortical cancer cell, anal cancer cell, bile duct cancer cell, bone cancer cell, bone metastasis cell, brain cancer cell, cervical cancer cell, non-Hodgkin's lymphoma cell, rectum cancer cell, esophageal cancer cell, eye cancer cell, gallbladder cancer cell, gastrointestinal carcinoid tumor cell, gestational trophoblastic disease cell, Hodgkin's disease cell, Kaposi's sarcoma cell, kidney cancer cell, laryngeal and hypopharyngeal cancer cell, leukemia cell, liver cancer cell, lung cancer cell, lung carcinoid tumor cell, malignant mesothelioma cell, metastatic cancer cell, multiple myeloma cell, myelodysplastic syndrome cell, nasal cavity and paranasal cancer cell, nasopharyngeal cancer cell, neuroblastoma cell, oral cavity and oropharyngeal cancer cell, osteosarcoma cell, ovarian cancer cell, pancreatic cancer cell, prostate cancer cell, breast cancer cell, colorectal cancer cell, colon cancer cell, bladder cancer cell, penile cancer cell, pituitary cancer cell, retinoblastoma cell, salivary gland cancer cell, sarcoma cell, skin cancer cell, stomach cancer cell, testicular cancer cell, thymus cancer cell, thyroid cancer cell, uterine sarcoma cell, vaginal cancer cell, vulva cancer cell, and Wilm's tumor cell. In some embodiments, the cancer cell is selected from the group consisting of prostate cancer cell, breast cancer cell, colorectal cancer cell, or pancreatic cancer cell.

In some embodiments, the cell is a human or non-human mammalian cell. In some embodiments, the cell is a human cell. In some embodiments, the cell is a chimp cell, a monkey cell, a dog cell, a pig cell, a rat cell, or a mouse cell.

In some embodiments, the cell is in vivo or in vitro. In some embodiments, the cell is in vivo. In some embodiments, the cell is in vitro.

In some embodiments, the cell comprises abnormal levels of Bcl-2, Nur77, or combinations thereof. In some embodiments, said abnormal levels comprise elvevated levels of Bcl-2, Nur77, or combinations thereof. In some embodiments, said abnormal levels comprise descreased levels of Bcl-2, Nur77, or combinations thereof. In some embodiments, said abnormal levels refer to activity levels or expression levels of Bcl-2, Nur77, or combinations thereof. In some embodiments, said abnormal levels refer to the activity levels of the Bcl-2 dependent apoptotic pathway.

### Methods of Dosing and Treatment Regimens

In one aspect, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compound disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01mg-5000 mg per day or from about 1 mg to about 1000 mg per day. In one embodiment, the desired dose is conveniently presented in a single dose or in divided doses.

### Combination Treatments

In certain instances, it is appropriate to administer at least one compound disclosed herein in combination with another therapeutic agent.

In one specific embodiment, a compound disclosed herein is co-administered with a second therapeutic agent, wherein the compound disclosed herein and the second therapeutic agent modulate different aspects of the disease, disorder or condition being treated, thereby providing a greater overall benefit than administration of either therapeutic agent alone.

For combination therapies described herein, dosages of the co-administered compounds vary depending on the type of co-drug(s) employed, on the specific drug(s) employed, on the disease or condition being treated and so forth. In additional embodiments, when co-administered with one or more other therapeutic agents, the compound provided herein is administered either simultaneously with the one or more other therapeutic agents, or sequentially. If administration is simultaneous, the multiple therapeutic agents are, by way of example only, provided in a single, unified form, or in multiple forms.

In some embodiments, the second therapeutic agent is a chemotherapeutic agent. In some embodiments, examples of the second therapeutic agent include, but are not limited to, alkylating agents, antibiotic agents, antimetabolic agents, hormonal agents, plant-derived agents, and biologic agents. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Examples of such hormonal agents are synthetic estrogens (e.g. diethylstilbestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). Examples of biologic agents include, but are not limited to, immuno-modulating proteins such as cytokines, monoclonal antibodies against tumor antigens, tumor suppressor genes, and cancer vaccines.

In some embodiments, the second therapeutic agent is radiation therapy. In some embodiments, a compound disclosed herein is administered in combination with radiation therapy.

### EXAMPLES

### Preparation of Compounds

### Abbreviations

DMSO: Dimethyl sulfoxide
ESI: Electrospray ionization
EtOH: Ethanol
H—X: Any protic acid, e.g., HCl, HBr, HNO₃
¹H NMR: Proton nuclear magnetic resonance
¹³C NMR: Carbon nuclear magnetic resonance
HRMS: High resolution mass spectrometry
h or hr(s): Hour(s)
MeSO₃⁻: Methanesulfonate
min(s): Minutes
rt: Room temperature
TFA: Trifluoroacetic acid

### Example 1: General Procedure A

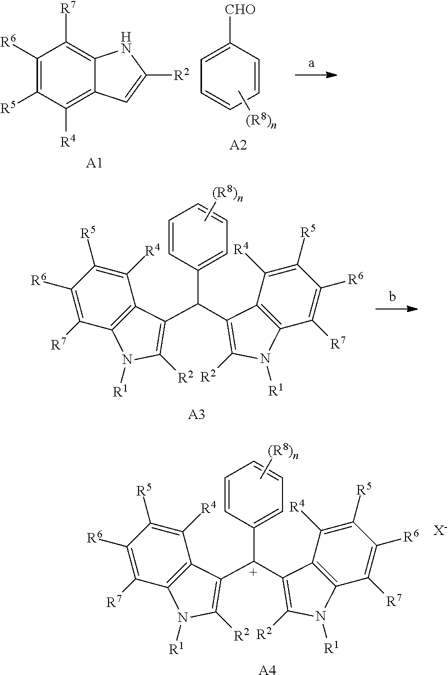

Reagents and Conditions: (a) CeCl₃.7H₂O, NaI, silica gel, CH₃CN; (b) 1-butanol, Activated Carbon, H—X

Step a: In a round-bottom flask, CeCl₃.7H₂O (1 equiv), Nal (1 equiv), silica gel, and CH₃CN were added sequentially. The mixture was stirred at room temperature for 12 h. Solvent was evaporated under reduced pressure and gave a yellow powder. After the completion of the catalyst activation, indole derivative A1 (2 equiv), benzaldehyde derivative A2 (1 equiv) and CH₃CN were added to the round-bottom flask containing the yellow powder. The mixture was stirred at room temperature for 2 h. Then, silica gel was filtered out, and the mixed solvent was evaporated under reduced pressure to obtain a crude product. The crude product was purified by silica-gel column chromatography.

Step b: Compound A3 (1 equiv), activated carbon (2 equiv), acid (H—X, 3 equiv) and 1-butanol were added to a round-bottom flask. The mixture was stirred at room temperature for 21 h. Then, the activated carbon was filtered off and the mixture was extracted with 1-butanol and water. The organic layer was evaporated under reduced pressure to obtain a crude product. Finally, the crude product was washed with ethyl ether.

### Example 2: General Procedure B

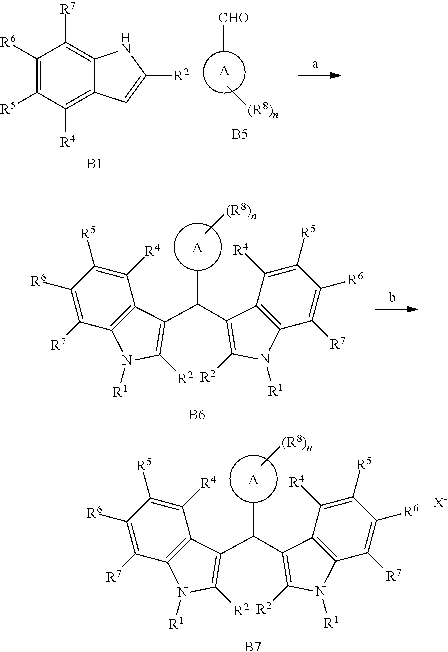

### Reagents and Conditions: (a) HCl, MeOH; (b) Pyridinium Dichromate, H—X, MeOH

Step a: Indole derivative B1 (2 equiv) and aldehyde derivative B5 (1 equiv) were added to a flask containing methanol, HCl (37%, 0.1 equiv) ten-fold dilution was added to the mixture. The mixture was stirred at room temperature until the reaction was completed. Then, reaction solution was neutralized by 5% NaOH, and the methanol was evaporated under reduced pressure to obtain a crude product which was purified by recrystallization (CH₃OH/H₂O) or by silica-gel column chromatography.

Step b: Compound B6 (1 equiv), pyridinium dichromate (1.4 equiv) and acid (H—X, 5 equiv) were added to a flask containing methanol. The mixture was stirred at room temperature for 5 h. Then, the methanol was evaporated under reduced pressure to obtain a mixture, and the mixture was extracted with 1-butanol and water. The organic layer was evaporated under reduced pressure to obtain a crude product. Finally, the crude product was purified by silica-gel column chromatography.

### Example 3: General Procedure C

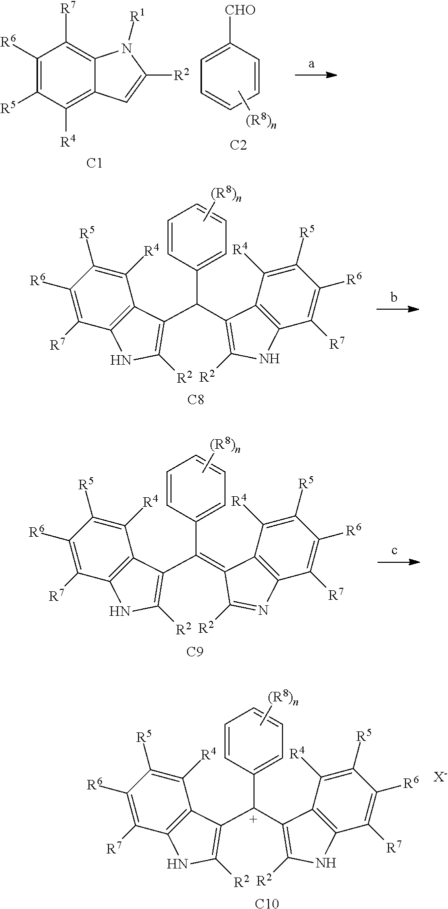

### Reagents and Conditions: (a) HCl, MeOH; (b) DDQ, Anhydrous Acetonitrile; (c) H—X, MeOH

Step a: Indole derivative Cl (2 equiv) and benzaldehyde derivative C2 (1 equiv) were added to a flask containing methanol, HCl (37%, 0.1 equiv) ten-fold dilution was added to the mixture. The mixture was stirred at room temperature until the reaction completed. Then, reaction solution was neutralized by 5% NaOH, and the methanol was evaporated under reduced pressure to obtain a crude product which was purified by recrystallization (CH₃OH/H₂O) or by column chromatography.

Step b: Compound C8 (1 equiv) was dissolved in acetonitrile, DDQ (0.6 equiv) solution of acetonitrile was dropwise and slowly added to the solution. This reaction was allowed for 2 h, the solvent was evaporated under reduced pressure and the residue was washed with saturation Na₂CO₃, and the crude product was purified by silica-gel column chromatography (CH₂Cl₂/MeOH), yielded as a red powder.

Step c: Compound C9 (1 equiv) and acid (H—X, 5 equiv) were added to a flask containing methanol. The mixture was stirred at room temperature for 1 h. Then, the methanol was evaporated under reduced pressure to obtain a mixture, and the mixture was extracted with 1-butanol and water. The organic layer was evaporated under reduced pressure to obtain a red product.

### Example 4: General Procedure D

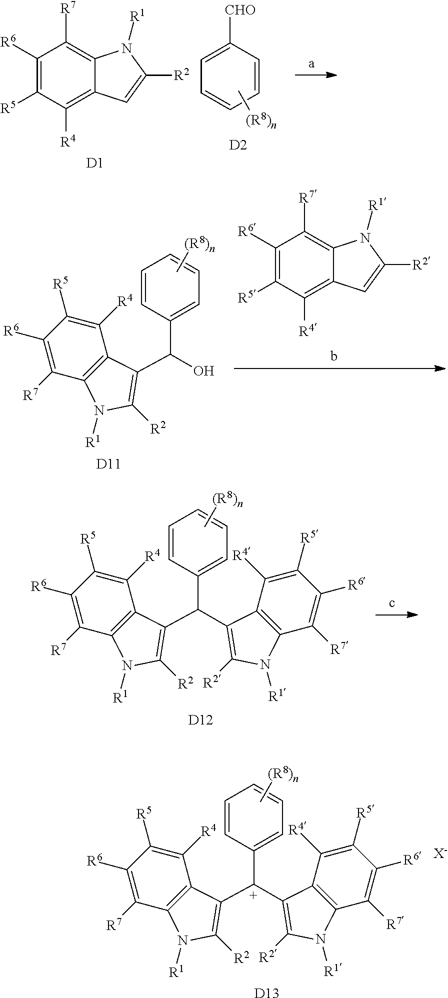

Reagents and Conditions: (a) Tetramethylguanidine, H₂O; (b) 2,2,2-trifluoroethanol, Reflux; (c) Pyridinium Dichromate, H—X, MeOH

Step a: Indole derivative D1 (1 equiv), aldehyde derivative D2 (1 equiv), and tetramethyl guanidine (0.2 equiv) were added to a round bottom flask. Pure water was added, and the reaction was stirred vigorously at room temperature for 12 h. The reaction mixture was extracted with ethyl acetate. The organic phase was collected, dried over anhydrous sodium thiosulfate and purified by silica-gel column chromatography, to give a white product.

Step b: Compound Dll (1 equiv) and indole derivative D1′ (1.5 equiv) were added to a round bottom flask equipped with trifluoroethanol. The reaction was stirred at 50° C. for 4 h, and the solvent was evaporated under reduced pressure to obtain a crude product which was purified by silica-gel column chromatography.

Step c: Compound D12 (1 equiv), acid (H—X, 5 equiv) and pyridinium dichromate (1.4 equiv) were added into a round-bottomed flask equipped with methanol. The mixture was stirred at room temperature for 5 h. Then, the methanol was evaporated under reduced pressure to obtain a mixture, and the mixture was extracted with 1-butanol and water. The organic layer was evaporated under reduced pressure to obtain a crude product. The crude product was purified by silica-gel column chromatography.

### Example 5: Bis(1H-indol-3-yl)(4-(trifluoromethyl)phenyl)methylium methanesulfonate, Compound 1

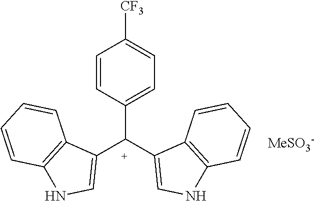 [SMILES: "MeSO3-" ; FC(F)(F)c1ccc([C+](c2c[nH]c3ccccc23)c2c[nH]c3ccccc23)cc1]

Bis(1H-indol-3-yl)(4-(trifluoromethyl)phenyl)methylium methanesulfonate was synthesized using General Procedure B, Example 2. Methanesulfonic acid was used in Step b. The product was obtained as red solid in 85% yield; ¹H NMR (600 MHz, methanol-d₄) δ ppm 3.18 (s, 3H), 7.48 (br. s., 2H), 8.00 (t, J=7.52 Hz, 2H), 8.23 (t, J=7.61 Hz, 2H), 8.55 (d, J=8.07 Hz, 2H), 8.71 (d, J=7.89 Hz, 2H), 8.87 (d, J=8.07 Hz, 2H), 9.50 (br. s., 2H), 14.90 (br. s., 2H). ¹³C NMR (151 MHz, methanol-d₄) δ ppm 115.96 (4C), 122.55 (2C), 122.90 (2C), 124.27 (1C), 125.93 (4C), 127.40 (4C), 133.38-134.67 (1C), 141.09 (2C), 149.48 (3C), 168.04 (1C). HRMS (ESI, m/z): 389.1258.

### Example 6: Bis(1H-indol-3-yl)(4-(trifluoromethyl)phenyl)methylium chloride, Compound 2

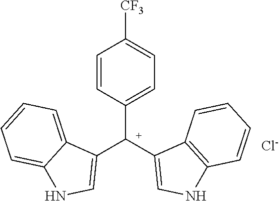 [SMILES: FC(F)(F)c1ccc([C+](c2c[nH]c3ccccc23)c2c[nH]c3ccccc23)cc1 ; [Cl-]]

Bis(1H-indol-3-yl)(4-(trifluoromethyl)phenyl)methylium chloride was synthesized using General Procedure B, Example 2. HCl (37%) was used in Step b. The product was obtained as red solid in 86% yield; ¹H NMR (600 MHz, DMSO-d₆) δ ppm 6.63 (br. s., 2H), 7.16 (t, J=7.5 Hz, 2H), 7.39 (t, J=7.5 Hz, 2H), 7.75 (d, J=7.9 Hz, 2H), 7.88 (d, J=7.7 Hz, 2H), 8.04 (d, J=7.9 Hz, 2H), 8.69 (br. s., 2H). ¹³C NMR (151 MHz, DMSO-d₆) δ ppm 115.2 (4C), 121.6 (q, J=5.5 Hz, 2C), 122.0 (2C), 124.6 (q, J=271.8 Hz),125.1 (4C), 126.5 (4C), 133.0 (q, J=31.9 Hz), 140.3 (2C), 148.4 (3C), 167.2. HRMS (ESI, m/z): 389.1258.

### Example 7: Bis(1H-indol-3-yl)(4-(trifluoromethyl)phenyl)methylium p-toluenesulfonate, Compound 3

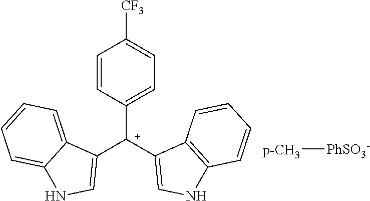

Bis(1H-indol-3-yl)(4-(trifluoromethyl)phenyl)methylium p-toluenesulfonate was synthesized using General Procedure B, Example 2. p-Toluenesulfonic acid was used in Step b. The product was obtained as red solid in 66% yield; ¹H NMR (600 MHz, DMSO-d₆) δ ppm 2.28 (s, 3H), 6.67 (d, J=7.89 Hz, 2H), 7.07-7.18 (m, 4H), 7.37 (t, J=7.61 Hz, 2H), 7.51 (d, J=8.07 Hz, 2H), 7.70 (d, J=8.07 Hz, 2H), 7.86 (d, J=8.07 Hz, 2H), 8.03 (d, J=8.25 Hz, 2H), 8.57 (s, 2H).

### Example 8: Bis(1H-indol-3-yl)(4-(trifluoromethyl)phenyl)methylium sulfate, Compound 4

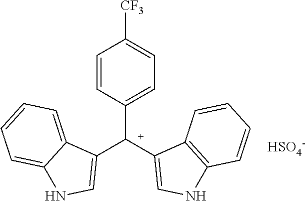

Bis(1H-indol-3-yl)(4-(trifluoromethyl)phenyl)methylium sulfate was synthesized using General Procedure B, Example 2. Sufuric acid was used in Step b. The product was obtained as red solid in 61% yield; ¹H NMR (600 MHz, DMSO-d₆) δ ppm 6.67 (br. s., 2H), 7.12 (br. s., 2H), 7.35 (d, J=6.05 Hz, 2H), 7.68 (br. s., 2H), 7.84 (d, J=6.42 Hz, 2H), 8.02 (d, J=6.97 Hz, 2H), 8.49 (br. s., 2H).

### Example 9: Bis(1H-indol-3-yl)(4-chlorophenyl)methylium methanesulfonate, Compound 5

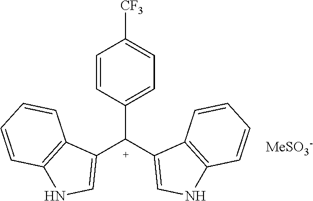 [SMILES: "MeSO3-" ; FC(F)(F)c1ccc([C+](c2c[nH]c3ccccc23)c2c[nH]c3ccccc23)cc1]

Bis(1H-indol-3-yl)(4-chlorophenyl)methylium methanesulfonate was synthesized using General Procedure A, Example 1. Methanesulfonic acid was used in Step b. The product was obtained as red solid in 17% yield; ¹H NMR (600 MHz, methanol-d₄) δ ppm 2.70 (s, 3H), 6.91 (br. s., 2H), 7.19 (t, J=7.7 Hz, 2H), 7.43 (t, J=7.7 Hz, 2H), 7.63-7.75 (m, 6H), 8.35 (br. s., 2H). ¹³C NMR (151 MHz, methanol-d₄) δ ppm 38.1, 113.9 (4C), 121.6 (2C), 122.2 (2C), 124.5 (4C), 126.1 (4C), 129.4 (br. s., 2C), 139.9, 140.3, 146.4 (2C). HRMS (ESI, m/z): 355.0995.

### Example 10: Bis(1H-indol-3-yl)(4-chlorophenyl)methylium chloride, Compound 6

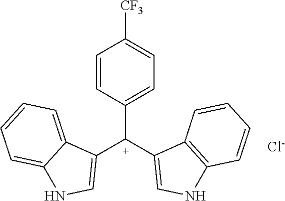 [SMILES: FC(F)(F)c1ccc([C+](c2c[nH]c3ccccc23)c2c[nH]c3ccccc23)cc1 ; [Cl-]]

Bis(1H-indol-3-yl)(4-chlorophenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 13% yield; ¹H NMR (600 MHz, DMSO-d₆) δ ppm 6.74 (br. s., 2H), 7.18 (t, J=7.6 Hz, 2H), 7.40 (t, J=7.6 Hz, 2H), 7.71 (d, J=8.1 Hz, 2H), 7.74 (d, J=8.1 Hz, 2H), 7.78 (d, J=8.4 Hz, 2H), 8.67 (br. s., 2H). ¹³C NMR (151 MHz, DMSO-d₆) δ ppm 115.0 (4C), 121.6 (2C), 121.8 (2C), 124.9 (4C), 126.3 (4C), 130.0 (2C), 139.1 (1C), 140.3 (1C), 148.1 (2C).

### Example 11: Bis(1H-indol-3-yl)(4-methoxyphenyl)methylium methanesulfonate, Compound 7

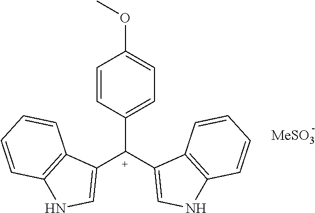 [SMILES: COc1ccc([C+](c2c[nH]c3ccccc23)c2c[nH]c3ccccc23)cc1]

Bis(1H-indol-3-yl)(4-methoxyphenyl)methylium methanesulfonate was synthesized using General Procedure A, Example 1. Methanesulfonic acid was used in Step b. The product was obtained as red solid in 20.2% yield; ¹H NMR (600 MHz, methanol-d₄) δ ppm 2.69 (s, 3H), 3.99 (s, 3H), 6.97 (br.s., 2H), 7.15 (t, J=7.2 Hz, 2H), 7.22 (d, J=7.0 Hz, 2H), 7.40 (t, J=7.3 Hz, 2H), 7.68 (d, J=8.1 Hz, 4H), 8.21 (br. s., 2H).¹³C NMR (151 MHz, methanol-d₄) δ ppm 38.2, 55.2, 113.7 (4C), 114.7 (4C), 124.1 (4C), 125.7 (4C), 129.5 (2C), 139.6, 145.2 (2C), 165.9. HRMS (ESI, m/z): 351.1491.

### Example 12: Bis(1H-indol-3-yl)(4-(methoxycarbonyl)phenyl)methylium methanesulfonate, Compound 8

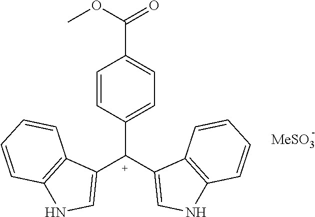 [SMILES: COC(=O)c1ccc([C+](c2c[nH]c3ccccc23)c2c[nH]c3ccccc23)cc1]

Bis(1H-indol-3-yl)(4-(methoxycarbonyl)phenyl)methylium methanesulfonate was synthesized using General Procedure A, Example 1. Methanesulfonic acid was used in Step b. The product was obtained as red solid in 18.1% yield; ¹H NMR (600 MHz, methanol-d₄) δ ppm 2.70, (s, 3H), 4.00 (s, 3H), 6.82 (br. s., 2H), 7.16 (t, J=7.2 Hz, 2H), 7.42 (t, J=7.3 Hz, 2H), 7.70 (d, J=8.1 Hz, 2H), 7.78 (d, J=7.7 Hz, 2H), 8.27 (d, J=8.1 Hz, 2H), 8.41 (br. s., 2H). ¹³C NMR (151 MHz, methanol-d₄) δ ppm 38.1 (br. s.), 51.8, 114.0 (4C), 121.6 (br. s., 2C), 122.3 (2C), 124.6 (4C), 126.2 (4C), 129.8 (br. s., 2C), 134.2, 139.9, 146.7 (2C), 166.1. HRMS (ESI, m/z): 379.1438.

### Example 13: Bis(1H-indol-3-yl)(4-(methoxycarbonyl)phenyl)methylium chloride, Compound 9

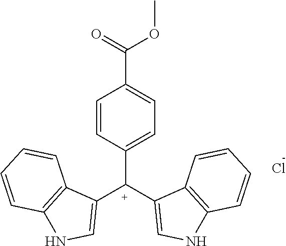 [SMILES: COC(=O)c1ccc([C+](c2c[nH]c3ccccc23)c2c[nH]c3ccccc23)cc1 ; Cl]

Bis(1H-indol-3-yl)(4-(methoxycarbonyl)phenyl)methylium chloride was synthesized using General Procedure B, Example 2. HCl (37%) was used and the crude product was purified by ion exchange column chromatography (H₂O/EtOH/NaCl) in Step b. The product was obtained as red solid in 30% yield; ¹H NMR (600 MHz, DMSO-d₆) δ ppm 3.96 (s, 3H), 6.63 (br. s., 2H), 7.15 (t, J=7.6 Hz, 2H), 7.39 (t, J=7.7 Hz, 2H), 7.74 (d, J=8.1 Hz, 2H), 7.82 (d, J=8.1 Hz, 2H), 8.23 (d, J=8.1 Hz, 2H), 8.74 (br. s., 2H). ¹³C NMR (151 MHz, DMSO-d₆) δ ppm 53.1, 115.1 (4C), 121.6 (2C), 121.9 (2C), 125.0 (4C), 126.4 (4C), 130.4 (2C), 133.6, 140.3, 148.3 (2C), 166.2, 167.9.

### Example 14: Bis(5-fluoro-1H-indol-3-yl)(4-(trifluoromethyl)phenyl)methylium methanesulfonate, Compound 10

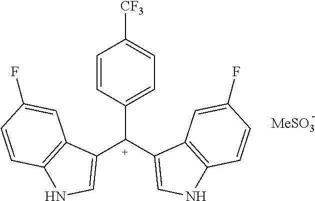 [SMILES: Fc1ccc2[nH]cc([C+](c3ccc(C(F)(F)F)cc3)c3c[nH]c4ccc(F)cc34)c2c1]

Bis(5-fluoro-1H-indol-3-yl)(4-(trifluoromethyl)phenyl)methylium methanesulfonate was synthesized using General Procedure A, Example 1. Methanesulfonic acid was used in Step b. The product was obtained as red solid in 58% yield; ¹H NMR (600 MHz methanol-d₄) δ ppm 2.70 (s, 3H), 6.45 (br. s., 2H), 7.24 (td, J=9.0, 2.2 Hz, 2H), 7.73 (dd, J=8.8, 4.4 Hz, 2H), 7.88 (d, J=8.1 Hz, 2H), 8.03 (d, J=8.1 Hz, 2H), 8.47 (br. s., 2H). ¹³C NMR (151 MHz, methanol-d₄) δ ppm 38.1, 107.5 (d, J=27.5 Hz, 2C), 111.8 (d, J=9.9 Hz, 2C), 114.2 (d, J=25.3 Hz, 2C), 115.7 (d, J=9.9 Hz, 2C), 121.9 (d, J=3.3 Hz, 2C), 123.8 (q, J=272.9 Hz), 126.0 (q, J=3.3 Hz, 2C), 127.3 (d, J=16.5 Hz, 2C), 129.5 (2C), 134.6 (q, J=33.0 Hz), 136.4, 148.0 (br. s., 2C), 160.6 (d, J=241.0 Hz, 2C). HRMS (ESI, m/z): 425.1065.

### Example 15: Bis(1-methyl-1H-indol-3-yl)(4-(trifluoromethyl)phenyl)methylium methanesulfonate, Compound 11

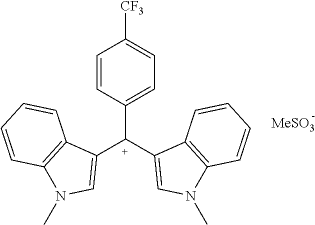 [SMILES: Cn1cc([C+](c2ccc(C(F)(F)F)cc2)c2cn(C)c3ccccc23)c2ccccc21]

Bis(1-methyl-1H-indol-3-yl)(4-(trifluoromethyl)phenyl)methylium methanesulfonate was synthesized using General Procedure B, Example 2. Methanesulfonic acid was used in Step b. The product was obtained as red solid in 71% yield; ¹H NMR (600 MHz, methanol-d₄) δ ppm 2.69 (br. s., 3H), 4.15 (s, 6H), 6.83 (br. s., 2H), 7.24 (t, J=7.3 Hz, 2H), 7.51 (t, J=7.5 Hz, 2H), 7.78 (d, J=8.1 Hz, 2H), 7.88 (d, J=7.3 Hz, 2H), 7.99 (d, J=7.3 Hz, 2H), 8.45 (br. s., 2H). ¹³C NMR (151 MHz, methanol-d₄) δ ppm 34.1 (2C) 38.1, 112.5 (4C), 121.1 (2C), 121.8 (2C), 123.9 (q, J=272.9 Hz), 125.1 (4C), 125.9 (q, J=4.4 Hz, 2C), 126.3 (4C), 134.2 (q, J=34.1 Hz), 140.9, 149.2 (2C). HRMS (ESI, m/z): 417.1575.

### Example 16: Bis(1-methyl-1H-indol-3-yl)(4-(trifluoromethyl)phenyl)methylium chloride, Compound 12

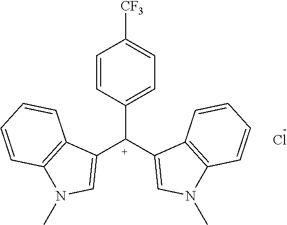 [SMILES: Cn1cc([C+](c2ccc(C(F)(F)F)cc2)c2cn(C)c3ccccc23)c2ccccc21 ; Cl]

Bis(1-methyl-1H-indol-3-yl)(4-(trifluoromethyl)phenyl)methylium chloride was synthesized using General Procedure B, Example 2. Methanesulfonic acid was used and the crude product purified by ion exchange column chromatography (H₂O/EtOH/NaCl) in Step b. The product was obtained as red solid in 37% yield; ¹H NMR (600 MHz, methanol-d₄) δ ppm 4.16 (s, 6H), 6.81 (br. s., 2H), 7.23 (t, J=7.6 Hz, 2H), 7.50 (t, J=7.7 Hz, 2H), 7.78 (d, J=8.1 Hz, 2H), 7.88 (d, J=8.1 Hz, 2H), 7.99 (d, J=8.3 Hz, 2H), 8.51 (br. s., 2H). ¹³C NMR (151 MHz, methanol-d₄) δ ppm 34.1 (2C), 112.5 (4C), 121.0 (2C), 121.7 (2C), 123.9 (q, J=272.9 Hz), 125.1 (4C), 125.9 (q, J=4.4 Hz, 2C), 126.3 (4C), 134.1 (q, J=33.0 Hz), 140.9, 149.3 (2C), 166.1.

### EXAMPLE 17: Bis(2-methyl-1H-indol-3-yl)(4-(trifluoromethyl)phenyl)methylium methanesulfonate, Compound 13

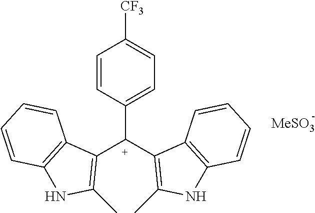 [SMILES: Cc1[nH]c2ccccc2c1[C+](c1ccc(C(F)(F)F)cc1)c1c(C)[nH]c2ccccc12]

Bis(2-methyl-1H-indol-3-yl)(4-(trifluoromethyl)phenyl)methylium methanesulfonate was synthesized using General Procedure A, Example 1. Methanesulfonic acid was used in Step b. The product was obtained as red solid in 3% yield; ¹H NMR (600 MHz, methanol-d₄) δ ppm 2.16 (s, 6H), 2.74 (br. s., 3H), 6.73 (d, J=8.1 Hz, 2H), 7.10 (t, J=7.5 Hz, 2H), 7.34 (t, J=7.7 Hz, 2H), 7.56 (d, J=8.1 Hz, 2H), 7.75 (d, J=8.1 Hz, 2H), 7.93 (d, J=8.1 Hz, 2H). ¹³C NMR (151 MHz, methanol-d₄) δ ppm 14.0 (2C), 38.1, 112.9 (2C), 120.8 (2C), 121.6 (2C), 123.8 (q, J=275.1 Hz), 124.5 (2C), 125.7 (2C), 126.1 (q, J=4.4 Hz, 2C), 127.5 (2C), 133.4 (2C), 134.3 (q, J=33.0 Hz), 138.0 (2C), 143.3, 156.8(2C). HRMS (ESI, m/z): 417.1568.

### Example 18: Bis(2-methyl-1H-indol-3-yl)(4-(trifluoromethyl)phenyl)methylium chloride, Compound 14

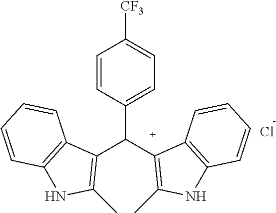 [SMILES: Cc1[nH]c2ccccc2c1C(c1ccc(C(F)(F)F)cc1)c1c(C)[nH]c2ccccc12 ; Cl]

Bis(2-methyl-1H-indol-3-yl)(4-(trifluoromethyl)phenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 35% yield; ¹H NMR (600 MHz, DMSO-d₆) δ ppm 2.11 (br. s., 6H), 6.61 (d, J=7.3 Hz, 2H), 7.07 (t, J=6.7 Hz, 2H), 7.31 (t, J=7.2 Hz, 2H), 7.60 (d, J=7.3 Hz, 2H), 7.77 (d, J=7.2 Hz, 2H), 7.96 (d, J=7.3 Hz, 2H). ¹³C NMR (151 MHz, DMSO-d₆) δ ppm 15.5 (2C), 114.0 (2C), 121.1 (2C), 121.4 (2C), 124.5 (q, J=272.9 Hz), 124.9 (2C), 126.0 (2C), 126.6 (q, J=4.4 Hz, 2C), 127.7 (2C), 133.1 (q, J=33.0 Hz), 134.0 (2C), 138.3 (2C), 143.5, 157.3 (2C), 165.8.

### Example 19: Bis(5-methyl-1H-indol-3-yl)(4-(trifluoromethyl)phenyl)methylium methanesulfonate, Compound 15

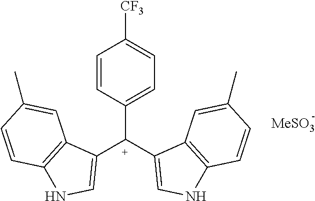 [SMILES: Cc1ccc2[nH]cc([C+](c3ccc(C(F)(F)F)cc3)c3c[nH]c4ccc(C)cc34)c2c1]

Bis(5-methyl-1H-indol-3-yl)(4-(trifluoromethyl)phenyl)methylium methanesulfonate was synthesized using General Procedure A, Example 1. Methanesulfonic acid was used in Step b. The product was obtained as red solid in 5% yield; ¹H NMR (600 MHz, methanol-d₄) δ ppm 2.22 (s, 6H), 2.70 (s, 3H), 6.59 (br. s., 2H), 7.27 (d, J=7.7 Hz, 2H), 7.57 (d, J=8.1 Hz, 2H), 7.85 (d, J=7.7 Hz, 2H), 7.99 (d, J=8.1 Hz, 2H), 8.26 (br. s., 2H). ¹³C NMR (151 MHz, methanol-d₄) δ ppm 20.2 (2C), 38.1, 113.7 (2C), 121.9 (d, J=4.4 Hz, 2C), 122.1 (2C), 123.9 (q, J=272.9 Hz), 125.0 (2C), 125.7 (2C), 126.4 (2C), 127.4 (2C), 129.4 (2C), 134.0 (q, J=33.0 Hz), 134.7 (2C), 138.4 , 146.4 (2C). HRMS (ESI, m/z) found.417.1575.

### Example 20: Bis(5-methyl-1H-indol-3-yl)(4-(trifluoromethyl)phenyl)methylium chloride, Compound 16

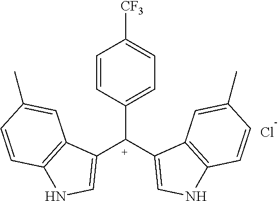 [SMILES: Cc1ccc2[nH]cc([C+](c3ccc(C(F)(F)F)cc3)c3c[nH]c4ccc(C)cc34)c2c1 ; Cl]

Bis(5-methyl-1H-indol-3-yl)(4-(trifluoromethyl)phenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 24% yield; ¹H NMR (600 MHz, DMSO-d₆) δ ppm 2.15 (s, 6H), 6.39 (br. s., 2H), 7.23 (d, J=7.9 Hz, 2H), 7.61 (d, J=8.6 Hz, 2H), 7.85 (d, J=7.7 Hz, 2H), 8.04 (d, J=7.9 Hz, 2H), 8.56 (br. s., 2H). ¹³C NMR (151 MHz, DMSO-d₆) δ ppm 21.7 (2C), 114.8 (2C), 121.5 (2C), 124.4 (q, J=272.9 Hz), 125.2, 126.5 (q, J=4.4 Hz, 2C), 127.1, 127.6 (2C), 129.1, 129.5, 132.8 (q, J=31.9 Hz), 132.5, 133.2, 134.3 (2C), 138.4 (2C), 139.3, 147.9 (2C), 166.7.

### Example 21: Bis(6-methyl-1H-indol-3-yl)(4-(trifluoromethyl)phenyl)methylium methanesulfonate, Compound 17

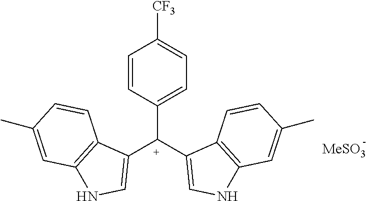

Bis(6-methyl-1H-indol-3-yl)(4-(trifluoromethyl)phenyl)methylium methanesulfonate was synthesized using General Procedure A, Example 1. Methanesulfonic acid was used in Step b. The product was obtained as red solid in 10% yield; ¹H NMR (600 MHz, methanol-d₄) δ ppm 2.47 (s, 6H), 2.70 (s, 3H), 6.71 (d, J=7.3 Hz, 2H), 7.02 (d, J=8.1 Hz, 2H), 7.49 (s, 2H), 7.82 (d, J=8.1 Hz, 2H), 7.95 (d, J=7.7 Hz, 2H), 8.27 (br. s., 2H). ¹³C NMR (151 MHz, methanol-d₄) δ ppm 20.2 (2C), 38.1, 114.0 (2C), 121.4 (br. s., 2C), 122.5 (2C), 123.9 (q, J=272.9 Hz), 124.0 (2C), 125.7 (q, J=4.4 Hz, 2C), 126.0 (4C), 133.3-133.5 (m, 2C), 134.0 (q, J=33.0 Hz), 137.0 (2C), 140.7, 146.6 (2C). HRMS (ESI, m/z): found.417.1574.

### Example 22: Bis(6-methyl-1H-indol-3-yl)(4-(trifluoromethyl)phenyl)methylium chloride, Compound 18

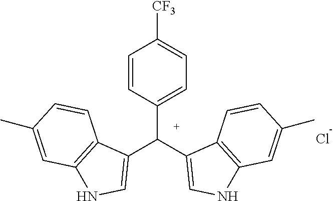

Bis(6-methyl-1H-indol-3-yl)(4-(trifluoromethyl)phenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 14% yield; ¹H NMR (600 MHz, DMSO-d₆) δ ppm 2.40 (s, 6H), 6.51 (br. s., 2H), 6.99 (d, J=8.1 Hz, 2H), 7.51 (s, 2H), 7.82 (d, J=7.7 Hz, 2H), 8.02 (d, J=8.1 Hz, 2H), 8.54 (br. s., 2H). ¹³C NMR (151 MHz, DMSO-d₆) δ ppm 21.6 (2C), 114.9 (2C), 121.5 (2C), 121.9 (2C), 124.3 (q, J=272.9 Hz), 124.3 (2C), 126.4 (2C), 126.5 (q, J=5.5 Hz, 2C), 129.1, 132.0, 132.1, 132.9 (q, J=31.9 Hz), 136.5 (2C), 140.6 (2C), 148.1 (2C), 167.5.

### Example 23: Bis(7-methyl-1H-indol-3-yl)(4-(trifluoromethyl)phenyl)methylium methanesulfonate, Compound 19

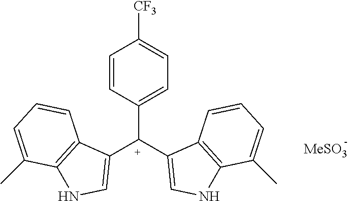

Bis(7-methyl-1H-indol-3-yl)[4-(trifluoromethyl)phenyl]methylium methanesulfonate was synthesized using General Procedure A, Example 1. Methanesulfonic acid was used in Step b. The product was obtained as red solid in 4% yield; ¹H NMR (600 MHz, methanol-d₄) δ ppm 2.63 (s, 6H), 2.69 (s, 3H), 6.66 (br. s., 2H), 7.08 (t, J=7.7 Hz, 2H), 7.24 (d, J=7.3 Hz, 2H), 7.86 (d, J=7.7 Hz, 2H), 7.98 (d, J=8.1 Hz, 2H), 8.38 (br. s., 2H). ¹³C NMR (151 MHz, methanol-d₄) δ ppm 15.3 (2C), 38.1, 119.3 (2C), 122.8 (2C), 123.9 (q, J=271.8 Hz), 124.3 (2C), 124.8 (4C), 125.8 (br. s., 2C), 126.2 (q, J=4.4 Hz, 2C), 127.1 (4C), 134.2 (q, J=33.0 Hz), 139.2, 146.6 (2C). HRMS (ESI, m/z): 417.1574.

### Example 24: Bis(7-methyl-1H-indol-3-yl)(4-(trifluoromethyl)phenyl)methylium chloride, Compound 20

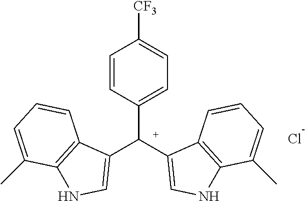

Bis(7-methyl-1H-indol-3-yl)[4-(trifluoromethyl)phenyl]methylium chloride was synthesized using General Procedure B, Example 2. Methanesulfonic acid was used and the crude product was purified by ion exchange column chromatography (H₂O/EtOH/NaCl) in Step b. The product was obtained as red solid in 20% yield; ¹H NMR (600 MHz, DMSO-d₆) δ ppm 2.62 (s, 6H), 6.48 (br. s., 2H), 7.07 (t, J=7.6 Hz, 2H), 7.22 (d, J=7.2 Hz, 2H), 7.88 (d, J=7.9 Hz, 2H), 8.05 (d, J=8.3 Hz, 2H), 8.65 (br. s., 2H). ¹³C NMR (151 MHz, DMSO-d₆) δ ppm 17.1 (2C), 119.2 (2C), 122.6 (2C), 124.7 (q, J=272.9 Hz), 125.0 (2C), 125.1 (2C), 126.4-126.8 (m, 4C), 127.3 (2C), 129.1, 132.0, 132.1, 133.0 (q, J=33.0 Hz), 139.6 (2C), 148.3 (2C), 166.9.

### Example 25: Bis(6-fluoro-1H-indol-3-yl)(4-(trifluoromethyl)phenyl)methylium methanesulfonate, Compound 21

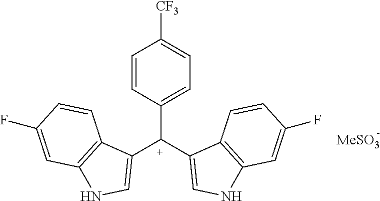 [SMILES: Fc1ccc2c([C+](c3ccc(C(F)(F)F)cc3)c3c[nH]c4cc(F)ccc34)c[nH]c2c1]

Bis(6-fluoro-1H-indol-3-yl)(4-(trifluoromethyl)phenyl)methylium methanesulfonate was synthesized using General Procedure A, Example 1. Methanesulfonic acid was used in Step b. The product was was obtained as red solid in 7% yield; ¹H NMR (600 MHz, methanol-d₄) δ ppm 2.70 (s, 3H), 6.83 (br. s., 2H), 7.00 (t, J=8.62 Hz, 2H), 7.47 (dd, J=8.44, 1.83 Hz, 2H), 7.89 (d, J=7.70 Hz, 2H), 8.01 (d, J=8.07 Hz, 2H), 8.47 (br. s., 2H). ¹³C NMR (151 MHz, methanol-d₄) δ ppm 38.1, 100.9 (d, J=26.4 Hz, 2C), 112.8 (d, J=24.2 Hz, 2C), 122.2 (2C), 122.7 (d, J=2.2 Hz, 2C), 123.0 (q, J=271.8 Hz), 123.0 (q, J=3.3 Hz, 2C), 123.1 (d, J=2.2 Hz, 2C), 126.0 (br. s., 4C), 134.5 (q, J=33.0 Hz), 140.6 (d, J=12.1 Hz, 2C), 148.2, 161.6 (d, J=244.3 Hz, 2C). HRMS (ESI, m/z): 425.1072.

### Example 26: Bis(6-fluoro-1H-indol-3-yl)(4-(trifluoromethyl)phenyl)methylium chloride, Compound 22

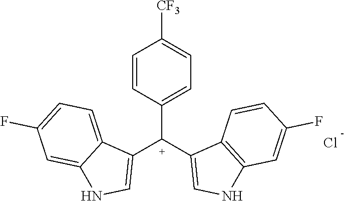 [SMILES: Fc1ccc2c([C+](c3ccc(C(F)(F)F)cc3)c3c[nH]c4cc(F)ccc34)c[nH]c2c1 ; Cl]

Bis(6-fluoro-1H-indol-3-yl)(4-(trifluoromethyl)phenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 24% yield; ¹H NMR (600 MHz, DMSO-d₆) δ ppm 6.66 (br. s., 2H), 7.09 (td, J=9.1, 2.3 Hz, 2H), 7.59 (dd, J=8.8, 2.38 Hz, 2H), 7.91 (d, J=8.1 Hz, 2H), 8.07 (d, J=8.3 Hz, 2H), 8.75 (br. s., 2H). ¹³C NMR (151 MHz, DMSO-d₆) δ ppm 101.9 (d, J=27.5 Hz, 2C), 113.2 (d, J=24.2 Hz, 2C), 121.9 (2C), 123.0-123.4 (m, 4C), 124.3 (q, J=271.8 Hz), 126.7 (4C), 129.5 (2C), 133.2 (q, J=31.9 Hz), 140.9 (d, J=12.1 Hz, 2C), 149.8, 160.9 (d, J=244.3 Hz, 2C), 167.2.

### EXAMPLE 27: Bis(5-chloro-1H-indol-3-yl)(4-(trifluoromethyl)phenyl)methylium methanesulfonate, Compound 23

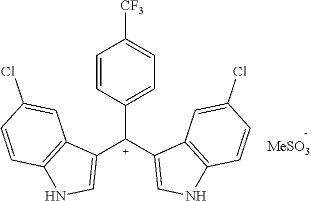 [SMILES: "MeSO3" ; FC(F)(F)c1ccc([C+](c2c[nH]c3ccc(Cl)cc23)c2c[nH]c3ccc(Cl)cc23)cc1]

Bis(5-chloro-1H-indol-3 -yl)(4-(trifluoromethyl)phenyl)methylium methanesulfonate was synthesized using General Procedure A, Example 1. Methanesulfonic acid was used in Step b. The product was obtained as red solid in 5% yield; ¹H NMR (600 MHz, methanol-d₄) δ ppm 2.70 (s, 3H), 6.73 (br. s., 2H), 7.46 (d, J=8.44 Hz, 2H), 7.72 (d, J=8.44 Hz, 2H), 7.90 (d, J=7.70 Hz, 2H), 8.04 (d, J=8.07 Hz, 2H), 8.51 (br. s., 2H). ¹³C NMR (151 MHz, methanol-d₄) δ ppm 38.1, 115.5 (4C), 121.4 (q, J=4.4 Hz, 2C), 121.6 (4C), 123.8 (q, J=272.9 Hz), 126.0 (br. s., 2C), 126.5 (4C), 130.7 (2C), 134.7 (q, J=31.9 Hz), 138.4, 148.1 (2C). HRMS (ESI, m/z): 457.0472.

### Example 28: Bis(6-chloro-1H-indol-3-yl)(4-(trifluoromethyl)phenyl)methylium methanesulfonate, Compound 24

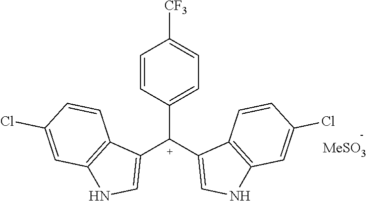 [SMILES: "MeSO3" ; FC(F)(F)c1ccc([C+](c2c[nH]c3cc(Cl)ccc23)c2c[nH]c3cc(Cl)ccc23)cc1]

Bis(6-chloro-1H-indol-3-yl)(4-(trifluoromethyl)phenyl)methylium methanesulfonate was synthesized using General Procedure A, Example 1. Methanesulfonic acid was used in Step b. The product was obtained as red solid in 8% yield; ¹H NMR (600 MHz, methanol-d₄) δ ppm 2.70 (s, 3H), 6.81 (br. s., 2H), 7.22 (d, J=8.07 Hz, 2H), 7.75 (s, 2H), 7.89 (d, J=8.07 Hz, 2H), 8.00 (d, J=8.07 Hz, 2H), 8.49 (br. s., 2H). ¹³C NMR (151 MHz, methanol-d₄) δ ppm 38.1, 114.2 (4C), 122.2 (4C), 122.7 (q, J=4.4 Hz, 2C), 123.8 (q, J=272.9 Hz), 125.1 (4C), 126.0 (br. s., 2C), 132.0 (2C), 134.6 (q, J=34.1 Hz) 140.6, 148.3 (2C). HRMS (ESI, m/z): 457.0472.

### Example 29: Bis(5-bromo-1H-indol-3-yl)(4-(trifluoromethyl)phenyl)methylium methanesulfonate, Compound 25

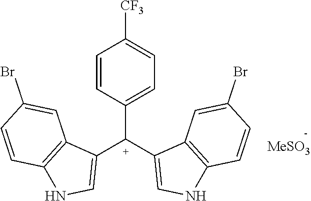 [SMILES: "MeSO3" ; FC(F)(F)c1ccc([C+](c2c[nH]c3ccc(Br)cc23)c2c[nH]c3ccc(Br)cc23)cc1]

Bis(5-bromo-1H-indol-3-yl)(4-(trifluoromethyl)phenyl)methylium methanesulfonate was synthesized using General Procedure A, Example 1. Methanesulfonic acid was used in Step b. The product was obtained as red solid in 3% yield; ¹H NMR (600 MHz, methanol-d₄) δ ppm 2.77 (br. s., 3H), 6.89 (br. s., 2H), 7.59 (d, J=8.1 Hz, 2H), 7.66 (d, J=8.4 Hz, 2H), 7.90 (d, J=7.3 Hz, 2H), 8.04 (d, J=7.7 Hz, 2H), 8.48 (br. s., 2H). ¹³C NMR (151 MHz, methanol-d₄) δ ppm 31.7, 115.8 (4C), 118.2 (2C), 121.5 (4C), 123.8 (q, J=270.7 Hz), 124.6 (q, J=4.4 Hz, 2C), 126.0 (br. s., 4C), 129.2 (2C), 134.7 (q, J=34.1 Hz), 138.7, 148.0 (br. s., 2C). HRMS (ESI, m/z): 546.9443.

### Example 30: Bis(5-fluoro-1H-indol-3-yl)(4-chlorophenyl)methylium methanesulfonate, Compound 26

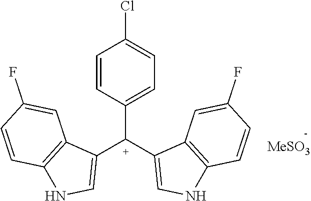 [SMILES: "MeSO3" ; Fc1ccc2[nH]cc([C+](c3ccc(Cl)cc3)c3c[nH]c4ccc(F)cc34)c2c1]

Bis(5-fluoro-1H-indol-3-yl)(4-chlorophenyl)methylium methanesulfonate was synthesized using General Procedure A, Example 1. Methanesulfonic acid was used in Step b. The product was obtained as red solid in 12% yield; ¹H NMR (600 MHz, methanol-d₄) δ ppm 2.70 (s, 3H), 6.52 (br. s., 2H), 7.23 (t, J=8.1 Hz, 2H), 7.69 (d, J=8.1 Hz, 2H), 7.72 (dd, J=8.6, 4.2 Hz, 2H), 7.75 (d, J=8.1 Hz, 2H), 8.43 (br. s., 2H). ¹³C NMR (151 MHz, methanol-d₄) δ ppm 38.1, 107.4 (d, J=26.4 Hz, 2C), 114.1 (d, J=27.5 Hz, 4C), 115.5 (d, J=9.9 Hz, 4C), 121.6 (br. s., 2C), 129.6 (br. s., 2C), 136.3 (2C), 140.8, 147.6 (br. s., 2C), 159.8, 160.6 (d, J=247.6Hz, 2C). HRMS (ESI, m/z): 391.0806.

### Example 31: Bis(5-methyl-1H-indol-3-yl)(4-(methoxycarbonyl)phenyl)methylium methanesulfonate, Compound 27

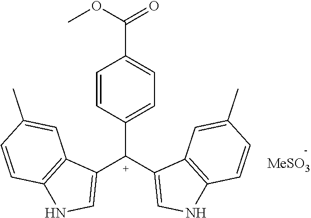 [SMILES: "MeSO3" ; COC(=O)c1ccc([C+](c2c[nH]c3ccc(C)cc23)c2c[nH]c3ccc(C)cc23)cc1]

Bis(5-methyl-1H-indol-3-yl)(4-(methoxycarbonyl)phenyl)methylium methanesulfonate was synthesized using General Procedure A, Example 1. Methanesulfonic acid was used in Step b. The product was obtained as red solid in 17% yield; ¹H NMR (600 MHz, methanol-d₄) δ ppm 2.19 (s, 6H), 2.69 (s, 3H), 4.00 (s, 3H), 6.58 (s, 2H), 7.25 (d, J=8.1 Hz, 2H), 7.56 (d, J=8.4 Hz, 2H), 7.73 (d, J=7.3 Hz, 2H), 8.25 (d, J=8.1 Hz, 4H). ¹³C NMR (151 MHz, methanol-d₄) δ ppm 20.3 (2C), 38.1, 51.8, 113.6 (4C), 120.6 (2C), 122.0 (4C), 127.4 (4C), 129.6 (2C), 134.0, 134.7 (2C), 138.0, 146.3 (2C), 166.1. HRMS (ESI, m/z): 403.1477.

### Example 32: (1-Methyl-1H-indol-3-yl)(1H-indol-3-yl)(4-(trifluoromethyl)phenyl)methylium methanesulfonate, Compound 28

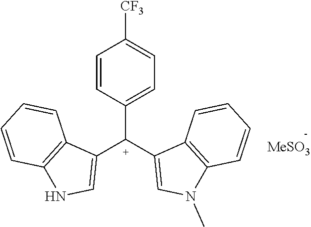 [SMILES: "MeSO3" ; Cn1cc([C+](c2ccc(C(F)(F)F)cc2)c2c[nH]c3ccccc23)c2ccccc21]

(1-Methyl-1H-indol-3-yl)(1H-indol-3-yl)(4-(trifluoromethyl)phenyl)methylium methanesulfonate was synthesized using General Procedure D, Example 4. Methanesulfonic acid was used in Step c. The product was obtained as red solid in 53% yield; ¹H NMR (600 MHz, DMSO-d₆) δ ppm 2.35 (s, 3H), 4.08 (s, 3H), 6.52-6.83 (m, 2H), 7.18 (t, J=7.43 Hz, 1H), 7.26 (t, J=7.43 Hz, 1H), 7.41 (t, J=7.52 Hz, 1H), 7.51 (t, J=7.52 Hz, 1H), 7.76 (d, J=8.07 Hz, 1H), 7.83-7.96 (m, 3H), 8.07 (d, J=8.07 Hz, 2H), 8.54-8.94 (m, 2H), 14.12 (br. s, 1H). HRMS (ESI, m/z): 403.1477.

### Example 33: Bis(2-methyl-1H-indol-3-yl)(4-fluorophenyl)methylium chloride, Compound 29

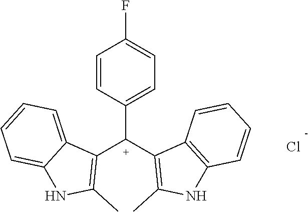 [SMILES: Cc1[nH]c2ccccc2c1[C+](c1ccc(F)cc1)c1c(C)[nH]c2ccccc12 ; Cl]

Bis(2-methyl-1H-indol-3-yl)(4-fluorophenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 35% yield; ¹H NMR (600 MHz, DMSO-d₆) δ ppm 2.11 (s, 6H), 6.64 (d, J=7.9 Hz, 2H), 7.06 (t, J=7.5 Hz, 2H), 7.29 (t, J=7.6 Hz, 2H), 7.46 (t, J=8.4 Hz, 2H), 7.56-7.65 (m, 4H). ¹³C NMR (151 MHz, DMSO-d₆) δ ppm 15.3 (2C), 113.8 (2C), 117.3 (d, J=22.0 Hz, 2C), 121.0 (2C), 121.1 (2C), 124.6 (2C), 125.7 (2C), 127.8 (2C), 136.2 (2C), 136.3 (d, J=9.9 Hz, 2C), 138.1 (2C), 156.7, 166.1 (d, J=219.0 Hz), 167.1.

### Example 34: Bis(1H-indol-3-yl)(4-hydroxyphenyl)methylium chloride, Compound 30

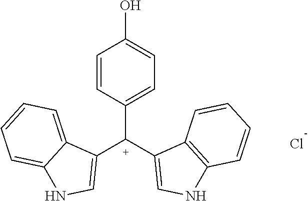 [SMILES: Oc1ccc([C+](c2c[nH]c3ccccc23)c2c[nH]c3ccccc23)cc1 ; Cl]

Bis(1H-indol-3-yl)(4-hydroxyphenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 23% yield; ¹H NMR (600 MHz, DMSO-d₆) δ ppm 6.90 (br. s., 2H), 7.07-7.21 (m, 4H), 7.37 (t, J=7.7 Hz, 2H), 7.57 (d, J=7.0 Hz, 2H), 7.72 (d, J=8.1 Hz, 2H), 8.51 (br. s., 2H), 11.34 (br. s., 1H). ¹³C NMR (151 MHz, DMSO-d₆) δ ppm 114.7 (4C), 117.1 (4C), 121.2 (2C), 124.3 (4C), 125.8 (4C), 132.0, 139.9, 146.4 (2C).

### Example 35: Bis(6-methoxy-1H-indol-3-yl)(4-(trifluoromethyl)phenyl)methylium chloride, Compound 31

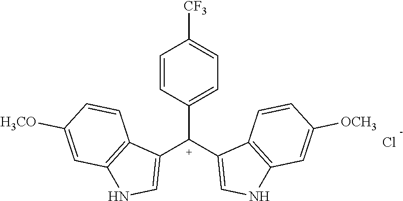 [SMILES: COc1ccc2c([C+](c3ccc(C(F)(F)F)cc3)c3c[nH]c4cc(OC)ccc34)c[nH]c2c1 ; Cl]

Bis(6-methoxy-1H-indol-3-yl)(4-(trifluoromethyl)phenyl)methylium chloride was synthesized using General Procedure B, Example 2. HCl (37%) was used in Step b. The product was obtained as red solid in 63% yield; ¹H NMR (600 MHz, DMSO-d₆) δ ppm 3.83 (s, 6H), 6.54 (br. s., 2H), 6.81 (br. s., 2H), 7.20 (br. s., 2H), 7.86 (br. s., 2H), 8.03 (br. s., 2H), 8.55 (br. s., 2H), 14.20 (br. s., 2H). ¹³C NMR (151 MHz, DMSO-d₆) δ ppm 29.48 (1C), 56.18 (2C), 98.53 (4C), 113.72 (4C), 120.02-120.14 (1C), 122.18 (2C), 122.56 (1C), 125.09-125.43 (1C), 126.43-126.68 (1C), 132.42-132.71 (1C), 141.66 (1C), 148.25 (2C), 158.72 (4C). HRMS (ESI, m/z): 449.1475.

### Example 36: Tris(1H-indol-3-yl)methylium chloride, Compound 32

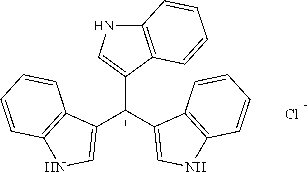 [SMILES: c1ccc2c([C+](c3c[nH]c4ccccc34)c3c[nH]c4ccccc34)c[nH]c2c1 ; Cl]

Tris(1H-indol-3-yl)methylium chloride was synthesized using General Procedure B, Example 2. HCl (37%) was used in Step b. The product was obtained as red solid in 92% yield; ¹H NMR (600 MHz, DMSO-d₆) δ ppm 6.69-7.16 (m, 6H), 7.33 (t, J=7.89 Hz, 3H), 7.75 (d, J=8.07 Hz, 3H), 8.40 (s, 3H), 13.81 (br. s., 3H). ¹³C NMR (151 MHz, DMSO-d₆) δ ppm 35.14 (1C), 114.33 (3C), 120.75 (3C), 123.51 (3C), 125.11 (3C), 127.55 (3C), 139.52 (3C), 143.77 (3C), 160.83 (3C). HRMS (ESI, m/z): 360.1495.

### Example 37: Bis(1H-indol-3-yl)((1,1′-biphenyl)-4-yl)methylium methanesulfonate, Compound 33

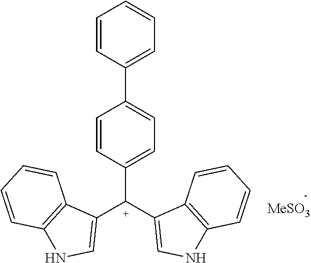 [SMILES: "MeSO3" ; c1ccc(-c2ccc([C+](c3c[nH]c4ccccc34)c3c[nH]c4ccccc34)cc2)cc1]

Bis(1H-indol-3-yl)((1,1′-biphenyl)-4-yl)methylium methanesulfonate was synthesized using General Procedure B, Example 2. HCl (37%) was used in Step b. The product was obtained as red solid in 88% yield; ¹H NMR (600 MHz, DMSO-d₆) δ ppm 2.33-2.40 (m, 3H), 6.83 (br. s., 2H), 7.18 (t, J=7.61 Hz, 2H), 7.41 (t, J=7.61 Hz, 2H), 7.48-7.53 (m, 1H), 7.58 (t, J=7.70 Hz, 2H), 7.74 (d, J=8.07 Hz, 2H), 7.79 (d, J=7.89 Hz, 2H), 7.93 (d, J=7.34 Hz, 2H), 8.06 (d, J=8.25 Hz, 2H), 8.67 (br. s., 2H), 13.98 (br. s., 2H). ¹³C NMR (151 MHz, DMSO-d₆) δ ppm 114.95 (2C), 121.71 (1C), 121.92 (2C), 124.82 (2C), 126.32 (2C), 127.57 (3C), 127.75 (2C), 129.38 (2C), 129.76 (3C), 138.77 (2C), 140.14 (2C), 145.45 (1C), 147.88 (2C). HRMS (ESI, m/z): 397.1703.

### Example 38: Bis(1H-indol-3-yl)(4-tert-butylphenyl)methylium methanesulfonate, Compound 34

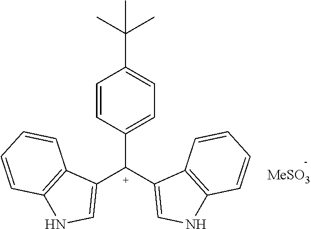 [SMILES: "MeSO3" ; CC(C)(C)c1ccc([C+](c2c[nH]c3ccccc23)c2c[nH]c3ccccc23)cc1]

Bis(1H-indol-3-yl)(4-tert-butylphenyl)methylium methanesulfonate was synthesized using General Procedure B, Example 2. HCl (37%) was used in Step b. The product was obtained as red solid in 81% yield; ¹H NMR (600 MHz, DMSO-d₆) δ ppm 1.43 (s, 9H), 2.33 (s, 3H), 6.74 (br. s., 2H), 7.16 (t, J=7.61 Hz, 2H), 7.40 (t, J=7.61 Hz, 2H), 7.62 (d, J=8.25 Hz, 2H), 7.73 (dd, J=8.16, 3.76 Hz, 4H), 8.57 (br. s., 2H), 13.90 (br. s., 2H). ¹³C NMR (151 MHz, DMSO-d₆) δ ppm 31.42 (3C), 35.66 (1C), 114.92 (3C), 121.87 (2C), 124.72 (3C), 126.23 (3C), 126.66 (2C), 140.05 (2C), 147.76 (2C), 157.99 (2C). HRMS (ESI, m/z): 377.2013.

### Example 39: Bis(1H-indol-3-yl)(4-nitrophenyl)methylium methanesulfonate, Compound 35

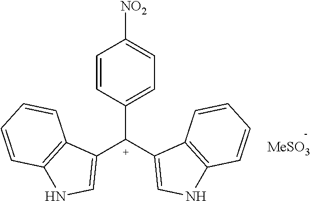 [SMILES: "MeSO3" ; O=[N+]([O-])c1ccc([C+](c2c[nH]c3ccccc23)c2c[nH]c3ccccc23)cc1]

Bis(1H-indol-3-yl)(4-nitrophenyl)methylium methanesulfonate was synthesized using General Procedure B, Example 2. HCl (37%) was used in Step b. The product was obtained as red solid in 74% yield; ¹H NMR (600 MHz, DMSO-d₆) δ ppm 2.35 (s, 3H), 6.70 (br. s., 2H), 7.17 (t, J=7.52 Hz, 2H), 7.42 (t, J=7.61 Hz, 2H), 7.73 (d, J=8.07 Hz, 2H), 7.97 (d, J=8.44 Hz,2H), 8.50 (d, J=8.44 Hz, 2H), 8.66-8.95 (m, 2H), 14.14 (br. s., 2H). ¹³C NMR (151 MHz, DMSO-d₆) δ ppm 115.17 (3C), 121.89 (2C), 122.06 (2C), 124.83 (2C), 125.21 (2C), 126.63 (3C), 140.40 (2C), 145.57 (1C), 148.87 (2C), 150.45 (2C), 166.10 (1C). HRMS (ESI, m/z): 366.1241.

### Example 40: Bis(1H-indol-3-yl)(4-fluorophenyl)methylium methanesulfonate, Compound 36

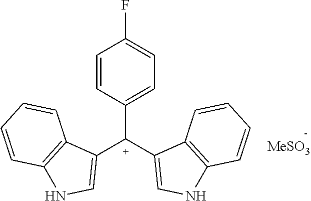 [SMILES: "MeSO3" ; Fc1ccc([C+](c2c[nH]c3ccccc23)c2c[nH]c3ccccc23)cc1]

Bis(1H-indol-3-yl)(4-fluorophenyl)methylium methanesulfonate was synthesized using General Procedure B, Example 2. HCl (37%) was used in Step b. The product was obtained as red solid in 80% yield; ¹H NMR (600 MHz, DMSO-d₆) δ ppm 2.33-2.37 (m, 3H), 6.76 (br. s., 2H), 7.19 (t, J=7.61 Hz, 2H), 7.38-7.45 (m, 2H), 7.56 (t, J=8.71 Hz, 2H), 7.73 (d, J=8.07 Hz, 2H), 7.78 (dd, J=8.34, 5.41 Hz, 2H), 8.65 (br. s., 2H), 14.00 (br. s., 2H). ¹³C NMR (151 MHz, DMSO-d₆) δ ppm 114.99 (3C), 117.12 (1C), 117.26 (1C), 121.63 (1C), 121.98 (1C), 124.87 (3C), 126.34 (3C), 126.96 (1C), 135.94 (1C), 140.23 (2C), 148.10 (2C), 165.17 (1C), 166.85 (1C), 168.22 (1C). HRMS (ESI, m/z): 339.1296.

### Example 41: Bis(1H-indol-3-yl)(4-ethylphenyl)methylium methanesulfonate, Compound 37

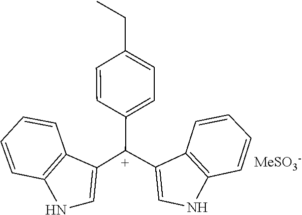 [SMILES: "MeSO3-" ; CCc1ccc([C+](c2c[nH]c3ccccc23)c2c[nH]c3ccccc23)cc1]

Bis(1H-indol-3-yl)(4-ethylphenyl)methylium methanesulfonate was synthesized using General Procedure B, Example 2. HCl (37%) was used in Step b, the product was obtained as red solid in 68% yield; ¹H NMR (600 MHz, DMSO-d₆) δ ppm 2.35 (s, 3H), 2.85 (q, J=7.64 Hz, 2H), 6.76 (br. s., 2H), 7.16 (t, J=7.61 Hz, 2H), 7.40 (t, J=7.61 Hz, 2H), 7.55 (d, J=8.07 Hz, 2H), 7.62 (d, J=7.89 Hz, 2H), 7.72 (d, J=8.07 Hz, 2H), 8.60 (br. s., 2H), 13.93 (br. s., 2H). HRMS (ESI, m/z): 349.1701.

### Example 42: Bis(4-methyl-1H-indol-3-yl)(4-(trifluoromethyl)phenyl)methylium chloride, Compound 38

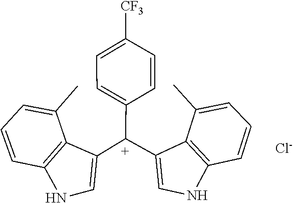 [SMILES: Cc1cccc2[nH]cc([C+](c3ccc(C(F)(F)F)cc3)c3c[nH]c4cccc(C)c34)c12 ; [Cl-]]

Bis(4-methyl-1H-indol-3-yl)(4-(trifluoromethyl)phenyl)methylium chloride was synthesized using General Procedure B, Example 2. HCl (37%) was used in Step b, the product was obtained as red solid in 84% yield; ¹H NMR (600 MHz, DMSO-d₆) δ ppm 1.54 (br. s., 6H), 7.08 (d, J=6.42 Hz, 2H), 7.37 (t, J=7.15 Hz, 2H), 7.59 (d, J=7.52 Hz, 2H), 7.68 (br. s., 2H), 8.01 (d, J=6.60 Hz, 2H), 8.34 (br. s., 2H), 14.07 (br. s., 2H). HRMS (ESI, m/z): 417.1575.

### Example 43: Bis(1H-indol-3-yl)(4-(trifluoromethoxy)phenyl)methylium chloride, Compound 39

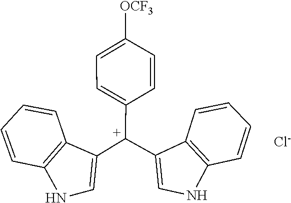 [SMILES: FC(F)(F)Oc1ccc([C+](c2c[nH]c3ccccc23)c2c[nH]c3ccccc23)cc1 ; [Cl-]]

Bis(1H-indol-3-yl)(4-(trifluoromethoxy)phenyl)methylium chloride was synthesized using General Procedure B, Example 2. HCl (37%) was used in Step b, the product was obtained as red solid in 87% yield; ¹H NMR (600 MHz, DMSO-d₆) δ ppm 6.71 (br. s., 2H), 7.18 (t, J=6.79 Hz, 2H), 7.35-7.45 (m, 2H), 7.68 (d, J=7.52 Hz, 2H), 7.74 (d, J=7.52 Hz, 2H), 7.82 (d, J=7.52 Hz, 2H), 8.66 (br. s., 2H), 14.29 (br. s., 2H). ¹³C NMR (151 MHz, DMSO-d₆) δ ppm 115.13 (4C), 119.63 (1C), 121.34 (1C), 121.65 (4C), 121.99 (2C), 124.90 (4C), 126.39 (4C), 126.85 (1C), 135.17-135.88 (1C), 140.43 (2C), 148.18 (2C), 152.37 (1C). HRMS (ESI, m/z): 405.1211.

### Example 44: Bis(1H-indol-3-yl)(pyridin-3-yl)methylium chloride, Compound 40

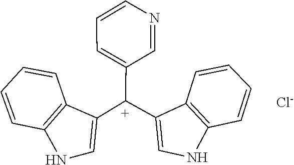 [SMILES: c1cncc([C+](c2c[nH]c3ccccc23)c2c[nH]c3ccccc23)c1 ; [Cl-]]

Bis(1H-indol-3-yl)(pyridin-3-yl)methylium chloride was synthesized using General Procedure B, Example 2. HCl (37%) was used in Step b. the product was obtained as red solid in 92% yield; ¹H NMR (600 MHz, DMSO-d₆) δ ppm 6.72 (br. s., 2H), 7.18 (br. s., 2H), 7.41 (br. s., 2H), 7.75 (br. s., 2H), 7.84 (br. s., 1H), 8.23 (br. s., 1H), 8.69 (br. s., 2H), 8.89 (br. s., 1H), 9.07 (br. s., 1H), 14.55 (br. s., 2H). HRMS (ESI, m/z): 322.1341.

### Example 45: Bis(1H-indol-3-yl)(2-(trifluoromethyl)phenyl)methylium chloride, Compound 41

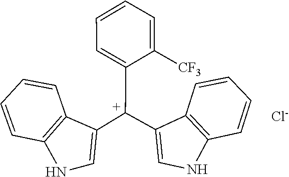 [SMILES: FC(F)(F)c1ccccc1[C+](c1c[nH]c2ccccc12)c1c[nH]c2ccccc12 ; [Cl-]]

Bis(1H-indol-3-yl)(2-(trifluoromethyl)phenyl)methylium chloride was synthesized using General Procedure B, Example 2. HCl (37%) was used in Step b. the product was obtained as red solid in 95% yield; ¹H NMR (600 MHz, DMSO-d₆) δ ppm 6.20 (br. s., 2H), 7.06 (br. s., 2H), 7.30 (br. s., 2H), 7.54-7.78 (m, 3H), 7.97 (br. s., 2H), 8.09 (br. s., 1H), 8.92 (br. s., 2H), 14.61 (br. s., 2H). ¹³C NMR (151 MHz, DMSO-d₆) δ ppm 49.32 (1C), 115.28 (2C), 121.04 (2C), 121.66 (2C), 125.37 (2C), 126.48 (2C), 126.93 (2C), 128.41 (1C), 131.31 (1C), 132.71 (1C), 134.29-134.82 (1C), 134.56 (1C), 137.09 (1C), 140.12 (s, 2C), 148.17 (2C), 165.53 (1C). HRMS (ESI, m/z): 389.1262.

### Example 46: Bis(1H-indol-3-yl)(3-(trifluoromethyl)phenyl)methylium chloride, Compound 42

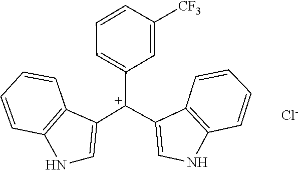 [SMILES: FC(F)(F)c1cccc([C+](c2c[nH]c3ccccc23)c2c[nH]c3ccccc23)c1 ; [Cl-]]

Bis(1H-indol-3-yl)(3-(trifluoromethyl)phenyl)methylium chloride was synthesized using General Procedure B, Example 2. HCl (37%) was used in Step b. the product was obtained as red solid in 89% yield; ¹H NMR (600 MHz, DMSO-d₆) δ ppm 6.58-6.65 (m, 2H), 7.15 (t, J=7.61 Hz, 2H), 7.39 (t, J=7.61 Hz, 2H), 7.76 (d, J=8.25 Hz, 2H), 7.93 (t, J=7.20 Hz, 1H), 7.98 (dd, J=7.89, 1.00 Hz, 1H), 8.02 (s, 1H), 8.22 (d, J=7.89 Hz, 1H), 8.70 (br. s., 2H), 14.62 (br. s., 2H). ¹³C NMR (151 MHz, DMSO-d₆) δ ppm 115.27 (2C), 121.44 (2C), 121.95 (2C), 123.35 (1C), 124.81 (2C), 125.10-125.22 (1C), 126.34 (2C), 126.90 (2C), 129.23 (1C), 129.86 (1C), 131.09 (1C), 136.72 (1C), 140.70 (2C), 148.37 (2C), 166.19 (1C). HRMS (ESI, m/z): 389.1264.

### Example 47: Bis(4-fluoro-1H-indol-3-yl)(4-(trifluoromethyl)phenyl)methylium chloride, Compound 43

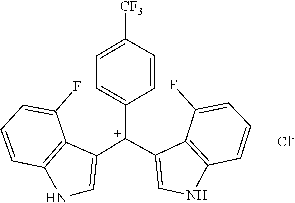 [SMILES: Fc1cccc2[nH]cc([C+](c3ccc(C(F)(F)F)cc3)c3c[nH]c4cccc(F)c34)c12 ; [Cl-]]

Bis(4-fluoro-1H-indol-3-yl)(4-(trifluoromethyl)phenyl)methylium chloride was synthesized using General Procedure B, Example 2. HCl (37%) was used in Step b. the product was obtained as red solid in 91% yield; ¹H NMR (600 MHz, DMSO-d₆) δ ppm 7.04 (br. s., 2H), 7.44 (br. s., 2H), 7.61 (br. s., 2H), 7.78 (br. s., 2H), 7.92 (br. s., 2H), 8.65 (br. s., 2H), 14.78 (br. s, 2H). ¹³C NMR (151 MHz, DMSO-d₆) δ ppm 111.04-112.23 (4C), 115.11 (1C), 115.22 (1C), 121.51 (1C), 123.53 (1C), 125.34 (1C), 125.80 (2C), 127.90 (2C), 132.22-132.96 (1C), 133.82 (2C), 143.24 (1C), 144.56 (1C), 150.20 (2C), 155.16 (1C), 156.83 (1C), 167.28 (1C). HRMS (ESI, m/z): 425.1075.

### Example 48: Bis(1H-indol-3-yl)(2,4-bis(trifluoromethyl)phenyl)methylium chloride, Compound 44

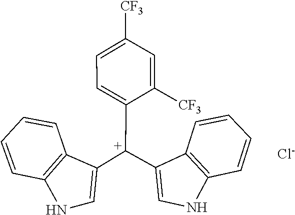 [SMILES: FC(F)(F)c1ccc([C+](c2c[nH]c3ccccc23)c2c[nH]c3ccccc23)c(C(F)(F)F)c1 ; [Cl-]]

Bis(1H-indol-3-yl)(2,4-bis(trifluoromethyl)phenyl)methylium chloride was synthesized using General Procedure B, Example 2. HCl (37%) was used in Step b. the product was obtained as red solid in 94% yield; ¹H NMR (600 MHz, DMSO-d₆) δ ppm 6.39 (br. s., 2H), 7.17 (br. s., 2H), 7.38 (br. s., 2H), 7.72 (d, J=7.15 Hz, 2H), 8.00 (d, J=6.60 Hz, 1H), 8.40 (d, J=6.60 Hz, 1H), 8.49 (br. s., 1H), 8.91 (br. s., 2H), 14.55 (br. s., 2H). ¹³C NMR (151 MHz, DMSO-d₆) δ ppm 49.08 (1C), 115.41 (4C), 121.23 (1C), 121.71 (1C), 122.19-122.84 (1C), 124.01-124.68 (1C), 125.56 (4C), 126.50 (1C), 126.58 (4C), 131.24 (1C), 140.45 (2C), 141.19 (1C), 148.76 (2C), 162.47 (1C). HRMS (ESI, m/z): 457.1135.

### Example 49: Bis(6-hydroxy-1H-indol-3-yl)(4-(trifluoromethyl)phenyl)methylium chloride, Compound 45

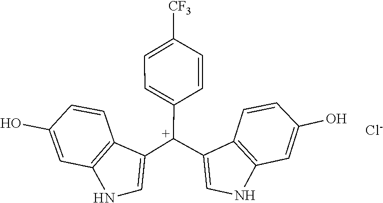 [SMILES: Oc1ccc2c([C+](c3ccc(C(F)(F)F)cc3)c3c[nH]c4cc(O)ccc34)c[nH]c2c1 ; [Cl-]]

Bis(6-hydroxy-1H-indol-3-yl)(4-(trifluoromethyl)phenyl)methylium chloride was synthesized using General Procedure C, Example 3. HCl (37%) was used in Step b. the product was obtained as red solid in 36% yield; ¹H NMR (600 MHz, DMSO-d₆) δ ppm 6.47 (br. s., 2H), 6.64 (d, J=8.07 Hz, 2H), 7.03-7.14 (m, 2H), 7.84 (d, J=7.70 Hz, 2H), 8.02 (d, J=7.89 Hz, 2H), 8.45 (br. s., 2H), 9.98 (br. s., 2H), 13.88 (br. s., 2H). ¹³C NMR (151 MHz, DMSO-d₆) δ ppm 29.34-29.67 (1C), 100.33 (2C), 114.24 (4C), 118.80 (1C), 122.35 (2C), 122.52-122.73 (1C), 123.51 (1C), 125.31 (1C), 126.34-126.62 (1C), 132.47 (1C), 132.69 (1C), 141.71 (2C), 147.72 (2C), 156.98 (4C). HRMS (EST, m/z): 421.1160.

### Example 50: Bis(5-hydroxy-1H-indol-3-yl)(4-(trifluoromethyl)phenyl)methylium chloride, Compound 46

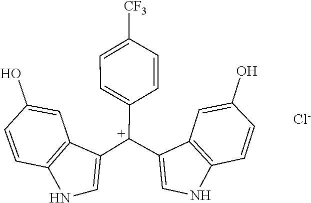 [SMILES: Oc1ccc2[nH]cc([C+](c3ccc(C(F)(F)F)cc3)c3c[nH]c4ccc(O)cc34)c2c1 ; [Cl-]]

Bis(5-hydroxy-1H-indol-3-yl)(4-(trifluoromethyl)phenyl)methylium chloride was synthesized using General Procedure C, Example 3. HCl (37%) was used in Step c. the product was obtained as red solid in 35% yield; ¹H NMR (600 MHz, DMSO-d₆) δ ppm 6.02 (br. s., 2H), 6.80-6.95 (m, 2H), 7.49 (d, J=8.62 Hz, 2H), 7.84 (d, J=7.34 Hz, 2H), 8.05 (d, J=7.70 Hz, 2H), 8.46 (br. s., 2H), 9.47 (br. s., 2H), 13.97 (br. s., 2H). ¹³C NMR (151 MHz, DMSO-d₆) δ ppm 29.47 (1C), 107.00 (1C), 115.35 (4C), 115.62 (4C), 121.40 (2C), 123.56 (1C), 125.37 (1C), 126.73 (1C), 128.05-128.45 (1C), 132.61 (1C), 132.82 (1C), 133.59 (2C), 146.78 (2C), 155.68 (2C). HRMS (ESI, m/z): 421.1158.

### Example 51: (1-Ethyl-1H-indol-3-yl)(1H-indol-3-yl)(4-(trifluoromethyl)phenyl)methylium chloride, Compound 47

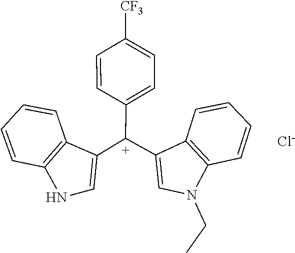 [SMILES: CCn1cc([C+](c2ccc(C(F)(F)F)cc2)c2c[nH]c3ccccc23)c2ccccc21 ; [Cl-]]

(1-Ethyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 52% yield; ¹H NMR (600 MHz, DMSO-d₆) δ ppm 1.56 (t, J=6.88 Hz, 3H), 4.55 (d, J=6.97 Hz, 2H), 6.64 (br. s., 2H), 7.16 (t, J=6.97 Hz, 1H), 7.22 (t, J=7.24 Hz, 1H), 7.39 (t, J=7.24 Hz, 1H), 7.46 (t, J=7.34 Hz, 1H), 7.78 (d, J=7.89 Hz, 1H), 7.87-7.95 (m, 2H), 8.06 (d, J=7.70 Hz, 2H), 8.62-8.99 (m, 2H), 14.85 (br. s., 1H). HRMS (ESI, m/z): 417.1577.

### Example 52: (1-Propyl-1H-indol-3-yl)(1H-indol-3-yl)(4-(trifluoromethyl)phenyl)methylium chloride, Compound 48

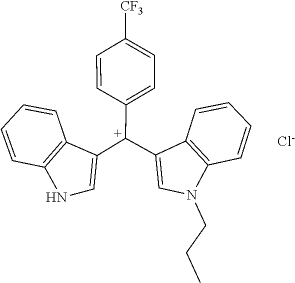 [SMILES: CCCn1cc([C+](c2ccc(C(F)(F)F)cc2)c2c[nH]c3ccccc23)c2ccccc21 ; [Cl-]]

(1-Propyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 71% yield; ¹H NMR (600 MHz, DMSO-d₆) δ ppm 0.95 (t, J=7.34 Hz, 3H), 1.35-1.45 (m, 2H), 4.51 (t, J=7.06 Hz, 2H), 6.68 (br. s., 2H), 7.18 (t, J=7.52 Hz, 1H), 7.23 (t, J=7.52 Hz, 1H), 7.76 (d, J=6.97 Hz, 1H), 7.91 (d, J=7.89 Hz, 2H), 7.92 (br. s., 1H), 8.06 (d, J=7.89 Hz, 2H), 8.70 (br. s., 1H), 8.81 (br. s., 1H), 14.56 (br. s., 1H). HRMS (ESI, m/z): 431.1735.

### Example 53: (1-Butyl-1H-indol-3-yl)(1H-indol-3-yl)(4-(trifluoromethyl)phenyl)methylium chloride, Compound 49

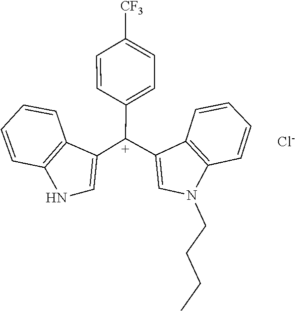 [SMILES: CCCCn1cc([C+](c2ccc(C(F)(F)F)cc2)c2c[nH]c3ccccc23)c2ccccc21 ; [Cl-]]

(1-Butyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 76% yield; ¹H NMR (600 MHz, DMSO-d₆) δ ppm 0.95 (t, J=7.34 Hz, 3H), 1.39 (sxt, J=7.41 Hz, 2H), 1.93 (quin, J=7.29 Hz, 2H), 4.51 (t, J=7.06 Hz, 2H), 6.51-6.82 (m, 2H), 7.18 (t, J=7.52 Hz, 1H), 7.23 (t, J=7.52 Hz, 1H), 7.41 (t, J=7.52 Hz, 1H), 7.47 (t, J=7.61 Hz, 1H), 7.76 (d, J=6.97 Hz, 1H), 7.91 (d, J=7.89 Hz, 2H), 7.94 (s, 1H), 8.06 (d, J=7.89 Hz, 2H), 8.56-8.96 (m, 2H), 14.58 (br. s., 1H). HRMS (ESI, m/z): 445.1889.

### Example 54: (1-Pentyl-1H-indol-3-yl)(1H-indol-3-yl)(4-(trifluoromethyl)phenyl)methylium chloride, Compound 50

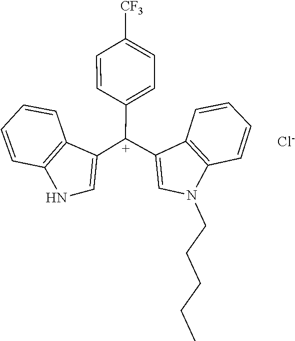 [SMILES: CCCCCn1cc([C+](c2ccc(C(F)(F)F)cc2)c2c[nH]c3ccccc23)c2ccccc21 ; [Cl-]]

(1-Pentyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 87% yield; ¹H NMR (600 MHz, DMSO-d₆) δ ppm 0.78-0.98 (m, 3H), 1.36 (d, J=3.67 Hz, 4H), 1.95 (br. s., 2H), 4.50 (t, J=7.06 Hz, 2H), 6.51-6.80 (m, 2H), 7.18 (t, J=7.34 Hz, 1H), 7.24 (t, J=7.52 Hz, 1H), 7.41 (t, J=7.61 Hz, 1H), 7.48 (t, J=7.61 Hz, 1H), 7.77 (d, J=7.15 Hz, 1H), 7.92 (dd, J=12.56, 8.16 Hz, 3H), 8.07 (d, J=8.07 Hz, 2H), 8.58-8.99 (m, 2H), 8.73-8.73 (m, 1H), 14.16-14.82 (m, 1H). HRMS (ESI, m/z): 459.2048.

### Example 55: Bis(1-pentyl-1H-indol-3-yl)(4-(trifluoromethyl)phenyl)methylium chloride, Compound 51

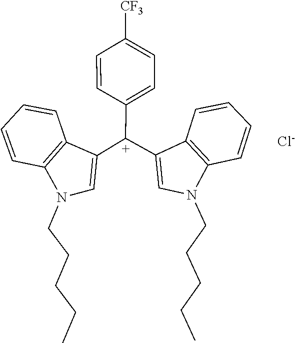 [SMILES: CCCCCn1cc([C+](c2ccc(C(F)(F)F)cc2)c2cn(CCCCC)c3ccccc23)c2ccccc21 ; [Cl-]]

Bis(1-pentyl-1H-indol-3-yl)(4-(trifluoromethyl)phenyl)methylium chloride was synthesized using General Procedure B, Example 2. HCl (37%) was used in Step b. the product was obtained as red solid in 41% yield; ¹H NMR (600 MHz, DMSO-d₆) δ ppm 0.89 (t, J=6.60 Hz, 6H), 1.36 (d, J=2.93 Hz, 8H), 1.96 (br. s., 4H), 4.51 (t, J=7.15 Hz, 4H), 6.53-6.90 (m, 2H), 7.25 (t, J=7.34 Hz, 2H), 7.49 (t, J=7.43 Hz, 2H), 7.92 (d, J=6.97 Hz, 2H), 7.95 (d, J=8.25 Hz, 2H), 8.07 (d, J=7.89 Hz, 2H), 8.82 (br. s., 2H). HRMS (ESI, m/z): 529.2832.

### Example 56: Bis(1H-indol-3-yl)(thiophen-2-yl)methyliumchloride, Compound 52

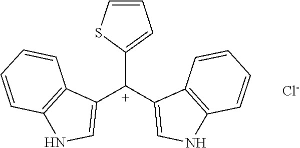 [SMILES: c1csc([C+](c2c[nH]c3ccccc23)c2c[nH]c3ccccc23)c1 ; [Cl-]]

Bis(1H-indol-3-yl)(thiophen-2-yl)methylium chloride was synthesized using General Procedure B, Example 2. HCl (37%) was used in Step b. the product was obtained as red solid in 77% yield; ¹H NMR (600 MHz, DMSO-d₆) δ ppm 6.82 (d, J=5.32 Hz, 2H), 7.11 (br. s., 2H), 7.38 (t, J=6.60 Hz, 2H), 7.62 (br. s., 1H), 7.76 (d, J=8.07 Hz, 2H), 7.97 (br. s., 1H), 8.59 (d, J=2.20 Hz, 2H), 8.61 (d, J=3.85 Hz, 1H), 12.74-15.63 (m, 1H). HRMS (ESI, m/z): 327.0954.

### Example 57: Bis(1H-indol-3-yl)(1-benzothiophen-3-yl)methylium chloride, Compound 53

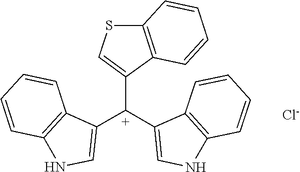 [SMILES: c1ccc2c([C+](c3c[nH]c4ccccc34)c3csc4ccccc34)c[nH]c2c1 ; [Cl-]]

Bis(1H-indol-3-yl)(1-benzothiophen-3-yl)methylium chloride was synthesized using General Procedure B, Example 2. HCl (37%) was used in Step b. the product was obtained as red solid in 39% yield; ¹H NMR (600 MHz, DMSO-d₆) δ ppm 6.65 (br. s., 2H), 7.13 (t, J=7.43 Hz, 2H), 7.38 (t, J=7.52 Hz, 2H), 7.65-7.69 (m, 3H), 7.69-7.72 (m, 2H), 7.74 (d, J=7.89 Hz, 2H), 7.88 (t, J=6.88 Hz, 1H), 8.68 (br. s., 2H), 14.39 (br. s., 2H). HRMS (ESI, m/z): 377.1108.

### Example 58: (6-Methyl-1H-indol-3-yl)(1H-indol-3-yl)(4-(trifluoromethyl)phenyl)methylium chloride, Compound 54

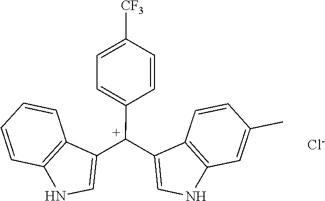 [SMILES: Cc1ccc2c([C+](c3ccc(C(F)(F)F)cc3)c3c[nH]c4ccccc34)c[nH]c2c1 ; [Cl-]]

(6-Methyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 83% yield; ¹H NMR (600 MHz, DMSO-d₆) δ ppm 2.40 (br. s., 3H), 6.40-6.73 (m, 2H), 6.98 (br. s., 1H), 7.14 (br. s., 1H), 7.37 (br. s., 1H), 7.53 (br. s., 1H), 7.72 (br. s., 1H), 7.85 (br. s., 2H), 8.02 (br. s., 2H), 8.62 (br. s., 2H), 14.42 (br. s., 2H). ¹³C NMR (151 MHz, DMSO-d₆) δ ppm 21.70 (1C), 115.16 (3C), 121.54 (3C), 122.46 (1C), 123.48 (1C), 124.45 (1C), 124.80 (2C), 125.29 (1C), 126.13-126.90 (3C), 132.63 (1C), 132.85 (1C), 136.49 (2C), 140.46 (1C), 141.23 (1C), 147.74 (1C), 148.90 (1C), 165.71 (1C). HRMS (ESI, m/z): 403.1419.

### Example 59: (7-Methyl-1H-indol-3-yl)(1H-indol-3-yl)(4-(trifluoromethyl)phenyl)methylium chloride, Compound 55

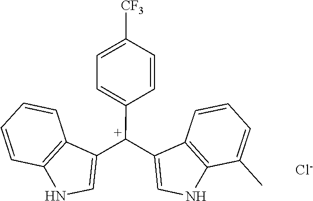 [SMILES: Cc1cccc2c([C+](c3ccc(C(F)(F)F)cc3)c3c[nH]c4ccccc34)c[nH]c12 ; [Cl-]]

(7-Methyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 80% yield; ¹H NMR (600 MHz, DMSO-d₆) δ ppm 2.57-2.68 (m, 3H), 6.46 (br. s., 1H), 6.65 (br. s., 1H), 7.05 (br. s., 1H), 7.10-7.27 (m, 2H), 7.39 (br. s., 1H), 7.74 (br. s., 1H), 7.87 (br. s., 2H), 8.04 (br. s., 2H), 8.64 (br. s., 2H), 14.43 (br. s., 2H). ¹³C NMR (151 MHz, DMSO-d₆) δ ppm 17.26 (1C), 115.34 (1C), 119.18 (1C), 121.66 (1C), 122.33 (1C), 122.51 (1C), 123.51 (1C), 124.88 (1C), 124.96 (1C), 125.07 (1C), 125.31 (1C), 126.41 (1C), 126.58 (2C), 126.86 (2C), 127.18 (1C), 132.63 (1C), 132.84 (1C), 133.45-133.74 (1C), 140.16 (1C), 141.00 (1C), 148.14 (1C), 148.61 (1C). HRMS (ESI, m/z): 403.1420.

### Example 60: (4-Methyl-1H-indol-3-yl)(1H-indol-3-yl)(4-(trifluoromethyl)phenyl)methylium chloride, Compound 56

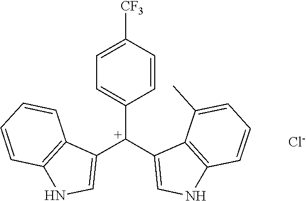 [SMILES: Cc1cccc2[nH]cc([C+](c3ccc(C(F)(F)F)cc3)c3c[nH]c4ccccc34)c12 ; [Cl-]]

(4-Methyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 75% yield; ¹H NMR (600 MHz, DMSO-d₆) δ ppm 1.42 (br. s., 3H), 6.80 (br. s., 1H), 7.04 (br. s., 1H), 7.17 (br. s., 1H), 7.31-7.45 (m, 2H), 7.60 (br. s., 2H), 7.75 (br. s., 2H), 8.02 (br. s., 2H), 8.38 (br. s., 1H), 8.64 (br. s., 1H), 14.39 (br. s., 1H). HRMS (ESI, m/z): 403.1420.

### Example 61: (5-Methyl-1H-indol-3-yl)(1H-indol-3-yl)(4-(trifluoromethyl)phenyl)methylium chloride, Compound 57

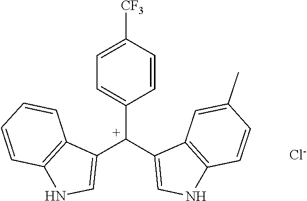 [SMILES: Cc1ccc2[nH]cc([C+](c3ccc(C(F)(F)F)cc3)c3c[nH]c4ccccc34)c2c1 ; [Cl-]]

(5-Methyl-1H-indol-3-yl)(1H-indol-3-yl)(4-(trifluoromethyl)phenyl)methylium chloride was synthesized using General Procedure D, Example 4. HCl (37%) was used in Step c. the product was obtained as red solid in 81% yield; ¹H NMR (600 MHz, DMSO-d₆) δ ppm 2.16 (s, 3H), 6.41 (br. s., 1H), 6.66 (br. s., 1H), 7.18 (t, J=7.24 Hz, 1H), 7.24 (d, J=7.70 Hz, 1H), 7.40 (t, J=7.15 Hz, 1H), 7.61 (d, J=8.07 Hz, 1H), 7.73 (d, J=7.89 Hz, 1H), 7.88 (d, J=7.52 Hz, 2H), 8.06 (d, J=7.52 Hz, 2H), 8.64 (d, J=19.81 Hz, 2H), 14.27 (br. s., 2H). HRMS (ESI, m/z): 403.1419.

### Example 62: Bis(1H-indol-3-yl)(phenyl)methylium methanesulfonate, Compound 58

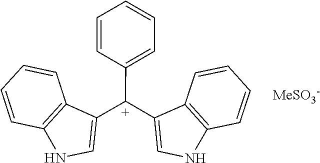 [SMILES: "MeSO3-" ; c1ccc([C+](c2c[nH]c3ccccc23)c2c[nH]c3ccccc23)cc1]

Bis(1H-indol-3-yl)(phenyl)methylium methanesulfonate was synthesized using General Procedure A, Example 1. Methanesulfonic acid was used in Step b. The product was obtained as red solid in 18% yield; ¹H NMR (600 MHz, methanol-d₄) δ ppm 2.70 (s, 3H), 6.84 (d, J=8.1 Hz, 2H), 7.12 (t, J=7.5 Hz, 2H), 7.39 (t, J=7.5 Hz, 2H), 7.53-7.80 (m, 6H), 7.84 (br. s., 1H), 8.28 (br. s., 2H). ¹³C NMR (151 MHz, methanol-d₄) δ ppm 38.1, 114.0 (4C), 121.6 (2C), 122.3 (2C), 124.3 (2C), 125.9 (2C), 126.7 (2C), 129.0 (2C), 133.1, 133.5 (2C), 140.2, 146.3 (2C). HRMS (ESI, m/z): 321.1383.

### Example 63: Bis(1H-indol-3-yl)(phenyl)methylium chloride, Compound 59

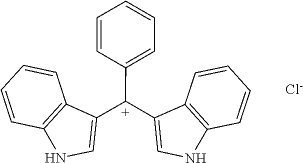 [SMILES: c1ccc([C+](c2c[nH]c3ccccc23)c2c[nH]c3ccccc23)cc1 ; [Cl-]]

Bis(1H-indol-3-yl)(phenyl)methylium choride was synthesized using General Procedure A, Example 1. HCl (37%) acid was used in Step b.

### Example 64: 3-(Bis(1H-indol-3-yl)metheyliumyl)-1-ethylpyridin-1-ium dichloride, Compound 60

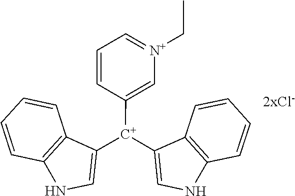

3-(Bis(1H-indol-3 -yl)metheyliumyl)-1-ethylpyridin-1-ium chloride was synthesized using General Procedure A, Example 1. HCl (37%) acid was used in Step b. The product was obtained as red solid in 2% yield; HRMS (ESI, m/z): 175.5864.

### Example 65: Bis(6-carboxy-1H-indol-3-yl)(4-(trifluoromethyl)phenyl)methylium chloride, Compound 61

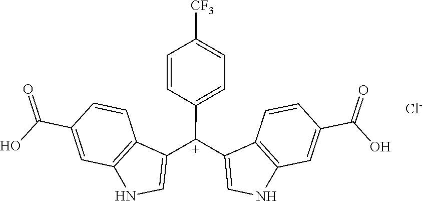 [SMILES: O=C(O)c1ccc2c([C+](c3ccc(C(F)(F)F)cc3)c3c[nH]c4cc(C(=O)O)ccc34)c[nH]c2c1 ; [Cl-]]

Bis(6-carboxy-1H-indol-3-yl)(4-(trifluoromethyl)phenyl)methylium chloride was synthesized using General Procedure A, Example 1. HCl (37%) acid was used in Step b. The product was obtained as red solid in 93.4% yield; ¹H NMR (600 MHz, DMSO-d₆) δ ppm 6.96 (br. s., 2H), 7.68 (br. s., 2H), 7.74 (br. s., 2H), 7.93 (br. s., 2H), 8.07 (br. s., 2H), 8.31 (br. s., 2H), 8.88 (br. s., 2H), 12.99(br. s., 2H), 14.90(br. s., 2H). HRMS (ESI, m/z): 477.1060.

### Example 66: Bis(7-fluoro-1H-indol-3-yl)(4-(trifluoromethyl)phenyl)methylium chloride, Compound 62

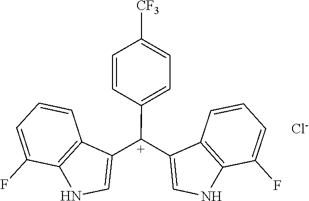 [SMILES: Fc1cccc2c([C+](c3ccc(C(F)(F)F)cc3)c3c[nH]c4c(F)cccc34)c[nH]c12 ; [Cl-]]

Bis(7-fluoro-1H-indol-3-yl)(4-(trifluoromethyl)phenyl)methylium chloride was synthesized using General Procedure A, Example 1. HCl (37%) was used in Step b. HCl (37%) was used in Step b. The product was obtained as red solid in 92.5% yield; 1H NMR (600 MHz, DMSO-d₆) δ ppm 6.48 (br. s., 2H), 7.15 (br. s., 2H), 7.26 (br. s., 2H), 7.90 (br. s., 2H), 8.05 (br. s., 2H), 8.69 (br. s., 2H), 14.00 (br. s., 2H). HRMS (ESI, m/z): 425.1073.

### Example 67: Bis(1H-indol-3-yl)(furan-2-yl)methylium chloride, Compound 63

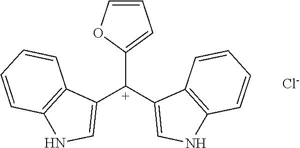 [SMILES: c1coc([C+](c2c[nH]c3ccccc23)c2c[nH]c3ccccc23)c1 ; [Cl-]]

Bis(1H-indol-3-yl)(furan-2-yl)methylium chloride was synthesized using General Procedure A, Example 1. HCl (37%) was used in Step b. The product was obtained as red solid in 88.3% yield; ¹H NMR (600 MHz, DMSO-d₆) δ ppm 6.75 (br. s, 2H), 7.19 (t, J=7.61 Hz, 2H), 7.42 (t, J=7.61 Hz, 2H), 7.73-7.80 (m, 3H), 8.14 (d, J=7.89 Hz, 1H), 8.71 (br. s., 2H), 8.81 (br. s., 1H), 8.99-9.06 (m, 1H), 14.47 (br. s., 2H). ¹³C NMR (151 MHz, DMSO-d₆) δ ppm 49.05 (1C), 115.17 (3C), 121.49-121.76 (1C), 121.95 (2C), 124.83 (2C), 124.99 (3C), 126.44 (3C), 140.34 (2C), 148.27 (br. s., 2C), 153.88 (s, 2C).

### Example 68: Bis(1H-indol-3-yl)(4-methylphenyl)methylium methanesulfonate, Compound 64

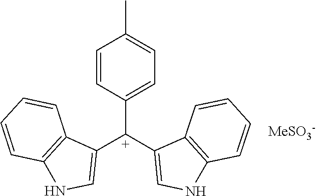 [SMILES: "MeSO3-" ; Cc1ccc([C+](c2c[nH]c3ccccc23)c2c[nH]c3ccccc23)cc1]

Bis(1H-indol-3-yl)(4-methylphenyl)methylium methanesulfonate was synthesized using General Procedure B, Example 2. Methanesulfonic acid was used in Step b. The product was obtained as red solid in 38.3% yield; ¹H NMR (600 MHz, DMSO-d₆) δ ppm 2.35 (s, 3H), 2.55 (s, 3H), 6.77 (d, J=6.97 Hz, 2H), 7.16 (t, J=7.52 Hz, 2H), 7.39 (t, J=7.61 Hz, 2H), 7.52 (d, J=7.89 Hz, 2H), 7.59 (d, J=7.70 Hz, 2H), 7.72 (d, J=7.89 Hz, 2H), 8.60 (br. s., 2H), 13.98 (br. s., 2H).

### Example 69: (1-Methyl-1H-indol-3-yl)(1H-indol-3-yl)(4-(trifluoromethyl)phenyl)methylium chloride, Compound 65

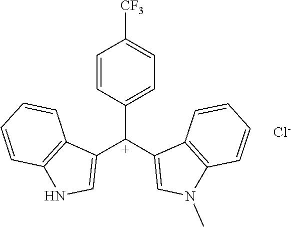 [SMILES: Cn1cc([C+](c2ccc(C(F)(F)F)cc2)c2c[nH]c3ccccc23)c2ccccc21 ; [Cl-]]

(1-Methyl-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 78.3% yield; ¹H NMR (600 MHz, DMSO-d₆) δ ppm 4.12 (s, 3H), 6.58-6.74 (m, 2H), 7.17 (t, J=7.52 Hz, 1H), 7.25 (t, J=7.61 Hz, 1H), 7.40 (t, J=7.52 Hz, 2H), 7.49 (t, J=7.61 Hz, 1H), 7.75 (d, J=7.89 Hz, 1H), 7.85 (d, J=8.25 Hz, 1H), 7.90 (d, J=7.89 Hz, 4H), 8.06 (d, J=8.07 Hz, 4H), 8.59-8.92 (m, 2H), 14.01 (br. s., 2H).

### Example 70: Bis(1H-indol-3-yl)(4-hydroxyphenyl)methylium methanesulfonate, Compound 66

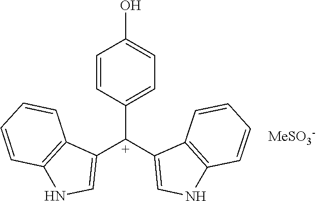 [SMILES: "MeSO3-" ; Oc1ccc([C+](c2c[nH]c3ccccc23)c2c[nH]c3ccccc23)cc1]

Bis(1H-indol-3-yl)(4-hydroxyphenyl)methylium methanesulfonate was synthesized using General Procedure C, Example 3. Methanesulfonic acid was used in Step c. The product was obtained as red solid in 56% yield; ¹H NMR (600 MHz, DMSO-d₆) δ ppm 2.39 (s, 3H), 6.90 (br. s., 2H), 7.08 (d, J=8.25 Hz, 2H), 7.16 (t, J=7.24 Hz, 2H), 7.38 (t, J=7.61 Hz, 2H), 7.59 (d, J=6.05 Hz, 2H), 7.72 (d, J=8.07 Hz, 2H), 8.51 (br. s., 2H), 11.14(br. s., 1H), 13.77 (br. s., 2H).

### Example 71: Bis(6-chloro-1H-indol-3-yl)(4-(trifluoromethyl)phenyl)methylium chloride, Compound 67

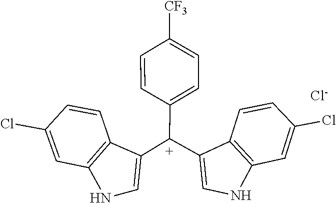 [SMILES: FC(F)(F)c1ccc([C+](c2c[nH]c3cc(Cl)ccc23)c2c[nH]c3cc(Cl)ccc23)cc1 ; [Cl-]]

Bis(6-chloro-1H-indol-3-yl)(4-(trifluoromethyl)phenyl)methylium chloride was synthesized using General Procedure B, Example 2. Methanesulfonic acid was used and the crude product was purified by ion exchange column chromatography (H₂O/EtOH/NaCl) in Step b. The product was obtained as red solid in 20.5% yield; ¹H NMR (600 MHz, DMSO-d₆) δ ppm 6.67 (br. s., 2H), 7.25 (d, J=8.44 Hz, 2H), 7.83 (s, 2H), 7.90 (d, J=8.07 Hz, 2H), 8.05 (d, J=8.07 Hz, 2H), 8.74 (br. s., 2H).

### Example 72: (5-Hydroxy-1H-indol-3-yl)(1H-indol-3-yl)(4-(trifluoromethyl)phenyl)methylium chloride, Compound 68

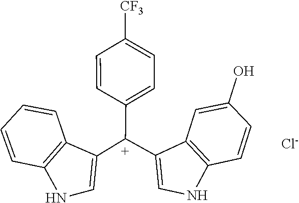 [SMILES: Oc1ccc2[nH]cc([C+](c3ccc(C(F)(F)F)cc3)c3c[nH]c4ccccc34)c2c1 ; [Cl-]]

(5-Hydroxy-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 43% yield; ¹H NMR (600 MHz, DMSO-d₆) δ ppm 6.09 (br. s., 1H), 6.60 (br. s., 1H), 6.87 (d, J=8.62 Hz, 1H), 7.16 (br. s., 1H), 7.37 (s, 1H), 7.51 (d, J=8.62 Hz, 1H), 7.71 (d, J=7.89 Hz, 1H), 7.86 (d, J=7.89 Hz, 2H), 8.05 (d, J=8.07 Hz, 2H), 8.58 (br. s., 2H), 9.55 (br. s., 1H), 14.16 (br. s., 2H).

### Example 73: (6-Hydroxy-1H-indol-3-yl)(1H-indol-3-yl)(4-(trifluoromethyl)phenyl)methylium chloride, Compound 69

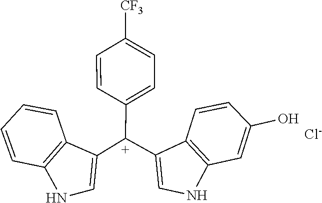 [SMILES: Oc1ccc2c([C+](c3ccc(C(F)(F)F)cc3)c3c[nH]c4ccccc34)c[nH]c2c1 ; [Cl-]]

(6-Hydroxy-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 40% yield; ¹H NMR (600 MHz, DMSO-d₆) δ ppm 6.49 (br. s., 1H), 6.62 (br. s., 1H), 6.66 (d, J=7.52 Hz, 1H), 7.12 (br. s., 1H), 7.15 (t, J=7.43 Hz, 1H), 7.38 (t, J=7.61 Hz, 1H), 7.70 (d, J=8.07 Hz, 1H), 7.87 (d, J=7.70 Hz, 2H), 8.04 (d, J=8.07 Hz, 2H), 8.58 (br. s., 2H), 10.08 (br. s., 1H), 14.09 (br. s., 2H).

### Example 74: (6-Fluoro-1H-indol-3-yl)(1H-indol-3-yl)(4-(trifluoromethyl)phenyl)methylium chloride, Compound 70

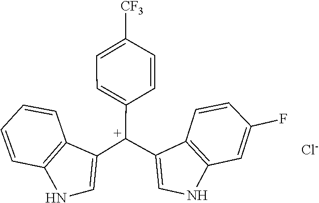 [SMILES: Fc1ccc2c([C+](c3ccc(C(F)(F)F)cc3)c3c[nH]c4ccccc34)c[nH]c2c1 ; [Cl-]]

(6-Fluoro-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 49.5% yield; ¹H NMR (600 MHz, DMSO-d₆) δ ppm 6.51-6.70 (m, 2H), 7.01 (br. s., 1H), 7.14 (br. s., 1H), 7.37 (br. s., 1H), 7.53 (br. s., 1H), 7.72 (br. s., 1H), 7.85 (br. s., 2H), 8.01 (br. s., 2H), 8.55-8.79 (m, 2H), 14.57(br. s., 2H).

### Example 75: (7-Fluoro-1H-indol-3-yl)(1H-indol-3-yl)(4-(trifluoromethyl)phenyl)methylium chloride, Compound 71

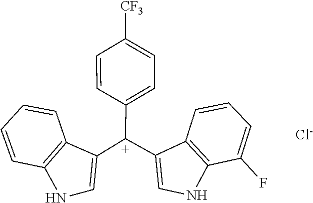 [SMILES: Fc1cccc2c([C+](c3ccc(C(F)(F)F)cc3)c3c[nH]c4ccccc34)c[nH]c12 ; [Cl-]]

(7-Fluoro-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 79% yield; ¹H NMR (600 MHz, DMSO-d₆) δ ppm 6.43 (br. s., 1H), 6.72 (d, J=2.38 Hz, 1H), 7.15 (td, J=7.89, 4.95 Hz, 1H), 7.21 (t, J=7.52 Hz, 1H), 7.24-7.30 (m, 1H), 7.44 (t, J=7.61 Hz, 1H), 7.75 (d, J=8.07 Hz, 1H), 7.91 (d, J=7.89 Hz, 2H), 8.06 (d, J=8.07 Hz, 2H), 8.68 (br. s., 1H), 8.81 (br. s., 1H), 14.57 (br. s., 2H).

### Example 76: (4-Fluoro-1H-indol-3-yl)(1H-indol-3-yl)(4-(trifluoromethyl)phenyl)methylium chloride, Compound 72

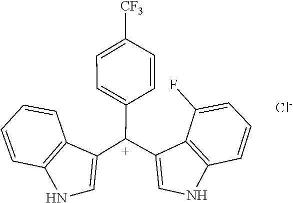 [SMILES: Fc1cccc2[nH]cc([C+](c3ccc(C(F)(F)F)cc3)c3c[nH]c4ccccc34)c12 ; [Cl-]]

(4-Fluoro-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 60% yield; ¹H NMR (600 MHz, DMSO-d₆) δ ppm 6.78 (br. s., 1H), 6.97-7.05 (m, 1H), 7.20 (t, J=7.24 Hz, 1H), 7.39-7.46 (m, 2H), 7.59 (d, J=7.52 Hz, 1H), 7.74 (d, J=7.52 Hz, 1H), 7.86 (d, J=7.70 Hz, 2H), 8.00 (d, J=7.70 Hz, 2H), 8.54 (s, 1H) 8.75 (br. s., 1H), 14.44 (br. s., 2H).

### Example 77: (5-Fluoro-1H-indol-3-yl)(1H-indol-3-yl)(4-(trifluoromethyl)phenyl)methylium chloride, Compound 73

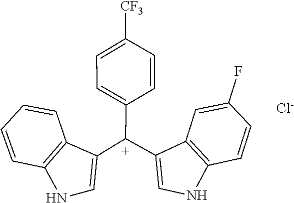 [SMILES: Fc1ccc2[nH]cc([C+](c3ccc(C(F)(F)F)cc3)c3c[nH]c4ccccc34)c2c1 ; [Cl-]]

(5-Fluoro-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 60% yield; 1H NMR (600 MHz, DMSO-d₆) δ ppm 6.27 (br. s., 1H), 6.69 (d, J=8.07 Hz, 1H), 7.19-7.24 (m, 1H), 7.28 (br. s., 1H), 7.43 (t, J=7.34 Hz, 1H), 7.73-7.79 (m, 2H), 7.90 (d, J=7.70 Hz, 2H), 8.07 (d, J=7.70 Hz, 2H), 8.70 (br. s., 1H), 8.75 (br. s., 1H), 14.43 (br. s., 2H).

### Example 78: Bis(1-phenyl-1H-indol-3-yl)(4-(trifluoromethyl)phenyl)methylium chloride, Compound 74

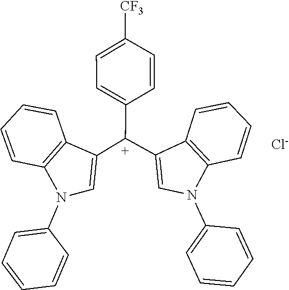 [SMILES: FC(F)(F)c1ccc([C+](c2cn(-c3ccccc3)c3ccccc23)c2cn(-c3ccccc3)c3ccccc23)cc1 ; [Cl-]]

Bis(1-phenyl-1H-indol-3-yl)(4-(trifluoromethyl)phenyl)methylium chloride was synthesized using General Procedure B, Example 2. HCl (37%) was used in Step b. The product was obtained as red solid in 20% yield; 1H NMR (600 MHz, DMSO-d₆) δ ppm 6.76 (br. s., 2H), 7.47-7.59 (m, 4H), 7.69 (m, 4H), 7.76 (m, 4H), 7.91 (d, J=6.60 Hz, 4H), 8.13 (br. s., 4H), 9.30 (br. s., 2H).

### Example 79: (1-Phenyl-1H-indol-3-yl)(1H-indol-3-yl)(4-(trifluoromethyl)phenyl)methylium chloride, Compound 75

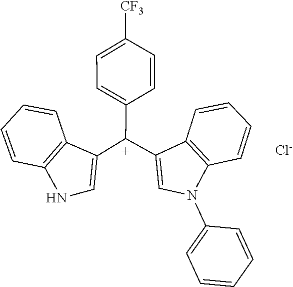 [SMILES: FC(F)(F)c1ccc([C+](c2c[nH]c3ccccc23)c2cn(-c3ccccc3)c3ccccc23)cc1 ; [Cl-]]

(1-Phenyl-1H-indol-3-yl)(1H-indol-3-yl)(4-(trifluoromethyl)phenyl)methylium chloride was synthesized using General Procedure D, Example 4. HCl (37%) was used in Step b. The product was obtained as red solid in 94.5% yield; 1H NMR (600 MHz, DMSO-d₆) δ ppm 6.68 (br. s., 2H), 7.17-7.30 (m, 2H), 7.45 (br. s., 2H), 7.63-7.70 (m, 2H), 7.71-7.80 (m, 3H), 7.90 (d, J=6.24 Hz, 2H), 8.01 (d, J=7.34 Hz, 2H), 8.09 (d, J=6.42 Hz, 2H), 8.96 (br. s., 1H), 9.15 (br. s., 1H), 14.87 (br. s., 1H).

### Example 80: Bis(5-(benzyloxy)-1H-indol-3-yl)(4-(trifluoromethyl)phenyl)methylium chloride, Compound 76

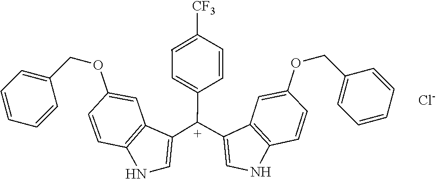 [SMILES: FC(F)(F)c1ccc([C+](c2c[nH]c3ccc(OCc4ccccc4)cc23)c2c[nH]c3ccc(OCc4ccccc4)cc23)cc1 ; [Cl-]]

Bis(5-(benzyloxy)-1H-indol-3-yl)(4-(trifluoromethyl)phenyl)methylium chloride was synthesized using General Procedure B, Example 2. HCl (37%) was used in Step b. The product was obtained as red solid in 95.6% yield; ¹H NMR (600 MHz, DMSO-d₆) δ ppm 4.73 (s, 4H), 6.12(br. s., 2H), 7.06-7.22 (m, 6H), 7.24-7.37 (m, 6H), 7.65 (d, J=8.44 Hz, 2H), 7.86 (d, J=7.70 Hz, 2H), 8.00-8.16 (d, J=7.20 Hz, 2H), 8.55 (br. s., 2H), 14.20 (br. s., 2H).

### Example 81: (5-(Benzyloxy)-1H-indol-3-yl)(1H-indol-3-yl)(4-(trifluoromethyl)phenyl)methylium chloride, Compound 77

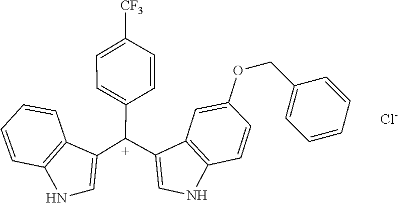 [SMILES: FC(F)(F)c1ccc([C+](c2c[nH]c3ccccc23)c2c[nH]c3ccc(OCc4ccccc4)cc23)cc1 ; [Cl-]]

(5-(Benzyloxy)-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 95.8% yield; ¹H NMR (600 MHz, DMSO-d₆) δ ppm 4.71 (s, 2H), 6.07 (br. s., 1H), 6.70 (br. s., 1H), 7.10 (d, J=8.07 Hz, 1H), 7.15 (d, J=6.97 Hz, 2H), 7.21 (br. s., 1H), 7.26-7.35 (m, 3H), 7.42 (m, 1H), 7.63 (d, J=8.62 Hz, 1H), 7.75 (d, J=7.52 Hz, 1H), 7.88 (d, J=7.52 Hz, 2H), 8.07 (d, J=7.34 Hz, 2H), 8.63 (br. s., 2H), 14.28 (br. s., 2H).

### Example 82: (6-(Benzyloxy)-1H-indol-3-yl)(1H-indol-3-yl)(4-(trifluoromethyl)phenyl)methylium chloride, Compound 78

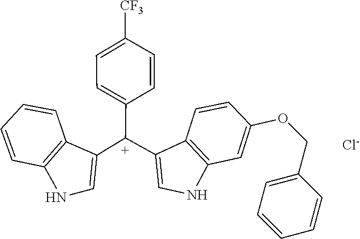 [SMILES: FC(F)(F)c1ccc([C+](c2c[nH]c3ccccc23)c2c[nH]c3cc(OCc4ccccc4)ccc23)cc1 ; [Cl-]]

(6-(Benzyloxy)-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 71.3% yield; ¹H NMR (600 MHz, DMSO-d₆) δ ppm 5.20 (s, 2H), 6.53-6.71 (m, 2H), 6.90 (d, J=8.25 Hz, 1H), 7.17 (m, 1H), 7.29 (br. s., 1H), 7.35 (m, 1H), 7.37-7.43 (m, 3H), 7.47 (d, J=6.79 Hz, 2H), 7.72 (d, J=7.52 Hz, 1H), 7.88 (d, J=6.79 Hz, 2H), 8.04 (d, J=7.34 Hz, 2H), 8.62 (br. s., 2H), 14.22 (br. s., 2H).

### Example 83: (7-Methoxy-1H-indol-3-yl)(1H-indol-3-yl)(4-(trifluoromethyl)phenyl)methylium chloride, Compound 79

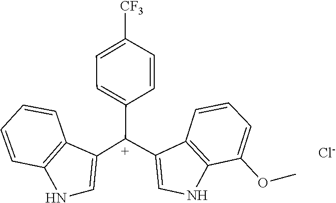 [SMILES: COc1cccc2c([C+](c3ccc(C(F)(F)F)cc3)c3c[nH]c4ccccc34)c[nH]c12 ; [Cl-]]

(7-Methoxy-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 92% yield; 1H NMR (600 MHz, DMSO-d₆) δ ppm 6.18 (br. s., 1H), 6.66 (s, 1H), 7.02 (br. s., 1H), 7.10 (s, 1H), 7.18 (br. s., 1H), 7.41 (br. s., 1H), 7.74 (d, J=5.87 Hz, 1H), 7.89 (br. s., 2H), 8.05 (d, J=5.69 Hz, 2H), 8.53 (br. s., 1H), 8.62-8.80 (m, 1H), 14.38 (br. s., 2H).

### Example 84: (6-Methoxy-1H-indol-3-yl)(1H-indol-3-yl)(4-(trifluoromethyl)phenyl)methylium chloride, Compound 80

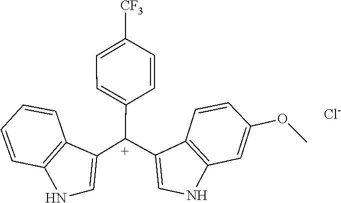 [SMILES: COc1ccc2c([C+](c3ccc(C(F)(F)F)cc3)c3c[nH]c4ccccc34)c[nH]c2c1 ; [Cl-]]

(6-Methoxy-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 40.4% yield; 1H NMR (600 MHz, DMSO-d₆) δ ppm 3.83 (s, 3H), 6.56 (br. s., 1H), 6.65 (br. s., 1H), 6.79-6.85 (m, 1H), 7.17 (t, J=7.52 Hz, 1H), 7.20 (d, J=1.28 Hz, 1H), 7.39 (t, J=7.43 Hz, 1H), 7.71 (d, J=8.07 Hz, 1H), 7.88 (d, J=7.70 Hz, 2H), 8.04 (d, J=7.89 Hz, 2H), 8.62 (br. s., 2H), 14.17 (br. s., 2H), 14.29-14.31 (m, 1H).

### Example 85: (5-Methoxy-1H-indol-3-yl)(1H-indol-3-yl)(4-(trifluoromethyl)phenyl)methylium chloride, Compound 81

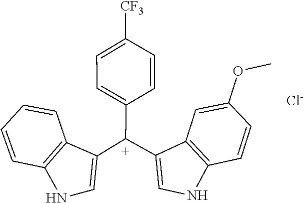 [SMILES: COc1ccc2[nH]cc([C+](c3ccc(C(F)(F)F)cc3)c3c[nH]c4ccccc34)c2c1 ; [Cl-]]

(5-Methoxy-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in 97.2% yield;1H NMR (600 MHz, DMSO-d₆) δ ppm 3.35 (br. s., 3H), 5.96 (br. s., 1H), 6.70 (br. s., 1H), 7.01 (d, J=7.89 Hz, 1H), 7.19 (t, J=6.42 Hz, 1H), 7.40 (t, J=6.51 Hz, 1H), 7.63 (d, J=8.25 Hz, 1H), 7.73 (d, J=7.52 Hz, 1H), 7.89 (d, J=7.15 Hz, 2H), 8.07 (d, J=7.34 Hz, 2H), 8.61 (br. s., 1H), 8.63 (br. s., 1H), 14.33 (br. s., 2H).

### Example 86: Bis(1H-indol-3-yl)(4-carboxyphenyl)methylium methanesulfonate, Compound 82

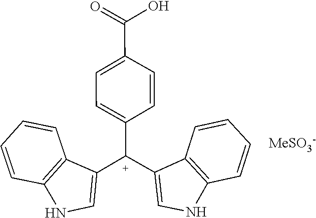 [SMILES: "MeSO3-" ; O=C(O)c1ccc([C+](c2c[nH]c3ccccc23)c2c[nH]c3ccccc23)cc1]

Bis(1H-indol-3-yl)(4-carboxyphenyl)methylium methanesulfonate was synthesized using General Procedure A, Example 1. Methanesulfonic acid was used in Step b.

### Example 87: Bis(5-methyl-1H-indol-3-yl)(4-chlorophenyl)methylium methanesulfonate, Compound 83

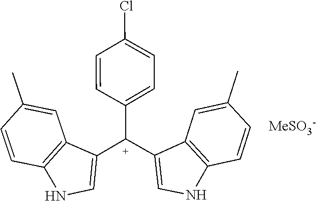 [SMILES: "MeSO3-" ; Cc1ccc2[nH]cc([C+](c3ccc(Cl)cc3)c3c[nH]c4ccc(C)cc34)c2c1]

Bis(5-methyl-1H-indol-3-yl)(4-chlorophenyl)methylium methanesulfonate was synthesized using General Procedure A, Example 1. Methanesulfonic acid was used in Step b.

### Example 88: Bis(1H-indol-3-yl)(4-carboxyphenyl)methylium chloride, Compound 84

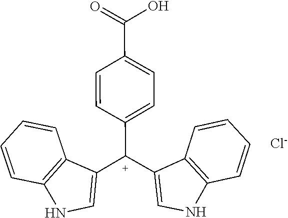 [SMILES: O=C(O)c1ccc([C+](c2c[nH]c3ccccc23)c2c[nH]c3ccccc23)cc1 ; [Cl-]]

Bis(1H-indol-3-yl)(4-carboxyphenyl)methylium chloride was synthesized using General Procedure D, Example 4. HCl (37%) was used in Step c. The product was obtained as red solid in 56% yield; 1H NMR (600 MHz, DMSO-d6) δ 14.21 (br. s, 2H), 13.56 (br. s., 1H), 8.67 (br. s., 2H), 8.20 (d, J=8.25 Hz, 2H), 7.79 (d, J=8.07 Hz, 2H), 7.71 (d, J=8.07 Hz, 2H), 7.39 (t, J=7.61 Hz, 2H), 7.15 (t, J=7.61 Hz, 2H), 6.66 (br. s., 2H). FIRMS (ESI, m/z): 365.1272.

### Example 89: Bis(1-allyl-1H-indol-3-yl)(4-(trifluoromethyl)phenyl)methylium chloride, Compound 85

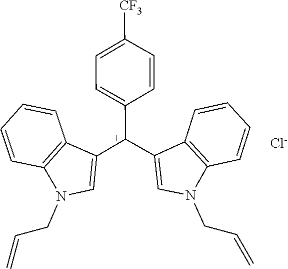 [SMILES: C=CCn1cc([C+](c2ccc(C(F)(F)F)cc2)c2cn(CC=C)c3ccccc23)c2ccccc21 ; [Cl-]]

Bis(1-allyl-1H-indol-3-yl)(4-(trifluoromethyl)phenyl)methylium chloride was synthesized using General Procedure D, Example 4. HCl (37%) was used in Step c. The product was obtained as red solid in yield 79%. ¹H NMR (600 MHz, DMSO-d₆) δ 8.86 (br. s., 2H), 8.06 (br. s., 2H), 7.93 (br. s., 2H), 7.84 (br. s., 2H), 7.46 (br. s., 2H), 7.24 (br. s., 2H), 6.70 (br. s., 1H), 6.16 (br. s., 2H), 5.74 (br. s., 1H), 5.40 (d, J=16.32 Hz, 2H), 5.35 (d, J=7.34 Hz, 2H), 5.20 (br. s., 4H).

### Example 90: (1-Allyl-1H-indol-3-yl)(1H-indol-3-yl)(4-(trifluoromethyl)phenyl)methylium chloride, Compound 86

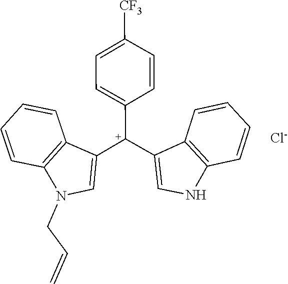 [SMILES: C=CCn1cc([C+](c2ccc(C(F)(F)F)cc2)c2c[nH]c3ccccc23)c2ccccc21 ; [Cl-]]

(1-Allyl-1H-indol-3-yl)(1H-indol-3-yl)(4-(trifluoromethyl)phenyl)methylium chloride was synthesized using General Procedure D, Example 4. HCl (37%) was used in Step c. The product was obtained as red solid in yield 70%. ¹H NMR (600 MHz, DMSO-d₆) δ 14.62 (br. s., 1H), 8.80 (br. s., 2H), 8.07 (d, J=7.70 Hz, 2H), 7.92 (d, J=7.70 Hz, 2H), 7.84 (d, J=8.25 Hz, 1H), 7.77 (d, J=7.34 Hz, 1H), 7.46 (t, J=7.43 Hz, 1H), 7.42 (t, J=7.34 Hz, 1H), 7.23 (t, J=7.34 Hz, 1H), 7.19 (t, J=7.24 Hz, 1H), 6.68 (br. s., 2H), 6.11-6.24 (m, 1H), 5.41 (d, J=17.06 Hz, 1H), 5.36 (d, J=10.27 Hz, 1H), 5.19 (d, J=3.30 Hz, 2H). ¹³C NMR (151 MHz, DMSO-d₆) δ 166.0, 149.4, 148.4, 140.0, 139.5, 132.7, 132.1, 126.2, 126.0, 125.0, 124.8, 124.8, 123.0, 121.9, 121.5, 120.3, 119.5, 114.8, 113.6, 50.2.

### Example 91: (7-Fluoro-1-methyl-1H-indol-3-yl)(1H-indol-3-yl)(4-(trifluoromethyl)phenyl)-methylium chloride, Compound 87

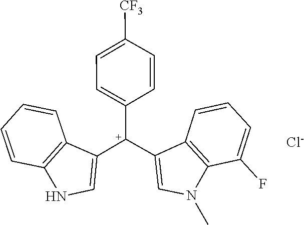 [SMILES: Cn1cc([C+](c2ccc(C(F)(F)F)cc2)c2c[nH]c3ccccc23)c2cccc(F)c21 ; [Cl-]]

(7-Fluoro-1-methyl-1H-indol-3-yl)(1H-indol-3-yl)(4-(trifluoromethyl)phenyl)-methylium chloride was synthesized using General Procedure D, Example 4. HCl (37%) was used in Step c. The product was obtained as red solid in yield 51%. ¹H NMR (600 MHz, DMSO-d₆) δ 14.74 (br. s., 1H), 8.78 (br. s., 1H), 8.69 (br. s., 1H), 8.07 (d, J=7.89 Hz, 2H), 7.90 (d, J=7.70 Hz, 2H), 7.77 (d, J=7.52 Hz, 1H), 7.44 (t, J=7.34 Hz, 1H), 7.28-7.34 (m, 1H), 7.20-7.23 (m, 1H), 7.19 (d, J=4.58 Hz, 1H), 6.71 (br. s., 1H), 6.47 (br. s., 1H), 4.23 (s, 3H).

### Example 92: (6-Fluoro-1-methyl-1H-indol-3-yl)(1H-indol-3-yl)(4-(trifluoromethyl)phenyl)-methylium chloride, Compound 88

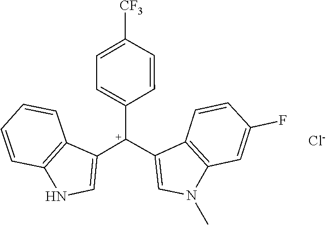 [SMILES: Cn1cc([C+](c2ccc(C(F)(F)F)cc2)c2c[nH]c3ccccc23)c2ccc(F)cc21 ; [Cl-]]

(6-Fluoro-1-methyl-1H-indol-3-yl)(1H-indol-3-yl)(4-(trifluoromethyl)phenyl)-methylium chloride was synthesized using General Procedure D, Example 4. HCl (37%) was used in Step c. The product was obtained as red solid in yield 51%. ¹H NMR (600 MHz, DMSO-d₆) δ 14.59 (br. s., 1H), 8.76 (br. s., 2H), 8.07 (d, J=7.15 Hz, 2H), 7.90 (d, J=6.79 Hz, 2H), 7.84 (d, J=8.25 Hz, 1H), 7.75 (d, J=6.60 Hz, 1H), 7.42 (br. s., 1H), 7.19 (br. s., 1H), 7.14 (t, J=8.07 Hz, 1H), 6.66 (br. s., 2H), 4.08 (br. s., 3H).

### Example 93: (7-Fluoro-1H-indol-3-yl)(1-methyl-1H-indol-3-yl)(4-(trifluoromethyl)phenyl)-methylium chloride, Compound 89

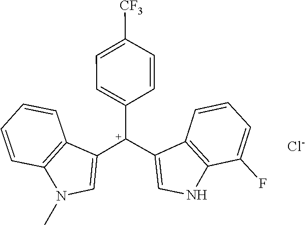 [SMILES: Cn1cc([C+](c2ccc(C(F)(F)F)cc2)c2c[nH]c3c(F)cccc23)c2ccccc21 ; [Cl-]]

(7-Fluoro-1H-indol-3-yl)(1-methyl-1H-indol-3-yl)(4-(trifluoromethyl)phenyl)-methylium chloride was synthesized using General Procedure D, Example 4. HCl (37%) was used in Step c. The product was obtained as red solid in yield 98%. ¹H NMR (600 MHz, DMSO-d₆) δ 14.54 (br. s., 1H), 8.85 (br. s., 1H), 8.65 (br. s., 1H), 8.06 (d, J=7.52 Hz, 2H), 7.90 (d, J=7.34 Hz, 2H), 7.87 (d, J=7.89 Hz, 1H), 7.51 (t, J=6.97 Hz, 1H), 7.21-7.31 (m, 2H), 7.13 (d, J=4.40 Hz, 1H), 6.76 (d, J=16.87 Hz, 1H), 6.42 (br. s., 1H), 4.12 (s, 3H). ¹³C NMR (151 MHz, DMSO-d₆) δ 151.7, 150.5, 148.8, 147.2, 140.7, 132.5, 132.3, 128.0, 127.3, 126.4, 126.0, 125.4, 124.8, 124.6, 121.4, 113.4, 110.9, 110.8, 35.0.

### Example 94: (6-Fluoro-1H-indol-3-yl)(1-methyl-1H-indol-3-yl)(4-(trifluoromethyl)phenyl)-methylium chloride, Compound 90

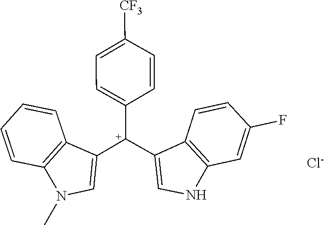 [SMILES: Cn1cc([C+](c2ccc(C(F)(F)F)cc2)c2c[nH]c3cc(F)ccc23)c2ccccc21 ; [Cl-]]

(6-Fluoro-1H-indol-3-yl)(1-methyl-1H-indol-3-yl)(4-(trifluoromethyl)phenyl)-methylium chloride was synthesized using General Procedure D, Example 4. HCl (37%) was used in Step c. The product was obtained as red solid in yield 82%. ¹H NMR (600 MHz, DMSO-d₆) δ 14.20-14.70 (m, 1H), 8.84 (br. s., 1H), 8.68 (br. s., 1H), 8.07 (d, J=7.70 Hz, 2H), 7.90 (d, J=7.70 Hz, 2H), 7.87 (d, J=7.89 Hz, 1H), 7.58 (d, J=7.52 Hz, 1H), 7.51 (t, J=7.43 Hz, 1H), 7.28 (t, J=7.24 Hz, 1H), 7.07 (t, J=8.34 Hz, 1H), 6.67-6.77 (m, 1H), 6.64 (br. s., 1H), 4.12 (s, 3H). ¹³C NMR (151 MHz, DMSO-d₆) δ 161.0, 159.4, 151.4, 147.8, 140.8, 140.1, 132.7, 132.5, 126.4, 126.1, 125.5, 124.7, 122.9, 121.0, 120.5, 113.5, 112.4, 112.3, 101.2, 101.1, 35.1.

### Example 95: (3-Bromophenyl)di(1H-indol-3-yl)methylium chloride, Compound 91

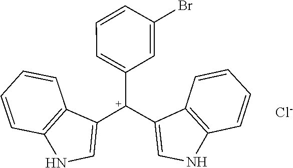 [SMILES: Brc1cccc([C+](c2c[nH]c3ccccc23)c2c[nH]c3ccccc23)c1 ; [Cl-]]

(3-Bromophenyl)di(1H-indol-3-yl)methylium chloride was synthesized using General Procedure D, Example 4. HCl (37%) was used in Step c. The product was obtained as red solid in yield 91%. ¹H NMR (600 MHz, DMSO-d₆) δ 14.29 (br. s., 2H), 8.70 (br. s., 2H), 8.07 (d, J=7.70 Hz, 1H), 7.89 (s, 1H), 7.73 (d, J=8.07 Hz, 2H), 7.67-7.70 (m, 1H), 7.63-7.67 (m, 1H), 7.40 (t, J=7.61 Hz, 2H), 7.19 (t, J=7.52 Hz, 2H), 6.70 (br. s., 2H).

### Example 96: (3-Chlorophenyl)di(1H-indol-3-yl)methylium chloride, Compound 92

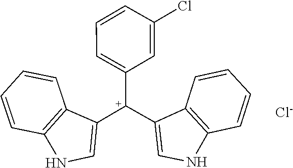 [SMILES: Clc1cccc([C+](c2c[nH]c3ccccc23)c2c[nH]c3ccccc23)c1 ; [Cl-]]

(3-Chlorophenyl)di(1H-indol-3-yl)methylium chloride was synthesized using General Procedure D, Example 4. HCl (37%) was used in Step c. The product was obtained as red solid in yield 93%. ¹⁻H NMR (400 MHz, DMSO-d₆) δ 14.14 (br. s., 2H), 8.62 (br. s., 2H), 7.92 (br. s., 1H), 7.73 (br. s., 3H), 7.64 (br. s., 1H), 7.39 (br. s., 2H), 7.17 (br. s., 2H), 6.72 (br. s., 2H). ¹³C NMR (151 MHz, DMSO-d6) d 147.7, 141.1, 133.9, 132.3, 131.1, 126.6, 125.7, 124.1, 121.7, 121.0, 114.9.

### Example 97: (2-Chlorophenyl)di(1H-indol-3-yl)methylium chloride, Compound 93

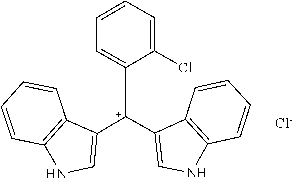 [SMILES: Clc1ccccc1[C+](c1c[nH]c2ccccc12)c1c[nH]c2ccccc12 ; [Cl-]]

(2-Chlorophenyl)di(1H-indol-3-yl)methylium chloride was synthesized using General Procedure D, Example 4. HCl (37%) was used in Step c. The product was obtained as red solid in yield 80%. ¹H NMR (400 MHz, DMSO-d₆) δ 14.59 (br. s., 2H), 8.94 (br. s., 2H), 7.84 (br. s., 2H), 7.73 (br. s., 2H), 7.67 (br. s., 1H), 7.61 (br. s., 1H), 7.37 (br. s., 2H), 7.14 (br. s., 2H), 6.46 (br. s., 2H). ¹³C NMR (151 MHz, DMSO-d6) Shift 164.1, 147.4, 139.6, 136.2, 133.3, 131.8, 131.5, 130.8, 128.5, 126.2, 125.8, 124.7, 121.0, 120.2, 114.5.

### Example 98: (2-Hydroxyphenyl)di(1H-indol-3-yl)methylium chloride, Compound 94

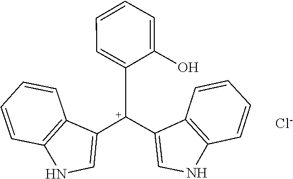 [SMILES: Oc1ccccc1[C+](c1c[nH]c2ccccc12)c1c[nH]c2ccccc12 ; [Cl-]]

(2-Hydroxyphenyl)di(1H-indol-3-yl)methylium chloride was synthesized using General Procedure D, Example 4. HCl (37%) was used in Step c. The product was obtained as red solid in yield 30%. ¹H NMR (600 MHz, DMSO-d6) δ 14.10 (br. s., 2H), 10.36 (br. s., 1H), 8.71 (br. s., 2H), 7.68 (d, J=8.07 Hz, 2H), 7.63 (t, J=7.79 Hz, 1H), 7.36 (t, J=7.61 Hz, 2H), 7.28 (d, J=6.79 Hz, 1H), 7.20 (d, J=8.25 Hz, 1H), 7.15 (t, J=7.70 Hz, 2H), 7.04 (t, J=7.52 Hz, 1H), 6.79 (d, J=6.05 Hz, 2H).

### Example 99: (3-Hydroxyphenyl)di(1H-indol-3-yl)methylium chloride, Compound 95

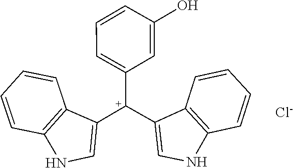 [SMILES: Oc1cccc([C+](c2c[nH]c3ccccc23)c2c[nH]c3ccccc23)c1 ; [Cl-]]

(3-Hydroxyphenyl)di(1H-indol-3-yl)methylium chloride was synthesized using General Procedure D, Example 4. HCl (37%) was used in Step c. The product was obtained as red solid in yield 65%. ¹H NMR (400 MHz, DMSO-d₆) δ 14.28 (br. s., 2H), 10.17 (br. s., 1H), 8.65 (br. s., 2H), 7.73 (br. s., 2H), 7.50 (br. s., 1H), 7.39 (br. s., 2H), 7.30 (br. s., 1H), 7.17 (br. s., 2H), 7.07 (br. s., 1H), 7.04 (br. s., 1H), 6.77 (br. s., 2H). ¹³C NMR (151 MHz, DMSO-d6) δ 169.2, 157.9, 147.0, 139.6, 130.5, 126.6, 125.6, 124.2, 121.1, 121.1, 120.4, 114.3.

### Example 100: (3-Fluorophenyl)di(1H-indol-3-yl)methylium chloride, Compound 96

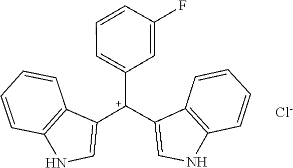 [SMILES: Fc1cccc([C+](c2c[nH]c3ccccc23)c2c[nH]c3ccccc23)c1 ; [Cl-]]

(3-Fluorophenyl)di(1H-indol-3-yl)methylium chloride was synthesized using General Procedure D, Example 4. HCl (37%) was used in Step c. The product was obtained as red solid in yield 91%. ¹H NMR (600 MHz, DMSO-d₆) δ 14.42 (br. s., 2H), 8.72 (br. s., 2H), 7.74 (d, J=6.05 Hz, 4H), 7.58 (d, J=8.80 Hz, 1H), 7.52 (d, J=5.69 Hz, 1H), 7.40 (t, J=6.60 Hz, 2H), 7.18 (t, J=6.60 Hz, 2H), 6.68 (br. s., 2H). ¹³C NMR (151 MHz, DMSO-d₆) δ 167.6, 163.8, 161.6, 147.5, 140.3, 132.4, 126.9, 126.4, 124.9, 121.9, 121.6, 120.4, 115.1.

### Example 101: Di(1H-indol-3-yl)(m-tolyl)methylium chloride, Compound 97

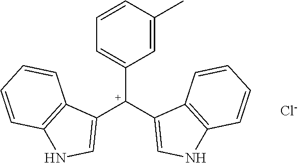 [SMILES: Cc1cccc([C+](c2c[nH]c3ccccc23)c2c[nH]c3ccccc23)c1 ; [Cl-]]

Di(1H-indol-3-yl)(m-tolyl)methylium chloride was synthesized using General Procedure D, Example 4. HCl (37%) was used in Step c. The product was obtained as red solid in yield 85%. ¹H NMR (400 MHz, DMSO-d₆) δ 14.26 (br. s., 2H), 8.64 (br. s., 2H), 7.73 (br. s., 3H), 7.60 (br. s., 1H), 7.50 (d, J=10.00 Hz, 2H), 7.40 (br. s., 2H), 7.16 (br. s., 2H), 6.72 (br. s., 2H), 2.42 (br. s., 3H).

### Example 102: Di(1H-indol-3-yl)(3-methoxyphenyl)methylium chloride, Compound 98

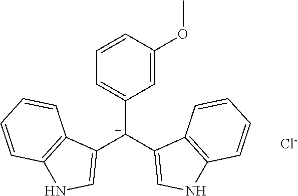 [SMILES: COc1cccc([C+](c2c[nH]c3ccccc23)c2c[nH]c3ccccc23)c1 ; [Cl-]]

Di(1H-indol-3-yl)(3-methoxyphenyl)methylium chloride was synthesized using General Procedure D, Example 4. HCl (37%) was used in Step c. The product was obtained as red solid in yield 76%. ¹H NMR (400 MHz, DMSO-d₆) δ 14.20 (s, 2H), 8.66 (br. s., 2H), 7.75 (br. s., 2H), 7.63 (br. s., 1H), 7.44-7.49 (m, 1H), 7.40 (br. s., 2H), 7.22 (br. s., 2H), 7.18 (br. s., 2H), 6.75 (br. s., 2H).

### Example 103: (2-Fluorophenyl)di(1H-indol-3-yl)methylium chloride, Compound 99

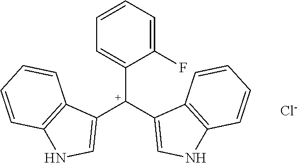 [SMILES: Fc1ccccc1[C+](c1c[nH]c2ccccc12)c1c[nH]c2ccccc12 ; [Cl-]]

(2-Fluorophenyl)di(1H-indol-3-yl)methylium chloride was synthesized using General Procedure D, Example 4. HCl (37%) was used in Step c. The product was obtained as red solid in yield 56%. ¹H NMR (600 MHz, DMSO-d₆) δ 8.38 (br. s., 2H), 7.74-7.81 (m, 1H), 7.59 (d, J=7.89 Hz, 2H), 7.51-7.54 (m, 1H), 7.47-7.50 (m, 1H), 7.43-7.46 (m, 1H), 7.27 (t, J=7.52 Hz, 2H), 7.03 (t, J=7.61 Hz, 2H), 6.64 (d, J=7.70 Hz, 2H).

### Example 104: Di(1H-indol-3-yl)(6-(trifluoromethyl)pyridin-3-yl)methylium chloride, Compound 100

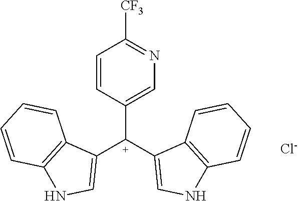 [SMILES: FC(F)(F)c1ccc([C+](c2c[nH]c3ccccc23)c2c[nH]c3ccccc23)cn1 ; [Cl-]]

Di(1H-indol-3-yl)(6-(trifluoromethyl)pyridin-3-yl)methylium chloride was synthesized using General Procedure D, Example 4. HCl (37%) was used in Step c. The product was obtained as red solid in yield 80%.'H NMR (600 MHz, DMSO-d6) ppm 14.60 (br. s., 2H) 8.98 (br. s., 1H) 8.70 (br. s., 2H) 8.40 (br. s., 1H) 8.23 (br. s., 1H) 7.78 (d, J=4.40 Hz, 2H) 7.45 (br. s., 2H) 7.22 (br. s., 2H) 6.76 (br. s., 2H). ¹³C NMR (151 MHz, DMSO-d6) 162.9, 149.8, 149.5, 148.9, 140.4, 126.7, 126.2, 125.2, 122.9, 122.3, 121.6, 121.5, 121.0, 115.3.

### Example 105: (4-Hydroxy-3-(trifluoromethyl)phenyl)di(1H-indol-3-yl)methylium chloride, Compound 101

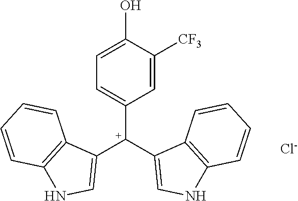 [SMILES: Oc1ccc([C+](c2c[nH]c3ccccc23)c2c[nH]c3ccccc23)cc1C(F)(F)F ; [Cl-]]

(4-Hydroxy-3-(trifluoromethyl)phenyl)di(1H-indol-3-yl)methylium chloride was synthesized using General Procedure D, Example 4. HCl (37%) was used in Step c. The product was obtained as red solid in yield 73%. 1H NMR (600 MHz, DMSO-d6) δ 14.12 (br. s., 2H), 11.63-13.36 (m, 1H), 8.57 (br. s., 2H), 7.81 (br. s., 2H), 7.74 (d, J=6.97 Hz, 2H), 7.47 (d, J=7.52 Hz, 1H), 7.40 (br. s., 2H), 7.19 (br. s., 2H), 6.87 (br. s., 2H).

### Example 106: (4-Fluoro-3-(trifluoromethyl)phenyl)di(1H-indol-3-yl)methylium chloride, Compound 102

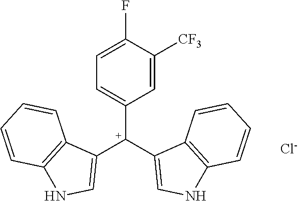 [SMILES: Fc1ccc([C+](c2c[nH]c3ccccc23)c2c[nH]c3ccccc23)cc1C(F)(F)F ; [Cl-]]

(4-Fluoro-3-(trifluoromethyl)phenyl)di(1H-indol-3-yl)methylium chloride was synthesized using General Procedure D, Example 4. HCl (37%) was used in Step c. The product was obtained as red solid in yield 82%. ¹H NMR (600 MHz, DMSO-d6) δ 14.42 (br. s., 2H), 8.66 (br. s., 2H), 8.06 (br. s., 2H), 7.83 (br. s., 1H), 7.74 (d, J=4.03 Hz, 2H), 7.41 (br. s., 2H), 7.18 (br. s., 2H), 6.75 (br. s., 2H).

### Example 107: (3-Fluoro-4-(trifluoromethyl)phenyl)di(1H-indol-3-yl)methylium chloride, Compound 103

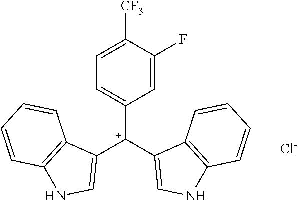 [SMILES: Fc1cc([C+](c2c[nH]c3ccccc23)c2c[nH]c3ccccc23)ccc1C(F)(F)F ; [Cl-]]

(3-Fluoro-4-(trifluoromethyl)phenyl)di(1H-indol-3-yl)methylium chloride was synthesized using General Procedure D, Example 4. HCl (37%) was used in Step c. The product was obtained as red solid in yield 78%. 1H NMR (600 MHz, DMSO-d6) δ 14.54 (br. s., 2H), 8.71 (br. s., 2H), 8.08 (br. s., 1H), 7.93 (d, J=10.64 Hz, 1H), 7.75 (d, J=6.79 Hz, 2H), 7.70 (d, J=5.50 Hz, 1H), 7.42 (br. s., 2H), 7.20 (br. s., 2H), 6.74 (br. s., 2H).

### Example 108: (4-(2,2-Difluoroethoxy)phenyl)di(1H-indol-3-yl)methylium chloride, Compound 104

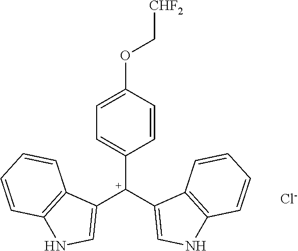 [SMILES: FC(F)COc1ccc([C+](c2c[nH]c3ccccc23)c2c[nH]c3ccccc23)cc1 ; [Cl-]]

(4-(2,2-Difluoroethoxy)phenyl)di(1H-indol-3-yl)methylium chloride was synthesized using General Procedure D, Example 4. HCl (37%) was used in Step c. The product was obtained as red solid in yield 86%. 1H NMR (600 MHz, DMSO-d6) δ 14.16 (br. s., 2H), 8.55 (br. s., 2H), 7.72 (d, J=7.15 Hz, 2H), 7.68 (d, J=6.79 Hz, 2H), 7.38 (br. s., 2H), 7.33 (d, J=7.89 Hz, 2H), 7.16 (t, J=6.51 Hz, 2H), 6.83 (br. s., 2H), 6.37-6.67 (m, 1H), 4.57 (t, J=13.94 Hz, 2H).

### Example 109: Di(1H-indol-3-yl)(4-(2,2,2-trifluoroethoxy)phenyl)methylium chloride, Compound 105

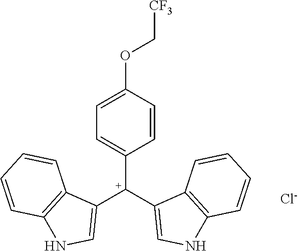 [SMILES: FC(F)(F)COc1ccc([C+](c2c[nH]c3ccccc23)c2c[nH]c3ccccc23)cc1 ; [Cl-]]

Di(1H-indol-3-yl)(4-(2,2,2-trifluoroethoxy)phenyl)methylium chloride was synthesized using General Procedure D, Example 4. HCl (37%) was used in Step c. The product was obtained as red solid in yield 84%. ¹H NMR (600 MHz, DMSO-d6) δ 14.20 (br. s., 2H), 8.56 (br. s., 2H), 7.72 (d, J=7.52 Hz, 4H), 7.38 (br. s., 4H), 7.16 (br. s., 2H), 6.81 (br. s., 2H), 5.03 (d, J=8.07 Hz, 2H).

### Example 110: Di(1H-indol-3-yl)(naphthalen-1-yl)methylium chloride, Compound 106

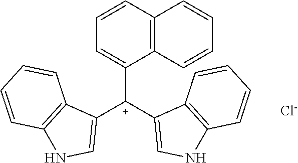 [SMILES: c1ccc2c([C+](c3c[nH]c4ccccc34)c3c[nH]c4ccccc34)cccc2c1 ; [Cl-]]

Di(1H-indol-3-yl)(naphthalen-1-yl)methylium chloride was synthesized using General Procedure D, Example 4. HCl (37%) was used in Step c. The product was obtained as red solid in yield 85.4%. ¹H NMR (600 MHz, DMSO-d₆) δ 14.49 (br. s., 2H), 8.96 (br. s., 2H), 8.40 (d, J=8.07 Hz, 1H), 8.17 (d, J=8.07 Hz, 1H), 7.79 (t, J=7.52 Hz, 1H), 7.73 (d, J=6.79 Hz, 1H), 7.69 (br. s., 1H), 7.68 (br. s., 2H), 7.56 (t, J=7.06 Hz, 1H), 7.32-7.38 (m, 1H), 7.23-7.31 (m, 2H), 6.93 (d, J=6.24 Hz, 2H), 6.13 (br. s., 2H). ¹³C NMR (151 MHz, DMSO-d₆) δ 167.1, 147.0, 139.7, 135.6, 133.3, 132.5, 130.5, 129.4, 128.8, 127.8, 126.9, 126.4, 126.0, 125.6, 124.4, 124.2, 122.3, 120.6, 114.5. HRMS (ESI): 371.1540.

### Example 111: Bis(1-ethyl-1H-indol-3-yl)(4-(trifluoromethyl)phenyl)methylium chloride, Compound 107

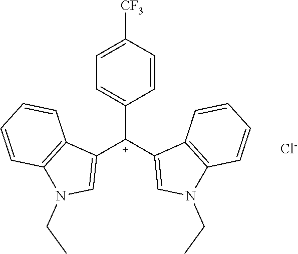 [SMILES: CCn1cc([C+](c2ccc(C(F)(F)F)cc2)c2cn(CC)c3ccccc23)c2ccccc21 ; [Cl-]]

Bis(1-ethyl-1H-indol-3-yl)(4-(trifluoromethyl)phenyl)methylium chloride was synthesized using General Procedure D, Example 4. HCl (37%) was used in Step c. The product was obtained as red solid in yield 88.7%. ¹H NMR (600 MHz, DMSO-d₆) δ 8.91 (br. s., 2H), 8.06 (br. s., 2H), 7.94 (br. s., 2H), 7.92 (br. s., 2H), 7.48 (br. s., 2H), 7.24 (br. s., 2H), 6.66 (br. s., 2H), 4.56 (br. s., 4H), 1.57 (br. s., 6H). ¹³C NMR (151 MHz, DMSO-d₆) δ 164.7, 149.1, 139.4, 132.5, 132.3, 126.6, 126.1, 126.0, 125.0, 124.7, 122.9, 121.3, 120.2, 113.3, 43.0, 14.6. HRMS (ESI): 445.1884.

### Example 112: (4-Cyanophenyl)di(1H-indol-3-yl)methylium chloride, Compound 108

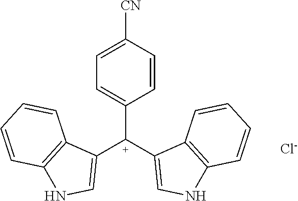 [SMILES: N#Cc1ccc([C+](c2c[nH]c3ccccc23)c2c[nH]c3ccccc23)cc1 ; [Cl-]]

(4-Cyanophenyl)di(1H-indol-3-yl)methylium chloride was synthesized using General Procedure D, Example 4. HCl (37%) was used in Step c. The product was obtained as red solid in yield 86%. ¹H NMR (600 MHz, DMSO-d₆) d=14.36 (br. s, 2H), 8.71 (br. s., 2H), 8.16 (d, J=7.7 Hz, 2H), 7.87 (d, J=7.7 Hz, 2H), 7.73 (d, J=8.1 Hz, 2H), 7.40 (t, J=7.5 Hz, 2H), 7.18 (t, J=7.4 Hz, 2H), 6.65 (br. s, 2H). ¹³C NMR (151 MHz, DMSO-d₆) d=166.4, 148.6, 142.7, 140.5, 133.7, 133.2, 126.7, 126.5, 125.1, 121.9, 121.7, 118.8, 115.5, 115.2. HRMS (ESI, m/z): 346.1336.

### Example 113: Di(1H-indol-3-yl)(4-(methylsulfonyl)phenyl)methylium chloride, Compound 109

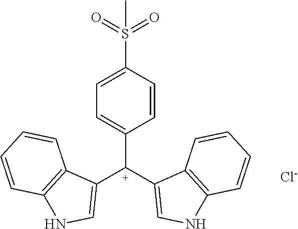 [SMILES: CS(=O)(=O)c1ccc([C+](c2c[nH]c3ccccc23)c2c[nH]c3ccccc23)cc1 ; [Cl-]]

Di(1H-indol-3-yl)(4-(methylsulfonyl)phenyl)methylium chloride was synthesized using General Procedure D, Example 4. HCl (37%) was used in Step c. The product was obtained as red solid in yield 89%. ¹H NMR (600 MHz, DMSO-d₆) d=14.35 (br. s, 2H), 8.69 (br. s., 2H), 8.21 (d, J=7.7 Hz, 2H), 7.94 (d, J=7.5 Hz, 2H), 7.74 (d, J=7.9 Hz, 2H), 7.41 (t, J=7.1 Hz, 2H), 7.18 (t, J=7.1 Hz, 2H), 6.67 (br. s, 2H), 3.43 (s, 3H). ¹³C NMR (151 MHz, DMSO-d₆) d=166.6, 148.6, 144.7, 143.1, 140.4, 133.8, 128.2, 126.7, 126.5, 125.1, 122.0, 121.8, 115.2, 43.9. HRMS (ESI, m/z): 399.1158.

### BIOLOGICAL EXAMPLES

### Example A-1: General Biological Methods

### Cell Culture

HCT116 colon cancer, MDA-MB-231, HS578T, BT549, MCF-7, and T47D breast cancer, HeLa ovarian cancer, and HEK293T embryonic cells were cultured in Dulbecco's modified Eagle's medium (DMEM), while ZR-75-1 breast cancer, HCC1937 and SW480 colon cancer cells were cultured in RPMI1640 medium containing 10% fetal bovine serum (FBS). Cell lines were obtained from American Type Culture Collection (ATCC). Sub-confluent cells with exponential growth were employed throughout the experiments. Cell transfection was carried out by using Lipofectamin 2000 according to the manufacture's instruction.

### Plasmids

Plasmids pcmv-myc-Nur77, GFP-Nur77, GFP-Nur77/LBD, GST-Bcl-2, pcmv-myc-Bcl-2, 3*Flag-cmv-Bcl-2 were constructed and used to evaluate and characterize compounds for their effect on Nur77/Bcl-2 interaction.

### Antibodies and Reagents

Anti-Myc (9E10) (Cat. Sc-40), anti-Ki67 (Cat. ab15580) and anti-Hsp60 (Cat. ab46798) antibodies from Abcam (UK); anti-b-actin (Cat. 4970S), anti-Cleaved caspase-3 (Cat. 9661S), and anti-Nur77 (Cat. 3960S) antibodies from Cell Signal Technology (Beverly, Mass., USA); anti-Nur77 (M-210) (Cat. sc-5569), anti-PCNA (Santa Cruz sc-7907), anti-a-tublin (Santa Cruz sc-8035), anti-Bcl-2 (Santa Cruz sc-783), anti-PARP (Santa Cruz sc-7150), and anti-GST (sc-138) antibodies from Santa Cruz Biotechnology (Santa Cruz, CA, USA); and anti-Flag (Cat. F1804) antibodies from Sigma (St. Louis, Mo., USA) were used.

### Statistical Analysis

Data were expressed as mean±SD. Each assay was repeated in triplicate in three independent experiments. The statistical significance of the differences among the means of several groups was determined using Student's t-test.

### Example A-2: GST-Pull Down

GST or GST-Bcl-2 fusion protein (0.5 mg) was immobilized on glutathione-Sepharose beads and incubated with purified His-Nur77-LBD (0.2 mg) in the presence of Compound 1 for 1 hour at room temperature to promote GST-Bcl-2 interaction with His-Nur77-LBD. Bound Nur77-LBD was analyzed by Western blotting.

### Example A-3: Western Blotting and Immunoprecipitation

Western blotting and co-immunopreciptations (co-IP) were performed. Briefly, cells transfected with or without expression vectors for Nur77 and Bcl-2 were treated with test compounds for 2 hours. The effect of compounds on the interaction of endogenous or transfected Nur77 and Bcl-2 was analyzed by co-immunoprecipitation using appropriate antibodies. Proteins immunoprecipitated from cells by an appropriate anti-Nur77 or anti-Bcl-2 antibody were analyzed by Western blotting.

### Example A-4: Cell Viability Determination and Cell Death Assay

Cell viability was analyzed by using a colorimetric 3-(4,5-dimethylthiazol-dimethylthiazol-2-yl)-2,5-diphenyletetrazolium Bromide (MTT) assay that determines mitochondrial dehydrogenase activities in the living cells with a colorimetric method. Cells treated with test compounds were incubated with MTT and their effect on the viability of the cells was assayed by the ability of treated cells to reduce the yellow tetrazolium dye MTT to its insoluble formazan, which has a purple color.

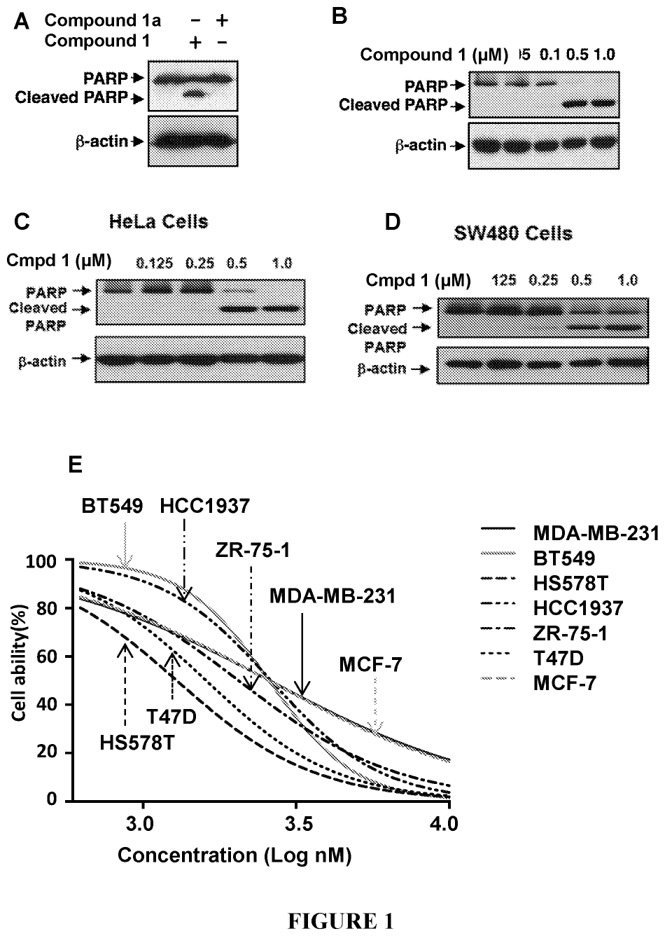

Compound 1 was effective in various breast cancer cell lines analyzed regardless of its hormone dependency, including MDA-MB-231 cells, BT549 cells, HCC 1937 cells, ZR-75-1 cells, T47D cells, and MCF-7 cells (FIG. 1E).

### Example A-5: Generation of Nur77 and Bcl-2 Knock-out Cells by CRISPR/Cas9 System

Knocking out Nur77 and Bcl-2 from HeLa cells employed the CRISPR/Cas9 system. gRNA targeting sequence of Nur77 (5′-ACCTTCATGGACGGCTACAC-3′) and Bcl-2 (5′-GAGAACAGGGTACGATAACC-3′) was cloned into gRNA cloning vector Px330 (Addgene, 71707) and confirmed by sequencing. To screen for cells lacking Nur77 or Bcl-2, HeLa cells were transfected with control vector and gRNA expression vectors, followed by G418 selection (0.5mg/m1). Single colonies were subjected to Western blotting using anti-Nur77 and anti-Bcl-2 antibody to select knockout cells.

### Example A-6: Apoptosis Assay

HCT116 cancer cells plated at a density of 1×10⁶ per well on six-well plates were treated with different concentration of test compounds for 6 hours, and then the suspension and the adherent cells were collected, stained with Annexin V-FITC for 15 minutes and with propodium iodide for 5 minutes, and analyzed immediately by cytoFLEX Flow Cytometry System (Beckman-Coulter, Miami, Fla., USA) using FITC and PC5.5 (Table 4).

TABLE 4

| Compound | Oxidized? | IC₅₀ |
|---|---|---|
| Compound 1 | Y | A |
| Compound 5 | Y | A |
| Compound 7 | Y | B |
| Compound 8 | Y | A |
| Compound 82 | Y | B |

A: IC₅₀ <0.5 μM;
B: ≥0.5 μM and <5 μM;
C ≥5 μM

### Oxidized DIMs Exhibits Superior Apoptotic Effect Over Non-Oxidized DIMs

The apoptotic effect of representative oxidized DIM analogs and non-oxidized DIM analogs were tested. Cell viability assays demonstrated that the oxidized forms of the DIM analogs had dramatically increased efficacy in inhibiting the growth of HCT116 cancer cells over their non-oxidized counterparts (Table 5). Non-oxidized species are labeled with a lower case “a” and contain a methine instead of an oxidized carbon atom, i.e. a CH instead of a C⁺.

For example, Compound 1 is

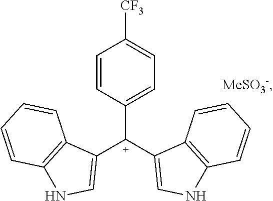

### and Compound 1a is

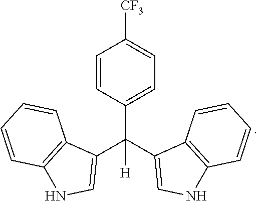 [SMILES: FC(F)(F)c1ccc(C(c2c[nH]c3ccccc23)c2c[nH]c3ccccc23)cc1]

TABLE 5

| Compound | Oxidized | IC₅₀ |
|---|---|---|
| Compound 1 | Y | A |
| Compound la | N | C |
| Compound 5 | Y | A |
| Compound 5a | N | C |
| Compound 7 | Y | B |
| Compound 7a | N | C |
| Compound 8 | Y | A |
| Compound 8a | N | C |
| Compound 28 | Y | A |
| Compound 28a | N | B |
| Compound 39 | Y | B |
| Compound 39a | N | C |
| Compound 47 | Y | A |
| Compound 47a | N | C |
| Compound 48 | Y | A |
| Compound 48a | N | C |
| Compound 70 | Y | A |
| Compound 70a | N | B |
| Compound 71 | Y | A |
| Compound 71a | N | B |
| Compound 73 | Y | A |
| Compound 73a | N | B |
| Compound 82 | Y | B |
| Compound 82a | N | C |
| Compound 88 | Y | A |
| Compound 88a | N | C |
| Compound 89 | Y | A |
| Compound 89a | N | B |
| Compound 103 | Y | A |
| Compound 103a | N | B |

A: IC₅₀ <0.5 μM;
B: ≥0.5 μM and <5 μM;
C ≥5 μM

Cell viability assays were also performed with MDA-MB-231cells and dramatically increased efficacy in inhibiting the growth of MDA-MB-231 cancer cells over their non-oxidized counterparts was observed (Table 6). Non-oxidized species are labeled with a lower case “a” and contain a methine instead of an oxidized carbon atom, i.e. a CH instead of a C⁺.

TABLE 6

| Compound | Oxidized? | IC₅₀ |
|---|---|---|
| Compound 1 | Y | A |
| Compound la | N | C |
| Compound 28 | Y | A |
| Compound 28a | N | B |
| Compound 39 | Y | B |
| Compound 39a | N | C |
| Compound 47 | Y | A |
| Compound 47a | N | B |
| Compound 48 | Y | A |
| Compound 48a | N | C |
| Compound 70 | Y | A |
| Compound 70a | N | C |
| Compound 71 | Y | A |
| Compound 71a | N | B |
| Compound 73 | Y | A |
| Compound 73a | N | B |
| Compound 88 | Y | A |
| Compound 88a | N | C |
| Compound 89 | Y | A |
| Compound 89a | N | C |
| Compound 103 | Y | A |
| Compound 103a | N | B |

A: IC₅₀ <0.5 μM;
B: ≥0.5 μM and <5 μM;
C ≥5 μM

### Example A-7: PARP Cleavage Assays

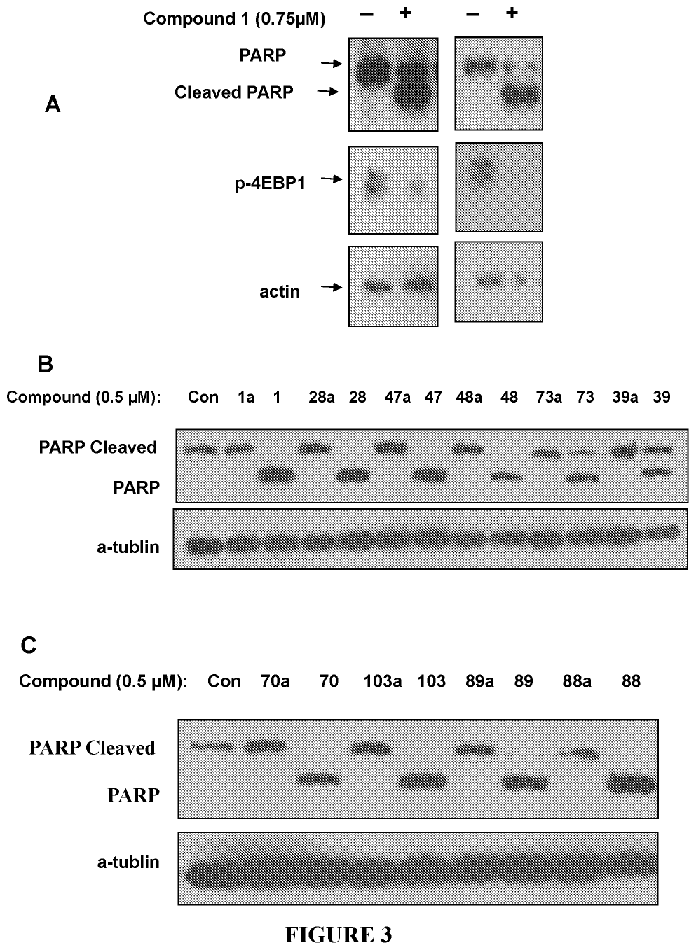

Treatment of HCT116 cells with 0.5 μM Compound 1 for 6 hours effectively induced PARP cleavage, an indication of apoptosis, as visualized by Western blotting. Compound 1a had no effect under the same conditions (FIG. 1A). Dose dependent study demonstrated that Compound 1 could induce PARP cleavage at submicromolar concentrations in HCT116 cells (FIG. 1B) and several other cancer cell lines, including HeLa cells and SW480 cells (FIGS. 1C-D). Furthermore, tested Compounds 10, 11, 15, 18, 21, 22, 24, 28, 39, 43, 46, 48, 49, 54, 55, 57, 62, 67, 71, 72, 73, and 74 were also able to effectively induce PARP cleavage in MDA-MB-231 cells after treatment of 1 μM Compound for 6 hours. Treatment of MDA-MB-231 cells with 0.5 μM Compound 1, 28, 47, 48, 73, 39, 70, 103, 89 and 88 for 6 hours effectively induced PARP cleavage,. Compound 1a, 28a, 47a, 48a, 73a, 39a, 70a, 103a, 89a and 88a had no effect under the same conditions (FIG. 3B-C).

### Example A-8: DAPI Staining for Apoptosis

The apoptotic effect of Compound 1 was demonstrated by its induction of extensive nuclear condensation and fragmentation revealed by DAPI staining in cells treated with 0.5 μM Compound 1 for 6 hours, as visualized by confocal microscopy.

In HCT116 cells, about 80% apoptotic cells were detected when cells were treated with Compound 1 compared to less than 5% when cells were not treated with Compound 1. Apoptotic cells were counted in 200 cells.

In HeLa cells, about 60% apoptotic cells were detected when cells were treated with Compound 1 compared to less than 5% when cells were not treated with Compound 1. Apoptotic cells were counted in 200 cells.

In SW480 cells, about 60% apoptotic cells were detected when cells were treated with Compound 1 compared to less than 5% when cells were not treated with Compound 1. Apoptotic cells were counted in 200 cells.

### Example A-9: Caspase 3 Cleavage Assay for Apoptosis

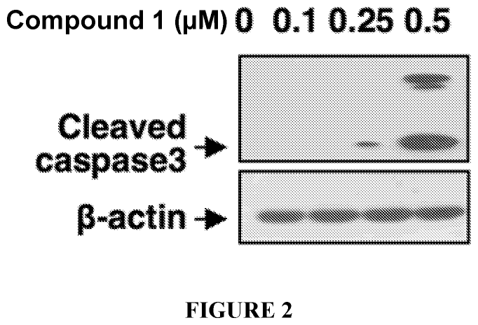

The apoptotic effect of Compound 1 was also demonstrated by its dose dependent induction of caspase 3 cleavage showed by Western blotting (FIG. 2).

### Example A-10: Flow Cytometry-based Annexin V/Propidium Iodide (PI) Apoptosis Assay

The effect of Compound 1 on cell death was further assessed using flow cytometry-based Annexin V/Propidium iodide (PI) apoptosis assay (see Example A-6).

Dose dependent study showed that about 31.63% of MDA-MB-231 cells were apoptotic when treated with 1 μM of Compound 1 for 6 hours, while only 1.31% of cells were apoptotic in vehicle control cells.

### Example A-11: mTOR Inhibition

Treatment of MDA-MB-231 cells with 0.75 μM Compound 1 for 6 hours reduced the expression levels of mTOR marker p-4EBP1 (FIG. 3). Furthermore, tested Compounds 1, 10, 11, 15, 18, 21, 22, 24, 28, 39, 43, 46, 48, 49, 54, 55, 57, 62, 67, 71, 72, 73, and 74 reduced the expression levels of mTOR markers, p-S6 and p-4EBP1 in MDA-MB-231 cells after treatment of 1 μM Compound for 6 hours.

### Example A-12: Determination of AIPm and ROS

Loss of mitochondrial membrane potential (Avm) represents one of the hallmarks of apoptosis. A JC-1 probe was employed to measure mitochondrial depolarization in cancer cells. MDA-MB-231 breast cancer cells were treated with different concentration of test compounds for 6 hours. JC-1 staining solution (5 μg/ml) was added at 37° C. for 20 min. After washing with PBS twice, mitochondrial membrane potentials were monitored by determining the relative amounts of dual emission from a multiple fluorescence reader. The fluorescence in cells was quantitatively analyzed by flow cytometry. Mitochondrial depolarization is depicted by an increase in the green/red fluorescence intensity ratio. ROS were monitored with the oxiadtion-senstive fluorescent probe 2′7′-dichlorodihydroflurescenin diacetate (DCF-DA).

In healthy cells with high Δψwm, JC-1 forms complexes with intense red fluorescence. However, in cells with low Δψm, JC-1 remains in the monomeric form with green fluorescence. Mitochondrial depolarization is depicted by an increase in the green/red fluorescence intensity ratio.

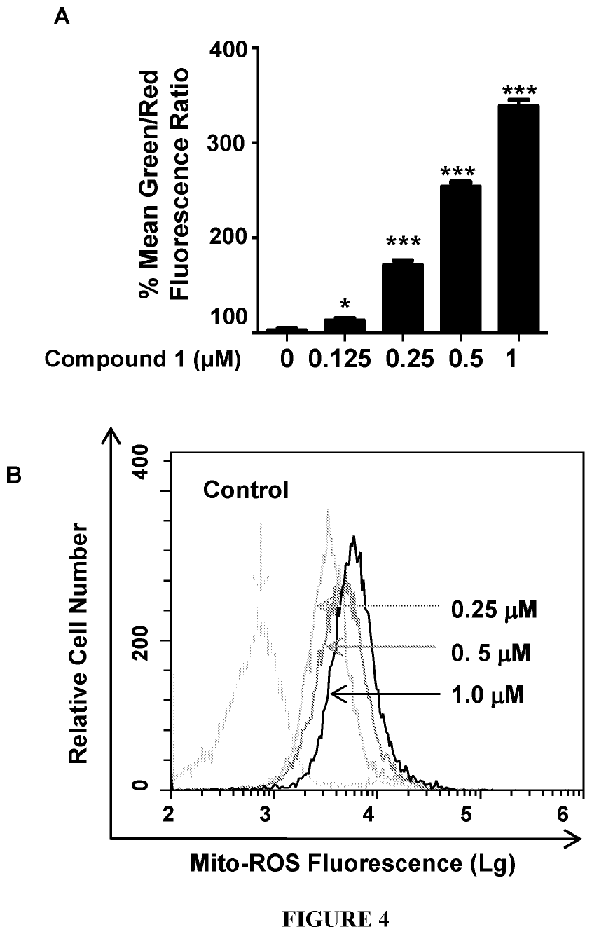

Analysis of both red and green fluorescence emissions by flow cytometry revealed a dose dependent induction of mitochondrial membrane dysfunction by Compound 1. After treatment with 1 μM of Compound 1 for 6 h, the green to red ratio increased from 100% to 345% (FIG. 4A). Mitochondrial dysfunction was also revealed by marked increase in intracellular mitochondrial reactive oxygen species (mito-ROS) in MDA-MB-231 cells exposed to Compound 1 in a dose dependent manner (FIG. 4B). Collectively, these data suggest that Compound 1 induced mitochondrion-related apoptosis in cancer cells.

### Example A-13: Immunostaining and Mitochondrial Targeting of Nur77

Cells were fixed in 3.7% paraformaldehyde. For mitochondrial staining, cells were incubated with anti-Hsp60 goat immunoglobulin G (IgG) (Santa Cruz Biotechnology, Santa Cruz, Calif.), followed by anti-goat IgG conjugated with Cy3. The nuclei were visualized by DAPI staining. Fluorescent images were collected and analyzed by using a fluorescence microscopy or MRC-1024 MP laser-scanning confocal microscope (Bio-Rad, Hercules, Calif.).

Immunostaining showed that Nur77 was mainly localized in the nucleus of HCT116 cells. However, it was predominantly cytoplasmic when cells were treated with 0.5 μM of Compound 1 for 2 hours. To confirm the effect of Compound 1 on Nur77 cytoplasmic localization, HEK293T cells were transfected with GFP-Nur77 and subsequently treated with 0.5 μM Compound 1. Transfected GFP-Nur77 resides in the nucleus, however it was diffusely distributed in both the cytoplasm and nucleus upon Compound 1 treatment.

### Example A-14: Cellular Fractionation

For cellular fractionation, cells were lysed in cold buffer A (10 mM HEPES-KOH (pH 7.9), 1.5 mM MgCl₂, 10 mM KCl, 0.5 mM dithiothreitol) with a cocktail of proteinase inhibitors on ice for 10 min as described. Cytoplasmic fraction was collected by centrifuging at 6000 rpm for 30 seconds. Pellets containing nuclei were resuspended in cold high-salt buffer C (20 mM HEPES-KOH (pH 7.9), 25% glycerol, 420 mM NaCl, 1.5 mM MgCl₂, 0.2 mM EDTA, 0.5 mM dithiothreitol) with a cocktail of proteinase inhibitors on ice for 30 minutes.

A significant amount of transfected GFP-Nur77 accumulated in the mitochondria-enriched heavy membrane (HM) fraction when cells were treated with Compound 1. Transfected GFP-Nur77-LBD was also affected by Compound 1, as it colocalized extensively with the mitochondria-specific Hsp60 protein, as revealed by confocal microscopy and co-accumulated with the Hsp60 protein in the heavy membrane fraction shown by cellular fractionation assay only in cells treated with Compound 1.

Mitochondria fractionation also revealed that Myc-Nur77 accumulated in the mitochondria-enriched heavy membrane (HM) fraction when cells were treated with 1μM Compound 1 or 28. Immunostaining showed that Myc-Nur77-LBD colocalized extensively with the mitochondria when cells were treated with Compound 1 and 28.

### Example A-15: Mitochondrial Targetting of Nur77 and Induction of Apoptosis

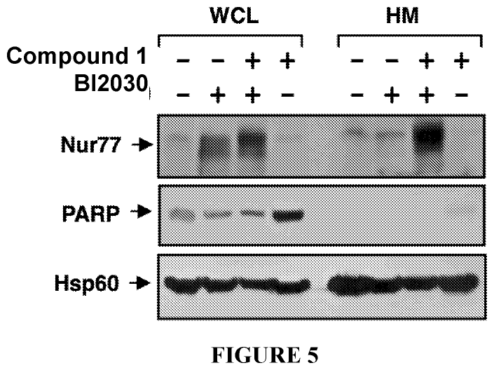

To further characterize the effect of Compound 1 on promoting Nur77 mitochondrial targeting, we investigated the effect of Compound 1 in combination with BI2030 on Nur77 mitochondrial targeting and apoptosis induction. BI2030, an analog of AHPN/CD437, can induce Nur77 expression but not its mitochondrial accumulation (FIG. 5). In contrast, Compound 1 was incapable of inducing Nur77 expression. When Compound 1 and B12030 were used together, most of the BI2030-induced Nur77 protein was found in the heavy membrane fraction, and formed punctate structure in the cytoplasm, as visualized by confocal microscopy. Thus, Compound 1 was able to induce mitochondrial targeting of BI2030-induced Nur77 protein.

We next examined whether Nur77 mitochondrial targeting induced by the combination of Compound 1/B12030 was apoptotic. HeLa cells were treated with either Compound 1 or BI2030 alone or in combination and apoptosis was examined by DAPI staining. About 7.2% or 9.1% of cells were apoptotic when treated with 0.5 μM Compound 1 or 1 μM BI2030, respectively, for 2 hours. However, about 35.5% apoptotic cells were detected when cells were co-treated with Compound 1 and BI2030, demonstrating a synergistic apoptosis induction. Apoptotic cells were counted in 200 cells.

These results revealed a unique ability of Compound 1 in inducing Nur77 mitochondrial targeting and apoptosis.

### Example A-16: Animal Studies

The protocols for animal studies were approved by the Animal Care and Use Committee of Xiamen University, and all mice were handled in accordance with the “Guide for the Care and Use of Laboratory Animals” and the “Principles for the Utilization and Care of Vertebrate Animals”.

### Example A-17: FRO Xenograft Nude Mouse Study

Male BALB/c nude mice (6 weeks old) were subcutaneously injected with log growth-phase of SW620 cells (1×10⁶ cells in 0.1 ml PBS). Mice were treated orally after 7 days of transplantation with Compound 1 once a day. Body weight and tumor size were measured every 3 days. Tumors were measured and weighted. Tissues isolated from the nude mice were fixed with 4% paraformaldehyde. TdT-mediated dUTP nick end labeling assay was performed according to the manufacturer's instructions (In situ Cell Death Detection Kit; Roche).

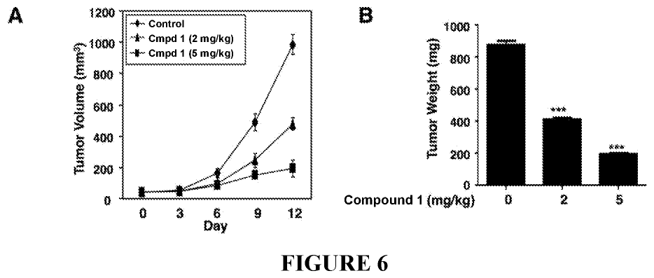

Administration of tumor-bearing nude mice with Compound 1 inhibited the growth of SW620 xenograft tumor in a dose and time-dependent manner (FIGS. 6A-6B). Additionally, TUNEL assay revealed extensive apoptosis in Compound 1 treated tumor specimens as compared to control tumor.

### Example A-18: MMTVPyMT Mice Breast Cancer Model

Female MMTV-PyMT mice of 12 weeks old were randomly divided into two groups (n=7 each), treated with a daily oral dose of test compound for 18 days. Standard histopathological analysis of tumor tissue was performed (see Example A-19). Test compounds were dissolved in DMSO and diluted with normal saline containing 5.0% (V/V) Tween-80 to a final concentration 0.5 mg/ml. Normal saline with DMSO and 5.0% Tween-80 was employed as the vehicle control.

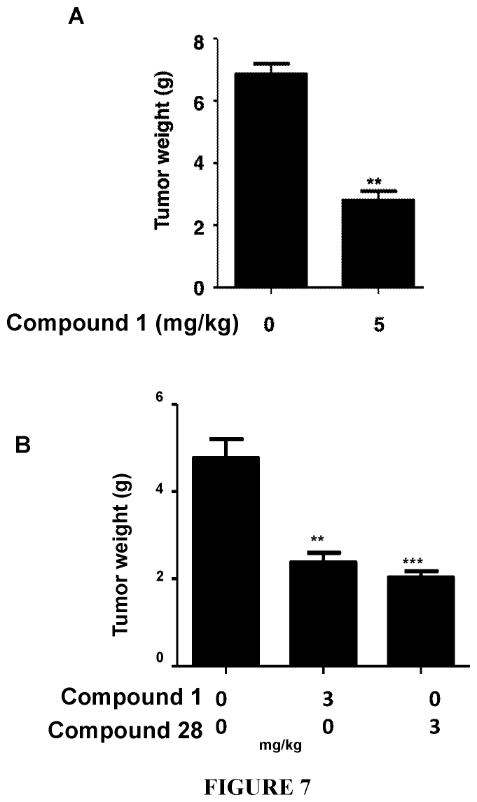

Administration of the MMTV-PyMT mice with Compound 1 (5 mg/kg), Compound 1 (3 mg/kg), or Compound 28 (3 mg/kg) inhibited the growth of PyMT mammary tumor (FIGS. 7A-7B). Western blotting of tumor tissues prepared from treated and non-treated mice revealed that the expression levels of two proliferation markers, PCNA and Ki67, were markedly reduced by Compound 1 (5 mg/kg). Immunostaining also showed a reduced expression of Ki67 and enhanced expression of cleaved caspase 3 in tumor tissue specimens prepared from mice treated with Compound 1 (5 mg/kg). Additionally, expression levels of mTOR markers, p-S6 and p-mTOR, were markedly reduced by both Compound 1 (3 mg/kg) and Compound 28 (3 mg/kg).

### Example A-19: Immunohistochemistry

4 μm thick sections were deparaffinized and rehydrated using xylene and a graded series of ethanol (100, 95, 85, 75, 50%), followed by washing in PBS. Antigen retrieval was performed in 10 mM sodium citrate buffer (pH 6.0), which was microwaved at 100° C. for 20 minutes. After rinsed twice in PBS, sections were blocked at room temperature for 1 hour by using 10% normal goat serum, followed by incubation with anti-ki-67, anti-cleaved-caspase-3, overnight (at least 17 hours) at 4° C. Colors were developed with a DAB horseradish peroxidase color development kit.

### Example A-20: Apoptotic Effect in Mouse Embryonic Fibroblasts (MEF)

### Nur77

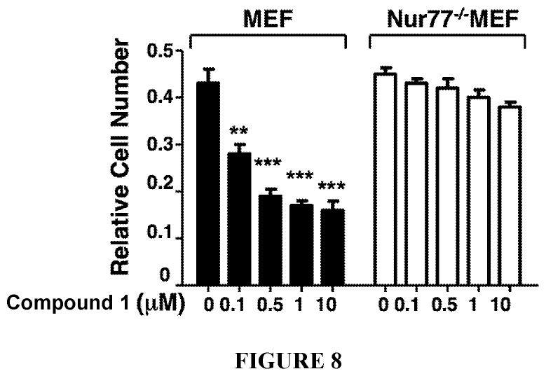

The apoptotic effect of Compound 1 was examined in mouse embryonic fibroblast (MEF) and MEF lacking Nur77 (Nur77^{-/-}MEF). Compound 1 dose-dependently inhibited the growth of MEFs, but such an inhibitory effect was significantly diminished in Nur77^{-/-}MEFs (FIG. 8). Induction of PARP cleavage (as performed in Example A-7) in MEFs by Compound 1 at 0.5 μM was also attenuated in Nur77^{-/-}MEFs.

### Bcl-2

The apoptotic effect of Compound 1 was also examined in mouse embryonic fibroblast (MEF) and MEF lacking Bcl-2 (Bcl-2^{-/-}MEF). Treating Bcl-2^{-/-}MEFs with Compound 1 at 0.5 μM had no apparent effect on PARP cleavage (as performed in Example A-7). Furthermore, DAPI staining confirmed that Bcl-2^{-/-}MEFs treated with Compound 1 at 0.5 μM were much more resistant than MEFs to apoptosis as 40% of MEFs displayed chromatin condensation and nuclear fragmentation, whereas only 14% of Bcl-2^{-/-}MEF cells exhibited similar apoptotic features. Apoptotic cells were counted in 200 cells.

In addition, Bcl-2^{-/-}MEF cells were more resistant than the parental MEF cells to Compound 1 in its induction of the mitochondrial membrane potential loss measured by JC1 staining and in the release of mitochondrial ROS (as performed in Example A-12). After treatment with 0.5 μM of Compound 1 for 6 h, the green to red ratio increased from 100% to 400% in MEF cells, while the ratio in Bcl-2^{-/-}HeLa cells only increased from 100% to 150%. Likewise, there was a smaller increase in intracellular mitochondrial reactive oxygen species (mito-ROS) in Bcl-2^{-/-}MEF cells than in regular MEF cells, when exposed to Compound 1.

### Example A-21: Apoptotic Effect in Genome Knockout HeLa Cells

### Nur77

The apoptotic effect of Compound lwas also evaluated in Nur77 genome knockout HeLa cells generated by CRISPR/Cas9 technology (Example A-5). Induction of PARP cleavage and caspase 3 activation by Compound 1 were strongly suppressed in Nur77^{-/-}HeLa cells (as performed in Examples A-7 and A-9). Annexin V/PI staining revealed a reduced apoptotic effect of Compound 1 in Nur77^{-/-}HeLa cells than in the parental HeLa cells (from 35.55% to 3.25%).

Furthermore, Nur77^{-/-}HeLa cells were more resistant than the parental HeLa cells to Compound 1 in its induction of the mitochondrial membrane potential loss measured by JC1 staining and in the release of mitochondrial ROS (as performed in Example A-12). After treatment with 1 μM of Compound 1 for 6 h, the green to red ratio increased from 100% to 190% in HeLa cells, while there was no significant change in Nur77^{-/-} HeLa cells. Likewise, there was a smaller increase in intracellular mitochondrial reactive oxygen species (mito-ROS) in Nur77^{-/-} HeLa cells than in regular HeLa cells, when exposed to Compound 1.

### Bcl-2

The apoptotic effect of Compound lwas also evaluated in Bcl-2 genome knockout HeLa cells generated by CRISPR/Cas9 technology (Example A-5). Induction of PARP cleavage and caspase 3 activation by Compound 1 were strongly suppressed in Bcl-2^{-/-}HeLa cells (as performed in Examples A-7 and A-9). Annexin V/PI staining also revealed a reduced apoptotic effect of Compound 1 in Bcl-2^{-/-}HeLa cells than in the parental HeLa cells (from 24.59% to 7.95%).

In addition, Bcl-2^{-/-}HeLa cells were more resistant than the parental HeLa cells to Compound 1 in its induction of the mitochondrial membrane potential loss measured by JC1 staining and in the release of mitochondrial ROS (as performed in Example A-12). After treatment with 0.5 μM of Compound 1 for 6 h, the green to red ratio increased from 100% to 195% in HeLa cells, while there was no change in Bcl-2^{-/-}HeLa cells. Likewise, there was a smaller increase in intracellular mitochondrial reactive oxygen species (mito-ROS) in Bcl-2^{-/-} HeLa cells than in regular HeLa cells, when exposed to Compound 1.

### Example A-22: Apoptotic Effect in HEK293T Cells Overexpressing Nur77-LBD

The ligand-binding domain (LBD) of Nur77, Nur77-LBD, was transfected into HEK293T cells. Transfection of Nur77-LBD enhanced the apoptotic effect of Compound 1, with 36% of the transfected HEK293T cells undergoing apoptosis, while 4.5% of the non-transfected cells were apoptotic, as measured by confocal microscopy.

Together, the results of examples A-20, A-21, and A-22 demonstrate that Compound 1 targets Nur77 to induce cancer cell apoptosis.

### Example A-23: Circular Dichroism (CD)

Stock solutions of 100 mM test compound in ethanol were added to 0.5 ml of 1 μM purified His-proteins in PBS, pH 7.6. CD spectra were obtained in a 0.2 cm path length cell at temperature between 25° C. to 65° C. by using an AVIV 62 DS spectropolarimeter for a wavelength range at 220 nm. Three spectra were corrected for background and averaged for each sample. The K_{d} was determined using nonlinear regression analysis for a one-site binding model (χ2>0.98).

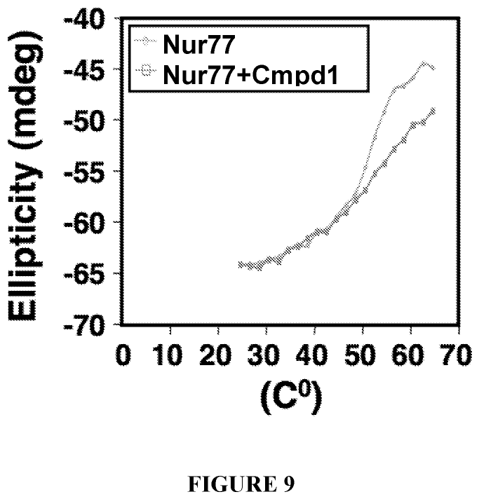

Protein spectra of purified Nur77-LBD at temperature 50° C. and above changed substantially after Nur77-LBD was incubated with Compound 1, demonstrating a direct binding of Nur77-LBD protein by Compound 1 (FIG. 9).

### Example A-24: Surface Plasmon Resonance (SPR)

The binding kinetics between Nur77-LBD and test compounds was analyzed at 25° C. on a BlAcore T200 machine with CMS chips (GE Healthcare). PBSP was used for all measurements. For SPR measurements, Nur77-LBD proteins were purified. A blank channel was used as negative control. About 10,000 response units of Nur77-LBD were immobilized on the chips. When the data collection was finished in each cycle, the sensor surface was regenerated with Glycine-HCl 2.5. A serial of concentrations ranging from 0.15 to 5.0 μM were designed for the experiment. Sensograms were fit globally with BIAcore T200 analysis using 1:1 Langumuir binding mode.

Surface plasma resonance (SPR) analysis revealed high affinity binding of Compound 1 to the Nur77-LBD protein with K_{d} of 0.1 μM.

### Example A-25: Mammalian One Hybrid Assay

HEK293T cells were co-transfected with pG5 Luciferase reporter together with the plasmid encoding RXRa-LBD fused with the Gal4 DNA-binding domain and other expression plasmids. After transfection, cells were treated with DMSO or a test compound, and assayed by using the Dual-Luciferase Reporter Assay System (Promega). Transfection efficiency was normalized to Renilla luciferase activity.

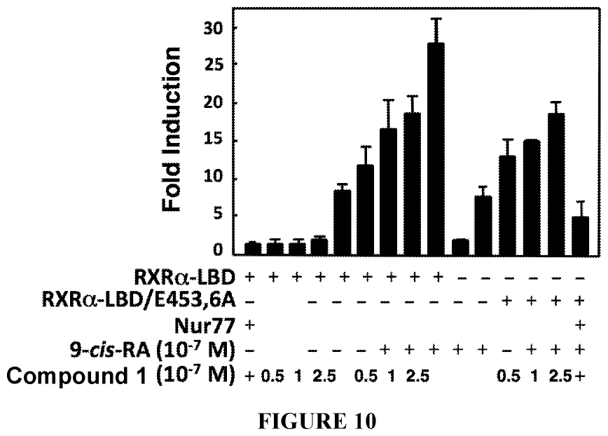

Cotransfection of pBind-RXRa-LBD and Nur77 strongly activate the reporter transcriptional activity when cells were treated with 9-cis-RA, a RXRa ligand (FIG. 10). Compound 1 also dose dependently induced the reporter activity. To exclude the possibility that Compound 1 acted on RXRa, Glu453 and Glu456 in the RXRa activation function 2 (AF2) region were substituted with Ala and the resulting mutant, RXRa-LBD/E453,6A, was used. As expected, 9-cis-RA failed to induce the reporter activity in cells transfected with Nur77 and pBind-RXRa-LBD-E453,6A. However, Compound 1 could still activate the reporter gene transcription, demonstrating that Compound 1 induced reporter gene transcription through Nur77 binding but not RXRα.

### Example A-26: Compounds Promote Nur77 Interaction with Bcl-2

A hallmark of the Nur77 mitochondrial apoptotic pathway is the interaction of Nur77 with Bcl-2, which converts Bcl-2 from an antiapoptotic protein to an inducer of apoptosis. Therefore, it was investigated whether the binding of test compounds to Nur77 enhanced the Nur77 interaction with Bcl-2.

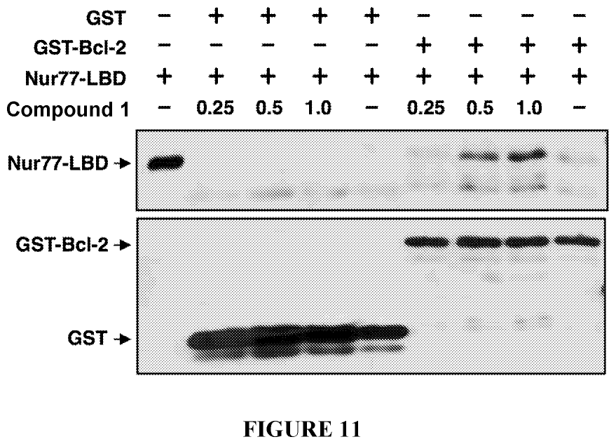

In vitro GST-pull down assays (Example A-2) showed that Nur77-LBD was pulled down by Compound 1 in a Compound 1 concentration dependent manner (FIG. 11). Cell-based Co-IP (Example A-3) showed that Nur77 or Nur77-LBD transfected in HEK293T cells interacted with Bcl-2 when cells were treated with Compound 1. Endogenous Nur77 could be also specifically immunoprecipitated together with endogenous Bcl-2 by anti-Bcl-2 antibody only when cells were treated with Compound 1. Moreover, confocal microscopy analysis revealed that Compound 1 promoted extensive colocalization of transfected GFP-Nur77 and GFP-Nur77-LBD respectively with Bcl-2 in cells.

Furthermore, cell-based Co-IP showed that Nur77 transfected in HeLa cells interacted with Bcl-2 when cells were treated with Compound 1 or 28. Moreover, confocal microscopy analysis revealed that Compound 1 and 28 promoted extensive colocalization of transfected GFP-Nur77 with Bcl-2 in cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A compound of Formula (I): 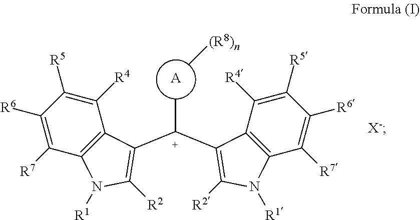
wherein:
Ring A is aryl or heteroaryl;
each R⁸ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)H, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, alkylene, cycloalkyl, heterocycloalkyl, aryl, and heteroaryl is independently unsubstituted or substituted with one, two, or three R¹⁰;
or two R⁸ on adjacent atoms are taken together with the atoms to which they are attached to form cycloalkyl or heterocycloalkyl, each of which is independently unsubstituted or substituted with one, two, or three halogen, C₁-C₆ alkyl, or C₁-C₆ haloalkyl;
n is an integer of 0-5;
R¹ and R^{1′} are each independently hydrogen, —S(═O)₂R^{a}, —S(═O)₂N(R^{b})₂, —C(═O)R^{b}, —C(═O)OR^{b}, —C(═O)N(R^{b})₂, C₁-C₈ alkyl, C₂-C₈ alkenyl, C₂-C₈ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein alkyl, alkenyl, alkynyl, alkylene, cycloalkyl, heterocycloalkyl, aryl, and heteroaryl is independently unsubstituted or substituted with one, two, or three R¹¹;
R² and R^{2′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, alkylene, cycloalkyl, heterocycloalkyl, aryl, and heteroaryl is independently unsubstituted or substituted with one, two, or three R¹²;
R⁴, R^{4′}, R⁵, R^{5′}, R⁶, R^{6′}, R⁷, and R^{7′} are each independently hydrogen, halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₂-C₆ alkenyl, C₂-C₆ alkynyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, alkylene, cycloalkyl, heterocycloalkyl, aryl, and heteroaryl is independently unsubstituted or substituted with one, two, or three R¹³;
R⁴, and R⁵, R⁵, and R⁶, R⁶, R⁷, R^{4′} and and R^{5′}, R^{5′}, and R^{6′}, or R^{6′} and R^{7′} are taken together with the atoms to which they are attached to form cycloalkyl, heterocycloalkyl, aryl, or heteroaryl; wherein the each cycloalkyl, heterocycloalkyl, aryl, of and heteroaryl is independently unsubstituted or substituted with one, two, or three R¹³;
X⁻ is a suitable anion;
each R¹⁰, R¹¹, R¹², and R¹³ is independently halogen, —CN, —OH, —OR^{a}, —SH, —SR^{a}, —S(═O)R^{a}, —NO₂, —N(R^{b})₂, —S(═O)₂R^{a}, —NHS(═O)₂R^{a}, —S(═O)₂N(R^{b})₂, —C(═O)R^{a}, —OC(═O)R^{a}, —C(═O)OR^{b}, —OC(═O)OR^{b}, —C(═O)N(R^{b})₂, —OC(═O)N(R^{b})₂, —NR^{b}C(═O)N(R^{b})₂, —NR^{b}C(═O)R^{a}, —NR^{b}C(═O)OR^{b}, C₁-C₆ alkyl, C₁-C₆ haloalkyl, C₁-C₆ hydroxyalkyl, C₁-C₆ heteroalkyl, cycloalkyl, heterocycloalkyl, phenyl, benzyl, or monocyclic 5- or 6-membered heteroaryl; wherein each cycloalkyl, heterocycle, phenyl, benzyl, and heteroaryl is independently unsubstituted or substituted with one, two, or three halogen, C₁-C₆ alkyl, or C₁-C₆ haloalkyl;
each R^{a} is independently C₁-C₆ alkyl, C₂-C₆ alkenyl, C₂-C₆ alkynyl, C₁-C₆heteroalkyl, cycloalkyl, heterocycloalkyl, aryl, heteroaryl, —C₁-C₆ alkylene(aryl), —C₁-C₆ alkylene(heteroaryl), —C₁-C₆ alkylene(cycloalkyl), or —C₁-C₆ alkylene(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl; and
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl;
or two R^{b} groups on a nitrogen atom are taken together with the nitrogen atom to which they are attached to form heterocycloalkyl which is unsubstituted or substituted with one, two, or three halogen, C₁-C₆ alkyl, or C₁-C₆ haloalkyl; and
wherein when Ring A is phenyl and n is 0, at least one of R¹, R^{1′}, R², R^{2′}, R⁴, R^{4′}, R⁵, R^{5′}, R⁶, R^{6′}, R⁷, and R^{7′} is not hydrogen.

2-6. (canceled)

7. The compound of claim 1, wherein X⁻ is Cl⁻, Br⁻, I⁻, ClO₄⁻, HSO₄⁻, NO₃⁻, H₂PO₄⁻, HC(═O)O⁻, CH₃C(═O)O⁻, CF₃C(═O)O⁻, C₆H₅C(═O)O⁻, CH₃S(═O)₂O⁻, CF₃S(═O)₂O⁻, C₆H₅S(═O)₂O⁻, p-CH₃—C₆H₄S(═O)₂O⁻, or BF₄⁻.

8-9. (canceled)

10. The compound of claim 1, wherein Ring A is phenyl, naphthyl, or fluorenyl.

11-13. (canceled)

14. The compound of claim 1, wherein Ring A is pyrrolyl, furanyl, thiophenyl, pyrazolyl, imidazolyl, triazolyl, oxazolyl, isoxazolyl, thiazolyl, isothiazolyl, oxadiazolyl, or thiadiazolyl.

15. (canceled)

16. The compound of claim 1, wherein Ring A is pyridinyl, pyridazinyl, pyrimidinyl, pyrazinyl, or triazinyl.

17-18. (canceled)

19. The compound of claim 1, wherein Ring A is indolyl, isoindolyl, indolizinyl, indazolyl, benzimidazolyl, azaindolyl, azaindazolyl, purinyl, benzofuranyl, isobenzofuranyl, benzo[b]thiophenyl, benzo[c]thiophenyl, benzoxazolyl, benzisoxazolyl, benzthiazolyl, benzisothiazolyl, quinolinyl, isoquinolinyl, quinoxalinyl, phthalizinyl quinazolinyl, cinnolinyl, naphthyridinyl, pyridopyrimidinyl, pyridopyrazinyl, or pteridinyl.

20-22. (canceled)

23. The compound of claim 1, wherein each R⁸ is independently —F, —Cl, —OH, —OCH₃, —OCF₃, —NO₂, —N(Et)₂, —C(═O)H, —C(═O)OCH₃, —C(═O)OH, methyl, ethyl, tert-butyl, —CF₃, or phenyl.

24. (canceled)

25. The compound of claim 1, wherein two R⁸ on adjacent atoms are taken together with the atoms to which they are attached to form 1,3-dioxolanyl or 1,4-dioxanyl.

26. The compound of claim 1, wherein n is an integer of 0, 1, 2, or 3.

27. The compound of claim 1, wherein

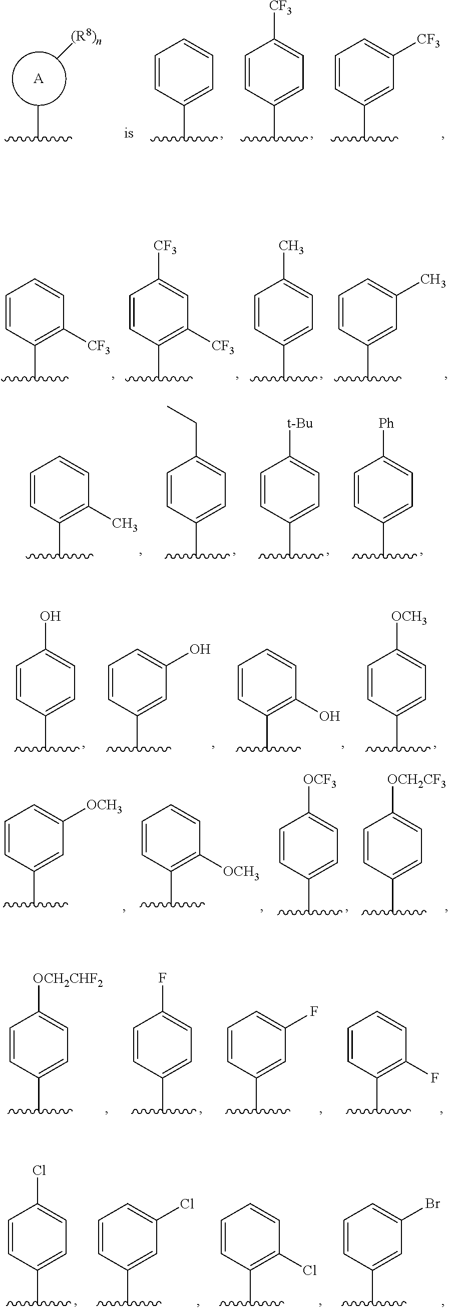

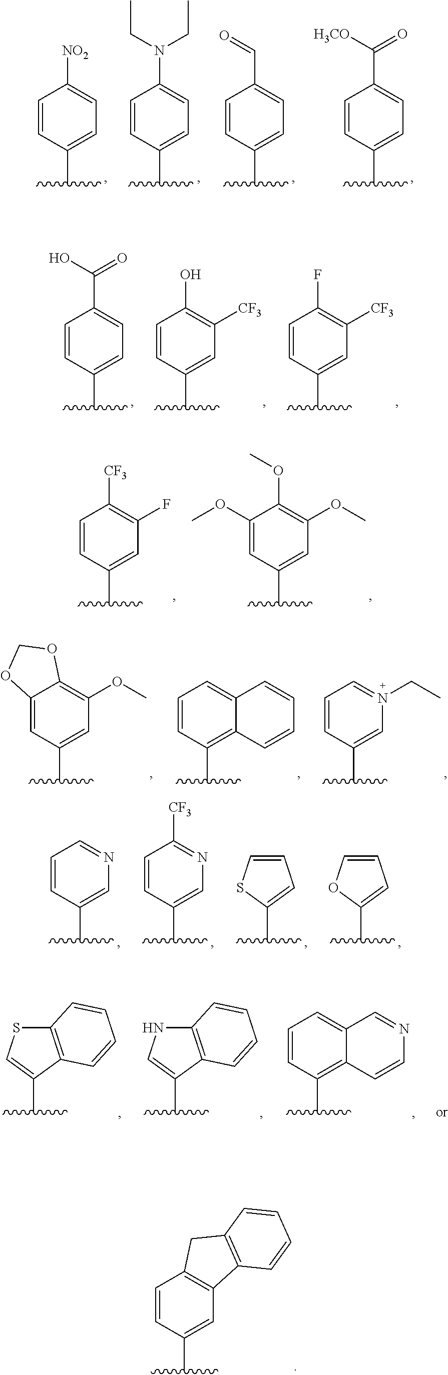

28-30. (canceled)

31. The compound of claim 1, wherein R¹ and R^{1′} are each independently hydrogen, methyl, ethyl, n-propyl, iso-propyl, n-butyl, iso-butyl, sec-butyl, tert-butyl, n-pentyl, iso-pentyl, n-hexyl, iso-hexyl, n-heptyl, n-octyl, or phenyl.

32-38. (canceled)

39. The compound of claim 1, wherein R² and R^{2′} are each independently hydrogen, methyl, ethyl, n-propyl, iso-propyl, n-butyl, iso-butyl, sec-butyl, tert-butyl, n-pentyl, iso-pentyl, or n-hexyl.

40-43. (canceled)

44. The compound of claim 1, wherein R⁴, R^{4′}, R⁵, R^{5′}, R⁶, R^{6′}, R⁷, and R^{7′} are each independently hydrogen, —F, —Cl, —Br, —OH, —OCH₃, —OBn, —C(═O)OH, or methyl.

45-51. (canceled)

52. The compound of claim 1, wherein the compound of is represented by Formula (IIIa), Formula (IIIc), Formula (IIId), Formula (IIIe), or Formula (IIIf): 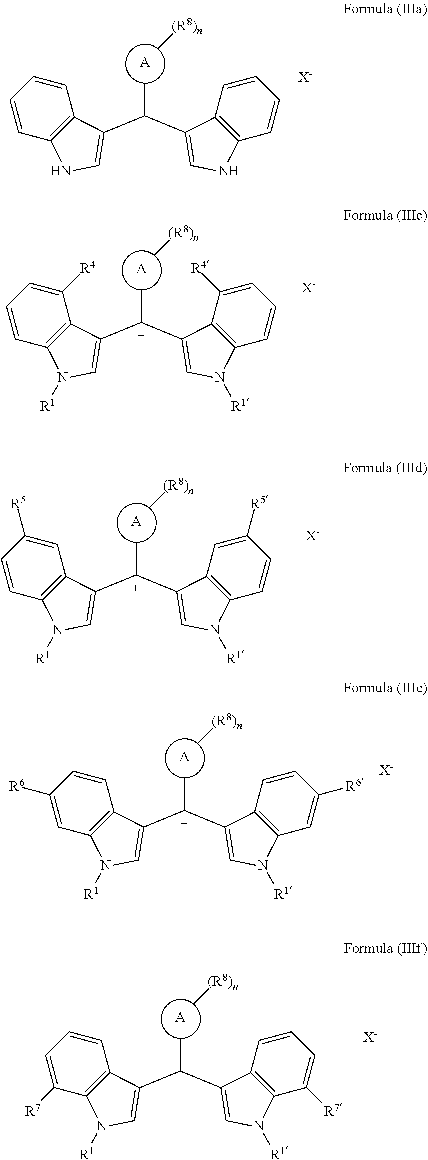
wherein Ring A is phenyl or heteroaryl.

53. A compound of:
(7-methyl-1H-indol-3-yl)(1H-indol-3-yl)(4-(trifluoromethyl)phenyl)methylium chloride;
(6-methyl-1H-indol-3-yl)(1H-indol-3-yl)(4-(trifluoromethyl)phenyl)methylium chloride;
(5-methyl-1H-indol-3-yl)(1H-indol-3-yl)(4-(trifluoromethyl)phenyl)methylium chloride;
(4-methyl-1H-indol-3-yl)(1H-indol-3-yl)(4-(trifluoromethyl)phenyl)methylium chloride;
bis(6-hydroxy-1H-indol-3-yl)(4-(trifluoromethyl)phenyl)methylium chloride;
bis(5-hydroxy-1H-indol-3-yl)(4-(trifluoromethyl)phenyl)methylium chloride;
3 -(bis(1H-indol-3 -yl)metheyliumyl)-1-ethylpyridin-1-ium dichloride;
bis(6-carboxy-1H-indol-3-yl)(4-(trifluoromethyl)phenyl)methylium chloride;
bis(7-fluoro-1H-indol-3-yl)(4-(trifluoromethyl)phenyl)methylium chloride;
bis(6-methoxy-1H-indol-3-yl)(4-(trifluoromethyl)phenyl)methylium chloride;
bis(1H-indol-3-yl)(4-(trifluoromethoxy)phenyl)methylium chloride;
bis(4-methyl-1H-indol-3-yl)(4-(trifluoromethyl)phenyl)methylium chloride;
bis(1H-indol-3-yl)(pyridin-3-yl)methylium chloride;
bis(1H-indol-3-yl)(2,4-bis(trifluoromethyl)phenyl)methylium chloride;
bis(1H-indol-3-yl)(2-(trifluoromethyl)phenyl)methylium chloride;
bis(1H-indol-3-yl)(3-(trifluoromethyl)phenyl)methylium chloride;
bis(4-fluoro-1H-indol-3-yl)(4-(trifluoromethyl)phenyl)methylium chloride;
bis(1-pentyl-1H-indol-3-yl)(4-(trifluoromethyl)phenyl)methylium chloride;
(1-pentyl-1H-indol-3-yl)(1H-indol-3-yl)(4-(trifluoromethyl)phenyl)methylium chloride;
(1-butyl-1H-indol-3-yl)(1H-indol-3-yl)(4-(trifluoromethyl)phenyl)methylium chloride;
(1-propyl-1H-indol-3-yl)(1H-indol-3-yl)(4-(trifluoromethyl)phenyl)methylium chloride;
(1-ethyl-1H-indol-3-yl)(1H-indol-3-yl)(4-(trifluoromethyl)phenyl)methylium chloride;
bis(1H-indol-3-yl)(1-benzothiophen-3-yl)methylium chloride;
bis(1H-indol-3-yl)(thiophen-2-yl)methylium chloride;
bis(1H-indol-3-yl)(furan-2-yl)methylium chloride;
tris(1H-indol-3-yl)methylium chloride;
bis(1H-indol-3-yl)(4-fluorophenyl)methylium methanesulfonate;
bis(1H-indol-3-yl)(4-tert-butylphenyl)methylium methanesulfonate;
bis(1H-indol-3-yl)(4-ethylphenyl)methylium methanesulfonate;
bis(1H-indol-3-yl)(4-nitrophenyl)methylium methanesulfonate;
bis(1H-indol-3-yl)((1,1′-biphenyl)-4-yl)methylium methanesulfonate;
bis(1H-indol-3-yl)(4-methylphenyl)methylium methanesulfonate;
(1-methyl-1H-indol-3 -yl)(1H-indol-3 -yl)(4-(trifluoromethyl)phenyl)methylium methanesulfonate;
(1-methyl-1H-indol-3-yl)(1H-indol-3-yl)(4-(trifluoromethyl)phenyl)methylium chloride;
bis(1H-indol-3-yl)(4-hydroxyphenyl)methylium chloride;
bis(1H-indol-3-yl)(4-hydroxyphenyl)methylium methanesulfonate;
bis(2-methyl-1H-indol-3-yl)(4-fluorophenyl)methylium chloride;
bis(1H-indol-3-yl)(phenyl)methylium methanesulfonate;
bis(1H-indol-3-yl)(phenyl)methylium chloride;
bis(1H-indol-3-yl)(4-methoxyphenyl)methylium methanesulfonate;
bis(5-methyl-1H-indol-3-yl)(4-(methoxycarbonyl)phenyl)methylium methanesulfonate;
bis(5-fluoro-1H-indol-3-yl)(4-chlorophenyl)methylium methanesulfonate;
bis(1-methyl-1H-indol-3-yl)(4-(trifluoromethyl)phenyl)methylium methanesulfonate;
bis(1-methyl-1H-indol-3-yl)(4-(trifluoromethyl)phenyl)methylium chloride;
bis(1H-indol-3-yl)(4-chlorophenyl)methylium methanesulfonate;
bis(1H-indol-3-yl)(4-chlorophenyl)methylium chloride;
bis(1H-indol-3-yl)(4-(methoxycarbonyl)phenyl)methylium methanesulfonate;
bis(1H-indol-3-yl)(4-(methoxycarbonyl)phenyl)methylium chloride;
bis(1H-indol-3-yl)(4-(trifluoromethyl)phenyl)methylium methanesulfonate;
bis(1H-indol-3-yl)(4-(trifluoromethyl)phenyl)methylium chloride;
bis(1H-indol-3-yl)(4-(trifluoromethyl)phenyl)methylium p-toluenesulfonate;
bis(1H-indol-3-yl)(4-(trifluoromethyl)phenyl)methylium hydrogensulfate;
bis(7-methyl-1H-indol-3-yl)(4-(trifluoromethyl)phenyl)methylium methanesulfonate;
bis(7-methyl-1H-indol-3-yl)(4-(trifluoromethyl)phenyl)methylium chloride;
bis(6-methyl-1H-indol-3-yl)(4-(trifluoromethyl)phenyl)methylium methanesulfonate;
bis(6-methyl-1H-indol-3-yl)(4-(trifluoromethyl)phenyl)methylium chloride;
bis(5-methyl-1H-indol-3-yl)(4-(trifluoromethyl)phenyl)methylium methanesulfonate;
bis(5-methyl-1H-indol-3-yl)(4-(trifluoromethyl)phenyl)methylium chloride;
bis(6-chloro-1H-indol-3-yl)(4-(trifluoromethyl)phenyl)methylium methanesulfonate;
bis(6-chloro-1H-indol-3-yl)(4-(trifluoromethyl)phenyl)methylium chloride;
bis(6-fluoro-1H-indol-3-yl)(4-(trifluoromethyl)phenyl)methylium methanesulfonate;
bis(6-fluoro-1H-indol-3-yl)(4-(trifluoromethyl)phenyl)methylium chloride;
bis(5-fluoro-1H-indol-3-yl)(4-(trifluoromethyl)phenyl)methylium methanesulfonate;
bis(5-chloro-1H-indol-3-yl)(4-(trifluoromethyl)phenyl)methylium methanesulfonate;
bis(5-bromo-1H-indol-3-yl)(4-(trifluoromethyl)phenyl)methylium methanesulfonate;
bis(2-methyl-1H-indol-3-yl)(4-(trifluoromethyl)phenyl)methylium methanesulfonate;
bis(2-methyl-1H-indol-3-yl)(4-(trifluoromethyl)phenyl)methylium chloride;
(5-hydroxy-1H-indol-3-yl)(1H-indol-3-yl)(4-(trifluoromethyl)phenyl)methylium chloride;
(6-hydroxy-1H-indol-3-yl)(1H-indol-3-yl)(4-(trifluoromethyl)phenyl)methylium chloride;
(6-fluoro-1H-indol-3-yl)(1H-indol-3-yl)(4-(trifluoromethyl)phenyl)methylium chloride;
(7-fluoro-1H-indol-3-yl)(1H-indol-3-yl)(4-(trifluoromethyl)phenyl)methylium chloride;
(4-fluoro-1H-indol-3-yl)(1H-indol-3-yl)(4-(trifluoromethyl)phenyl)methylium chloride;
(5-fluoro-1H-indol-3-yl)(1H-indol-3-yl)(4-(trifluoromethyl)phenyl)methylium chloride;
bis(1-phenyl-1H-indol-3-yl)(4-(trifluoromethyl)phenyl)methylium chloride;
(1H-indol-3-yl)(1-phenyl-1H-indol-3-yl)(4-(trifluoromethyl)phenyl)methylium chloride;
bis(5-(benzyloxy)-1H-indol-3-yl)(4-(trifluoromethyl)phenyl)methylium chloride;
(5-(benzyloxy)-1H-indol-3-yl)(1H-indol-3-yl)(4-(trifluoromethyl)phenyl)methylium chloride;
(6-(benzyloxy)-1H-indol-3-yl)(1H-indol-3-yl)(4-(trifluoromethyl)phenyl)methylium chloride;
(1H-indol-3-yl)(7-methoxy-1H-indol-3-yl)(4-(trifluoromethyl)phenyl)methylium chloride;
(1H-indol-3-yl)(6-methoxy-1H-indol-3-yl)(4-(trifluoromethyl)phenyl)methylium chloride;
(1H-indol-3-yl)(5-methoxy-1H-indol-3-yl)(4-(trifluoromethyl)phenyl)methylium chloride;
bis(1H-indol-3-yl)(4-carboxyphenyl)methylium methanesulfonate;
bis(5-methyl-1H-indol-3-yl)(4-chlorophenyl)methylium methanesulfonate;
bis(1H-indol-3-yl)(4-carboxyphenyl)methylium chloride;
bis(1-allyl-1H-indol-3-yl)(4-(trifluoromethyl)phenyl)methylium chloride;
(1-allyl-1H-indol-3-yl)(1H-indol-3-yl)(4-(trifluoromethyl)phenyl)methylium chloride;
(7-fluoro-1-methyl-1H-indol-3-yl)(1H-indol-3-yl)(4-(trifluoromethyl)phenyl)-methylium chloride;
(6-fluoro-1 -methyl-1H-indol-3-yl)(1H-indol-3-yl)(4-(trifluoromethyl)phenyl)-methylium chloride;
(7-fluoro-1H-indol-3-yl)(1-methyl-1H-indol-3-yl)(4-(trifluoromethyl)phenyl)-methylium chloride;
(6-fluoro-1H-indol-3-yl)(1-methyl-1H-indol-3-yl)(4-(trifluoromethyl)phenyl)-methylium chloride;
(3-bromophenyl)di(1H-indol-3-yl)methylium chloride; (3-chlorophenyl)di(1H-indol-3-yl)methylium chloride;
(2-chlorophenyl)di(1H-indol-3-yl)methylium chloride; (2-hydroxyphenyl)di(1H-indol-3-yl)methylium chloride;
(3 -hydroxyphenyl)di(1H-indol-3 -yl)methylium chloride; (3 -fluorophenyl)di(1H-indol-3-yl)methylium chloride;
di(1H-indol-3-yl)(m-tolyl)methylium chloride; di(1H-indol-3-yl)(3-methoxyphenyl)methylium chloride;
(2-fluorophenyl)di(1H-indol-3-yl)methylium chloride;
di(1H-indol-3-yl)(6-(trifluoromethyl)pyridin-3-yl)methylium chloride;
(4-hydroxy-3-(trifluoromethyl)phenyl)di(1H-indol-3-yl)methylium chloride;
(4-fluoro-3-(trifluoromethyl)phenyl)di(1H-indol-3-yl)methylium chloride;
(3-fluoro-4-(trifluoromethyl)phenyl)di(1H-indol-3-yl)methylium chloride;
(4-(2,2-difluoroethoxy)phenyl)di(1H-indol-3-yl)methylium chloride; or di(1H-indol-3-yl)(4-(2,2,2-trifluoroethoxy)phenyl)methylium chloride.

54. A pharmaceutical composition comprising the compound of claim 1, and a pharmaceutically acceptable excipient.

55. A method of treating a disease in a mammal comprising administering the compound of claim 1 to the mammal in need thereof.

56. (canceled)

57. The method of claim 55, wherein the disease is a cancer.

58-69. (canceled)

70. A method of inducing apoptosis in a cell, the method comprising contacting the cell with the compound of claim 1.

71-82. (canceled)

83. A method for modulating Nur77 activity in a cell, the method comprising contacting the cell with the compound of claim 1.

83-94. (canceled)
